SEQUENCE LISTING (11 sequences)

SEQ ID NO: 1 - protein, 183 aa, synthetic construct
GLNDIFEAQKIEWHETEYKLVVVGACGVGKSALTIQLIQNHFVDEYDPTIEDSYRKQVVIDGETSLLDILDTAGQEEYSAMRDQYMRTGEGFLLVFAINNTKSFEDIHHYREQIKRVKDSEDVPMVLVGNKSDLPSRTVDTKQAQDLARSYGIPFIETSAKTRQGVDDAFYTLVREIRKHKEK

SEQ ID NO: 2 - protein, 183 aa, synthetic construct
GLNDIFEAQKIEWHETEYKLVVVGADGVGKSALTIQLIQNHFVDEYDPTIEDSYRKQVVIDGETCLLDILDTAGQEEYSAMRDQYMRTGEGFLCVFAINNTKSFEDIHHYREQIKRVKDSEDVPMVLVGNKCDLPSRTVDTKQAQDLARSYGIPFIETSAKTRQGVDDAFYTLVREIRKHKEK

SEQ ID NO: 3 - protein, 183 aa, synthetic construct
GLNDIFEAQKIEWHETEYKLVVVGAVGVGKSALTIQLIQNHFVDEYDPTIEDSYRKQVVIDGETCLLDILDTAGQEEYSAMRDQYMRTGEGFLCVFAINNTKSFEDIHHYREQIKRVKDSEDVPMVLVGNKCDLPSRTVDTKQAQDLARSYGIPFIETSAKTRQGVDDAFYTLVREIRKHKEK

SEQ ID NO: 4 - protein, 183 aa, synthetic construct
GLNDIFEAQKIEWHETEYKLVVVGAGDVGKSALTIQLIQNHFVDEYDPTIEDSYRKQVVIDGETCLLDILDTAGQEEYSAMRDQYMRTGEGFLCVFAINNTKSFEDIHHYREQIKRVKDSEDVPMVLVGNKCDLPSRTVDTKQAQDLARSYGIPFIETSAKTRQGVDDAFYTLVREIRKHKEK

SEQ ID NO: 5 - protein, 172 aa, synthetic construct
GGGGSHMTEYKLVVVGAGGVGKSALTIQLIQNHFVDEYDPTIEDSYRKQVVIDGETCLLDILDTAGQEEYSAMRDQYMRTGEGFLCVFAINNTKSFEDIHQYREQIKRVKDSDDVPMVLVGNKCDLAARTVESRQAQDLARSYGIPYIETSAKTRQGVEDAFYTLVREIRQH

SEQ ID NO: 6 - protein, 176 aa, synthetic construct
GGGGMTEYKLVVVGAGGVGKSALTIQLIQNHFVDEYDPTIEDSYRKQVVIDGETCLLDILDTAGQEEYSAMRDQYMRTGEGFLCVFAINNSKSFADINLYREQIKRVKDSDDVPMVLVGNKCDLPTRTVDTKQAHELAKSYGIPFIETSAKTRQGVEDAFYTLVREIRQYRMKKLN

SEQ ID NO: 7 - protein, 524 aa, synthetic construct
MGSSHHHHHHSGENLYFQGSSGLNDIFEAQKIEWHESSEEQMRLPSADVYRFAEPDSEENIIFEENMQPKAGIPIIKAGTVIKLIERLTYHMYADPNFVRTFLTTYRSFCKPQELLSLIIERFEIPEPEPTEADRIAIENGDQPLSAELKRFRKEYIQPVQLRVLNVCRHWVEHHFYDFERDAYLLQRMEEFIGTVRGKAMKKWVESITKIIQRKKIARDNGPGHNITFQSSPPTVEWHISRPGHIETFDLLTLHPIEIARQLTLLESDLYRAVQPSELVGSVWTKEDKEINSPNLLKMIRHTTNLTLWFEKCIVETENLEERVAVVSRIIEILQVFQELNNFNGVLEVVSAMNSSPVYRLDHTFEQIPSRQKKILEEAHELSEDHYKKYLAKLRSINPPCVPFFGIYLTNILKTEEGNPEVLKRHGKELINFSKRRKVAEITGEIQQYQNQPYCLRVESDIKRFFENLNPMGNSMEKEFTDYLFNKSLEIEPRNPKPLPRFPKKYSYPLKSPGVRPSNPRPGT

SEQ ID NO: 8 - protein, 188 aa, synthetic construct
MASSHHHHHHSSENLYFQGMTEYKLVVVGACGVGKSALTIQLIQNHFVDEYDPTIEDSYRKQVVIDGETCLLDILDTAGQEEYSAMRDQYMRTGEGFLCVFAINNTKSFEDIHHYREQIKRVKDSEDVPMVLVGNKCDLPSRTVDTKQAQDLARSYGIPFIETSAKTRQGVDDAFYTLVREIRKHKEK

SEQ ID NO: 9 - protein, 188 aa, synthetic construct
MASSHHHHHHSSENLYFQGMTEYKLVVVGADGVGKSALTIQLIQNHFVDEYDPTIEDSYRKQVVIDGETCLLDILDTAGQEEYSAMRDQYMRTGEGFLCVFAINNTKSFEDIHHYREQIKRVKDSEDVPMVLVGNKCDLPSRTVDTKQAQDLARSYGIPFIETSAKTRQGVDDAFYTLVREIRKHKEK

SEQ ID NO: 10 - protein, 210 aa, synthetic construct
MGSSHHHHHHSSGLVPRGSHMASMTGGQQMGRGSENLYFQGMTEYKLVVVGAVGVGKSALTIQLIQNHFVDEYDPTIEDSYRKQVVIDGETCLLDILDTAGQEEYSAMRDQYMRTGEGFLCVFAINNTKSFEDIHHYREQIKRVKDSEDVPMVLVGNKCDLPSRTVDTKQAQDLARSYGIPFIETSAKTRQGVDDAFYTLVREIRKHKEK

SEQ ID NO: 11 - protein, 487 aa, synthetic construct
GEEQMRLPSADVYRFAEPDSEENIIFEENMQPKAGIPIIKAGTVIKLIERLTYHMYADPNFVRTFLTTYRSFCKPQELLSLIIERFEIPEPEPTEADRIAIENGDQPLSAELKRFRKEYIQPVQLRVLNVCRHWVEHHFYDFERDAYLLQRMEEFIGTVRGKAMKKWVESITKIIQRKKIARDNGPGHNITFQSSPPTVEWHISRPGHIETFDLLTLHPIEIARQLTLLESDLYRAVQPSELVGSVWTKEDKEINSPNLLKMIRHTTNLTLWFEKCIVETENLEERVAVVSRIIEILQVFQELNNFNGVLEVVSAMNSSPVYRLDHTFEQIPSRQKKILEEAHELSEDHYKKYLAKLRSINPPCVPFFGIYLTNILKTEEGNPEVLKRHGKELINFSKRRKVAEITGEIQQYQNQPYCLRVESDIKRFFENLNPMGNSMEKEFTDYLFNKSLEIEPRNPKPLPRFPKKYSYPLKSPGVRPSNPRPGT

(19) United States
(12) Patent Application Publication
(10) Pub. No.: US 2026/0083750 A1
(43) Pub. Date: Mar. 26, 2026

(54) SMALL MOLECULE INHIBITORS OF KRAS PROTEINS

(71) Applicant: MERCK SHARP & DOHME LLC, Rahway, NJ (US)

(72) Inventors: CHRISTIAN FISCHER, Natick, MA (US); TIMOTHY HENDERSON, Natick, MA (US); SHUHEI KAWAMURA, Cambridge, MA (US); XIAOSHEN MA, Boston, MA (US); MATTHEW J. MITCHELTREE, Jamaica Plain, MA (US); DAVID L. SLOMAN, Newton, MA (US); GIANNI CHESSARI, Cambridge (GB); YU KOBAYAKAWA, Ibaraki (JP); TAKAO UNO, Ibaraki (JP); TSUYOSHI OSHIMA, Ibaraki (JP); KEIICHI SUMIYAMA, Ibaraki (JP); TOSHIHIRO SAKAMOTO, Ibaraki (JP); KEI AKEMOTO, Ibaraki (JP); RISAKO MIURA, Ibaraki (JP)

(21) Appl. No.: 19/106,950

(22) PCT Filed: Aug. 24, 2023
(86) PCT No.: PCT/US23/72791

§ 371 Date: Feb. 26, 2025

Related U.S. Application Data
(60) Provisional application No. 63/373,625, filed on Aug. 26, 2022.

(51) Int. Cl. A61K 31/553; A61K 31/517; A61K 31/5377; A61K 31/5386; A61K 31/554; A61K 45/06; C07D 413/14; C07D 471/04; C07D 487/04; C07D 519/00

(52) U.S. Cl. CPC A61K 31/553; A61K 31/517; A61K 31/5377; A61K 31/5386; A61K 31/554; A61K 45/06; C07D 413/14; C07D 471/04; C07D 487/04; C07D 519/00

(57) ABSTRACT

Compounds of Formula (I) or their pharmaceutically acceptable salts can inhibit the G12C, G12D, G12V, and/or G13D mutants of Kirsten rat sarcoma (KRAS) protein and are expected to have utility as therapeutic agents, for example, for treating cancer. The disclosure also provides pharmaceutical compositions which comprise compounds of Formula (I) or pharmaceutically acceptable salts thereof. The disclosure also relates to methods for use of the compounds or their pharmaceutically acceptable salts in the therapy and prophylaxis of cancer and for preparing pharmaceuticals for this purpose.

### CROSS-REFERENCE

This application claims priority to U.S. Provisional Patent Application No. 63/373,625 filed Aug. 26, 2022, the contents of which are incorporated herein by reference in their entirety.

### FIELD OF THE INVENTION

The present disclosure relates to small molecule inhibitors of KRAS that inhibit, for example, the G12C mutant, G12D mutant, G12V mutant, G13D mutant, and the wild-type (WT) of Kirsten rat sarcoma (KRAS) protein and relates to a pharmaceutical composition comprising a compound of Formula (I) as well as methods of using such a compound for treatment of diseases, including cancers.

### BACKGROUND

RAS, which is a small monomeric GTP-binding protein having a molecular weight of about 21 kDa, acts as a molecular on/off switch. RAS can bind to GTP by binding to proteins of a guanine nucleotide exchange factor (GEF) (e.g., SOS1), which forces the release of a bound nucleotide, and releases GDP. When RAS binds to GTP, it becomes activated (turned on) and recruits and activates proteins necessary for the propagation of other receptors' signals, such as c-Raf and PI 3-kinase. RAS also possesses enzymatic activity with which it cleaves the terminal phosphate of the GTP nucleotide and converts the nucleotide into GDP. The rate of conversion is usually slow, but can be dramatically sped up by a protein of the GTPase-activating protein (GAP) class, such as RasGAP. When GTP is converted into GDP, RAS is deactivated (turned off).

The commonly known members of the RAS subfamily include HRAS, KRAS, and NRAS. Of these, mutations of KRAS are observed in many malignant tumors: in 86% of pancreatic ductal adenocarcinoma (PDAC), in 41% of colorectal cancers (CRC), and in 32% of lung adenocarcinoma (LUAD; a subtype of non-small-cell lung cancer (NSCLC)). The mutations often occur in the glycine residue at position 12 of KRAS (“G12”); the mutation at G12 dominates 91% (PDAC), 68% (CRC) and 85% (LUAD) of the total KRAS mutations, respectively. The distributions of amino acid substitutions at G12 vary among each tissue type. The most prevalent mutation in LUAD is the mutation into cysteine (“G12C”) (46%), while the predominant mutation in PDAC (45%) and CRC (45%) is the mutation into aspartic acid (“G12D”). The mutation at G12 into valine (“G12V”) is observed in a significant portion of G12 mutations in all of PDAC (35%), CRC (30%) and LUAD (23%). (Nature Reviews Drug Discovery, 19, 533-552, 2020).

Intense efforts in developing KRAS-G12C inhibitors are underway. Several covalent inhibitors which focus on the cysteine residue have been reported, and some of them have been subjected to clinical studies, such as AMG510 (NCT03600883), MRTX849 (NCT03785249) and JNJ-74699157 (NCT04006301). However, the KRAS-G12C mutation only accounts for a fraction of all KRAS mutations and is primarily found in LUAD. To effectively inhibit the other commonly-occurring KRAS mutated proteins, such as KRAS-G12D and KRAS-G12V, different approaches are needed as these mutants lack reactive cysteines in the active site (Nature Reviews Drug Discovery, 19, 533-552, 2020).

Studies have also indicated that gene amplification and high expression of WT KRAS in the absence of coding mutations can also occur in certain cancers. These amplifications were observed most frequently in esophageal, gastric and ovarian adenocarcinomas (Nature Medicine, 24, 968-977, 2018). Thus, effective inhibition of WT KRAS could provide a therapeutic benefit to patients suffering from such cancers.

### SUMMARY OF THE DISCLOSURE

The present disclosure provides small molecule inhibitors which modulate mutant and WT KRAS proteins and may be valuable pharmaceutically active compounds for the treatment of cancer. In some embodiments the disclosed compounds selectively inhibit the KRAS-G12C, KRAS-G12D and/or KRAS-G12V proteins. The compounds of Formula (I):

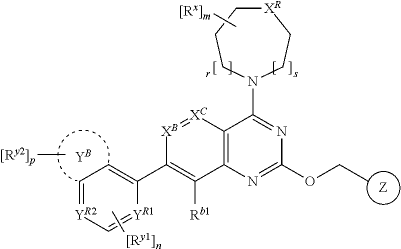

and their pharmaceutically acceptable salts, can modulate the activity of KRAS and thereby affect the signaling pathway which regulates cell growth, differentiation, and proliferation associated with oncological disorders. In certain embodiments, the compounds of Formula (I) can inhibit the KRAS-G12C, KRAS-G12D, KRAS-G12V, KRAS-G13D, and/or WT KRAS proteins. The disclosure furthermore provides processes for preparing compounds of Formula (I), methods for using such compounds to treat oncological disorders, and pharmaceutical compositions which comprise compounds of Formula (I).

### DETAILED DESCRIPTION OF THE INVENTION

### Compounds of the Disclosure

In one embodiment, the present disclosure provides a compound having structural Formula (I), or a pharmaceutically acceptable salt thereof, as shown above, wherein:
X^{R} is selected from the group consisting of —O—, —CH₂—, —S—, —S(O)—, and —S(O)₂—;
each R^{x} is independently selected from the group consisting of C₁-C₃ alkyl, C₁-C₃ fluoroalkyl, C₁-C₃ alkoxy, C₁-C₃ alkoxyalkyl, C₃-C₆ cycloalkyl, C₁-C₃ cyanoalkyl, fluoro, and cyano,
or alternatively, two R^{x}, when substituted on adjacent or hominal carbon atoms, can, together with the carbon atoms to which they are attached, form a ring C^{A},
wherein ring C^{A} is a 3- to 6-membered cycloalkyl or a saturated heterocycloalkyl ring containing one N atom,
wherein ring C^{A} is unsubstituted or substituted by 1 to 3 substitutions independently selected from the group consisting of C₁-C₃ alkyl, C₁-C₃ fluoroalkyl, C₁-C₃ alkoxy, C₃-C₆ cycloalkyl, fluoro, cyano, and —CH₂CH₂OCH₃;
X^{B} is C(R^{b2}) or N;
X^{C} is C(R^{b3}) or N;
R^{b1}, R^{b2}, and R^{b3} are independently selected from the group consisting of H, halo, cyano, C₁-C₃ fluoroalkyl, and C₂-C₃ alkynyl;
Ring Y^{B} is absent or present, and if present Y^{B} is a 5- or 6-membered heteroaryl containing 1 to 2 heteroatoms independently selected from the group consisting of N, O, and S;
y^{R1} is selected from the group consisting of C(H), C(R^{y1}), and N;
y^{R2} is selected from the group consisting of C(H), C(R^{y1}), and N;
R^{y1} and R^{y2} are independently selected from the group consisting of C₁-C₃ alkyl, halo, amino, cyano, C₁-C₃ fluoroalkyl, C₁-C₃ alkoxy, C₁-C₃ fluoroalkoxy, and C₂-C₄ fluoroalkynyl;
Ring Z is selected from the group consisting of.
(i) a 5- to 8-membered monocyclic- or bicyclic-heterocycloalkyl, wherein said heterocycloalkyl is saturated and contains 1 to 3 heteroatoms independently selected from the group consisting of N, S, and O, and wherein said heterocycloalkyl is unsubstituted or substituted with 1 to 2 substituents R^{ZHC} independently selected from the group consisting of halo, hydroxy, C₁-C₃ alkyl, C₁-C₃ fluoroalkyl, C₁-C₃ hydroxyalkyl, —C(H)(OH)CF₂H, —O—CH₂—O—(C₁-C₃ fluoroalkyl), and methylene(C₁-C₃ alkyl)(C₁-C₃ alkyl)carbamate;

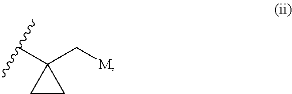

wherein M is selected from the group consisting of hydroxy, C₁-C₃ dialkylamino, and C₁-C₄ alkylamino, and wherein the cyclopropyl group is unsubstituted or independently substituted with up to 2 halo groups;

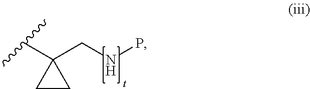

wherein P is:
3 to 8-membered cycloalkyl; or
4- to 8-membered monocyclic- or fused bicyclic- or bridged bicyclic-heterocycloalkyl, wherein said heterocycloalkyl is saturated and contains 1 to 2 heteroatoms independently selected from the group consisting of N and 0,
wherein said P is unsubstituted or substituted with 1 or 2 R^{P} substituents independently selected from the group consisting of halo, hydroxy, C₁-C₃ alkyl, C₁-C₃ fluoroalkyl, C₁-C₃ hydroxyalkyl, C₁-C₃ cyanoalkyl, carbamoyl, C₁-C₃ alkoxy, cyano, and —NHC(O)C₁-C₃alkyl, and wherein the cyclopropyl group is unsubstituted or independently substituted with up to 2 halo groups; and
(iv) a 4- to 8-membered monocyclic- or bicyclic-cycloalkyl, wherein said cycloalkyl is saturated and wherein said cycloalkyl is unsubstituted or independently substituted with 1-3 substituents R^{ZC} selected from the group consisting of halo, hydroxy, C₁-C₃ alkyl, C₁-C₃ fluoroalkyl, C₁-C₃ hydroxyalkyl, C₁-C₃ hydroxyfluoroalkyl, C₃-C₄ cycloalkyl, C₃-C₄ cyclofluoroalkyl, C₃-C₄ hydroxycycloalkyl, and C₃-C₄ hydroxycyclofluoroalkyl;
subscript m is 0, 1, 2 or 3;
subscript n is 0, 1, 2, or 3;
subscript p is 0, 1, or 2;
subscripts r and s are independently 0, 1 or 2, with the proviso that the sum of r and s is 1, 2 or 3;
subscript t is 0 or 1; and
   wherein when X^{B} is N and X^{C} is C(R^{b3}), then the group

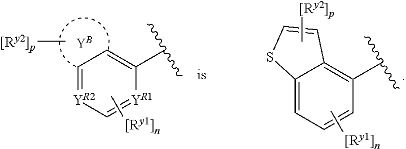

In another embodiment, the present disclosure provides a compound having structural Formula (I), or a pharmaceutically acceptable salt thereof, as shown above, wherein:
X^{R} is selected from the group consisting of —O—, —CH₂—, —S—, —S(O)—, and —S(O)₂—;
each R^{x} is independently selected from the group consisting of C₁-C₃ alkyl, C₁-C₃ fluoroalkyl, C₁-C₃ alkoxy, C₁-C₃ alkoxyalkyl, C₃-C₆ cycloalkyl, C₁-C₃ cyanoalkyl, fluoro, and cyano,
or alternatively, two R^{x} can, together with the carbon atom or atoms to which they are attached, form a ring C^{A},
wherein ring C^{A} is a 3- to 6-membered cycloalkyl or a saturated heterocycloalkyl ring containing one N atom,
wherein ring C^{A} is unsubstituted or substituted by 1 to 3 substitutions independently selected from the group consisting of C₁-C₃ alkyl, C₁-C₃ fluoroalkyl, C₁-C₃ alkoxy, C₃-C₆ cycloalkyl, fluoro, cyano, and —CH₂CH₂OCH₃;
X^{B} is C(R^{b2}) or N;
X^{C} is C(R^{b3}) or N;
R^{b1}, R^{b2}, and R^{b3} are independently selected from the group consisting of H, halo, cyano, C₁-C₃ fluoroalkyl, and C₂-C₃ alkynyl;
Ring Y^{B} is absent or present, and if present Y^{B} is a 5- or 6-membered heteroaryl containing 1 to 2 heteroatoms independently selected from the group consisting of N, O, and S;
y^{R1} is selected from the group consisting of C(H), C(R^{y1}), and N; y^{R2} is selected from the group consisting of C(H), C(R^{y1}), and N;
R^{y1} and R^{y2} are independently selected from the group consisting of C₁-C₃ alkyl, halo, amino, cyano, C₁-C₃ fluoroalkyl, C₁-C₃ alkoxy, C₁-C₃ fluoroalkoxy, and C₂-C₄ fluoroalkynyl;
Ring Z is selected from the group consisting of:
(i) a 5- to 8-membered monocyclic- or bicyclic-heterocycloalkyl, wherein said heterocycloalkyl is saturated and contains 1 to 3 heteroatoms independently selected from the group consisting of N, S, and O, and wherein said heterocycloalkyl is unsubstituted or substituted with 1 to 2 substituents R^{ZHC} independently selected from the group consisting of halo, hydroxy, C₁-C₃ alkyl, C₁-C₃ fluoroalkyl, C₁-C₃ hydroxyalkyl, —C(H)(OH)CF₂H, —O—CH₂—O—(C₁-C₃ fluoroalkyl), and methylene(C₁-C₃ alkyl)(C₁-C₃ alkyl)carbamate;

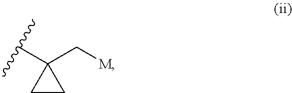

wherein M is selected from the group consisting of hydroxy, C₁-C₃ dialkylamino, and C₁-C₄ alkylamino, and wherein the cyclopropyl group is unsubstituted or independently substituted with up to 2 halo groups;

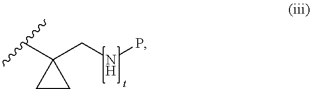

wherein P is:
3 to 8-membered cycloalkyl; or
4- to 8-membered monocyclic- or fused bicyclic- or bridged bicyclic-heterocycloalkyl, wherein said heterocycloalkyl is saturated and contains 1 to 2 heteroatoms independently selected from the group consisting of N and O,
wherein said P is unsubstituted or substituted with 1 or 2 RP substituents independently selected from the group consisting of halo, hydroxy, C₁-C₃ alkyl, C₁-C₃ fluoroalkyl, C₁-C₃ hydroxyalkyl, C₁-C₃ cyanoalkyl, carbamoyl, C₁-C₃ alkoxy, cyano, and —NHC(O)C₁-C₃alkyl, and wherein the cyclopropyl group is unsubstituted or independently substituted with up to 2 halo groups; and
(iv) a 4- to 8-membered monocyclic- or bicyclic-cycloalkyl, wherein said cycloalkyl is saturated and wherein said cycloalkyl is unsubstituted or independently substituted with 1-3 substituents R^{ZC} selected from the group consisting of halo, hydroxy, C₁-C₃ alkyl, C₁-C₃ fluoroalkyl, C₁-C₃ hydroxyalkyl, C₁-C₃ hydroxyfluoroalkyl, C₃-C₄ cycloalkyl, C₃-C₄ cyclofluoroalkyl, C₃-C₄ hydroxycycloalkyl, and C₃-C₄ hydroxycyclofluoroalkyl;
subscript m is 0, 1, 2 or 3;
subscript n is 0, 1, 2, or 3;
subscript p is 0, 1, or 2;
subscripts r and s are independently 0, 1 or 2;
subscript t is 0 or 1; and
   wherein when X^{B} is N and X^{C} is C(R^{b3}), then the group

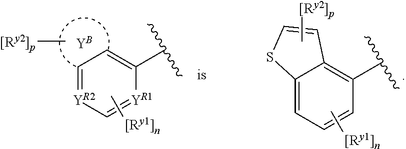

In another embodiment, the present disclosure provides a compound of Formula (I), or the pharmaceutically acceptable salt thereof, wherein subscript r and s are independently 1 or 2, with the proviso that the sum of r and s is 2 or 3.

In another embodiment, the present disclosure provides a compound of Formula (I), or a pharmaceutically acceptable salt thereof, wherein the group

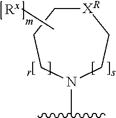

is selected from the group consisting of:

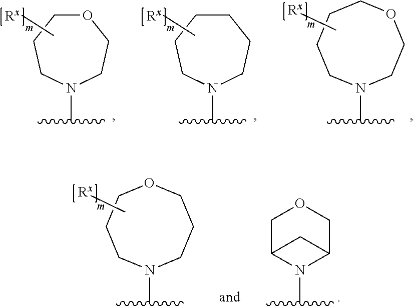

In another embodiment, the present disclosure provides a compound of Formula (I), or a pharmaceutically acceptable salt thereof, wherein the group

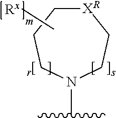

is selected from the group consisting of:

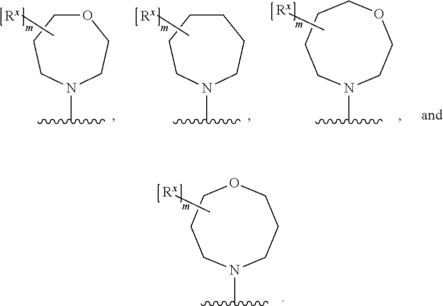

In another embodiment, the present disclosure provides a compound of Formula (I), or a pharmaceutically acceptable salt thereof, wherein the group

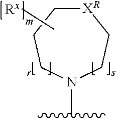

is selected from the group consisting of:

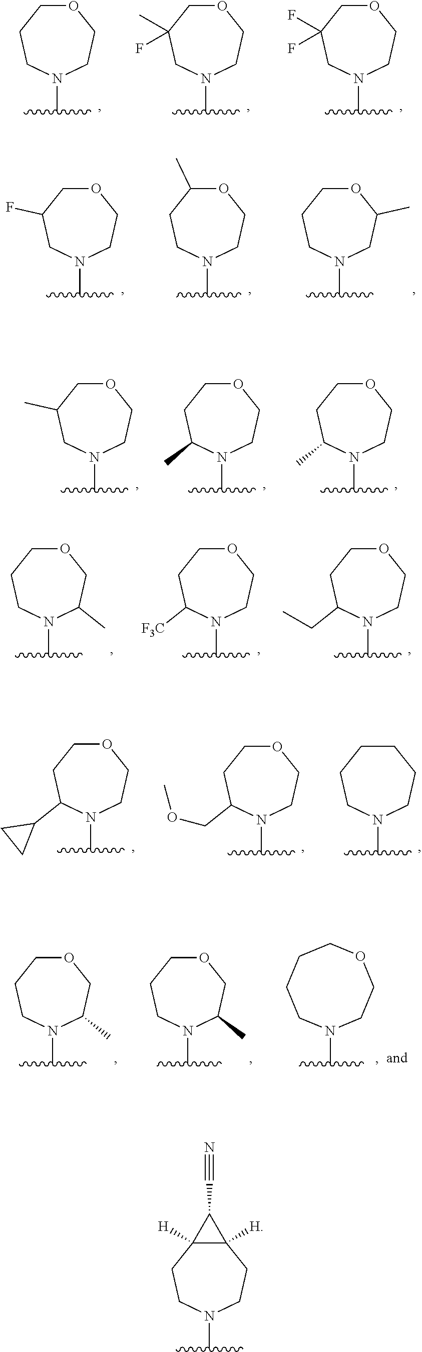

In another embodiment, the present disclosure provides a compound of Formula (I), or a pharmaceutically acceptable salt thereof, wherein:
(i) ring Y^{B} is present, Y^{R1} is C(H) or C(R^{y1}), and Y^{R2} is C(H) or C(R^{y1}); or
(ii) ring Y^{B} is absent, Y^{R1} is N, and Y^{R2} is C(H).

In another embodiment, the present disclosure provides a compound of Formula (I), or a pharmaceutically acceptable salt thereof, wherein:
(i) ring Y^{B} is present, Y^{R1}, and Y^{R2} is C(H) or C(F); or
(ii) ring Y^{B} is absent, Y^{R1} is N, and Y^{R2} is C(H).

In another embodiment, the present disclosure provides a compound of Formula (I), or a pharmaceutically acceptable salt thereof, wherein the group

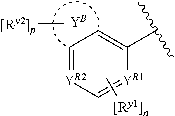

is selected from the group consisting of:

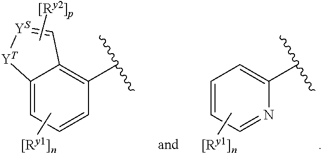

wherein Y^{S} is selected from the group consisting of N, O and S; and Y^{T} is selected from the group consisting of N(H), O and S.

In another embodiment, the present disclosure provides a compound of Formula (I), or a pharmaceutically acceptable salt thereof, wherein the group

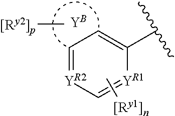

is selected from the group consisting of:

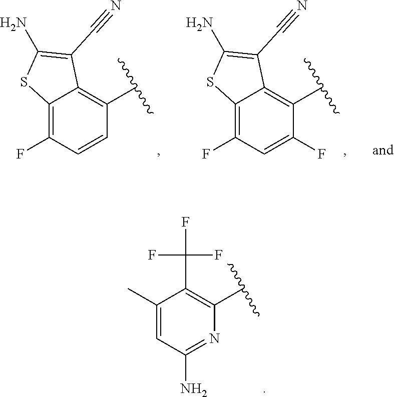 [SMILES: *c1ccc(F)c2sc(N)c(C#N)c12 ; *c1c(F)cc(F)c2sc(N)c(C#N)c12 ; *c1nc(N)cc(C)c1C(F)(F)F]

In another embodiment, the present disclosure provides a compound of Formula (I), or a pharmaceutically acceptable salt thereof, wherein X^{B} is C(R^{b2}).

In another embodiment, the present disclosure provides a compound of Formula (I), or a pharmaceutically acceptable salt thereof, wherein X^{C} is C(R^{b3}).

In another embodiment, the present disclosure provides a compound of Formula (I), or a pharmaceutically acceptable salt thereof, wherein X^{B} is N, X^{C} is C(R^{b3}), and the group

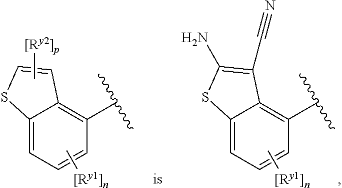

where R^{y1} is fluoro, and the subscript n is 1 or 2.

In another embodiment, the present disclosure provides a compound of Formula (I), or a pharmaceutically acceptable salt thereof, wherein ring Z is selected from the group consisting of:

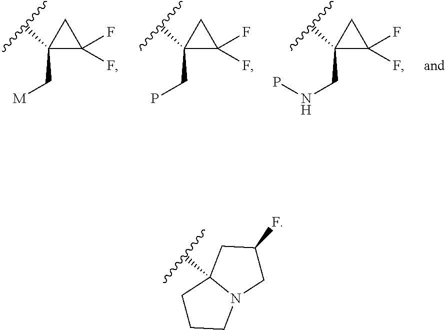

In specific embodiments, the present disclosure provides a compound as described in any one of Examples 1-53 as set forth below, or a pharmaceutically acceptable salt thereof.

In specific embodiments, the present disclosure provides a compound or a pharmaceutically acceptable salt thereof, the compound selected from the group consisting of:

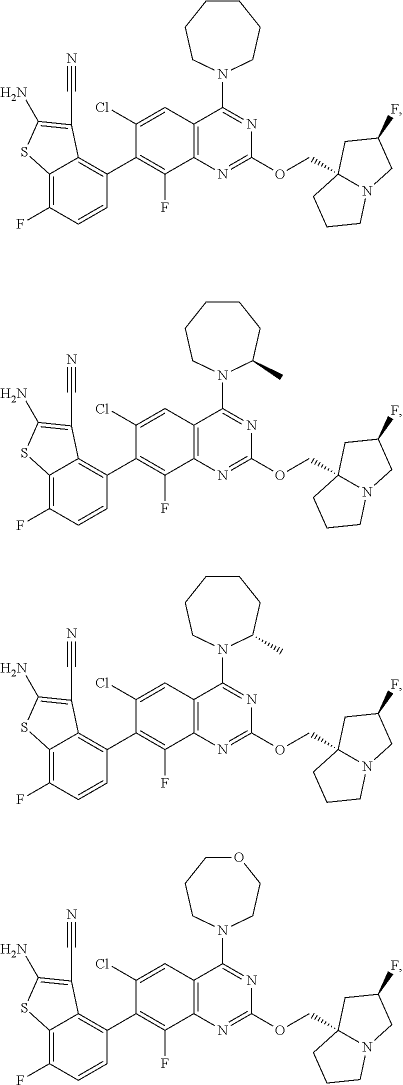 [SMILES: N#Cc1c(N)sc2c(F)ccc(-c3c(Cl)cc4c(N5CCCCCC5)nc(OC[C@@]56CCCN5C[C@H](F)C6)nc4c3F)c12 ; C[C@@H]1CCCCCN1c1nc(OC[C@@]23CCCN2C[C@H](F)C3)nc2c(F)c(-c3ccc(F)c4sc(N)c(C#N)c34)c(Cl)cc12 ; C[C@H]1CCCCCN1c1nc(OC[C@@]23CCCN2C[C@H](F)C3)nc2c(F)c(-c3ccc(F)c4sc(N)c(C#N)c34)c(Cl)cc12 ; N#Cc1c(N)sc2c(F)ccc(-c3c(Cl)cc4c(N5CCCOCC5)nc(OC[C@@]56CCCN5C[C@H](F)C6)nc4c3F)c12]

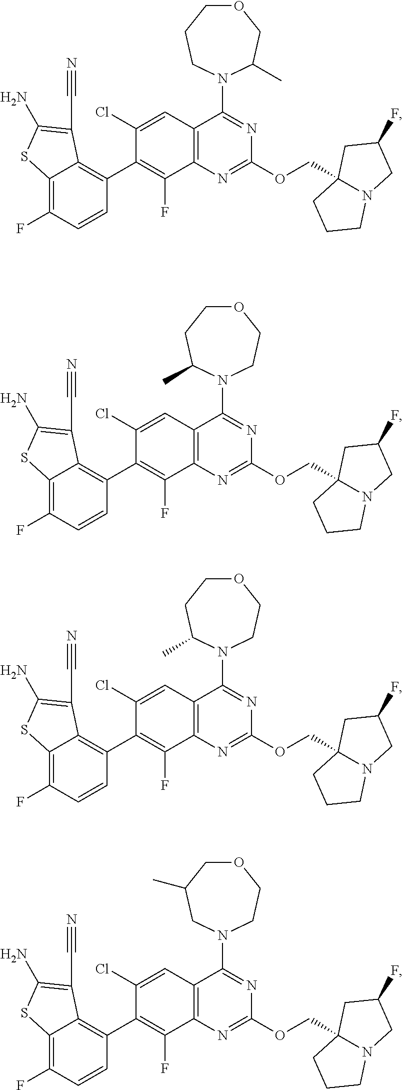 [SMILES: CC1COCCCN1c1nc(OC[C@@]23CCCN2C[C@H](F)C3)nc2c(F)c(-c3ccc(F)c4sc(N)c(C#N)c34)c(Cl)cc12 ; C[C@H]1CCOCCN1c1nc(OC[C@@]23CCCN2C[C@H](F)C3)nc2c(F)c(-c3ccc(F)c4sc(N)c(C#N)c34)c(Cl)cc12 ; C[C@@H]1CCOCCN1c1nc(OC[C@@]23CCCN2C[C@H](F)C3)nc2c(F)c(-c3ccc(F)c4sc(N)c(C#N)c34)c(Cl)cc12 ; CC1COCCN(c2nc(OC[C@@]34CCCN3C[C@H](F)C4)nc3c(F)c(-c4ccc(F)c5sc(N)c(C#N)c45)c(Cl)cc23)C1]

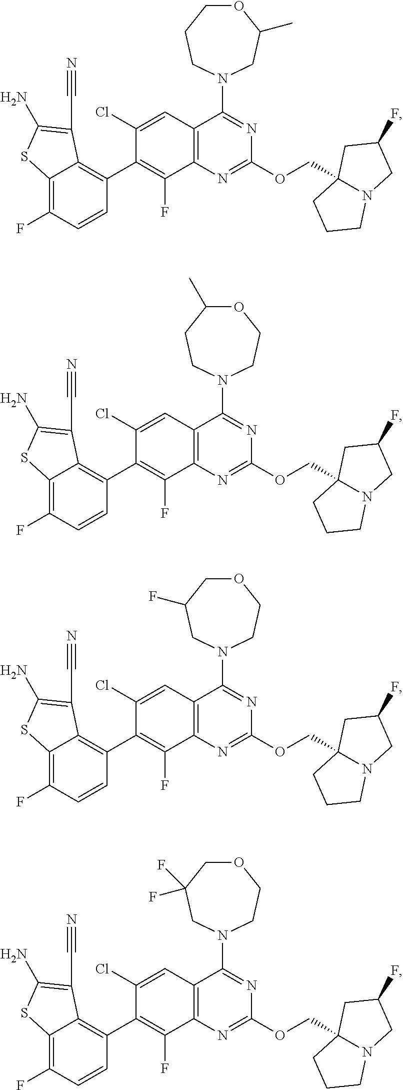 [SMILES: CC1CN(c2nc(OC[C@@]34CCCN3C[C@H](F)C4)nc3c(F)c(-c4ccc(F)c5sc(N)c(C#N)c45)c(Cl)cc23)CCCO1 ; CC1CCN(c2nc(OC[C@@]34CCCN3C[C@H](F)C4)nc3c(F)c(-c4ccc(F)c5sc(N)c(C#N)c45)c(Cl)cc23)CCO1 ; N#Cc1c(N)sc2c(F)ccc(-c3c(Cl)cc4c(N5CCOCC(F)C5)nc(OC[C@@]56CCCN5C[C@H](F)C6)nc4c3F)c12 ; N#Cc1c(N)sc2c(F)ccc(-c3c(Cl)cc4c(N5CCOCC(F)(F)C5)nc(OC[C@@]56CCCN5C[C@H](F)C6)nc4c3F)c12]

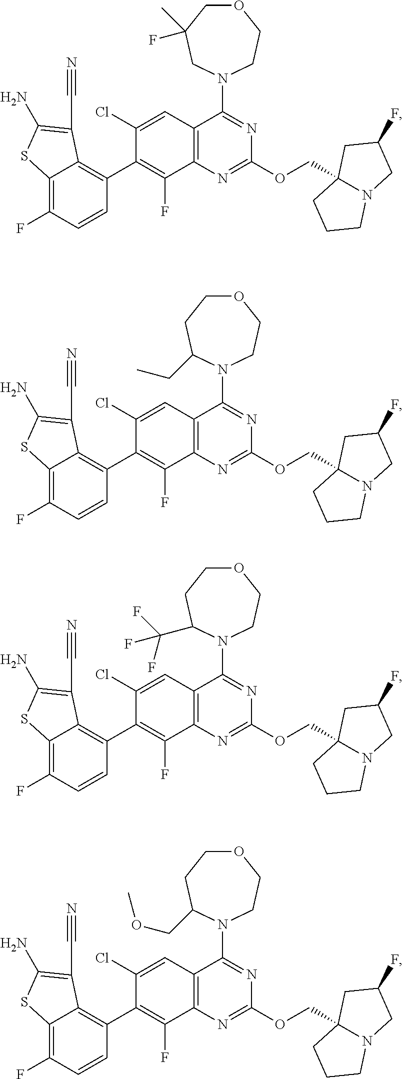 [SMILES: CC1(F)COCCN(c2nc(OC[C@@]34CCCN3C[C@H](F)C4)nc3c(F)c(-c4ccc(F)c5sc(N)c(C#N)c45)c(Cl)cc23)C1 ; CCC1CCOCCN1c1nc(OC[C@@]23CCCN2C[C@H](F)C3)nc2c(F)c(-c3ccc(F)c4sc(N)c(C#N)c34)c(Cl)cc12 ; N#Cc1c(N)sc2c(F)ccc(-c3c(Cl)cc4c(N5CCOCCC5C(F)(F)F)nc(OC[C@@]56CCCN5C[C@H](F)C6)nc4c3F)c12 ; COCC1CCOCCN1c1nc(OC[C@@]23CCCN2C[C@H](F)C3)nc2c(F)c(-c3ccc(F)c4sc(N)c(C#N)c34)c(Cl)cc12]

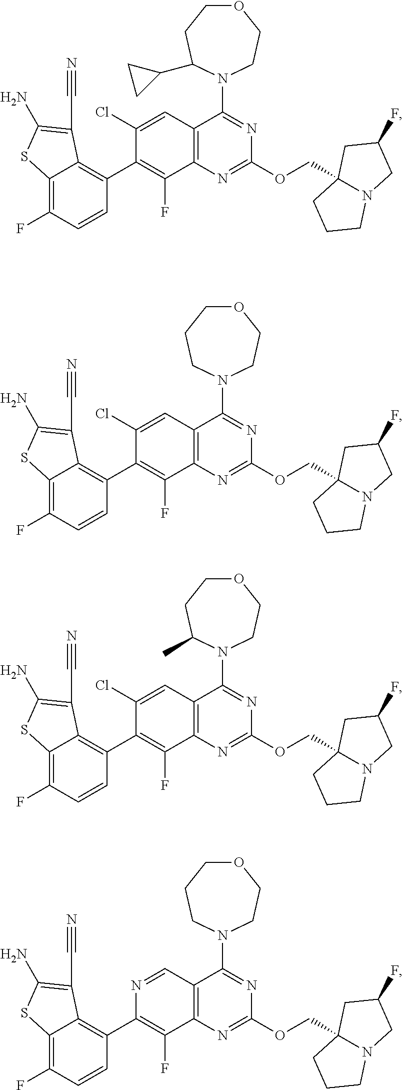 [SMILES: N#Cc1c(N)sc2c(F)ccc(-c3c(Cl)cc4c(N5CCOCCC5C5CC5)nc(OC[C@@]56CCCN5C[C@H](F)C6)nc4c3F)c12 ; N#Cc1c(N)sc2c(F)ccc(-c3c(Cl)cc4c(N5CCCOCC5)nc(OC[C@@]56CCCN5C[C@H](F)C6)nc4c3F)c12 ; C[C@H]1CCOCCN1c1nc(OC[C@@]23CCCN2C[C@H](F)C3)nc2c(F)c(-c3ccc(F)c4sc(N)c(C#N)c34)c(Cl)cc12 ; N#Cc1c(N)sc2c(F)ccc(-c3ncc4c(N5CCCOCC5)nc(OC[C@@]56CCCN5C[C@H](F)C6)nc4c3F)c12]

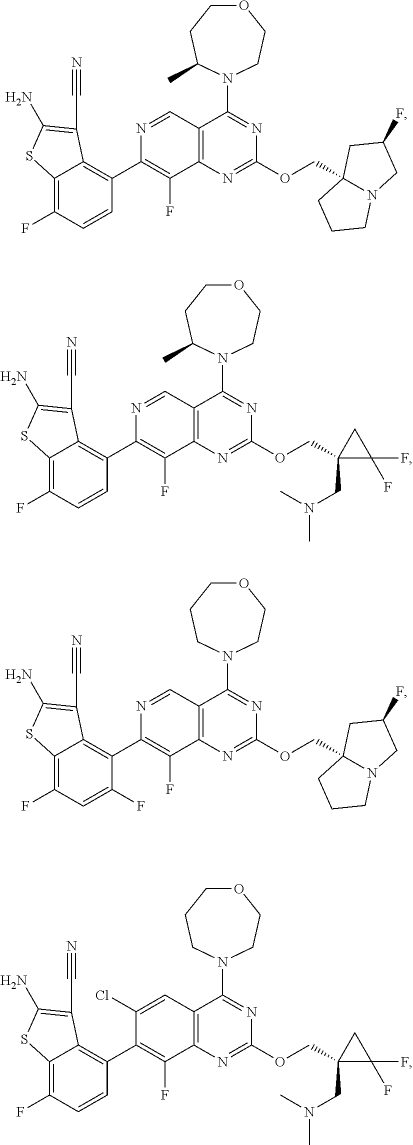 [SMILES: C[C@H]1CCOCCN1c1nc(OC[C@@]23CCCN2C[C@H](F)C3)nc2c(F)c(-c3ccc(F)c4sc(N)c(C#N)c34)ncc12 ; C[C@H]1CCOCCN1c1nc(OC[C@]2(CN(C)C)CC2(F)F)nc2c(F)c(-c3ccc(F)c4sc(N)c(C#N)c34)ncc12 ; N#Cc1c(N)sc2c(F)cc(F)c(-c3ncc4c(N5CCCOCC5)nc(OC[C@@]56CCCN5C[C@H](F)C6)nc4c3F)c12 ; CN(C)C[C@@]1(COc2nc(N3CCCOCC3)c3cc(Cl)c(-c4ccc(F)c5sc(N)c(C#N)c45)c(F)c3n2)CC1(F)F]

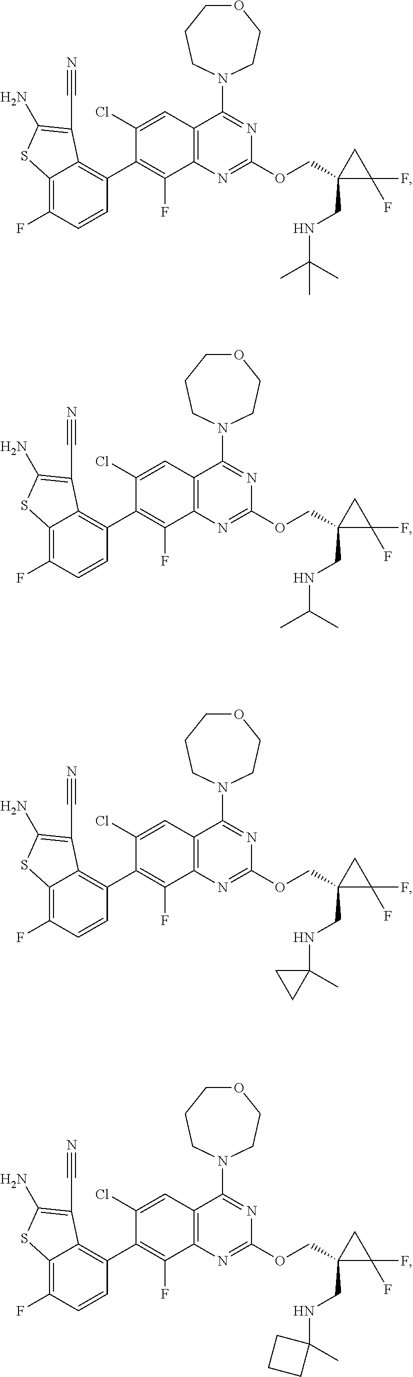 [SMILES: CC(C)(C)NC[C@@]1(COc2nc(N3CCCOCC3)c3cc(Cl)c(-c4ccc(F)c5sc(N)c(C#N)c45)c(F)c3n2)CC1(F)F ; CC(C)NC[C@@]1(COc2nc(N3CCCOCC3)c3cc(Cl)c(-c4ccc(F)c5sc(N)c(C#N)c45)c(F)c3n2)CC1(F)F ; CC1(NC[C@@]2(COc3nc(N4CCCOCC4)c4cc(Cl)c(-c5ccc(F)c6sc(N)c(C#N)c56)c(F)c4n3)CC2(F)F)CC1 ; CC1(NC[C@@]2(COc3nc(N4CCCOCC4)c4cc(Cl)c(-c5ccc(F)c6sc(N)c(C#N)c56)c(F)c4n3)CC2(F)F)CCC1]

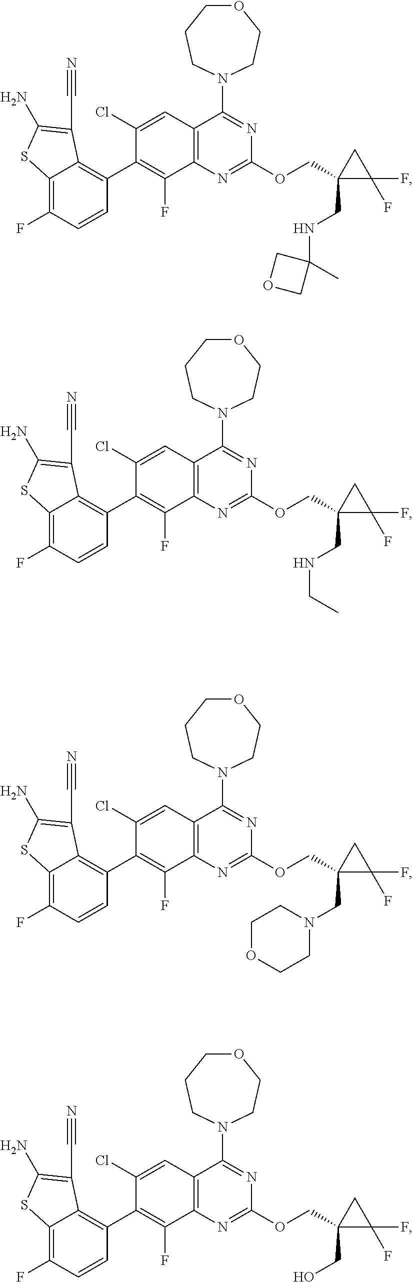 [SMILES: CC1(NC[C@@]2(COc3nc(N4CCCOCC4)c4cc(Cl)c(-c5ccc(F)c6sc(N)c(C#N)c56)c(F)c4n3)CC2(F)F)COC1 ; CCNC[C@@]1(COc2nc(N3CCCOCC3)c3cc(Cl)c(-c4ccc(F)c5sc(N)c(C#N)c45)c(F)c3n2)CC1(F)F ; N#Cc1c(N)sc2c(F)ccc(-c3c(Cl)cc4c(N5CCCOCC5)nc(OC[C@]5(CN6CCOCC6)CC5(F)F)nc4c3F)c12 ; N#Cc1c(N)sc2c(F)ccc(-c3c(Cl)cc4c(N5CCCOCC5)nc(OC[C@]5(CO)CC5(F)F)nc4c3F)c12]

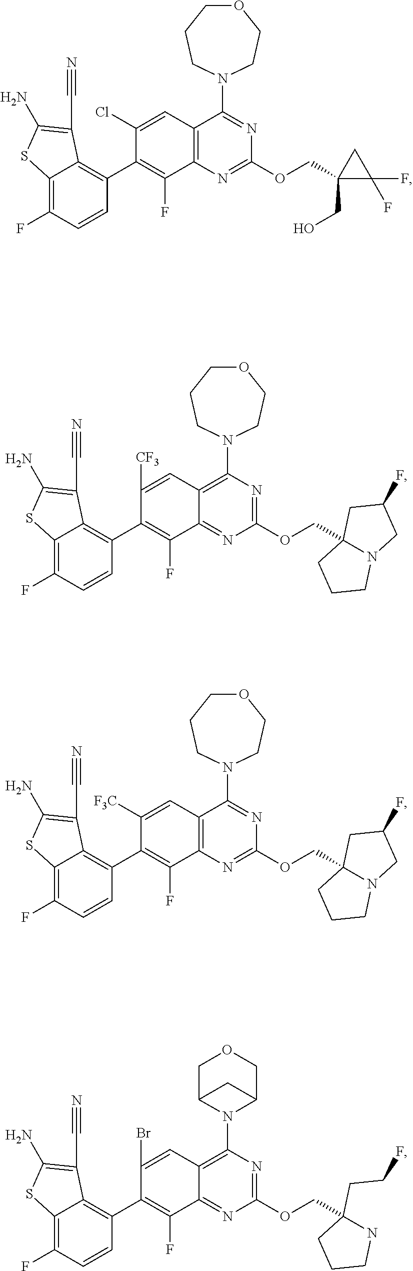

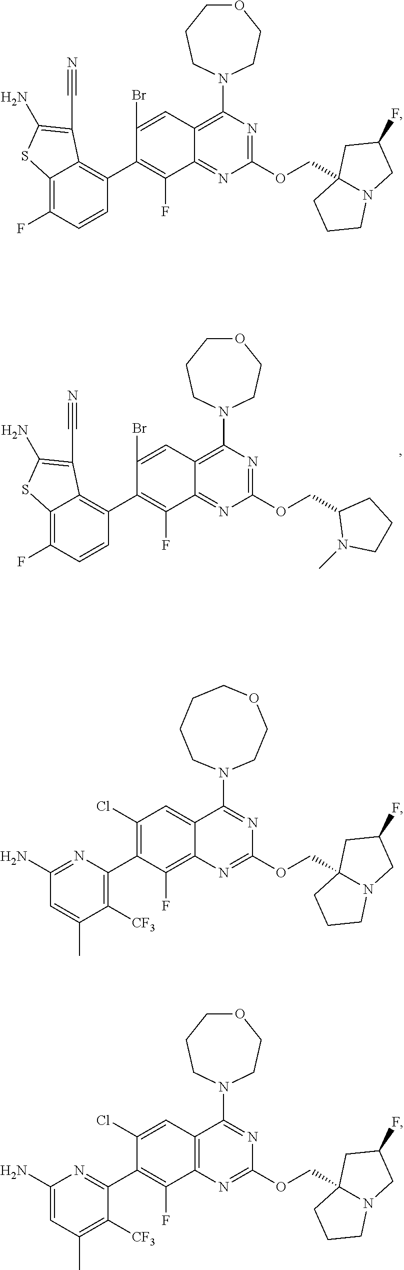 [SMILES: N#Cc1c(N)sc2c(F)ccc(-c3c(Br)cc4c(N5CCCOCC5)nc(OC[C@@]56CCCN5C[C@H](F)C6)nc4c3F)c12 ; CN1CCC[C@H]1COc1nc(N2CCCOCC2)c2cc(Br)c(-c3ccc(F)c4sc(N)c(C#N)c34)c(F)c2n1 ; Cc1cc(N)nc(-c2c(Cl)cc3c(N4CCCCOCC4)nc(OC[C@@]45CCCN4C[C@H](F)C5)nc3c2F)c1C(F)(F)F ; Cc1cc(N)nc(-c2c(Cl)cc3c(N4CCCOCC4)nc(OC[C@@]45CCCN4C[C@H](F)C5)nc3c2F)c1C(F)(F)F]

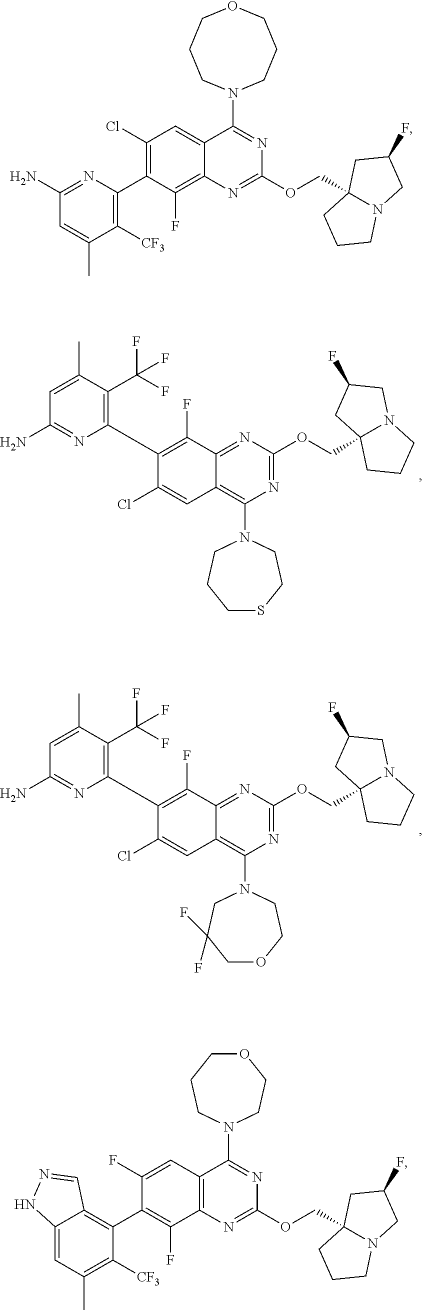 [SMILES: Cc1cc(N)nc(-c2c(Cl)cc3c(N4CCCOCCC4)nc(OC[C@@]45CCCN4C[C@H](F)C5)nc3c2F)c1C(F)(F)F ; Cc1cc(N)nc(-c2c(Cl)cc3c(N4CCCSCC4)nc(OC[C@@]45CCCN4C[C@H](F)C5)nc3c2F)c1C(F)(F)F ; Cc1cc(N)nc(-c2c(Cl)cc3c(N4CCOCC(F)(F)C4)nc(OC[C@@]45CCCN4C[C@H](F)C5)nc3c2F)c1C(F)(F)F ; Cc1cc2[nH]ncc2c(-c2c(F)cc3c(N4CCCOCC4)nc(OC[C@@]45CCCN4C[C@H](F)C5)nc3c2F)c1C(F)(F)F]

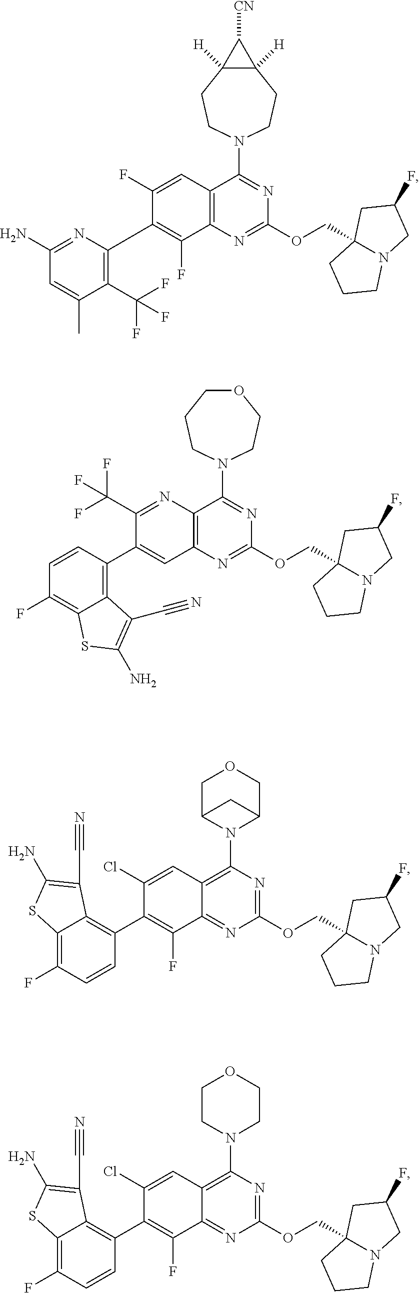 [SMILES: Cc1cc(N)nc(-c2c(F)cc3c(N4CC[C@@H]5[C@@H](C#N)[C@@H]5CC4)nc(OC[C@@]45CCCN4C[C@H](F)C5)nc3c2F)c1C(F)(F)F ; N#Cc1c(N)sc2c(F)ccc(-c3cc4nc(OC[C@@]56CCCN5C[C@H](F)C6)nc(N5CCCOCC5)c4nc3C(F)(F)F)c12 ; N#Cc1c(N)sc2c(F)ccc(-c3c(Cl)cc4c(N5C6COCC5C6)nc(OC[C@@]56CCCN5C[C@H](F)C6)nc4c3F)c12 ; N#Cc1c(N)sc2c(F)ccc(-c3c(Cl)cc4c(N5CCOCC5)nc(OC[C@@]56CCCN5C[C@H](F)C6)nc4c3F)c12]

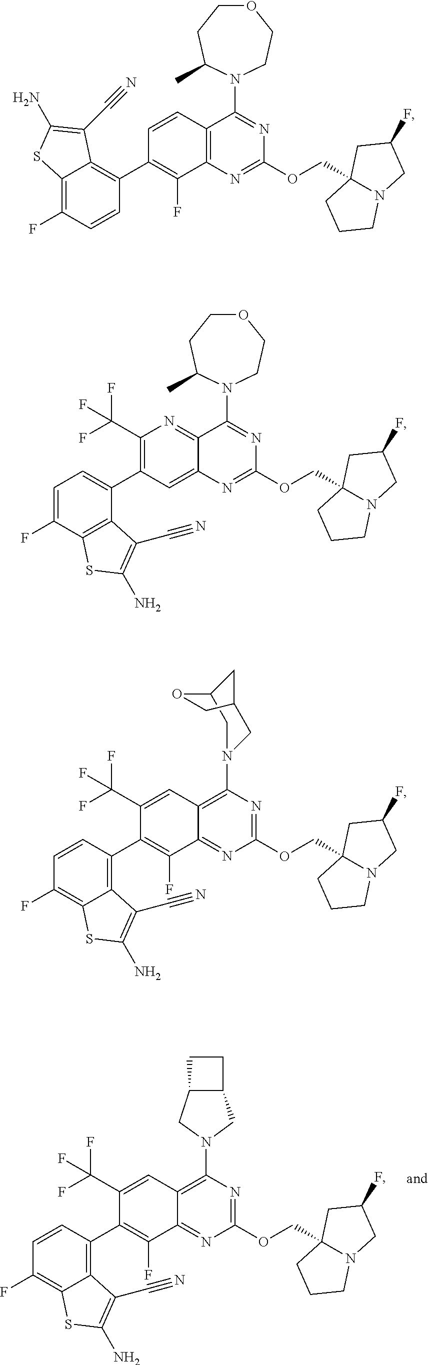 [SMILES: C[C@H]1CCOCCN1c1nc(OC[C@@]23CCCN2C[C@H](F)C3)nc2c(F)c(-c3ccc(F)c4sc(N)c(C#N)c34)ccc12 ; C[C@H]1CCOCCN1c1nc(OC[C@@]23CCCN2C[C@H](F)C3)nc2cc(-c3ccc(F)c4sc(N)c(C#N)c34)c(C(F)(F)F)nc12 ; N#Cc1c(N)sc2c(F)ccc(-c3c(C(F)(F)F)cc4c(N5CC6COC(C6)C5)nc(OC[C@@]56CCCN5C[C@H](F)C6)nc4c3F)c12 ; N#Cc1c(N)sc2c(F)ccc(-c3c(C(F)(F)F)cc4c(N5C[C@H]6CC[C@H]6C5)nc(OC[C@@]56CCCN5C[C@H](F)C6)nc4c3F)c12]

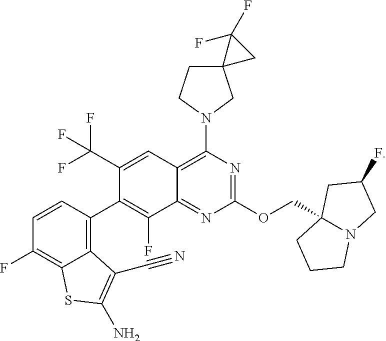 [SMILES: N#Cc1c(N)sc2c(F)ccc(-c3c(C(F)(F)F)cc4c(N5CCC6(C5)CC6(F)F)nc(OC[C@@]56CCCN5C[C@H](F)C6)nc4c3F)c12]

The present disclosure includes the pharmaceutically acceptable salts of the compounds defined herein, including the pharmaceutically acceptable salts of all structural formulas, embodiments and classes defined herein.

### Definitions

Unless defined otherwise, all technical and scientific terms used herein have the same meaning as is commonly understood by one of skill in the art to which this invention belongs.

As used throughout this disclosure, “compound(s) of Formula (I)”, “compound(s) disclosed herein”, “compound(s) described herein”, “compound(s) of the disclosure”, etc., are used interchangeably and are to be understood to include the disclosed compounds of Formula (I). The compounds of Formula (I) can form salts which are also within the scope of the present disclosure. Reference to a compound of the disclosure (or compound of Formula (I)) herein is understood to include reference to salts thereof, unless otherwise indicated.

“Alkenyl” means an aliphatic hydrocarbon group containing at least one carbon-carbon double bond and which may be straight or branched. Non-limiting examples include ethenyl, propenyl, and butenyl.

“Alkyl”, as well as other groups having the prefix “alk”, such as alkoxy, and the like, means carbon chains which may be linear or branched, or combinations thereof, containing the indicated number of carbon atoms. For instance, a C₁-C₆ alkyl means an alkyl group having one (i.e., methyl) up to 6 carbon atoms (i.e., hexyl). In particular embodiments, linear alkyl groups have 1-6 carbon atoms and branched alkyl groups have 3-7 carbon atoms. Examples of alkyl groups include methyl, ethyl, propyl, isopropyl, butyl, sec- and tert-butyl, pentyl, hexyl, heptyl, octyl, nonyl and the like.

“Alkoxy” and “alkyl-O—” are used interchangeably and refer to an alkyl group linked to oxygen. “Alkoxyalkyl” means an alkoxy-alkyl- group in which alkyl is as previously defined. The bond to the parent moiety is through a carbon atom of the alkyl group. Preferred alkoxyalkyls contain lower alkyl. Non-limiting examples of suitable alkoxyalkyl groups include methoxymethyl and methoxyethyl.

“Alkynyl” means an aliphatic hydrocarbon group containing at least one carbon-carbon triple bond and which may be straight or branched. Non-limiting examples include ethynyl, propynyl, and butynyl.

“Aryl” means a monocyclic, bicyclic or tricyclic carbocyclic aromatic ring or ring system containing 5-14 carbon atoms, wherein at least one of the rings is aromatic. Non-limiting examples include phenyl and naphthyl.

“Aminoalkyl” means -alkyl-NH₂ group in which the alkyl is as previously defined. The bond to the parent moiety is through a carbon atom of the alkyl component. Non-limiting examples of suitable aminoalkyl groups include aminomethyl and aminoethyl. “Alkylamino” means —NH-alkyl group in which the alkyl is as previously defined. The bond to the parent moiety is through the nitrogen of the amino component.

“Bicyclic ring system” refers to two joined rings. “Tricyclic ring system” refers to three joined rings. The rings may be fused, i.e., share two adjacent atoms, or “spirocyclic”, i.e., share only a single atom, or “bridged”, i.e., share three or more atoms with two bridgehead atoms being connected by a bridge containing at least one atom. Likewise the bicyclic or tricyclic rings may be aryl rings, heterocyclic rings, cycloalkyl rings, etc.

“Carbamoyl” means a H₂N—C(O)— group, which is the univalent group formed by loss of —OH group of carbamic acid. The bond to the parent group is through the carbon atom of the carbonyl component.

“Cyanoalkyl” means an -alkyl-CN group in which the alkyl is as previously defined. The bond to the parent moiety is through a carbon atom of the alkyl component. Non-limiting examples of suitable cyanoalkyl groups include cyanomethyl and 3-cyanopropyl.

“Cycloalkyl” means a saturated cyclic hydrocarbon radical. In particular embodiments, the cycloalkyl group has 3-12 carbon atoms, forming 1-3 carbocyclic rings, wherein cyclic systems having 2-3 rings can be fused. Examples of cycloalkyl include cyclopropyl, cyclobutyl, cyclopentyl, cyclohexyl, cycloheptyl, adamantyl, and the like. “Cyclofluoroalkyl” means a saturated cyclic hydrocarbon radical that is mono- or multiple-fluoro-substituted, e.g., doubly fluoro-substituted cyclopentyl. “Cycloalkoxy” refers to a cycloalkyl group linked through an oxygen to the parent moiety. “Cyclofluoroalkoxy” refers to a cyclofluoroalkyl group linked through an oxygen to the parent moiety.

“Dialkylamino” means an alkylamino as previously defined, wherein the amino atom is substituted by two alkyl substituents, which substitutions can be the same or different, e.g., —N(CH₃)₂ or —N(CH₃)(CH₂CH₃).

“Fluoroalkyl” includes mono-substituted as well as multiple fluoro-substituted alkyl groups, up to perfluoro substituted alkyl. For example, fluoromethyl, 1,1-difluoroethyl, trifluoromethyl or 1,1,1,2,2-pentafluorobutyl are included. “Fluoroalkenyl” includes mono-substituted as well as multiple fluoro-substituted alkenyl groups. “Fluoroalkynyl” includes mono-substituted as well as multiple fluoro-substituted alkynyl groups. “Fluoroalkoxy” includes mono-substituted as well as multiple fluoro-substituted “alkoxy” groups as previously defined.

“Halogen” or “halo”, unless otherwise indicated, includes fluorine (fluoro), chlorine (chloro), bromine (bromo) and iodine (iodo). In one embodiment, halo is fluoro (—F) or chloro (—Cl).

“Heteroaryl” refers to aromatic monocyclic, bicyclic and tricyclic ring structures in which one or more atoms in the ring, the heteroatom(s), is an element other than carbon. Heteroatoms are typically O, S, or N atoms. Examples of heteroaryl groups include pyrazolyl, oxadiazolonyl, pyridinyl, pyrimidinyl, pyrrolyl, pyridazinyl, isoxazolyl, thiazolyl, oxazolyl, indolyl, benzoxazolyl, benzothiazolyl, and imidazolyl.

“Heterocycloalkyl” or “heterocyclic ring” or “heterocycle” means a non-aromatic monocyclic, bicyclic, tricyclic or bridged ring system comprising about 3 to about 10 ring atoms, preferably about 5 to about 10 ring atoms, in which one or more of the atoms in the ring system is an element other than carbon, for example, nitrogen, oxygen, phosphorus or sulfur, alone or in combination. There are no adjacent oxygen and/or sulfur atoms present in the ring system. In some embodiments, heterocycloalkyls contain about 5 to about 6 ring atoms. The prefix aza, oxa, phospha or thia before the heterocyclyl root name means that at least a nitrogen, oxygen, phosphorus or sulfur atom respectively is present as a ring atom. In some embodiments, the nitrogen or sulfur atom of the heterocycloalkyl can be optionally oxidized to the corresponding N-oxide, S-oxide or S,S-dioxide. For instance, in some embodiments the heterocycloalkyl can contain N, S, S(O), S(O)₂ and/or O (which are referred to herein as “heteroatom groups”). Non-limiting examples of suitable monocyclic heterocyclyl rings include piperidyl, pyrrolidinyl, piperazinyl, morpholinyl, thiomorpholinyl, thiazolidinyl, 1,4-dioxanyl, tetrahydrofuranyl, tetrahydrothiophenyl, phosphorinane, phosphinane, 1-oxophosphinan-1-ium and the like. “Spiroheterocycloalkyl” refers to a fused ring system in which the rings share only a single atom and at least one of the rings is a heterocycloalkyl.

“Hydroxyalkyl” means a HO-alkyl- group in which alkyl is as previously defined. The bond to the parent moiety is through a carbon atom of the alkyl group. Preferred hydroxyalkyls contain lower alkyl. Non-limiting examples of suitable hydroxyalkyl groups include hydroxymethyl and 2-hydroxyethyl. “Hydroxyfluoroalkyl” means a HO-fluoroalkyl- group in which fluoroalkyl is as previously defined. “Hydroxycycloalkyl” means a HO-cycloalkyl- group in which cycloalkyl is as previously defined. “Hydroxycyclofluoroalkyl” means a HO-cyclofluoroalkyl-group in which cyclofluoroalkyl is as previously defined.

“Methylene(C₁-C₃ alkyl)(C₁-C₃ alkyl)carbamate” means having the structure of

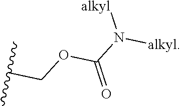

In other words, the carbamate group has alkyl groups, which can be the same or different, as previously defined, attached to the nitrogen atom.

When any variable (e.g., R^{x}) occurs more than one time in any constituent or in Formula (I) or other generic formulas herein, its definition on each occurrence is independent of its definition at every other occurrence. Combinations of substituents and/or variables are permissible only if such combinations result in stable compounds. In choosing compounds of the present disclosure, one of ordinary skill in the art will recognize that the various substituents, e.g., R^{x}, are to be chosen in conformity with well-known principles of chemical structure connectivity and stability. Unless expressly stated to the contrary, substitution by a named substituent is permitted on any atom in a ring (e.g., aryl, a heteroaryl ring, or a saturated heteroaryl ring) provided such ring substitution is chemically allowed and results in a stable compound. A “stable” compound is a compound which can be prepared and isolated and whose structure and properties remain or can be caused to remain essentially unchanged for a period of time sufficient to allow use of the compound for the purposes described herein (e.g., therapeutic or prophylactic administration to a subject).

The term “substituted” shall be deemed to include multiple degrees of substitution by a named substituent. Where multiple substituent moieties are disclosed or claimed, the substituted compound can be independently substituted by one or more of the disclosed or claimed substituent moieties, singly or plurally. By independently substituted, it is meant that the (two or more) substituents can be the same or different.

Unless expressly depicted or described otherwise, variables depicted in a structural formula with a “floating” bond, such as R^{X}, are permitted on any available carbon atom in the ring to which the variable is attached. When a moiety is noted as being “optionally substituted” in Formula (I) or any embodiment thereof, it means that Formula (I) or the embodiment thereof encompasses compounds that contain the noted substituent (or substituents) on the moiety and also compounds that do not contain the noted substituent (or substituents) on the moiety.

The wavy line [symbol] , as used herein, indicates a point of attachment to the rest of the compound.

The compounds of Formula (I) may contain one or more asymmetric centers and can thus occur as racemates and racemic mixtures, single enantiomers, diastereoisomeric mixtures and individual diastereoisomers. Centers of asymmetry that are present in the compounds of Formula (I) can all independently of one another have S configuration or R configuration. The compounds of Formula (I) include all possible enantiomers and diastereomers and mixtures of two or more stereoisomers, for example, mixtures of enantiomers and/or diastereomers, in all ratios. Thus, enantiomers are a subject of the disclosure in enantiomerically pure form, both as levorotatory and as dextrorotatory antipodes, in the form of racemates and in the form of mixtures of the two enantiomers in all ratios. In the case of a cis/trans isomerism, the disclosure includes both the cis form and the trans form as well as mixtures of these forms in all ratios. The present disclosure is meant to comprehend all such stereoisomeric forms of the compounds of Formula (I). Where a structural formula or chemical name specifies a particular configuration at a stereocenter, the enantiomer or stereoisomer of the compound resulting from that specified stereocenter is intended. Where a structural formula of the compounds of Formula (I) indicates a straight line at a chiral center, the structural formula includes both the S and R stereoisomers associated with the chiral center and mixtures thereof.

The compounds of Formula (I) may be separated into their individual diastereoisomers by, for example, fractional crystallization from a suitable solvent, for example, methanol or ethyl acetate or a mixture thereof, or via chiral chromatography using an optically active stationary phase. Absolute stereochemistry may be determined by X-ray crystallography of crystalline products or crystalline intermediates which are derivatized, if necessary, with a reagent containing an asymmetric center of known absolute configuration. Vibrational circular dichroism (VCD) may also be used to determine the absolute stereochemistry. Alternatively, any stereoisomer or isomers of the compounds of Formula (I) may be obtained by stereospecific synthesis using optically pure starting materials or reagents of known absolute configuration.

If desired, racemic mixtures of the compounds may be separated so that the individual enantiomers are isolated. The separation can be carried out by methods well known in the art, such as the coupling of a racemic mixture of compounds to an enantiomerically pure compound to form a diastereoisomeric mixture, followed by separation of the individual diastereoisomers by standard methods, such as fractional crystallization or chromatography. The coupling reaction is often the formation of salts using an enantiomerically pure acid or base. The diasteromeric derivatives may then be converted to the pure enantiomers by cleavage of the added chiral residue. The racemic mixture of the compounds can also be separated directly by chromatographic methods utilizing chiral stationary phases, which methods are well known in the art.

The compounds of Formula (I) which contain olefinic double bonds, unless specified otherwise, they are meant to include both E and Z geometric isomers.

Some of the compounds described herein may exist as tautomers which have different points of attachment of hydrogen accompanied by one or more double bond shifts. For example, a ketone and its enol form are keto-enol tautomers. The individual tautomers as well as mixtures thereof are encompassed by the compounds of Formula (I).

Some of the compounds of Formula (I) described herein may exist as atropisomers when the rotational energy barrier around a single bond is sufficiently high to prevent free rotation at a given temperature, thus allowing isolation of individual conformers with distinct properties. The individual atropisomers as well as mixtures thereof are encompassed with compounds of Formula (I) of the present disclosure. When resolved, individual atropisomers can be designated by established conventions such as those specified by the International Union of Pure Applied Chemistry (IUPAC) 2013 Recommendations.

In the compounds of Formula (I), the atoms may exhibit their natural isotopic abundances, or one or more of the atoms may be artificially enriched in a particular isotope having the same atomic number, but an atomic mass or mass number different from the atomic mass or mass number predominantly found in nature. The present disclosure as described and claimed herein is meant to include all suitable isotopic variations of the compounds of Formula (I) and embodiments thereof. For example, different isotopic forms of hydrogen (H) include protium (H) and deuterium (²H, also denoted herein as D). Protium is the predominant hydrogen isotope found in nature. Enriching for deuterium may afford certain therapeutic advantages, such as increasing in vivo half-life or reducing dosage requirements or may provide a compound useful as a standard for characterization of biological samples. Isotopically-enriched compounds can be prepared without undue experimentation by conventional techniques well known to those skilled in the art or by processes analogous to those described in the Schemes and Examples herein using appropriate isotopically-enriched reagents and/or intermediates.

The term “pharmaceutically acceptable salts” refers to salts prepared from pharmaceutically acceptable non-toxic bases or acids. When a compound of Formula (I) is acidic, its corresponding salt can be conveniently prepared from pharmaceutically acceptable non-toxic bases, including inorganic bases and organic bases. Salts derived from such inorganic bases include aluminum, ammonium, calcium, copper (ic and ous), ferric, ferrous, lithium, magnesium, manganese (ic and ous), potassium, sodium, zinc and the like salts. Preferred are the ammonium, calcium, magnesium, potassium and sodium salts. Salts prepared from pharmaceutically acceptable organic non-toxic bases include salts of primary, secondary, and tertiary amines derived from both naturally occurring and synthetic sources. Pharmaceutically acceptable organic non-toxic bases from which salts can be formed include, for example, arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, dicyclohexylamine, lysine, methylglucamine, morpholine, piperazine, piperidine, polyamine resins, procaine, purines, theobromine, triethylamine, trimethylamine, tripropylamine, tromethamine and the like.

When a compound of Formula (I) is basic, its corresponding salt can be conveniently prepared from pharmaceutically acceptable non-toxic inorganic and organic acids. Such acids include, for example, acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid and the like. Preferred are citric, hydrobromic, hydrochloric, maleic, phosphoric, sulfuric, and tartaric acids. If a compound of Formula (I) simultaneously contains acidic and basic groups in the molecule, the disclosure also includes, in addition to the salt forms mentioned, inner salts or betaines (zwitterions). Salts can be obtained from the compounds of Formula (I) by customary methods which are known to the person skilled in the art, for example, by combination with an organic or inorganic acid or base in a solvent or dispersant, or by anion exchange or cation exchange from other salts. The present disclosure also includes all salts of the compounds of Formula (I) which, owing to low physiological compatibility, are not directly suitable for use in pharmaceuticals but which can be used, for example, as intermediates for chemical reactions or for the preparation of pharmaceutically acceptable salts.

Furthermore, the compounds of Formula (I) may exist in amorphous form and/or one or more crystalline forms, and as such all amorphous and crystalline forms and mixtures thereof of the compounds of Formula (I), including the Examples, are intended to be included within the scope of the present disclosure. In addition, some of the compounds of Formula (I) may form solvates with water (i.e., a hydrate) or common organic solvents such as but not limited to ethyl acetate. Such solvates and hydrates, particularly the pharmaceutically acceptable solvates and hydrates, of the instant compounds are likewise encompassed within the scope of this disclosure, along with un-solvated and anhydrous forms.

Any pharmaceutically acceptable pro-drug modification of a compound of Formula (I) which results in conversion in vivo to a compound within the scope of this disclosure is also within the scope of this disclosure.

The terms “therapeutically effective (or efficacious) amount” and similar descriptions such as “an amount efficacious for treatment” or “an effective dose” are intended to mean that amount of a compound of Formula (I) that will elicit the biological or medical response of a tissue, a system, animal or human that is being sought by a researcher, veterinarian, medical doctor or other clinician. In a preferred embodiment, the term “therapeutically effective amount” means an amount of a compound of Formula (I) that alleviates at least one clinical symptom in a human patient. The terms “prophylactically effective (or efficacious) amount” and similar descriptions such as “an amount efficacious for prevention” are intended to mean that amount of a compound of Formula (I) that will prevent or reduce the risk of occurrence of the biological or medical event that is sought to be prevented in a tissue, a system, animal or human by a researcher, veterinarian, medical doctor or other clinician.

### Dosages of the Compounds of Formula (I)

The dosage regimen utilizing a compound of Formula (I) is selected in accordance with a variety of factors including type, species, age, weight, sex and medical condition of the patient; the severity of the condition to be treated; the potency of the compound chosen to be administered; the route of administration; and the renal and hepatic function of the patient. A consideration of these factors is well within the purview of the ordinarily skilled clinician for the purpose of determining the therapeutically effective or prophylactically effective dosage amount needed to prevent, counter, or arrest the progress of the condition. It is understood that a specific daily dosage amount can simultaneously be both a therapeutically effective amount, e.g., for treatment of an oncological condition, and a prophylactically effective amount, e.g., for prevention of an oncological condition.

While individual needs vary, determination of optimal ranges of effective amounts of the compounds of Formula (I) is within the skill of the art. For administration to a human in, for example, the curative or prophylactic treatment of the conditions and disorders identified herein, the typical dosages of the compounds of Formula (I) can be about 0.05 mg/kg/day to about 50 mg/kg/day, or at least 0.05 mg/kg, or at least 0.08 mg/kg, or at least 0.1 mg/kg, or at least 0.2 mg/kg, or at least 0.3 mg/kg, or at least 0.4 mg/kg, or at least 0.5 mg/kg, and any amount therebetween, to about 50 mg/kg or less, or about 40 mg/kg or less, or about 30 mg/kg or less, or about 20 mg/kg or less, or about 10 mg/kg or less and any amount therebetween, which can be, for example, about 2.5 mg/day (0.5 mg/kg×5 kg) to about 5000 mg/day (50 mg/kg×100 kg). For example, dosages of the compounds can be about 0.1 mg/kg/day to about 50 mg/kg/day, or about 0.05 mg/kg/day to about 10 mg/kg/day, or about 0.05 mg/kg/day to about 5 mg/kg/day, or about 0.05 mg/kg/day to about 3 mg/kg/day, or about 0.07 mg/kg/day to about 3 mg/kg/day, or about 0.09 mg/kg/day to about 3 mg/kg/day, or about 0.05 mg/kg/day to about 0.1 mg/kg/day, or about 0.1 mg/kg/day to about 1 mg/kg/day, or about 1 mg/kg/day to about 10 mg/kg/day, or about 1 mg/kg/day to about 5 mg/kg/day, or about 1 mg/kg/day to about 3 mg/kg/day, or about 3 mg/day to about 500 mg/day, or about 5 mg/day to about 250 mg/day, or about 10 mg/day to about 100 mg/day, or about 3 mg/day to about 10 mg/day, or about 100 mg/day to about 250 mg/day. Such doses may be administered in a single dose or may be divided into multiple doses.

### Pharmaceutical Compositions

The compounds of Formula (I) and their pharmaceutically acceptable salts can be administered to animals, preferably to mammals, and in particular to humans, as pharmaceuticals by themselves, in mixtures with one another or in the form of pharmaceutical compositions. The term “subject” or “patient” includes animals, preferably mammals and especially humans, who use the instant active agents for the prevention or treatment of a medical condition. Administering of the drug to the subject includes both self-administration and administration to the patient by another person. The subject may be in need of, or desire, treatment for an existing disease or medical condition, or may be in need of or desire prophylactic treatment to prevent or reduce the risk of occurrence of said disease or medical condition. As used herein, a subject “in need” of treatment of an existing condition or of prophylactic treatment encompasses both a determination of need by a medical professional as well as the desire of a patient for such treatment.

The present disclosure therefore also provides the compounds of Formula (I) and their pharmaceutically acceptable salts for use as pharmaceuticals, their use for modulating the activity of mutant and/or WT KRAS proteins and in particular their use in the therapy and prophylaxis of the below-mentioned diseases or disorders as well as their use for preparing medicaments for these purposes. In certain embodiments, the compounds of Formula (I) and their pharmaceutically acceptable salts inhibit the KRAS-G12C, KRAS-G12D, KRAS-G12V, and/or KRAS-G13D proteins.

Furthermore, the present disclosure provides pharmaceutical compositions which comprise as active component an effective dose of at least one compound of Formula (I) and/or a pharmaceutically acceptable salt thereof and a customary pharmaceutically acceptable carrier, i.e., one or more pharmaceutically acceptable carrier substances and/or additives.

Thus, the present disclosure provides, for example, said compound and its pharmaceutically acceptable salts for use as pharmaceutical compositions which comprise as active component an effective dose of at least one compound of Formula (I) and/or a pharmaceutically acceptable salt thereof and a customary pharmaceutically acceptable carrier, and the uses of said compound and/or a pharmaceutically acceptable salt thereof in the therapy or prophylaxis of the below-mentioned diseases or disorders, e.g., cancer, as well as their use for preparing medicaments for these purposes.

The pharmaceutical compositions according to the disclosure can be administered orally, for example, in the form of pills, tablets, lacquered tablets, sugar-coated tablets, granules, hard and soft gelatin capsules, aqueous, alcoholic or oily solutions, syrups, emulsions or suspensions, or rectally, for example, in the form of suppositories. Administration can also be carried out parenterally, for example subcutaneously, intramuscularly or intravenously in the form of solutions for injection or infusion.

Other suitable administration forms are, for example, percutaneous or topical administration, for example, in the form of ointments, tinctures, sprays or transdermal therapeutic systems, or, for example, microcapsules, implants or rods. The preferred administration form depends, for example, on the disease to be treated and on its severity.

The amount of active compound of a compound described herein and/or its pharmaceutically acceptable salts in the pharmaceutical composition normally is from 0.01 to 200 mg, or from 0.1 to 200 mg, or from 1 to 200 mg, per dose, but depending on the type of the pharmaceutical composition, it can also be higher. In some embodiments, the amount of active compound of a compound of Formula (I) and/or its pharmaceutically acceptable salts in the pharmaceutical composition is from 0.01 to 10 mg per dose. The pharmaceutical compositions usually comprise 0.5 to 90 percent by weight of at least one compound of Formula (I) and/or its pharmaceutically acceptable salts. The preparation of the pharmaceutical compositions can be carried out in a manner known per se. For this purpose, one or more compounds of Formula (I) and/or their pharmaceutically acceptable salts, together with one or more solid or liquid pharmaceutical carrier substances and/or additives (or auxiliary substances) and, if desired, in combination with other pharmaceutically active compounds having therapeutic or prophylactic action, are brought into a suitable administration form or dosage form which can then be used as a pharmaceutical in human or veterinary medicine.

For the production of pills, tablets, sugar-coated tablets and hard gelatin capsules, it is possible to use, for example, lactose, starch, for example, maize starch, or starch derivatives, talc, stearic acid or its salts, etc. Carriers for soft gelatin capsules and suppositories are, for example, fats, waxes, semisolid and liquid polyols, natural or hardened oils, etc. Suitable carriers for the preparation of solutions, for example, of solutions for injection, or of emulsions or syrups are, for example, water, physiologically acceptable sodium chloride solution, alcohols such as ethanol, glycerol, polyols, sucrose, invert sugar, glucose, mannitol, vegetable oils, etc. It is also possible to lyophilize the compounds of Formula (I) and their pharmaceutically acceptable salts and to use the resulting lyophilisates, for example, for preparing preparations for injection or infusion. Suitable carriers for microcapsules, implants or rods are, for example, copolymers of glycolic acid and lactic acid.

Besides the active compounds and carriers, the pharmaceutical compositions can also contain customary additives, for example, fillers, disintegrants, binders, lubricants, wetting agents, stabilizers, emulsifiers, dispersants, preservatives, sweeteners, colorants, flavorings, aromatizers, thickeners, diluents, buffer substances, solvents, solubilizers, agents for achieving a depot effect, salts for altering the osmotic pressure, coating agents and/or antioxidants.

### Methods of Using the Compounds of Formula (I)

The present application provides a method of inhibiting RAS-mediated cell signaling comprising contacting a cell with a compound of Formula (I) or a pharmaceutically acceptable salt thereof. Inhibition of RAS-mediated signal transduction can be assessed and demonstrated by a wide variety of ways known in the art. Non-limiting examples include (a) a decrease in GTPase activity of RAS; (b) a decrease in GTP binding affinity or an increase in GDP binding affinity; (c) an increase in K_{off} of GTP or a decrease in K_{off} of GDP; (d) a decrease in the levels of signaling transduction molecules downstream in the RAS pathway, such as a decrease in pMEK, pERK, or pAKT levels; and/or (e) a decrease in binding of RAS complex to downstream signaling molecules including but not limited to Raf. Kits and commercially available assays can be utilized for determining one or more of the above.

The present application also provides methods of using the compounds of Formula (I) (or their pharmaceutically acceptable salts) or pharmaceutical compositions containing such compounds to treat disease conditions, including but not limited to, conditions implicated by mutant KRAS proteins and/or amplification or over expression of WT KRAS protein (e.g., cancer), and in some embodiments the KRAS-G12C, KRAS-G12D, KRAS-G12V, and/or KRAS-G13D mutants.

In some embodiments, a method for treatment of cancer is provided, the method comprising administering a therapeutically effective amount a compound of Formula (I) (or a pharmaceutically acceptable salt thereof) or any of the foregoing pharmaceutical compositions comprising such a compound to a subject in need of such treatment. In some embodiments, the cancer is mediated by a KRAS mutation, e.g., the KRAS-G12C, KRAS-G12D, KRAS-G12V, and/or KRAS-G13D mutations. In various embodiments, the cancer is pancreatic cancer, colorectal cancer or lung cancer. In some embodiments, the cancer is gall bladder cancer, thyroid cancer, or bile duct cancer.

In some embodiments the present disclosure provides a method of treating a disorder in a subject in need thereof, wherein said method comprises determining if the subject has a KRAS mutation (e.g., KRAS-G12C, KRAS-G12D, KRAS-G12V, and/or KRAS-G13D mutations) and if the subject is determined to have the KRAS mutation, then administering to the subject a therapeutically effective amount of a compound of Formula (I) or a pharmaceutically acceptable salt thereof.

In some embodiments the present disclosure provides a method of treating a disorder in a subject in need thereof, wherein said method comprises determining if the subject has amplified and/or over expression of WT KRAS protein and if the subject is determined to have such features, then administering to the subject a therapeutically effective amount of a compound of Formula (I) or a pharmaceutically acceptable salt thereof.

The disclosed compounds inhibit anchorage-independent cell growth and therefore have the potential to inhibit tumor metastasis. Accordingly, another embodiment of the present disclosure provides a method for inhibiting tumor metastasis, the method comprising administering an effective amount a compound of Formula (I).

KRAS mutations have also been identified in hematological malignancies (e.g., cancers that affect blood, bone marrow and/or lymph nodes). Accordingly, certain embodiments are directed to administration of the compounds of Formula (I) (e.g., in the form of a pharmaceutical composition) to a subject in need of treatment of a hematological malignancy. Such malignancies include, but are not limited to leukemias and lymphomas. For example, the presently disclosed compounds can be used for treatment of diseases such as acute lymphoblastic leukemia (ALL), acute myelogenous leukemia (AML), chronic lymphocytic leukemia (CLL), small lymphocytic lymphoma (SLL), chronic myelogenous leukemia (CML), acute monocytic leukemia (AMoL) and/or other leukemias. In other embodiments, the compounds are useful for treatment of lymphomas such as Hodgkin's lymphoma or non-Hodgkin's lymphoma. In various embodiments, the compounds are useful for treatment of plasma cell malignancies such as multiple myeloma, mantle cell lymphoma, and Waldenstrom's macroglubunemia.

Determining whether a tumor or cancer comprises a KRAS mutation (e.g., the KRAS-G12C, KRAS-G12D and/or KRAS-G12V mutations) or WT KRAS can be undertaken by assessing the nucleotide sequence encoding the KRAS protein, by assessing the amino acid sequence of the KRAS protein, or by assessing the characteristics of a putative KRAS mutant or WT KRAS protein. The sequence of wild-type human KRAS is known in the art.

Methods for detecting a mutation in a KRAS nucleotide sequence or a WT KRAS nucleotide sequence are also known by those of skill in the art. These methods include, but are not limited to, polymerase chain reaction-restriction fragment length polymorphism (PCR-RFLP) assays, polymerase chain reaction-single strand conformation polymorphism (PCR-SSCP) assays, real-time PCR assays, PCR sequencing, mutant allele-specific PCR amplification (MASA) assays, direct sequencing, primer extension reactions, electrophoresis, oligonucleotide ligation assays, hybridization assays, TaqMan assays, SNP genotyping assays, high resolution melting assays and microarray analyses. In some embodiments, samples are evaluated for KRAS mutations (e.g., the KRAS-G12C, KRAS-G12D, KRAS-G12V, and/or KRAS-G13D mutations) by real-time PCR. In real-time PCR, fluorescent probes specific for the KRAS mutation are used. When a mutation is present, the probe binds and fluorescence is detected. In some embodiments, the KRAS mutation is identified using a direct sequencing method of specific regions (e.g., exon 2 and/or exon 3) in the KRAS gene.

Methods for detecting a mutation in a KRAS protein or a WT KRAS protein (e.g., the KRAS-G12C, KRAS-G12D, KRAS-G12V, KRAS-G13D mutations) are known by those of skill in the art. These methods include, but are not limited to, detection of a KRAS mutant or WT KRAS protein using a binding agent (e.g., an antibody) specific for the mutant or WT protein, protein electrophoresis and Western blotting, and direct peptide sequencing.

A number of tissue samples can be assessed for determining whether a tumor or cancer comprises a KRAS mutation (e.g., the KRAS-G12C, KRAS-G12D, KRAS-G12V, and/or KRAS-G13D mutations) or amplified/overexpressed WT KRAS. In some embodiments, the sample is taken from a subject having a tumor or cancer. In some embodiments, the sample is a fresh tumor/cancer sample. In some embodiments, the sample is a frozen tumor/cancer sample. In some embodiments, the sample is a formalin-fixed paraffin-embedded sample. In some embodiments, the sample is a circulating tumor cell (CTC) sample. In some embodiments, the sample is processed to a cell lysate. In some embodiments, the sample is processed to DNA or RNA.

The present application also provides a method of treating a hyperproliferative disorder comprising administering a therapeutically effective amount of a compound of Formula (I), or a pharmaceutically acceptable salt thereof to a subject in need thereof. In some embodiments, said method relates to the treatment of a subject who suffers from a cancer such as acute myeloid leukemia, cancer in adolescents, adrenocortical carcinoma childhood, AIDS-related cancers (e.g., lymphoma and Kaposi's Sarcoma), anal cancer, appendix cancer, astrocytomas, atypical teratoid, basal cell carcinoma, bile duct cancer, bladder cancer, bone cancer, brain stem glioma, brain tumor, breast cancer, bronchial tumors, Burkitt lymphoma, carcinoid tumor, atypical teratoid, embryonal tumors, germ cell tumor, primary lymphoma, cervical cancer, childhood cancers, chordoma, cardiac tumors, chronic lymphocytic leukemia (CLL), chronic myelogenous leukemia (CML), chronic myleoproliferative disorders, colon cancer, colorectal cancer, craniopharyngioma, cutaneous T-cell lymphoma, extrahepatic ductal carcinoma in situ (DCIS), embryonal tumors, CNS cancer, endometrial cancer, ependymoma, esophageal cancer, esthesioneuroblastoma, Ewing sarcoma, extracranial germ cell tumor, extragonadal germ cell tumor, eye cancer, fibrous histiocytoma of bone, gall bladder cancer, gastric cancer, gastrointestinal carcinoid tumor, gastrointestinal stromal tumors (GIST), germ cell tumor, gestational trophoblastic tumor, hairy cell leukemia, head and neck cancer, heart cancer, liver cancer, Hodgkin's lymphoma, hypopharyngeal cancer, intraocular melanoma, islet cell tumors, pancreatic neuroendocrine tumors, kidney cancer, laryngeal cancer, lip and oral cavity cancer, liver cancer, lobular carcinoma in situ (LCIS), lung cancer, lymphoma, metastatic squamous neck cancer with occult primary, midline tract carcinoma, mouth cancer; multiple endocrine neoplasia syndromes, multiple myeloma/plasma cell neoplasm, mycosis fungoides, myelodysplasia syndromes, myelodysplastic/myeloproliferative neoplasms, multiple myeloma, Merkel cell carcinoma, malignant mesothelioma, malignant fibrous histiocytoma of bone and osteosarcoma, nasal cavity and paranasal sinus cancer, nasopharyngeal cancer, neuroblastoma, non-Hodgkin's lymphoma, non-small cell lung cancer (NSCLC), oral cancer, lip and oral cavity cancer, oropharyngeal cancer, ovarian cancer, pancreatic cancer, papillomatosis, paraganglioma, paranasal sinus and nasal cavity cancer, parathyroid cancer, penile cancer, pharyngeal cancer, pleuropulmonary blastoma, primary central nervous system (CNS) lymphoma, prostate cancer, rectal cancer, transitional cell cancer, retinoblastoma, rhabdomyosarcoma, salivary gland cancer, skin cancer, stomach (gastric) cancer, small cell lung cancer; small intestine cancer, soft tissue sarcoma, T-Cell lymphoma, testicular cancer, throat cancer, thymoma and thymic carcinoma, thyroid cancer, transitional cell cancer of the renal pelvis and ureter, trophoblastic tumor, unusual cancers of childhood, urethral cancer, uterine sarcoma, vaginal cancer, vulvar cancer, or viral-induced cancer. In some embodiments, said method relates to the treatment of a non-cancerous hyperproliferative disorder such as benign hyperplasia of the skin (e.g., psoriasis), restenosis, or prostate (e.g., benign prostatic hypertrophy (BPH)).

In some embodiments, the methods for treatment are directed to treating lung cancers, and the methods comprise administering a therapeutically effective amount of the compounds of Formula (I) (or pharmaceutical composition comprising such compounds) to a subject in need thereof. In certain embodiments, the lung cancer is a non-small cell lung carcinoma (NSCLC), for example, adenocarcinoma, squamous-cell lung carcinoma or large-cell lung carcinoma. In some embodiments, the lung cancer is a small cell lung carcinoma. Other lung cancers which the compounds of Formula (I) may provide therapeutic benefit for include, but are not limited to, glandular tumors, carcinoid tumors and undifferentiated carcinomas.

The present disclosure also provides methods of modulating a mutant KRAS protein activity (e.g., activity resulting from the KRAS-G12C, KRAS-G12D, KRAS-G12V, and/or KRAS-G13D mutations) or a WT KRAS protein activity by contacting the protein with an effective amount of a compound of Formula (I). Modulation can be inhibiting or activating protein activity. In some embodiments, the present disclosure provides methods of inhibiting protein activity by contacting the mutant KRAS protein (e.g., KRAS-G12C, KRAS-G12D, KRAS-G12V, and/or KRAS-G13D mutants) or WT KRAS protein with an effective amount of a compound of Formula (I) in solution. In some embodiments, the present disclosure provides methods of inhibiting the mutant or WT KRAS protein activity by contacting a cell, tissue, or organ that expresses the protein of interest. In some embodiments, the disclosure provides methods of inhibiting protein activity in subjects including, but not limited to, rodents and mammals (e.g., humans) by administering into the subjects an effective amount of a compound of Formula (I).

### Combination Therapies

One or more additional pharmacologically active agents may be administered in combination with a compound of Formula (I) (or a pharmaceutically acceptable salt thereof). An additional active agent (or agents) is intended to mean a pharmaceutically active agent (or agents) that is active in the body, including pro-drugs that convert to pharmaceutically active form after administration, which are different from the compound of Formula (I). The additional active agents also include free-acid, free-base and pharmaceutically acceptable salts of said additional active agents. Generally, any suitable additional active agent or agents, including chemotherapeutic agents or therapeutic antibodies, may be used in any combination with the compound of Formula (I) in a single dosage formulation (e.g., a fixed dose drug combination), or in one or more separate dosage formulations which allows for concurrent or sequential administration of the active agents (co-administration of the separate active agents) to subjects. In addition, the compounds of Formula (I) (or pharmaceutically acceptable salts thereof) can be administered in combination with radiation therapy, hormone therapy, surgery or immunotherapy.

The present application also provides methods for combination therapies in which the additional active agent is known to modulate other pathways, or other components of the same pathway, or even overlapping sets of target enzymes which are used in combination with a compound of Formula (I), or a pharmaceutically acceptable salt thereof. In one embodiment, such therapy includes, but is not limited to, the combination of one or more compounds of Formula (I) with chemotherapeutic agents, immunotherapeutic agents, hormonal and anti-hormonal agents, targeted therapy agents, and anti-angiogenesis agents, to provide a synergistic or additive therapeutic effect. In another embodiment, such therapy includes radiation treatment to provide a synergistic or additive therapeutic effect.

Examples of additional active agents (i.e., additional anti-cancer agents) include chemotherapeutic agents (e.g., cytotoxic agents), immunotherapeutic agents, hormonal and anti-hormonal agents, targeted therapy agents, and anti-angiogenesis agents. Many anti-cancer agents can be classified within one or more of these groups. While certain anti-cancer agents have been categorized within a specific group(s) or subgroup(s) herein, many of these agents can also be listed within one or more other group(s) or subgroup(s), as would be presently understood in the art. It is to be understood that the classification herein of a particular agent into a particular group is not intended to be limiting. Many anti-cancer agents are presently known in the art and can be used in combination with the compounds of the present disclosure.

Further, an agent can be an agonist, antagonist, allosteric modulator, toxin or, more generally, may act to inhibit or stimulate its target (e.g., receptor or enzyme activation or inhibition). For example, suitable for use are one or more agents (e.g., antibodies, antigen binding regions, or soluble receptors) that specifically bind and inhibit the activity of growth factors, such as antagonists of hepatocyte growth factor (HGF, also known as Scatter Factor), and antibodies or antigen binding regions that specifically bind its receptor “c-met”.

In an embodiment, the additional anti-cancer agent is a chemotherapeutic agent, an immunotherapeutic agent, a hormonal agent, an anti-hormonal agent, a targeted therapy agent, or an anti-angiogenesis agent (or angiogenesis inhibitor). In an embodiment, the additional anti-cancer agent is selected from the group consisting of a chemotherapeutic agent, a mitotic inhibitor, a plant alkaloid, an alkylating agent, an anti-metabolite, a platinum analog, an enzyme, a topoisomerase inhibitor, a retinoid, an aziridine, an antibiotic, a hormonal agent, an anti-hormonal agent, an anti-estrogen, an anti-androgen, an anti-adrenal, an androgen, a targeted therapy agent, an immunotherapeutic agent, a biological response modifier, a cytokine inhibitor, a tumor vaccine, a monoclonal antibody, an immune checkpoint inhibitor, an anti-PD-1 agent, an anti-PD-L1 agent, a colony-stimulating factor, an immunomodulator, an immunomodulatory imide (IMiD), an anti-CTLA4 agent, an anti-LAGl agent, an anti-OX40 agent, a GITR agonist, a CAR-T cell, a BiTE, a signal transduction inhibitor, a growth factor inhibitor, a tyrosine kinase inhibitor, an EGFR inhibitor, a histone deacetylase (HDAC) inhibitor, a proteasome inhibitor, a cell-cycle inhibitor, an anti-angiogenesis agent, a matrix-metalloproteinase (MMP) inhibitor, a hepatocyte growth factor inhibitor, a TOR inhibitor, a KDR inhibitor, a VEGF inhibitor, a HIF-1α inhibitor, a HIF-2α inhibitor, a fibroblast growth factor (FGF) inhibitor, a RAF inhibitor, a MEK inhibitor, an ERK inhibitor, a PI3K inhibitor, an AKT inhibitor, an MCL-1 inhibitor, a BCL-2 inhibitor, an SHP2 inhibitor, a HER-2 inhibitor, a BRAF-inhibitor, a gene expression modulator, an autophagy inhibitor, an apoptosis inducer, an antiproliferative agent, and a glycolysis inhibitor.

In one embodiment, the additional anti-cancer agent(s) is a chemotherapeutic agent. Non-limiting examples of chemotherapeutic agents include mitotic inhibitors and plant alkaloids, alkylating agents, anti-metabolites, platinum analogs, enzymes, topoisomerase inhibitors, retinoids, aziridines, and antibiotics.

Non-limiting examples of mitotic inhibitors and plant alkaloids include taxanes such as cabazitaxel, docetaxel, larotaxel, ortataxel, paclitaxel, and tesetaxel; demecolcine; epothilone; eribulin; etoposide (VP-16); etoposide phosphate; navelbine; noscapine; teniposide; thaliblastine; vinblastine; vincristine; vindesine; vinflunine; and vinorelbine.

Non-limiting examples of alkylating agents include nitrogen mustards such as chlorambucil, chlornaphazine, cholophosphamide, cytophosphane, estramustine, ifosfamide, mannomustine, mechlorethamine, mechlorethamine oxide hydrochloride, melphalan, novembichin, phenesterine, prednimustine, tris(2-chloroethyl)amine, trofosfamide, and uracil mustard; alkyl sulfonates such as busulfan, improsulfan, and piposulfan; nitrosoureas such as carmustine, chlorozotocin, fotemustine, lomustine, nimustine, ranimustine, streptozotocin, and TA-07; ethylenimines and methylamelamines such as altretamine, thiotepa, triethylenemelamine, triethylenethiophosphaoramide, trietylenephosphoramide, and trimethylolomelamine; ambamustine; bendamustine; dacarbazine; etoglucid; irofulven; mafosfamide; mitobronitol; mitolactol; pipobroman; procarbazine; temozolomide; treosulfan; and triaziquone.

Non-limiting examples of anti-metabolites include folic acid analogues such as aminopterin, denopterin, edatrexate, methotrexate, pteropterin, raltitrexed, and trimetrexate; purine analogs such as 6-mercaptopurine, 6-thioguanine, fludarabine, forodesine, thiamiprine, and thioguanine; pyrimidine analogs such as 5-fluorouracil (5-FU), 6-azauridine, ancitabine, azacytidine, capecitabine, carmofur, cytarabine, decitabine, dideoxyuridine, doxifiuridine, doxifluridine, enocitabine, floxuridine, galocitabine, gemcitabine, and sapacitabine; 3-aminopyridine-2-carboxaldehyde thiosemicarbazone; broxuridine; cladribine; cyclophosphamide; cytarabine; emitefur; hydroxyurea; mercaptopurine; nelarabine; pemetrexed; pentostatin; tegafur; and troxacitabine.

Non-limiting examples of platinum analogs include carboplatin, cisplatin, dicycloplatin, heptaplatin, lobaplatin, nedaplatin, oxaliplatin, satraplatin, and triplatin tetranitrate.

Non-limiting examples of enzymes include asparaginase and pegaspargase.

Non-limiting examples of topoisomerase inhibitors include acridine carboxamide, amonafide, amsacrine, belotecan, elliptinium acetate, exatecan, indolocarbazole, irinotecan, lurtotecan, mitoxantrone, razoxane, rubitecan, SN-38, sobuzoxane, and topotecan.

Non-limiting examples of retinoids include alitretinoin, bexarotene, fenretinide, isotretinoin, liarozole, RII retinamide, and tretinoin.

Non-limiting examples of aziridines include benzodopa, carboquone, meturedopa, and uredopa.

Non-limiting examples of antibiotics include intercalating antibiotics; anthracenediones; anthracycline antibiotics such as aclarubicin, amrubicin, daunomycin, daunorubicin, doxorubicin, epirubicin, idarubicin, menogaril, nogalamycin, pirarubicin, and valrubicin; 6-diazo-5-oxo-L-norleucine; aclacinomysins; actinomycin; authramycin; azaserine; bleomycins; cactinomycin; calicheamicin; carabicin; carminomycin; carzinophilin; chromomycins; dactinomycin; detorubicin; esorubicin; esperamicins; geldanamycin; marcellomycin; mitomycins; mitomycin C; mycophenolic acid; olivomycins; novantrone; peplomycin; porfiromycin; potfiromycin; puromycin; quelamycin; rebeccamycin; rodorubicin; streptonigrin; streptozocin; tanespimycin; tubercidin; ubenimex; zinostatin; zinostatin stimalamer; and zorubicin.

In one embodiment, the additional anti-cancer agent(s) is a hormonal and/or anti-hormonal agent (i.e., hormone therapy). Non-limiting examples of hormonal and anti-hormonal agents include anti-androgens such as abiraterone, apalutamide, bicalutamide, darolutamide, enzalutamide, flutamide, goserelin, leuprolide, and nilutamide; anti-estrogens such as 4-hydroxy tamoxifen, aromatase inhibiting 4(5)-imidazoles, EM-800, fosfestrol, fulvestrant, keoxifene, LY 117018, onapristone, raloxifene, tamoxifen, toremifene, and trioxifene; anti-adrenals such as aminoglutethimide, dexaminoglutethimide, mitotane, and trilostane; androgens such as calusterone, dromostanolone propionate, epitiostanol, mepitiostane, and testolactone; abarelix; anastrozole; cetrorelix; deslorelin; exemestane; fadrozole; finasteride; formestane; histrelin (RL 0903); human chorionic gonadotropin; lanreotide; LDI 200 (Milkhaus); letrozole; leuprorelin; mifepristone; nafarelin; nafoxidine; osaterone; prednisone; thyrotropin alfa; and triptorelin.

In one embodiment, the additional anti-cancer agent(s) is an immunotherapeutic agent (i.e., immunotherapy). Non-limiting examples of immunotherapeutic agents include biological response modifiers, cytokine inhibitors, tumor vaccines, monoclonal antibodies, immune checkpoint inhibitors, colony-stimulating factors, and immunomodulators.

Non-limiting examples of biological response modifiers, including cytokine inhibitors (cytokines) such as interferons and interleukins, include interferon alfa/interferon alpha such as interferon alfa-2, interferon alfa-2a, interferon alfa-2b, interferon alfa-n1, interferon alfa-n3, interferon alfacon-1, peginterferon alfa-2a, peginterferon alfa-2b, and leukocyte alpha interferon; interferon beta such as interferon beta-1a, and interferon beta-1b; interferon gamma such as natural interferon gamma-1a, and interferon gamma-1b; aldesleukin; interleukin-1 beta; interleukin-2; oprelvekin; sonermin; tasonermin; and virulizin.

Non-limiting examples of tumor vaccines include APC 8015, AVICINE, bladder cancer vaccine, cancer vaccine (Biomira), gastrin 17 immunogen, Maruyama vaccine, melanoma lysate vaccine, melanoma oncolysate vaccine (New York Medical College), melanoma vaccine (New York University), melanoma vaccine (Sloan Kettering Institute), TICE® BCG (Bacillus Calmette-Guerin), and viral melanoma cell lysates vaccine (Royal Newcastle Hospital).

Non-limiting examples of monoclonal antibodies include abagovomab, adecatumumab, aflibercept, alemtuzumab, blinatumomab, brentuximab vedotin, C^{A} 125 MAb (Biomira), cancer MAb (Japan Pharmaceutical Development), daclizumab, daratumumab, denosumab, edrecolomab, gemtuzumab zogamicin, HER-2 and Fc MAb (Medarex), ibritumomab tiuxetan, idiotypic 105AD7 MAb (CRC Technology), idiotypic CEA MAb (Trilex), ipilimumab, lintuzumab, LYM-1-iodine 131 MAb (Techni clone), mitumomab, moxetumomab, ofatumumab, polymorphic epithelial mucin-yttrium 90 MAb (Antisoma), ranibizumab, rituximab, and trastuzumab.

Non-limiting examples of immune checkpoint inhibitors include anti-PD-1 agents or antibodies such as cemiplimab, nivolumab, and pembrolizumab; anti-PD-L1 agents or antibodies such as atezolizumab, avelumab, and durvalumab; anti-CTLA-4 agents or antibodies such as ipilumumab; anti-LAG1 agents; and anti-OX40 agents.

Non-limiting examples of colony-stimulating factors include darbepoetin alfa, epoetin alfa, epoetin beta, filgrastim, granulocyte macrophage colony stimulating factor, lenograstim, leridistim, mirimostim, molgramostim, nartograstim, pegfilgrastim, and sargramostim.

Non-limiting examples of additional immunotherapeutic agents include BiTEs, CAR-T cells, GITR agonists, imiquimod, immunomodulatory imides (IMiDs), mismatched double stranded RNA (Ampligen), resiquimod, SRL 172, and thymalfasin.

In one embodiment, the additional anti-cancer agent(s) is a targeted therapy agent (i.e., targeted therapy). Targeted therapy agents include, for example, monoclonal antibodies and small molecule drugs. Non-limiting examples of targeted therapy agents include signal transduction inhibitors, growth factor inhibitors, tyrosine kinase inhibitors, EGFR inhibitors, histone deacetylase (HDAC) inhibitors, proteasome inhibitors, cell-cycle inhibitors, angiogenesis inhibitors, matrix-metalloproteinase (MMP) inhibitors, hepatocyte growth factor inhibitors, TOR inhibitors, KDR inhibitors, VEGF inhibitors, fibroblast growth factors (FGF) inhibitors, MEK inhibitors, ERK inhibitors, PI3K inhibitors, AKT inhibitors, MCL-1 inhibitors, BCL-2 inhibitors, SHP2 inhibitors, HER-2 inhibitors, BRAF-inhibitors, BTK inhibitors (e.g., nemtabrutinib), gene expression modulators, autophagy inhibitors, apoptosis inducers, antiproliferative agents, and glycolysis inhibitors.

Non-limiting examples of signal transduction inhibitors include tyrosine kinase inhibitors, multiple-kinase inhibitors, anlotinib, avapritinib, axitinib, dasatinib, dovitinib, imatinib, lenvatinib, lonidamine, nilotinib, nintedanib, pazopanib, pegvisomant, ponatinib, vandetanib, and EGFR inhibitory agents.

Non-limiting examples of EGFR inhibitory agents include small molecule antagonists of EGFR such as afatinib, brigatinib, erlotinib, gefitinib, lapatinib, and osimertinib; and antibody-based EGFR inhibitors, including any anti-EGFR antibody or antibody fragment that can partially or completely block EGFR activation by its natural ligand. Antibody-based EGFR inhibitory agents may include, for example, those described in Modjtahedi, H., et al., 1993, Br. J. Cancer 67:247-253; Teramoto, T., et al., 1996, Cancer 77:639-645; Goldstein et al, 1995, Clin. Cancer Res. 1: 1311-1318; Huang, S. M., et al., 1999, Cancer Res. 15:59(8): 1935-40; and Yang, X., et al., 1999, Cancer Res. 59: 1236-1243; monoclonal antibody Mab E7.6.3 (Yang, 1999 supra); Mab C225 (ATCC Accession No. HB-8508), or an antibody or antibody fragment having the binding specificity thereof, specific antisense nucleotide or siRNA; afatinib, cetuximab; matuzumab; necitumumab; nimotuzumab; panitumumab; and zalutumumab.

Non-limiting examples of histone deacetylase (HDAC) inhibitors include belinostat, panobinostat, romidepsin, and vorinostat.

Non-limiting examples of proteasome inhibitors include bortezomib, carfilzomib, ixazomib, marizomib (salinosporamide a), and oprozomib.

Non-limiting examples of cell-cycle inhibitors, including CDK inhibitors, include abemaciclib, alvocidib, palbociclib, and ribociclib.

In one embodiment, the additional anti-cancer agent(s) is an anti-angiogenic agent (or angiogenesis inhibitor) including, but not limited to, matrix-metalloproteinase (MMP) inhibitors; VEGF inhibitors; EGFR inhibitors; TOR inhibitors such as everolimus and temsirolimus; PDGFR kinase inhibitory agents such as crenolanib; HIF-1α inhibitors such as PX 478; HIF-2α inhibitors such as belzutifan and the HIF-2α inhibitors described in WO 2015/035223; fibroblast growth factor (FGF) or FGFR inhibitory agents such as B-FGF and RG 13577; hepatocyte growth factor inhibitors; KDR inhibitors; anti-Ang1 and anti-Ang2 agents; anti-Tie2 kinase inhibitory agents; Tek antagonists (US 2003/0162712; U.S. Pat. No. 6,413,932); anti-TWEAK agents (U.S. Pat. No. 6,727,225); ADAM distintegrin domain to antagonize the binding of integrin to its ligands (US 2002/0042368); anti-eph receptor and/or anti-ephrin antibodies or antigen binding regions (U.S. Pat. Nos. 5,981,245; 5,728,813; 5,969,110; 6,596,852; 6,232,447; and 6,057,124); and anti-PDGF-BB antagonists as well as antibodies or antigen binding regions specifically binding to PDGF-BB ligands.

Non-limiting examples of matrix-metalloproteinase (MMP) inhibitors include MMP-2 (matrix-metalloproteinase 2) inhibitors, MMP-9 (matrix-metalloproteinase 9) inhibitors, prinomastat, RO 32-3555, and RS 13-0830. Examples of useful matrix metalloproteinase inhibitors are described, for example, in WO 96/33172, WO 96/27583, EP 1004578, WO 98/07697, WO 98/03516, WO 98/34918, WO 98/34915, WO 98/33768, WO 98/30566, EP 0606046, EP 0931788, WO 90/05719, WO 99/52910, WO 99/52889, WO 99/29667, WO 1999/007675, EP 1786785, EP 1181017, US 2009/0012085, U.S. Pat. Nos. 5,863,949, 5,861,510, and EP 0780386. Preferred MMP-2 and MMP-9 inhibitors are those that have little or no activity inhibiting MMP-1. More preferred, are those that selectively inhibit MMP-2 and/or MMP-9 relative to the other matrix-metalloproteinases (i.e., MAP-1, MMP-3, MMP-4, MMP-5, MMP-6, MMP-7, MMP-8, MMP-10, MMP-11, MMP-12, and MMP-13).

Non-limiting examples of VEGF and VEGFR inhibitory agents include bevacizumab, cediranib, CEP 7055, CP 547632, KRN 633, orantinib, pazopanib, pegaptanib, pegaptanib octasodium, semaxanib, sorafenib, sunitinib, VEGF antagonist (Borean, Denmark), and VEGF-TRAP™

The additional anti-cancer agent(s) may also be another anti-angiogenic agent including, but not limited to, 2-methoxyestradiol, AE 941, alemtuzumab, alpha-D148 Mab (Amgen, US), alphastatin, anecortave acetate, angiocidin, angiogenesis inhibitors, (SUGEN, US), angiostatin, anti-Vn Mab (Crucell, Netherlands), atiprimod, axitinib, AZD 9935, BAY RES 2690 (Bayer, Germany, BC 1 (Genoa Institute of Cancer Research, Italy), beloranib, benefin (Lane Labs, US), cabozantinib, CDP 791 (Celltech Group, UK), chondroitinase AC, cilengitide, combretastatin A4 prodrug, CP 564959 (OSI, US), CV247, CYC 381 (Harvard University, US), E 7820, EHT 0101, endostatin, enzastaurin hydrochloride, ER-68203-00 (IVAX, US), fibrinogen-E fragment, Flk-1 (ImClone Systems, US), forms of FLT 1 (VEGFR 1), FR-I 11142, GCS-100, GW 2286 (GlaxoSmithKline, UK), TL-8, ilomastat, IM-862, irsogladine, KM-2550 (Kyowa Hakko, Japan), lenalidomide, lenvatinib, MAb alpha5beta3 integrin, second generation (Applied Molecular Evolution, USA and Medlmmune, US), MAb VEGF (Xenova, UK), marimastat, maspin (Sosei, Japan), metastatin, motuporamine C, M-PGA, ombrabulin, OXI4503, PI 88, platelet factor 4, PPI 2458, ramucirumab, rBPI 21 and BPI-derived antiangiogenic (XOMA, US), regorafenib, SC-236, SD-7784 (Pfizer, US), SDX 103 (University of California at San Diego, US), SG 292 (Telios, US), SU-0879 (Pfizer, US), TAN-1120, TBC-1635, tesevatinib, tetrathiomolybdate, thalidomide, thrombospondin 1 inhibitor, Tie-2 ligands (Regeneron, US), tissue factor pathway inhibitors (EntreMed, US), tumor necrosis factor-alpha inhibitors, tumstatin, TZ 93, urokinase plasminogen activator inhibitors, vadimezan, vandetanib, vasostatin, vatalanib, VE-cadherin-2 antagonists, xanthorrhizol, XL 784 (Exelixis, US), ziv-aflibercept, and ZD 6126.

In embodiments, the additional anti-cancer agent(s) is an additional active agent that disrupts or inhibits RAS-RAF-ERK or PI3K-AKT-TOR signaling pathways or is a PD-1 and/or PD-L1 antagonist. In embodiments, the additional anti-cancer agent(s) is a RAF inhibitor, EGFR inhibitor, MEK inhibitor, ERK inhibitor, PI3K inhibitor, AKT inhibitor, TOR inhibitor, MCL-1 inhibitor, BCL-2 inhibitor, SHP2 inhibitor, proteasome inhibitor, or immune therapy, including monoclonal antibodies, immunomodulatory imides (IMiDs), anti-PD-1, anti-PDL-1, anti-CTLA4, anti-LAGl, and anti-OX40 agents, GITR agonists, CAR-T cells, and BiTEs.

Non-limiting examples of RAF inhibitors include dabrafenib, encorafenib, regorafenib, sorafenib, and vemurafenib.

Non-limiting examples of MEK inhibitors include binimetinib, CI-1040, cobimetinib, PD318088, PD325901, PD334581, PD98059, refametinib, selumetinib, and trametinib.

Non-limiting examples of ERK inhibitors include LY3214996, LTT462, MK-8353, SCH772984, ravoxertinib, ulixertinib, and an ERKi as described in WO 2017/068412.

Non-limiting examples of PI3K inhibitors include 17-hydroxywortmannin analogs (e.g., WO 06/044453); AEZS-136; alpelisib; AS-252424; buparlisib; CAL263; copanlisib; CUDC-907; dactolisib (WO 06/122806); demethoxyviridin; duvelisib; GNE-477; GSK1059615; IC87114; idelalisib; INK1117; LY294002; Palomid 529; paxalisib; perifosine; PI-103; PI-103 hydrochloride; pictilisib (e.g., WO 09/036,082; WO 09/055,730); PIK 90; PWT33597; SF1126; sonolisib; TGI 00-115; TGX-221; XL147; XL-765; wortmannin; and ZSTK474.

Non-limiting examples of AKT inhibitors include Akt-1-1 (inhibits Aktl) (Barnett et al. (2005) Biochem. J., 385 (Pt. 2), 399-408); Akt-1-1,2 (Barnett et al. (2005) Biochem. J. 385 (Pt. 2), 399-408); API-59CJ-Ome (e.g., Jin et al. (2004) Br. J. Cancer 91, 1808-12); 1-H-imidazo[4,5-c]pyridinyl compounds (e.g., WO05011700); indole-3-carbinol and derivatives thereof (e.g., U.S. Pat. No. 6,656,963; Sarkar and Li (2004) J Nutr. 134(12 Suppl), 3493S-3498S); perifosine, Dasmahapatra et al. (2004) Clin. Cancer Res. 10(15), 5242-52, 2004); phosphatidylinositol ether lipid analogues (e.g., Gills and Dennis (2004) Expert. Opin. Investig. Drugs 13, 787-97); triciribine (Yang et al. (2004) Cancer Res. 64, 4394-9); imidazooxazone compounds including trans-3-amino-1-methyl-3-[4-(3-phenyl-5H-imidazo[1,2-c]pyrido[3,4-e][1,3]oxazin-2-yl)phenyl]-cyclobutanol hydrochloride (WO 2012/137870); afuresertib; capivasertib; MK2206; patasertib, and those disclosed in WO 2011/082270 and WO 2012/177844.

Non-limiting examples of TOR inhibitors include deforolimus; ATP-competitive TORC1/TORC2 inhibitors, including PI-103, PP242, PP30, and Torin 1; TOR inhibitors in FKBP12 enhancer, rapamycins and derivatives thereof, including temsirolimus, everolimus, WO 9409010; rapalogs, e.g. as disclosed in WO 98/02441 and WO 01/14387, e.g. AP23573, AP23464, or AP23841; 40-(2-hydroxyethyl)rapamycin, 40-[3-hydroxy(hydroxymethyl)methylpropanoate]-rapamycin; 40-epi-(tetrazolyl)-rapamycin (also called ABT578); 32-deoxorapamycin; 16-pentynyloxy-32(S)-dihydrorapanycin, and other derivatives disclosed in WO 05/005434; derivatives disclosed in U.S. Pat. No. 5,258,389, WO 94/090101, WO 92/05179, U.S. Pat. Nos. 5,118,677, 5,118,678, 5,100,883, 5,151,413, 5,120,842, WO 93/111130, WO 94/02136, WO 94/02485, WO 95/14023, WO 94/02136, WO 95/16691, WO 96/41807, WO 96/41807 and U.S. Pat. No. 5,256,790; and phosphorus-containing rapamycin derivatives (e.g., WO 05/016252).

Non-limiting examples of MCL-1 inhibitors include AMG-176, MIK665, and S63845.

Non-limiting examples of SHP2 inhibitors include SHP2 inhibitors described in WO 2019/167000 and WO 2020/022323.

Additional non-limiting examples of anti-cancer agents that are suitable for use include 2-ethylhydrazide, 2,2′,2″-trichlorotriethylamine, ABVD, aceglatone, acemannan, aldophosphamide glycoside, alpharadin, amifostine, aminolevulinic acid, anagrelide, ANCER, ancestim, anti-CD22 immunotoxins, antitumorigenic herbs, apaziquone, arglabin, arsenic trioxide, azathioprine, BAM 002 (Novelos), bcl-2 (Genta), bestrabucil, biricodar, bisantrene, bromocriptine, brostallicin, bryostatin, buthionine sulfoximine, calyculin, cell-cycle nonspecific antineoplastic agents, celmoleukin, clodronate, clotrimazole, cytarabine ocfosfate, DA 3030 (Dong-A), defofamine, denileukin diftitox, dexrazoxane, diaziquone, dichloroacetic acid, dilazep, discodermolide, docosanol, doxercalciferol, edelfosine, eflornithine, EL532 (Elan), elfomithine, elsamitrucin, eniluracil, etanidazole, exisulind, ferruginol, folic acid replenisher such as frolinic acid, gacytosine, gallium nitrate, gimeracil/oteracil/tegafur combination (S-1), glycopine, histamine dihydrochloride, HIT diclofenac, HLA-B7 gene therapy (Vical), human fetal alpha fetoprotein, ibandronate, ibandronic acid, ICE chemotherapy regimen, imexon, iobenguane, IT-101 (CRLX101), laniquidar, LC 9018 (Yakult), leflunomide, lentinan, levamisole+fluorouracil, lovastatin, lucanthone, masoprocol, melarsoprol, metoclopramide, miltefosine, miproxifene, mitoguazone, mitozolomide, mopidamol, motexafin gadolinium, MX6 (Galderma), naloxone+pentazocine, nitracrine, nolatrexed, NSC 631570 octreotide (Ukrain), olaparib, P-30 protein, PAC-1, palifermin, pamidronate, pamidronic acid, pentosan polysulfate sodium, phenamet, picibanil, pixantrone, platinum, podophyllinic acid, porfimer sodium, PSK (Polysaccharide-K), rabbit antithymocyte polyclonal antibody, rasburiembodiment, retinoic acid, rhenium Re 186 etidronate, romurtide, samarium (153 Sm) lexidronam, sizofiran, sodium phenylacetate, sparfosic acid, spirogermanium, strontium-89 chloride, suramin, swainsonine, talaporfin, tariquidar, tazarotene, tegafur-uracil, temoporfin, tenuazonic acid, tetrachlorodecaoxide, thrombopoietin, tin ethyl etiopurpurin, tirapazamine, TLC ELL-12, tositumomab-iodine 131, trifluridine and tipiracil combination, troponin I (Harvard University, US), urethan, valspodar, verteporfin, zoledronic acid, and zosuquidar.

The present disclosure further provides a method for using the compounds of Formula (I) or pharmaceutical compositions provided herein, in combination with radiation therapy to treat cancer. Techniques for administering radiation therapy are known in the art, and these techniques can be used in the combination therapy described herein. The administration of the compound of Formula (I) in this combination therapy can be determined as described herein.

Radiation therapy can be administered through one of several methods, or a combination of methods, including, without limitation, external-beam therapy, internal radiation therapy, implant radiation, stereotactic radiosurgery, systemic radiation therapy, radiotherapy and permanent or temporary interstitial brachy therapy. The term “brachytherapy,” as used herein, refers to radiation therapy delivered by a spatially confined radioactive material inserted into the body at or near a tumor or other proliferative tissue disease site. The term is intended, without limitation, to include exposure to radioactive isotopes (e.g., At-211, I-131, I-125, Y-90, Re-186, Re-188, Sm-153, Bi-212, P-32, and radioactive isotopes of Lu). Suitable radiation sources for use as a cell conditioner of the present disclosure include both solids and liquids. By way of non-limiting example, the radiation source can be a radionuclide, such as I-125, I-131, Yb-169, Ir-192 as a solid source, I-125 as a solid source, or other radionuclides that emit photons, beta particles, gamma radiation, or other therapeutic rays. The radioactive material can also be a fluid made from any solution of radionuclide(s), e.g., a solution of I-125 or I-131, or a radioactive fluid can be produced using a slurry of a suitable fluid containing small particles of solid radionuclides, such as Au-198, Y-90. Moreover, the radionuclide(s) can be embodied in a gel or radioactive microspheres.

The present disclosure also provides methods for combination therapies in which the additional active agent is known to modulate other pathways, or other components of the same pathway, or even overlapping sets of target enzymes which are used in combination with a compound of Formula (I), or a pharmaceutically acceptable salt thereof. In one embodiment, such therapy includes, but is not limited to, the combination of one or more compounds of Formula (I) with chemotherapeutic agents, immunotherapeutic agents, hormonal therapy agents, therapeutic antibodies, targeted therapy agents, and radiation treatment, to provide a synergistic or additive therapeutic effect.

The compounds of the disclosure can be used in combination with the agents disclosed herein or other suitable agents, depending on the condition being treated. Hence, in some embodiments the one or more compounds of the disclosure will be co-administered with other agents as described above. When used in combination therapy, the compounds described herein are administered with the second agent simultaneously or separately. This administration in combination can include simultaneous administration of the two agents in the same dosage form, simultaneous administration in separate dosage forms, and separate administration. That is, a compound of Formula (I) and any of the agents described above can be formulated together in the same dosage form and administered simultaneously. Alternatively, a compound of Formula (I) and any of the agents described above can be simultaneously administered, wherein both the agents are present in separate formulations. In another alternative, a compound of Formula (I) can be administered just followed by and any of the agents described above, or vice versa. In some embodiments of the separate administration protocol, a compound of Formula (I) and any of the agents described above are administered a few minutes apart, or a few hours apart, or a few days apart.

As one aspect of the present disclosure contemplates the treatment of the disease/conditions with a combination of pharmaceutically active compounds that may be administered separately, the disclosure further relates to combining separate pharmaceutical compositions in kit form. The kit comprises two separate pharmaceutical compositions: a compound of Formula (I), and a second pharmaceutical compound. The kit comprises a container for containing the separate compositions such as a divided bottle or a divided foil packet. Additional examples of containers include syringes, boxes, and bags. In some embodiments, the kit comprises directions for the use of the separate components. The kit form is particularly advantageous when the separate components are preferably administered in different dosage forms (e.g., oral and parenteral), are administered at different dosage intervals, or when titration of the individual components of the combination is desired by the prescribing health care professional.

The present disclosure also provides for the compound of Formula (I), or the pharmaceutically acceptable salt thereof, for use in therapy, or use of the compound of Formula (I), or the pharmaceutically acceptable salt thereof, in therapy. The present disclosure also provides for the compound of Formula (I), or the pharmaceutically acceptable salt thereof, for use in treating cancer, or use of a compound of Formula (I), or the pharmaceutically acceptable salt thereof, for treating cancer. The present disclosure also provides for the compound of Formula (I), or the pharmaceutically acceptable salt thereof, for the preparation of a medicament for the treatment of cancer, or use of the compound of Formula (I), or the pharmaceutically acceptable salt thereof, for the preparation of a medicament for the treatment of cancer. The present disclosure also provides for the compound of Formula (I), or the pharmaceutically acceptable salt thereof, and an additional anti-cancer agent, for use in the treatment of cancer, or use of the compound of Formula (I), or the pharmaceutically acceptable salt thereof, and the additional anti-cancer agent for treating cancer. The disclosure also provides the compound of Formula (I), or the pharmaceutically acceptable salt thereof, and an additional anti-cancer agent, for the preparation of a medicament for the treatment of cancer, or use of the compound of Formula (I), or the pharmaceutically acceptable salt thereof, and the additional anti-cancer agent, for the preparation of a medicament for the treatment of cancer. The present disclosure also provides for a pharmaceutical composition comprising the compound of Formula (I), or the pharmaceutically acceptable salt thereof, for use in the treatment of cancer, or use of the pharmaceutical composition comprising the compound of Formula (I), or the pharmaceutically acceptable salt thereof, for treating cancer. The present disclosure also provides for a pharmaceutical composition comprising the compound of Formula (I), or the pharmaceutically acceptable salt thereof, and an additional anti-cancer agent, for use in the treatment of cancer, or use of the pharmaceutical composition comprising the compound of Formula (I), or the pharmaceutically acceptable salt thereof, and the additional anti-cancer agent, for treating cancer.

### Methods of Preparing the Compounds of the Disclosure

The compounds described herein can be prepared according to the procedures of the following schemes and examples, using appropriate materials and are further exemplified by the following specific examples. The compounds illustrated in the examples are not, however, to be construed as forming the only genus that is considered as the disclosure. The examples further illustrate details for the preparation of the compounds of the present disclosure. Those skilled in the art will readily understand that known variations of the conditions and processes of the following preparative procedures can be used to prepare these compounds. For instance, in some cases, the order of carrying out the steps of reaction schemes may be varied to facilitate the reaction or to avoid unwanted reaction products. These examples are provided for the purpose of further illustration only and are not intended to be limitations on the disclosure. Any intermediates described below may be referred to herein by their number preceded by “Int-.”

Throughout the synthetic schemes and examples, abbreviations and acronyms may be used with the following meanings unless otherwise indicated: Ac=acetyl; AcO=acetate; AcOH=acetic acid; aq.=aqueous; atm=atmosphere; Bn=benzyl; Boc=tert-butyloxycarbonyl; Bodipy-GDP=mixture of ((2R,3S,4R,5R)-5-(2-amino-6-oxo-1,6-dihydro-9H-purin-9-yl)-3-(((2-(3-(5,5-difluoro-7,9-dimethyl-5H-414,514-dipyrrolo[1,2-c:2′,1′-f][1,3,2]diazaborinin-3-yl)propanamido)ethyl)carbamoyl)oxy)-4-hydroxytetrahydrofuran-2-yl)methyl hydrogen diphosphate and ((2R,3R,4R,5R)-5-(2-amino-6-oxo-1,6-dihydro-9H-purin-9-yl)-4-(((2-(3-(5,5-difluoro-7,9-dimethyl-5H-414,514-dipyrrolo[1,2-c:2′,1′-f][1,3,2]diazaborinin-3-yl)propanamido)ethyl)carbamoyl)oxy)-3-hydroxytetrahydrofuran-2-yl)methyl hydrogen diphosphate (Invitrogen™, catalog number G22360); Boc₂O=di-tert-butyl dicarbonate; BOP=benzotriazol-1-yloxytris(dimethylamino)phosphonium hexafluorophosphate; B₂pin₂=bis(pinacolato)diboron; br s=broad singlet; Bu=butyl; tBu=tert-butyl; tBuO=tert-butoxide; cat.=catalyst; CDCl₃=deuterated chloroform; conc.=concentrated; CPhos Pd G3=[(2-dicyclohexylphosphino-2′,6′-bis(N,N-dimethylamino)-1,1′-biphenyl)-2-(2′-amino-1,1′-biphenyl)]palladium(II) methanesulfonate; CPhos Pd G4=Palladium, [2′-(dicyclohexylphosphino-wP)-N2,N2,N6,N6-tetramethyl[1,1′-biphenyl]-2,6-diamine](methanesulfonato-κO)[2′-(methylamino-κN)[1,1′-biphenyl]-2-yl-κC]-; DABCO=1,4-diazabicyclo[2.2.2]octane; DAST=(diethylamino)sulfur trifluoride; DCM=dichloromethane; DHP=3,4-dihydropyran; DIEA/DIPEA=N,N-diisopropylethylamine; DMA=N,N-dimethylacetamide; DMAP=dimethylaminopyridine; DMF=N,N-dimethylformamide; DMSO=dimethylsulfoxide; dppf=1,1′-bis(diphenylphosphino)ferrocene; DPEphosPdCl₂=dichlorobis(diphenylphosphinophenyl)ether palladium(II)=dichloro[bis(2-(diphenylphosphino)phenyl)ether]palladium(II); EDTA=ethylenediaminetetraacetic acid; equiv, eq.=equivalent(s); Et=ethyl; EtOAc=ethyl acetate; EtOH=ethanol; GDP=guanosine diphosphate; GTP=guanosine triphosphate; h=hour; HEPES=4-(2-hydroxyethyl)-1-piperazineethanesulfonic acid; HMDS=hexamethydisilazane; HPLC=High pressure liquid chromatography; Int=intermediate; LAH=lithium aluminum hydride; LCMS, LC/MS=liquid chromatography-mass spectrometry; min=minute; LDA=lithium diisopropylamide; LiHMDS=Lithium bis(trimethylsilyl)amide; M=Molar; m-CPBA or mCPBA=3-chlorobenzoperoxoic acid=m-chloroperoxybenzoic acid; Me=methyl; MeCN, ACN=acetonitrile; MeOH=methanol; MS=mass spectrometry; MsCl=methanesulfonyl chloride; Ms₂O=methanesulfonic anhydride; MTBE=methyl tert-butyl ether; N=Normal; NBS=N-bromosuccinimide; NCS=N-chlorosuccinimide; NMP=N-methyl-2-pyrrolidone; NMR=nuclear magnetic resonance; PMB=4-methoxybenzyl; Pet. ether=petroleum ether; Pd/C=palladium on carbon; Ph=phenyl; psi=pounds per square inch gauge; POCl₃=phosphorus(V) oxide chloride; PTLC, prep TLC=preparative thin layer chromatography; rac=racemic; RT=retention time; RP-HPLC=reverse phase HPLC; rt/RT=room temperature; RuPhos Pd G2=chloro(2-dicyclohexylphosphino-2′,6′-diisopropoxy-1,1′-biphenyl)[2-(2′-amino-1,1′-biphenyl)]palladium(II); sat.=saturated; SCN—CO₂Et=ethoxycarbonyl isothiocyanate; SFC=supercritical fluid chromatography; SOS=Son of Sevenless; TBAF=tetra-n-butylammonium fluoride; TBS=tert-butyldimethylsilyl; TEA=triethylamine; Tf₂O=trifluoromethanesulfonic anhydride; TFA=trifluoroacetic acid; THP=tetrahydropyran; TLC=thin layer chromatography; THE=tetrahydrofuran; TLC=thin layer chromatography; TMP=2,2,6,6-tetramethylpiperdine; Tos=4-toluenesulfonyl; TR-FRET=time-resolved fluorescence resonance energy transfer; TsOH=p-toluenesulfonic acid; TWEEN=polyoxyethylene (20) sorbitan monolaurate; VCD=vibrational circular dichroism; v, v/v=volume, volume to volume; w, w/w=weight, weight to weight, pm=micrometer.

### EXAMPLES

Concentration refers to the removal of the volatile components at reduced pressure (e.g., by rotary evaporation) unless otherwise noted. All temperatures are in degrees Celsius unless otherwise noted. Mass spectra (MS) were measured by electrospray ion-mass spectroscopy (ESI) in positive ion detection mode and m/z refers to the [M+H]⁺ ion unless otherwise noted. ¹H NMR spectra were recorded at 400-600 MHz at ambient temperature unless otherwise noted. Protons reported as 0.5 H are due to rotameric signals. RP-HPLC refers to reverse-phase HPLC on C18-functionalized preparative or semi-preparative columns with gradient elution using acetonitrile and water modified with trifluoroacetic acid or ammonium hydroxide as eluents and fractions were lyophilized or concentrated by rotary evaporation unless otherwise noted. Purification by column chromatography on silica gel was accomplished using a flash chromatography system (e.g., ISCO® or Biotage®) and commercial pre-packed silica gel columns with elution using the stated solvent systems. Compounds described herein were synthesized as the racemates unless otherwise noted in the experimental procedures and compound tables. Certain products/intermediates in the examples include indication of “Peak 1” and/or “Peak 2”, which refer to the order of elution of the indicated product/intermediate from the chromatography column (e.g., an SFC column) used to isolate the compound under the specified conditions. Thus, for example, Peak 1 refers to the first eluting compound, e.g., first eluting stereoisomer, under the specified conditions.

SFC Columns used in the chiral resolution of stereoisomers are summarized in the following Table:

| | SFC Column |
|---|---|
| SFC Column | Abbreviation |
| Daicel Chiralpak IC (21 mm × 250 mm, 5 um) | Column A |
| AD-H (21 mm × 250 mm, 5 um) | Column B |
| Phenomenex, LUX-2 (21 mm × 250 mm, 5 um) | Column C |
| Phenomenex, Cellulose-2 (25 mm × 250 mm, 10 um) | Column D |
| Daicel Chiralpak AS (50 mm × 250 mm, 10 um) | Column E |
| Regis, (R, R) Whelk-O1 (50 mm × 250 mm, 10 um) | Column F |
| Daicel Chiralpak AD (30 mm × 250 mm, 10 um) | Column G |
| Daicel Chiralpak OJ-H (21 mm × 250 mm, 5 um) | Column H |
| Phenomenex, LUX-4 (21 mm × 250 mm, 5 um) | Column I |
| Daicel Chiralpak AS (50 mm × 250 mm, 10 um) | Column J |
| Daicel Chiralpak IG (30 mm × 250 mm, 10 um) | Column K |
| Chiral Technologies, IB-N (21 mm × 250 mm, 5 um) | Column L |
| Daicel Chiralpak IA (21 mm × 250 mm, 5 um) | Column M |
| ES Industries, CCAF4 (21 mm × 250 mm, 5 um) | Column N |
| Phenomenex, Cellulose-2 (30 mm × 250 mm, 5 um) | Column O |
| Daicel Chiralpak IG-3 (4.6 mm × 100 mm, 3 um) | Column P |
| CCA (21 mm × 250 mm, 5 um) | Column Q |
| CCO (21 mm × 250 mm, 5 um) | Column R |
| CCC (21 mm × 250 mm, 5 um) | Column S |
| Daicel Chiralpak IC (30 mm × 250 mm, 10 um) | Column T |
| Chiral SFC OD-H (21 mm × 250 mm × 5 um) | Column U |
| SJ (21 mm × 250 mm × 5 um) | Column V |
| Phenomenex-Cellulose-2 (30 mm × 250 mm, 10 um) | Column W |

### Intermediates

### ((2R,7aS)-2-fluorotetrahydro-1H-pyrrolizin-7a(5H)-yl)methanol (Int-ag)

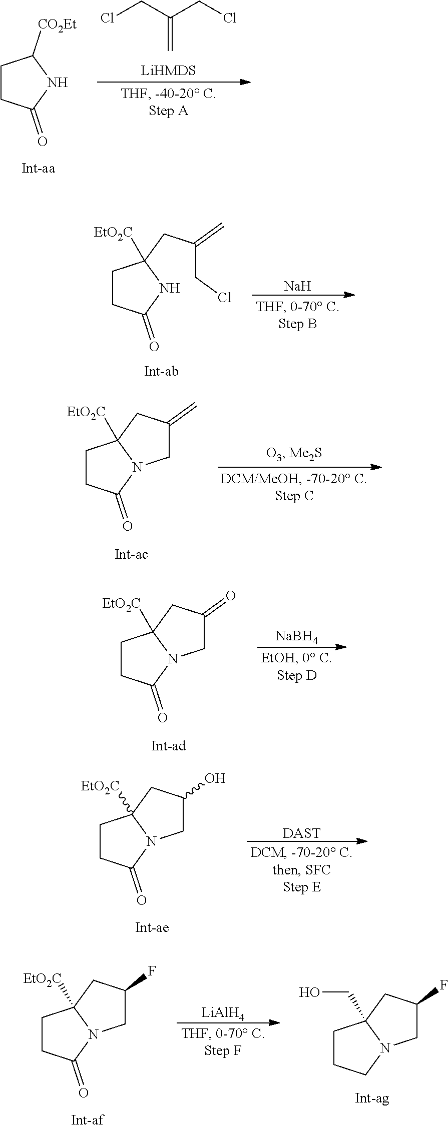

### Step A: Ethyl 2-(2-(chloromethyl)allyl)-5-oxopyrrolidine-2-carboxylate (Int-ab)

LiHMDS (1.00 M, 2.55 L) was added dropwise to a solution of ethyl 5-oxopyrrolidine-2-carboxylate (Int-aa) (200 g, 1.27 mol) and 3-chloro-2-(chloromethyl)prop-1-ene (255 g, 2.04 mol, 236 mL) in THF (2.00 L) at −40° C. under N₂. The mixture was stirred at 20° C. for 20 h. The reaction mixture was poured into sat. NH₄Cl solution (1.00 L), and the pH of the mixture was adjusted to 6-7 with 1 N HCl. The biphasic solution was extracted with EtOAc (500 mL×3). The organic layers were combined, washed with brine (600 mL), and concentrated under reduced pressure to give a crude residue. The crude material was purified by silica gel column chromatography (eluent:petroleum ether:ethyl acetate=50:1 to 1:1 gradient) to yield ethyl 2-(2-(chloromethyl)allyl)-5-oxopyrrolidine-2-carboxylate (Int-ab). ¹H NMR (400 MHz, CDCl₃) δ 5.06 (br d, J=17 Hz, 2H), 4.14-4.38 (m, 3H), 3.73 (br d, J=16 Hz, 1H), 3.06 (br d, J=16 Hz, 1H), 2.70-2.85 (m, 1H), 2.53-2.66 (m, 1H), 2.36-2.50 (m, 2H), 2.09-2.21 (m, 1H), 1.23-1.31 (m, 3H).

### Step B: Ethyl 2-methylene-5-oxotetrahydro-1H-pyrrolizine-7a(5H)-carboxylate (Int-ac)

A solution of ethyl 2-(2-(chloromethyl)allyl)-5-oxopyrrolidine-2-carboxylate (Int-ab) (500 g, 2.03 mol) in THE (500 mL) was added dropwise to a mixture of sodium hydride (97.7 g, 2.44 mol, 60.0% wt %) in THE (3.00 L) at 0° C. under nitrogen. The reaction mixture was stirred at 70° C. for 12 h under nitrogen. The reaction mixture was cooled and poured into sat. NH₄Cl solution (2.00 L) and stirred at 5° C. for 1 h. The biphasic mixture was extracted with EtOAc (600 mL×3). The combined organic layers were washed with brine (500 mL×2), dried over anhydrous Na₂SO₄, filtered, and concentrated under reduced pressure. The crude residue was purified by silica gel column chromatography (eluent: Petroleum ether:Ethyl acetate=50:1 to 1:1) to yield ethyl 2-methylene-5-oxotetrahydro-1H-pyrrolizine-7a(5H)-carboxylate (Int-ac). ¹H NMR (400 MHz, CDCl₃) δ 4.96-5.13 (m, 2H), 4.27 (br d, J=16 Hz, 1H), 4.19 (q, J=7 Hz, 2H), 3.71 (br d, J=16 Hz, 1H), 3.04 (d, J=16 Hz, 1H), 2.69-2.83 (m, 1H), 2.59 (ddd, J=2, 9, 13 Hz, 1H), 2.40-2.52 (m, 2H), 1.96-2.22 (m, 1H), 1.26 (t, J=7 Hz, 3H).

### Step C: Ethyl 2,5-dioxotetrahydro-1H-pyrrolizine-7a(5H)-carboxylate (Int-ad)

Ozone (239 mmol) (0.5-1 m³/h) was bubbled into a solution of ethyl 2-methylene-5-oxotetrahydro-1H-pyrrolizine-7a(5H)-carboxylate (Int-ac) (160 g, 765 mmol in DCM (1.60 L) and MeOH (160 mL) at −70° C. for 9 h. Nitrogen was bubbled through the reaction mixture to purge excess ozone. Then, dimethyl sulfide (76.0 g, 1.22 mol) was added to the mixture at −70° C. The reaction mixture was stirred at 20° C. for 14 h. The reaction mixture was concentrated under reduced pressure to give a residue. The crude residue was purified by silica gel column chromatography (eluent:Petroleum ether:Ethyl acetate=50:1 to 1:1) to yield ethyl 2,5-dioxotetrahydro-1H-pyrrolizine-7a(5H)-carboxylate (Int-ad). ¹H NMR (400 MHz, CDCl₃) δ 4.22 (q, J=7 Hz, 2H), 4.07-4.12 (m, 1H), 3.54 (dd, J=1, 18 Hz, 1H), 2.92-3.03 (m, 2H), 2.74-2.88 (m, 1H), 2.42-2.51 (m, 2H), 2.12-2.23 (m, 1H), 1.27 (t, J=7 Hz, 3H).

### Step D: Ethyl 2-hydroxy-5-oxotetrahydro-1H-pyrrolizine-7a(5H)-carboxylate (Int-ae)

To a solution of ethyl 2,5-dioxotetrahydro-1H-pyrrolizine-7a(5H)-carboxylate (Int-ad) (200 g, 947 mmol) in EtOH (2.00 L) at 0° C. under N₂ was added NaBH₄ (10.8 g, 284 mmol). The reaction mixture was stirred at 0° C. for 10 min. The reaction mixture was quenched by addition of sat. NH₄Cl (50.0 mL) at 5° C., and the mixture was stirred at 5° C. for 0.5 h. The reaction mixture was concentrated under reduced pressure. The crude residue was purified by silica gel column chromatography (eluent: Petroleum ether:Ethyl acetate=50:1 to 1:1) to yield ethyl 2-hydroxy-5-oxotetrahydro-1H-pyrrolizine-7a(5H)-carboxylate (Int-ae). ¹H NMR (400 MHz, CDCl₃) δ 4.54-4.70 (m, 1H), 4.16-4.31 (m, 2H), 3.93 (dd, J=6, 13 Hz, 1H), 3.09 (d, J=13 Hz, 1H), 2.75-2.90 (m, 1H), 2.39-2.63 (m, 4H), 2.01-2.13 (m, 1H), 1.83 (dd, J=6, 14 Hz, 1H), 1.29 (t, J=7 Hz, 3H).

### Step E: Ethyl (2R,7aS)-2-fluoro-5-oxotetrahydro-1H-pyrrolizine-7a(5H)-carboxylate (Int-af)

To a solution of ethyl 2-hydroxy-5-oxotetrahydro-1H-pyrrolizine-7a(5H)-carboxylate (Int-ae) (100 g, 468 mmol) in DCM (1 L) was added DAST (113 g, 703 mmol, 93 mL) dropwise at −70° C. under N₂. The reaction mixture was warmed to 20° C. and stirred for 16 h. The reaction was quenched by the addition of EtOH (50.0 mL) at 10° C., and then diluted with water (300 mL) and extracted with DCM (200 mL×2). The combined organic layers were washed with brine (200 mL), dried over anhydrous Na₂SO₄, filtered, and concentrated under reduced pressure. The crude product was combined from six identical reactions and purified by silica gel column chromatography (Petroleum ether:Ethyl Acetate=50:1 to 1:1). This material was further purified by prep-HPLC (C18, 0-100% MeCN/water with 0.05% HCl). The racemic mixture was resolved using chiral SFC (Column E; EtOH with 0.1% NH₄OH) to yield ethyl (2R,7aS)-2-fluoro-5-oxotetrahydro-1H-pyrrolizine-7a(5H)-carboxylate (Int-af, Peak 2). ¹H NMR (400 MHz, CDCl₃): δ 5.16-5.43 (m, 1H), 4.14-4.27 (m, 3H), 3.06-3.26 (m, 1H), 2.57-2.85 (m, 3H), 2.38-2.50 (m, 1H), 2.07-2.30 (m, 2H), 1.28 (t, J=7 Hz, 3H).

### Step F: ((2R,7aS)-2-fluorotetrahydro-1H-pyrrolizin-7a(5H)-yl)methanol (Int-ag)

A solution of ethyl (2R,7aS)-2-fluoro-5-oxotetrahydro-1H-pyrrolizine-7a(5H)-carboxylate (Int-af)(82.0 g, 381 mmol) in THE (300 mL) was added to the mixture of LAH (21.7 g, 571 mmol) in THE (520 mL) at 0° C. under nitrogen. The reaction mixture was warmed to 70° C. and stirred for 3 h. The reaction mixture was cooled to 0° C. and quenched by the addition of Na₂SO₄·10 H₂O at 0° C. under nitrogen. The reaction mixture was stirred at 20° C. for 0.5 h and then filtered. The filter cake was washed with EtOAc (600 mL×5), and the filtrate was dried over anhydrous Mg₂SO₄. The mixture was filtered and the filtrate concentrated under reduced pressure to give a residue. The crude residue was purified by silica gel column chromatography (SiO₂, DCM:Methanol=100:1 to 10:1) to give ((2R,7aS)-2-fluorotetrahydro-1H-pyrrolizin-7a(5H)-yl)methanol (Int-ag). ¹H NMR (400 MHz, CDCl₃): δ 5.06-5.34 (m, 1H), 3.25 (s, 2H), 3.08-3.23 (m, 3H), 2.85-3.08 (m, 2H), 2.00-2.12 (m, 2H), 1.74-1.93 (m, 4H).

### (R)-(1-((benzyloxy)methyl)-2,2-difluorocyclopropyl)methanol (Int-bf)

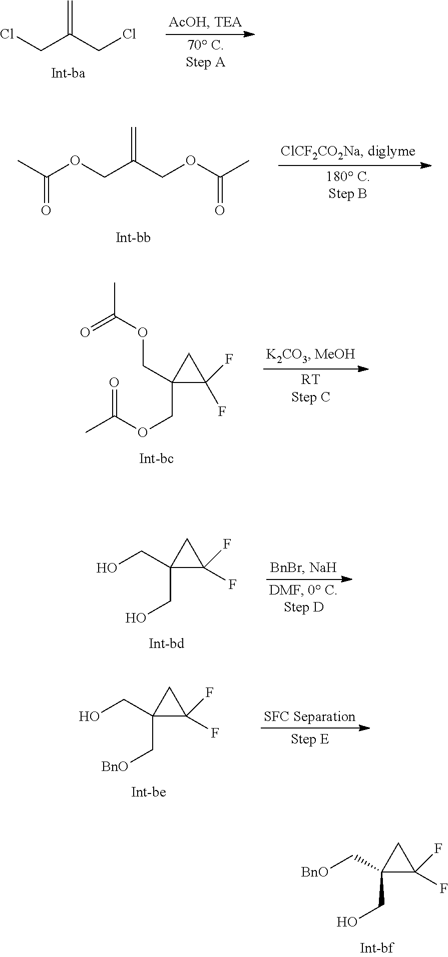

### Step A: 2-[(acetyloxy)methyl]prop-2-en-1-yl acetate (Int-bb)

A 5 L 4-necked round-bottom flask was charged with 3-chloro-2-(chloromethyl)prop-1-ene (Int-ba) (600. g, 4.80 mol), triethylamine (1.46 kg, 14.4 mol), and acetic acid (721 g, 12.0 mol). The resulting solution was stirred overnight at 70° C. The reaction mixture was cooled to room temperature and quenched by the addition of 3 L of water. The resulting solution was extracted with ethyl acetate (3×1 L), and the combined organic layers were washed with brine solution (2×1 L). The organic layers were dried over anhydrous sodium sulfate. The dried solution was filtered, and the filtrate was concentrated. The residue was purified by silica gel column with ethyl acetate/petroleum ether (1:6) to provide 2-[(acetyloxy)methyl]prop-2-en-1-yl acetate (Int-bb). ¹H NMR (400 MHz, CDCl3) δ 5.28 (s, 2H), 4.61 (s, 4H), 2.09 (s, 6H).

### Step B: [1-[(acetyloxy)methyl]-2,2-difluorocyclopropyl]methyl acetate (Int-bc)

Into a 20-L 4-necked round-bottom flask and maintained with an inert atmosphere of nitrogen was placed a solution of 2-[(acetyloxy)methyl]prop-2-en-1-yl acetate (Int-bb) (600. g, 3.48 mol) in diglyme (5 L). This was followed by the addition of a solution of ClCF₂CO₂Na (2.65 kg, 17.4 mol) in diglyme (5 L) dropwise with stirring at 180° C. over 5 h. The resulting solution was stirred for 1 h at 180° C. The reaction mixture was cooled to room temperature and quenched by the addition of H₂O (5 L). The resulting solution was extracted with petroleum ether (4×2 L), and the organic layers were combined. The combined organic layers were washed with water (3×2 L) and dried over anhydrous sodium sulfate. The dried solution was filtered, and the filtrate was concentrated to dryness to afford [1-[(acetyloxy)methyl]-2,2-difluorocyclopropyl]methyl acetate (Int-bc), which was used directly in the next step without purification.

### Step C: [2,2-difluoro-1-(hydroxymethyl)cyclopropyl]methanol (Int-bd)

Into a 20-L 4-necked round-bottom flask were placed [1-[(acetyloxy)methyl]-2,2-difluorocyclopropyl]methyl acetate (Int-bc) (800 g, 3.60 mol), MeOH (10 L) and K₂CO₃ (995 g, 7.20 mol). The resulting solution was stirred overnight at room temperature. The solids were filtered out. The filtrate was concentrated. The resulting mixture was then diluted by the addition of water (2 L). The resulting solution was extracted with ethyl acetate (5×1 L). The organic layers were combined and dried over anhydrous sodium sulfate. The dried solution was filtered, and the filtrate was concentrated under vacuum. The residue was purified by silica gel column chromatography with ethyl acetate/petroleum ether (1:1) to afford [2,2-difluoro-1-(hydroxymethyl)cyclopropyl]methanol (Int-bd). ¹H NMR (600 MHz, CDCl3) δ 3.91-3.86 (m, 2H), 3.83 (d, J=11.6 Hz, 2H), 2.30 (s, 2H), 1.32 (t, J=8.3 Hz, 2H).

### Step D: (1-((benzyloxy)methyl)-2,2-difluorocyclopropyl)methanol (Int-be)

A 500 mL single neck round bottom flask fitted with a pour-through nitrogen adapter was purged with nitrogen and then charged with sodium hydride (4.52 g, 113 mmol) and N,N-dimethylformamide (100 mL). The suspension was cooled to 0° C. Solid [2,2-difluoro-1-(hydroxymethyl)cyclopropyl]methanol (Int-bd) (12.0 g, 87 mmol) was added portion wise. The mixture was stirred while warming to RT for 1 h. The resultant reaction mixture was cooled to 0° C. and treated with a solution of benzyl bromide (10.3 mL, 87 mmol) in N,N-dimethylformamide (10 mL). The mixture was stirred at rt for 1 h and then treated with sat. aq. ammonium chloride (10 mL) and water (10 mL). The mixture was partitioned between ethyl acetate (75 mL) and water (75 mL). The organic layer was washed with 1 wt % aqueous LiCl (30 mL×3), dried with anhydrous sodium sulfate, filtered and the filtrate was concentrated. Purification by column chromatography on silica gel (EtOAc in hexanes, 0-40% gradient) afforded (1-((benzyloxy)methyl)-2,2-difluorocyclopropyl)methanol (Int-be). ¹H NMR (600 MHz, DMSO-d6) δ 7.39-7.25 (m, 5H), 4.90 (t, J=5.5 Hz, 1H), 4.52-4.46 (m, 2H), 3.66-3.61 (m, 1H), 3.60-3.55 (m, 1H), 3.52-3.46 (m, 2H), 1.46-1.35 (m, 2H).

### Step E: (R)-(1-((benzyloxy)methyl)-2,2-difluorocyclopropyl)methanol (Int-bf)

Racemic (1-((benzyloxy)methyl)-2,2-difluorocyclopropyl)methanol (Int-be) was resolved using SFC chiral chromatography (Column B; 5% MeOH w/0.1% NH₄OH and 5% H₂O) to yield (R)-(1-((benzyloxy)methyl)-2,2-difluorocyclopropyl)methanol (Int-bf, Peak 1) and (S)-(1-((benzyloxy)methyl)-2,2-difluorocyclopropyl)methanol (Int-bf, Peak 2). ¹H NMR (600 MHz, DMSO-d6) δ 7.39-7.25 (m, 5H), 4.90 (t, J=5.5 Hz, 1H), 4.52-4.46 (m, 2H), 3.66-3.61 (m, 1H), 3.60-3.55 (m, 1H), 3.52-3.46 (m, 2H), 1.46-1.35 (m, 2H). (Int-bf Peak 2)¹H NMR (600 MHz, DMSO-d6) δ 7.39-7.25 (m, 5H), 4.90 (t, J=5.5 Hz, 1H), 4.52-4.46 (m, 2H), 3.66-3.61 (m, 1H), 3.60-3.55 (m, 1H), 3.52-3.46 (m, 2H), 1.46-1.35 (m, 2H).

### (S)-(1-(((tert-butyldimethylsilyl)oxy)methyl)-2,2-difluorocyclopropyl)methanol (Int-cb)

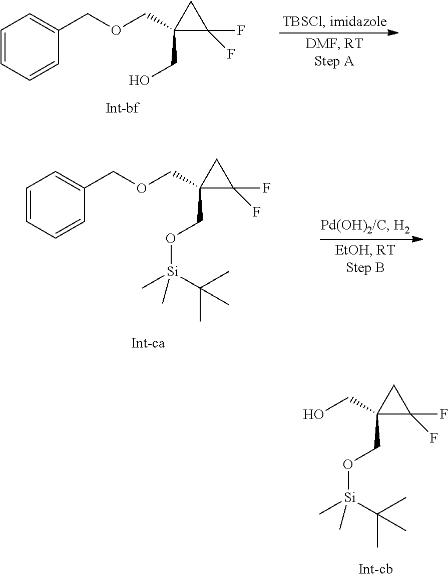 [SMILES: OC[C@@]1(COCc2ccccc2)CC1(F)F ; CC(C)(C)[Si](C)(C)OC[C@@]1(COCc2ccccc2)CC1(F)F ; CC(C)(C)[Si](C)(C)OC[C@@]1(CO)CC1(F)F]

### Step A: (S)-((1-((benzyloxy)methyl)-2,2-difluorocyclopropyl)methoxy)(tert-butyl)dimethylsilane (Int-ca)

To a solution of (R)-(1-((benzyloxy)methyl)-2,2-difluorocyclopropyl)methanol (Int-bf) (2.13 g, 9.33 mmol) in DMF (20 mL) were added imidazole (1.91 g, 28.0 mmol) and tert-butyldimethylsilane chloride (2.64 g, 17.5 mmol) at room temperature. After stirring the mixture at room temperature for 40 h, the reaction was quenched by the addition of H₂O. The reaction mixture was extracted with EtOAc, washed with brine, dried over Na₂SO₄, filtered and concentrated in vacuo. The residue was purified by flash silica gel chromatography (0-10%, EtOAc gradient in hexane) to afford (S)-((1-((benzyloxy)methyl)-2,2-difluorocyclopropyl)methoxy)(tert-butyl)dimethylsilane (Int-ca). ¹H NMR (400 MHz, CDCl₃) δ 7.38-7.27 (m, 5H), 4.55 (d, J=12.0 Hz, 1H), 4.51 (d, J=12.0 Hz, 1H), 3.75 (s, 2H), 3.65-3.54 (m, 2H), 1.31-1.19 (m, 2H), 0.88 (s, 9H), 0.05 (s, 3H), 0.05 (s, 3H).

### Step B: (S)-(1-(((tert-butyldimethylsilyl)oxy)methyl)-2,2-difluorocyclopropyl)methanol (Int-cb)

To a solution of (S)-((1-((benzyloxy)methyl)-2,2-difluorocyclopropyl)methoxy)(tert-butyl)dimethylsilane (Int-ca) (500 mg, 1.46 mmol) in ethanol (7.0 mL) was added Pd(OH)₂/C (513 mg, 0.730 mmol). The reaction mixture was purged with H₂ gas and vacuum three times and stirred at room temperature for 1 h. The reaction mixture was filtered and concentrated in vacuo to afford (S)-(1-(((tert-butyldimethylsilyl)oxy)methyl)-2,2-difluorocyclopropyl)methanol (Int-cb). ¹H NMR (400 MHz, CDCl₃) δ 3.91-3.74 (m, 4H), 2.60 (t, J=6.0 Hz, 1H), 1.37-1.29 (m, 1H), 1.26-1.18 (m, 1H), 0.91 (s, 9H), 0.10 (s, 3H), 0.09 (s, 3H).

### Tert-butyl 3-cyano-4-(5,5-dimethyl-1,3,2-dioxaborinan-2-yl)-7-fluorobenzo[b]thiophen-2-yl)carbamate (Int-df)

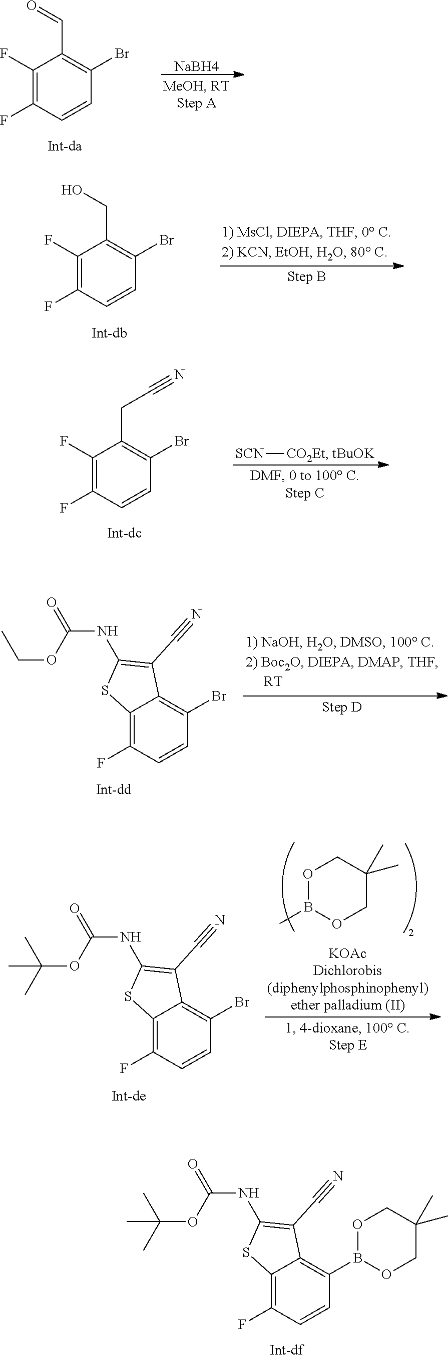

### Step A: (6-bromo-2,3-difluorophenyl)methanol (Int-db)

To a solution of 6-bromo-2,3-difluorobenzaldehyde (Int-da) (5.00 g, 22.6 mmol) and in MeOH (100 mL) was added sodium borohydride (5.00 g, 22.0 mmol) at 0° C. After stirring the mixture at room temperature for 1 h, the reaction was quenched by the addition of H₂O. The reaction mixture was extracted with EtOAc, washed with brine, dried over Na₂SO₄, filtered and concentrated in vacuo to afford (6-bromo-2,3-difluorophenyl)methanol (Int-db), which was used in the next step without further purification. ¹H NMR (400 MHz, CDCl₃) δ 7.37-7.31 (m, 1H), 7.09-7.02 (m, 1H), 4.86 (dd, J=6.8, 2.4 Hz, 2H), 2.11 (t, J=6.8 Hz, 1H).

### Step B: 2-(6-bromo-2,3-difluorophenyl)acetonitrile (Int-dc)

To a solution of (6-bromo-2,3-difluorophenyl)methanol (Int-db) (3.00 g, 13.5 mmol) in THF (30 mL) were added N,N-diisopropylethylamine (2.81 mL, 16.1 mmol) and methanesulfonyl chloride (1.15 mL, 14.8 mmol) at 0° C. After stirring the mixture at room temperature for 15 h, the reaction mixture was quenched by the addition of H₂O. The reaction mixture was extracted with EtOAc, washed with brine, dried over Na₂SO₄, filtered and concentrated in vacuo to afford a benzyl chloride compound, which was used in the next step without further purification.

To a solution of the benzyl chloride compound in EtOH (30 mL) and H₂O (6 mL) was added potassium cyanide (818 mg, 12.6 mmol). After stirring the mixture at 80° C. for 1 h, the reaction mixture was cooled to room temperature and removed EtOH in vacuo. The reaction mixture was extracted with EtOAc, washed with brine, dried over Na₂SO₄, filtered and concentrated in vacuo. The residue was purified by flash silica gel chromatography (0-20%, EtOAc gradient in hexane) to afford 2-(6-bromo-2,3-difluorophenyl)acetonitrile (Int-dc). ¹H NMR (400 MHz, CDCl₃) δ 7.43-7.38 (m, 1H), 7.17-7.09 (m, 1H), 4.89 (d, J=2.0 Hz, 2H).

### Step C: Ethyl (4-bromo-3-cyano-7-fluorobenzo[b]thiophen-2-yl)carbamate (Int-dd)

To a solution of 2-(6-bromo-2,3-difluorophenyl)acetonitrile (Int-dc) (2.00 g, 8.62 mmol) in DMF (20 mL) was added potassium tert-butoxide (1.02 g, 9.05 mmol) at 0° C. After stirring the mixture at 0° C. for 10 min, to the reaction mixture was added ethoxycarbonyl isothiocyanate (1.07 mL, 9.05 mmol). The reaction mixture was stirred at room temperature for 1 h, and then, heated at 100° C. for 30 min. The mixture was then cooled to 0° C. and H₂O was added slowly with stirring. The resulting precipitate was collected by filtration, rinsed with H₂O and hexane, and dried in vacuo to afford ethyl (4-bromo-3-cyano-7-fluorobenzo[b]thiophen-2-yl)carbamate (Int-dd). ESI-MS m/z [M−H]⁻ 341, 343.

### Step D: Tert-butyl (4-bromo-3-cyano-7-fluorobenzo[b]thiophen-2-yl)carbamate (Int-de)

To a solution of ethyl (4-bromo-3-cyano-7-fluorobenzo[b]thiophen-2-yl)carbamate (Int-dd) (2.51 g, 7.31 mmol) in DMSO (10 mL) was added 5.0 M aqueous solution of NaOH (8.00 mL, 40.0 mmol). After stirring the mixture at 100° C. for 13 h, the mixture was then cooled to room temperature and H₂O was added slowly with stirring. The resulting precipitate was collected by filtration, rinsed with H₂O and hexane, and dried in vacuo to afford an amine compound.

To a solution of the amine compound in THE (40 mL) were added N,N-diisopropylethylamine (1.54 mL, 8.85 mmol), DMAP (36.0 mg, 0.295 mmol) and di-tert-butyl dicarbonate (1.42 g, 6.49 mmol). After stirring the mixture at room temperature for 13 h, H₂O and EtOAc were added, and then the resulting precipitate was collected by filtration, rinsed with H₂O and hexane, and dried in vacuo to afford tert-butyl (4-bromo-3-cyano-7-fluorobenzo[b]thiophen-2-yl)carbamate (Int-de). The filtrate layers were extracted with EtOAc, washed with brine, dried over Na₂SO₄, filtered and concentrated in vacuo. The residue was purified by flash silica gel chromatography (0-10%, MeOH gradient in EtOAc) to afford tert-butyl (4-bromo-3-cyano-7-fluorobenzo[b]thiophen-2-yl)carbamate (Int-de). ¹H NMR (400 MHz, CDCl₃) δ 7.96 (s, 1H), 7.54-7.49 (m, 1H), 6.94-6.88 (m, 1H), 1.59 (s, 9H).

### Step E: Tert-butyl (3-cyano-4-(5,5-dimethyl-1,3,2-dioxaborinan-2-yl)-7-fluorobenzo[b]thiophen-2-yl)carbamate (Int-df)

To a solution of tert-butyl (4-bromo-3-cyano-7-fluorobenzo[b]thiophen-2-yl)carbamate (Int-de) (1.00 g, 2.69 mmol) and bis(neopentyl glycolato)diboron (1.83 g, 8.08 mmol) in 1,4-dioxane (15 mL) was added potassium acetate (793 mg, 8.08 mmol). After stirring the mixture at room temperature for 1 h, to the mixture was added dichlorobis(diphenylphosphinophenyl)ether palladium (II) (193 mg, 0.269 mmol), and then, heated at 100° C. for 2 h. The reaction mixture was cooled to room temperature and H₂O was added. The reaction mixture was extracted with EtOAc, washed with brine, dried over Na₂SO₄, filtered and concentrated in vacuo. The residue was purified by flash silica gel chromatography (0-30%, EtOAc gradient in hexane) to afford tert-butyl (3-cyano-4-(5,5-dimethyl-1,3,2-dioxaborinan-2-yl)-7-fluorobenzo[b]thiophen-2-yl)carbamate (Int-df). ¹H NMR (400 MHz, DMSO-d₆) δ 11.6 (s, 1H), 7.61-7.56 (m, 1H), 7.22-7.15 (m, 1H), 3.77 (s, 4H), 1.52 (s, 9H), 1.02 (s, 6H).

### Tert-butyl (4-bromo-3-cyano-5,7-difluorobenzo[b]thiophen-2-yl)carbamate (Int-ef)

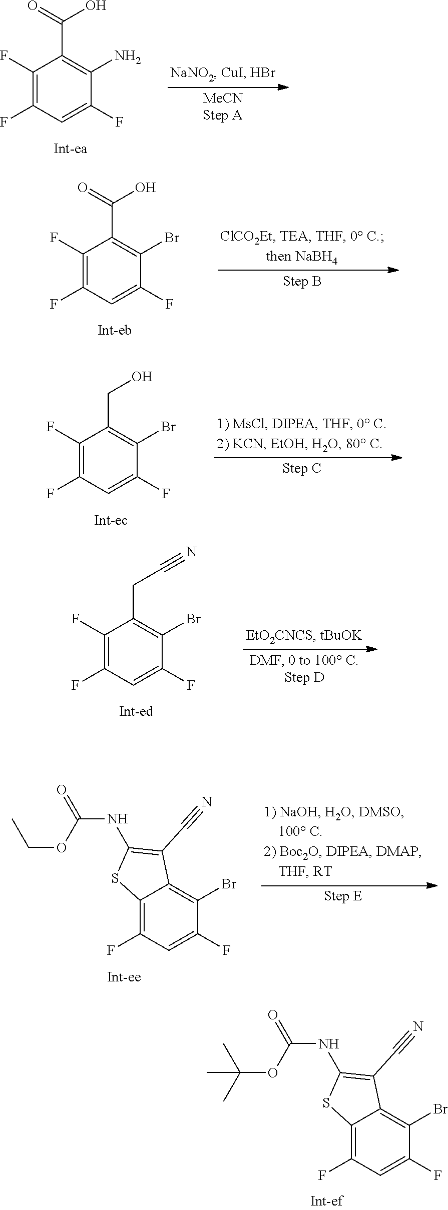 [SMILES: Nc1c(F)cc(F)c(F)c1C(=O)O ; O=C(O)c1c(F)c(F)cc(F)c1Br ; OCc1c(F)c(F)cc(F)c1Br ; N#CCc1c(F)c(F)cc(F)c1Br ; CCOC(=O)Nc1sc2c(F)cc(F)c(Br)c2c1C#N ; CC(C)(C)OC(=O)Nc1sc2c(F)cc(F)c(Br)c2c1C#N]

### Step A: 2-bromo-3,5,6-trifluorobenzoic acid (Int-eb)

To a solution of 2-amino-3,5,6-trifluorobenzoic acid (Int-ea) (5.00 g, 26.2 mmol) in MeCN (60 mL) was slowly added HBr (47%, 60.0 mL, 527 mmol) at 0° C. To the resulting solution a solution of NaNO₂ (1.99 g, 28.8 mmol) in water (5.0 mL) was added dropwise over 10 min. After stirring the mixture at 0° C. for 30 min, to the reaction mixture was added copper(I) bromide (4.50 g, 31.4 mmol) portionwise over 10 min. After stirring the reaction mixture at 70° C. for 2 h, the reaction mixture was cooled to 0° C. and removed solvent in vacuo. The residue was resolved with EtOAc, washed with brine, dried over Na₂SO₄, filtered and concentrated in vacuo. The residue was purified by flash silica gel chromatography (0-20%, EtOAc gradient in hexane) to afford 2-bromo-3,5,6-trifluorobenzoic acid (Int-eb). ESI-MS m/z [M−H]⁻ 253, 255. ¹H NMR (400 MHz, DMSO-d₆) δ 14.69 (br, 1H), 7.97-7.87 (m, 1H)

### Step B: (2-bromo-3,5,6-trifluorophenyl)methanol (Int-ec)

To a solution of 2-bromo-3,5,6-trifluorobenzoic acid (Int-eb) (6.42 g, 25.2 mmol) in THE (100 mL) were added triethylamine (5.26 mL, 37.8 mmol) and ethyl chloroformate (2.64 mL, 27.7 mmol) at 0° C. After stirring the mixture at room temperature for 3 h, to the reaction mixture was added ethyl chloroformate (2.64 mL, 27.7 mmol) and stirred at room temperature for 1 h. The reaction was quenched by the addition of H₂O and the reaction mixture was extracted with EtOAc, washed with brine, dried over Na₂SO₄, filtered and concentrated in vacuo to afford an acid anhydride compound, which was used in the next step without further purification.

To a solution of the acid anhydride compound in THE (48 mL) and H₂O (20 mL) was added NaBH₄ (2.86 g, 75.5 mmol) at 0° C. After stirring the mixture at room temperature for 2 h, the solvent was removed in vacuo. The residue was resolved with EtOAc, washed with brine, dried over Na₂SO₄, filtered and concentrated in vacuo. The residue was purified by flash silica gel chromatography (0-20%, EtOAc gradient in hexane) to afford (2-bromo-3,5,6-trifluorophenyl)methanol (Int-ec). ¹H NMR (400 MHz, CDCl₃) δ 7.07-7.01 (m, 1H), 4.89 (dd, J=6.8, 2.4 Hz, 2H), 2.10 (t, J=6.8 Hz, 1H).

### Step C: 2-(2-bromo-3,5,6-trifluorophenyl)acetonitrile (Int-ed)

To a solution of (2-bromo-3,5,6-trifluorophenyl)methanol (Int-ec) (4.57 g, 19.0 mmol) in THE (40 mL) were added N,N-diisopropylethylamine (4.95 mL, 28.4 mmol) and methanesulfonyl chloride (2.00 mL, 25.8 mmol) at 0° C. After stirring the mixture at room temperature for 7 h, the reaction mixture was quenched by the addition of H₂O. The reaction mixture was extracted with EtOAc, washed with brine, dried over Na₂SO₄, filtered and concentrated in vacuo to afford a benzyl chloride compound, which was used without further purification.

To a solution of the benzyl chloride compound in EtOH (50 mL) was added potassium cyanide (1.36 g, 21.0 mmol) in H₂O (10 mL). After stirring the mixture at 80° C. for 2 h, the reaction mixture was cooled to room temperature and removed EtOH in vacuo. The reaction mixture was extracted with EtOAc, washed with brine, dried over Na₂SO₄, filtered and concentrated in vacuo. The residue was purified by flash silica gel chromatography (0-30%, EtOAc gradient in hexane) to afford 2-(2-bromo-3,5,6-trifluorophenyl)acetonitrile (Int-ed). ¹H NMR (400 MHz, CDCl₃) δ 7.15-7.09 (m, 1H), 3.93 (d, J=2.0 Hz, 2H).

### Step D: Ethyl (4-bromo-3-cyano-5,7-difluorobenzo[b]thiophen-2-yl)carbamate (Int-ee)

To a solution of 2-(2-bromo-3,5,6-trifluorophenyl)acetonitrile (Int-ed) (3.58 g, 14.3 mmol) in DMF (36 mL) was added potassium tert-butoxide (1.69 g, 15.0 mmol) at 0° C. After stirring the mixture at 0° C. for 30 min, to the reaction mixture was added ethoxycarbonyl isothiocyanate (1.77 mL, 15.0 mmol). The reaction mixture was stirred at room temperature for 1 h, and then, heated at 100° C. for 2 h. The mixture was then cooled to 0° C. and H₂O was added slowly with stirring. The resulting precipitate was collected by filtration, rinsed with H₂O and hexane, and dried in vacuo to afford ethyl (4-bromo-3-cyano-5,7-difluorobenzo[b]thiophen-2-yl)carbamate (Int-ee). ESI-MS m/z [M−H]⁻ 359, 361. ¹H NMR (400 MHz, DMSO-d₆) δ 12.21 (br, 1H), 7.61-7.28 (m, 1H), 4.29 (q, J=7.2 Hz, 2H), 1.30 (t, J=7.2 Hz, 3H).

### Step E: Tert-butyl (4-bromo-3-cyano-5,7-difluorobenzo[b]thiophen-2-yl)carbamate (Int-ef)

To a solution of ethyl (4-bromo-3-cyano-5,7-difluorobenzo[b]thiophen-2-yl)carbamate (Int-ee) (3.53 g, 9.77 mmol) in DMSO (16 mL) was added 5.0 M aqueous solution of NaOH (10.0 mL, 50.0 mmol). After stirring the mixture at 100° C. for 12 h, the mixture was then cooled to room temperature and H₂O was added slowly with stirring. The resulting precipitate was collected by filtration, rinsed with H₂O and hexane, and dried in vacuo to afford the corresponding amine.

To a solution of amine compound in THE (44 mL) were added N,N-diisopropylethylamine (2.63 mL, 15.1 mmol), DMAP (46.1 mg, 0.377 mmol) and di-tert-butyl dicarbonate (3.00 mL, 8.79 mmol). After stirring the mixture at room temperature for 15 h, The reaction mixture was extracted with EtOAc, washed with brine, dried over Na₂SO₄, filtered and concentrated in vacuo. The residue was suspended with diisopropyl ether and filtered to tert-butyl (4-bromo-3-cyano-5,7-difluorobenzo[b]thiophen-2-yl)carbamate (Int-ef). ESI-MS m/z [M+H]⁺ 389, 391. ¹H NMR (400 MHz, DMSO-d₆) δ 11.94 (br, 1H), 7.43-7.28 (m, 1H), 1.48 (s, 9H).

### 4-(7-chloro-8-fluoro-2-(((2R,7aS)-2-fluorotetrahydro-1H-pyrrolizin-7a(5H)-yl)methoxy)pyrido[4,3-d]pyrimidin-4-yl)-1,4-oxazepane (Int-fc)

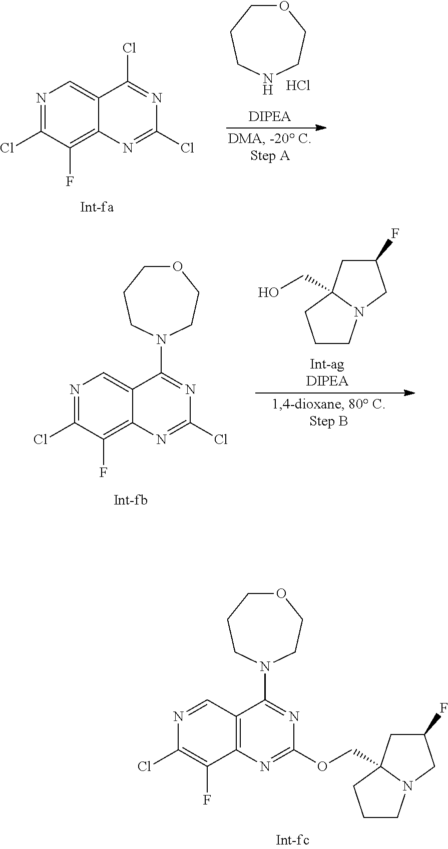 [SMILES: Fc1c(Cl)ncc2c(Cl)nc(Cl)nc12 ; C1CNCCOC1 ; Cl ; Fc1c(Cl)ncc2c(N3CCCOCC3)nc(Cl)nc12 ; OC[C@@]12CCCN1C[C@H](F)C2 ; Fc1c(Cl)ncc2c(N3CCCOCC3)nc(OC[C@@]34CCCN3C[C@H](F)C4)nc12]

### Step A: 4-(2,7-dichloro-8-fluoropyrido[4,3-d]pyrimidin-4-yl)-1,4-oxazepane (Int-fb)

To a solution of 2,4,7-trichloro-8-fluoropyrido[4,3-d]pyrimidine (Int-fa) (1.18 g, 4.65 mmol) in N,N-dimethylacetamide (12 ml) was added 1,4-oxazepane hydrochloride (576 mg, 4.19 mmol) and N,N-diisopropylethylamine (4.1 ml, 23.3 mmol) at −20° C. The mixture was stirred at −20° C. for 0.5 h under nitrogen atmosphere. The reaction mixture was diluted with water and ethyl acetate. The organic layer was washed with brine, dried over MgSO₄, filtered and concentrated in vacuo. The residue was purified by flash silica chromatography (eluent, 12-100% EtOAc in hexane) to obtain 4-(2,7-dichloro-8-fluoropyrido[4,3-d]pyrimidin-4-yl)-1,4-oxazepane (Int-fb). ESI-MS m/z [M+H]⁺ 317, 319, 321. ¹H NMR (400 MHz, CDCl₃) δ 8.94 (s, 1H), 4.19-4.15 (m, 4H), 4.02-4.00 (m, 2H), 3.86-3.84 (m, 2H), 2.24-2.18 (m, 2H).

### Step B: 4-(7-chloro-8-fluoro-2-(((2R,7aS)-2-fluorotetrahydro-1H-pyrrolizin-7a(5H)-yl)methoxy)pyrido[4,3-d]pyrimidin-4-yl)-1,4-oxazepane (Int-fc)

To a solution of 4-(2,7-dichloro-8-fluoropyrido[4,3-d]pyrimidin-4-yl)-1,4-oxazepane (Int-fb) (400 mg, 1.26 mmol) and ((2R,7aS)-2-fluorotetrahydro-1H-pyrrolizin-7a(5H)-yl)methanol (Int-ag) (602 mg, 3.78 mmol) in 1,4-dioxane (8.0 ml) was added N,N-diisopropylethylamine (0.65 ml, 3.78 mmol) at room temperature. The mixture was stirred at 80° C. for 14 h under nitrogen atmosphere. The reaction mixture was cooled to room temperature and diluted with water and ethyl acetate. The organic layer was dried over MgSO₄, filtered and concentrated in vacuo. The residue was purified by flash NH-silica chromatography (12-100% EtOAc in hexane) to obtain 4-(7-chloro-8-fluoro-2-(((2R,7aS)-2-fluorotetrahydro-1H-pyrrolizin-7a(5H)-yl)methoxy)pyrido[4,3-d]pyrimidin-4-yl)-1,4-oxazepane (Int-fc). ESI-MS m/z [M+H]⁺ 440, 442. ¹H NMR (400 MHz, CDCl₃) δ 8.81 (s, 1H), 5.34-5.13 (m, 1H), 4.26-4.24 (m, 1H), 4.15-4.10 (m, 5H), 3.99-3.97 (m, 2H), 3.85-3.82 (m, 2H), 3.27-2.90 (m, 4H), 2.32-2.04 (m, 5H), 2.00-1.74 (m, 3H).

### 4-(azepan-1-yl)-7-bromo-2,6-dichloro-8-fluoroquinazoline (Int-gb)

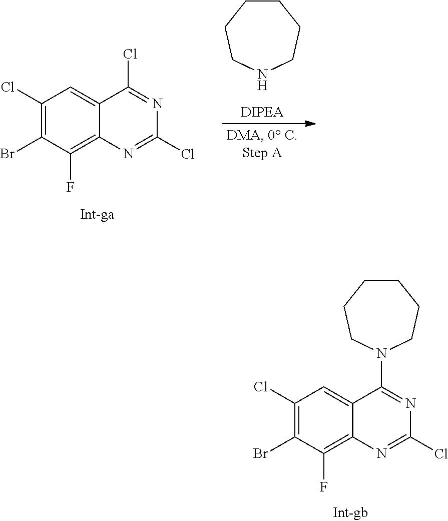 [SMILES: Fc1c(Br)c(Cl)cc2c(Cl)nc(Cl)nc12 ; C1CCCNCC1 ; Fc1c(Br)c(Cl)cc2c(N3CCCCCC3)nc(Cl)nc12]

### Step A: 4-(azepan-1-yl)-7-bromo-2,6-dichloro-8-fluoroquinazoline (Int-gb)

To a solution of 7-bromo-2,4,6-trichloro-8-fluoroquinazoline (Int-ga) (300 mg, 0.91 mmol) in N,N-dimethylacetamide (3.0 ml) was added azepane (81.1 mg, 0.82 mmol) and N,N-diisopropylethylamine (0.79 ml, 4.54 mmol) at 0° C. The mixture was stirred at 0° C. for 0.5 h under an atmosphere of nitrogen. The reaction mixture was diluted with water and ethyl acetate. The organic layer was washed with brine, dried over MgSO₄, filtered and concentrated in vacuo. The residue was purified by flash silica chromatography (12-100% EtOAc in hexane) to obtain 4-(azepan-1-yl)-7-bromo-2,6-dichloro-8-fluoroquinazoline (Int-gb). ESI-MS m/z [M+H]⁺ 392, 394, 396, 397. 1H NMR (400 MHz, DMSO-d6) δ 8.11 (d, J=2.0 Hz, 1H), 3.94-3.91 (m, 4H), 1.89 (br s, 4H), 1.57-1.55 (m, 4H).

### Tert-butyl (4-(6-chloro-2-(((R)-2,2-difluoro-1-(hydroxymethyl)cyclopropyl)methoxy)-8-fluoro-4-(1,4-oxazepan-4-yl)quinazolin-7-yl)-3-cyano-7-fluorobenzo[b]thiophen-2-yl)carbamate (Int-hb)

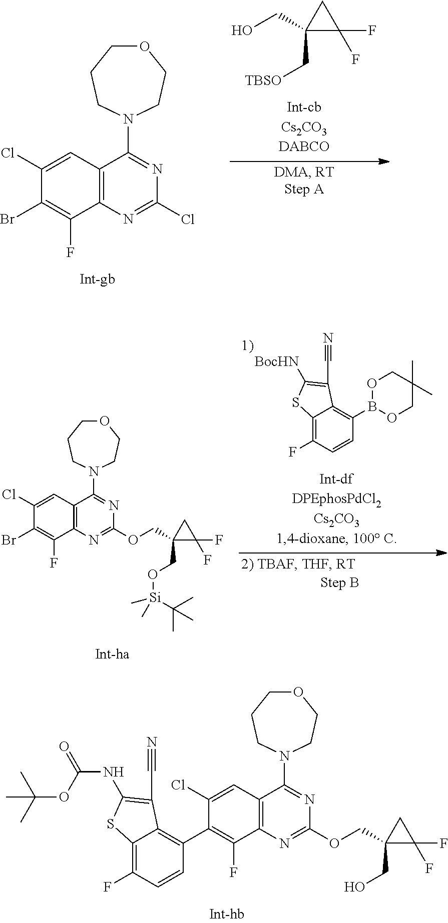 [SMILES: Fc1c(Br)c(Cl)cc2c(N3CCCOCC3)nc(Cl)nc12 ; CC(C)(C)[Si](C)(C)OC[C@@]1(CO)CC1(F)F ; CC(C)(C)[Si](C)(C)OC[C@@]1(COc2nc(N3CCCOCC3)c3cc(Cl)c(Br)c(F)c3n2)CC1(F)F ; CC1(C)COB(c2ccc(F)c3sc(NC(=O)OC(C)(C)C)c(C#N)c23)OC1 ; CC(C)(C)OC(=O)Nc1sc2c(F)ccc(-c3c(Cl)cc4c(N5CCCOCC5)nc(OC[C@]5(CO)CC5(F)F)nc4c3F)c2c1C#N]

### Step A: (S)-4-(7-bromo-2-((1-(((tert-butyldimethylsilyl)oxy)methyl)-2,2-difluorocyclopropyl)methoxy)-6-chloro-8-fluoroquinazolin-4-yl)-1,4-oxazepane (Int-ha)

To a solution of 4-(7-bromo-2,6-dichloro-8-fluoroquinazolin-4-yl)-1,4-oxazepane (Int-gb) (450 mg, 1.14 mmol) and (S)-(1-(((tert-butyldimethylsilyl)oxy)methyl)-2,2-difluorocyclopropyl)methanol (Int-cb) (343 mg, 1.36 mmol) in DMA (9.0 mL) were added cesium carbonate (1.11 g, 3.42 mmol) and 1,4-diazabicyclo[2.2.2]octane (25.6 mg, 0.228 mmol) at room temperature. After stirring the mixture at room temperature for 14 h, the reaction was quenched by the addition of H₂O. The reaction mixture was extracted with EtOAc, washed with brine, dried over Na₂SO₄, filtered and concentrated in vacuo. The residue was purified by flash silica gel chromatography (5-30%, EtOAc gradient in hexane) to obtain (S)-4-(7-bromo-2-((1-(((tert-butyldimethylsilyl)oxy)methyl)-2,2-difluorocyclopropyl)methoxy)-6-chloro-8-fluoroquinazolin-4-yl)-1,4-oxazepane (Int-ha). ESI-MS m/z [M+H]⁺ 610, 612.

### Step B: Tert-butyl (4-(6-chloro-2-(((R)-2,2-difluoro-1-(hydroxymethyl)cyclopropyl)methoxy)-8-fluoro-4-(1,4-oxazepan-4-yl)quinazolin-7-yl)-3-cyano-7-fluorobenzo[b]thiophen-2-yl)carbamate (Int-hb)

To a solution of (S)-4-(7-bromo-2-((1-(((tert-butyldimethylsilyl)oxy)methyl)-2,2-difluorocyclopropyl)methoxy)-6-chloro-8-fluoroquinazolin-4-yl)-1,4-oxazepane (Int-ha) (484 mg, 0.792 mmol), tert-butyl (3-cyano-4-(5,5-dimethyl-1,3,2-dioxaborinan-2-yl)-7-fluorobenzo[b]thiophen-2-yl)carbamate (Int-df) (641 mg, 1.58 mmol) and dichlorobis(diphenylphosphinophenyl)ether palladium (II) (113 mg, 0.158 mmol) in 1,4-dioxane (8.0 mL) was added cesium carbonate (645 mg, 1.98 mmol). After stirring the mixture at 100° C. for 3 h, the reaction mixture was cooled to room temperature, diluted with EtOAc, filtered and concentrated in vacuo. The residue was purified by flash NH-silica gel chromatography (0-30%, MeOH gradient in EtOAc) to afford a coupling product.

To a solution of the coupling product in THE (3 mL) was added tetra-n-butylammonium fluoride (3.00 mL, 3.00 mmol, 1M in THF) and the mixture was stirred at room temperature for 2 h. The mixture was diluted with ethyl acetate, washed with water and concentrated under reduced pressure. The residue was purified by flash NH-silica gel chromatography (0-50%, MeOH gradient in EtOAc) to afford tert-butyl (4-(6-chloro-2-(((R)-2,2-difluoro-1-(hydroxymethyl)cyclopropyl)methoxy)-8-fluoro-4-(1,4-oxazepan-4-yl)quinazolin-7-yl)-3-cyano-7-fluorobenzo[b]thiophen-2-yl)carbamate (Int-hb). ESI-MS m/z [M+H]⁺ 708, 710. ¹H NMR (400 MHz, DMSO-d₆) δ 11.78 (s, 1H), 8.03 (s, 1H), 7.49-7.38 (m, 2H), 5.09 (t, J=5.6 Hz, 1H), 4.56 (d, J=11.0 Hz, 1H), 4.42-4.35 (m, 1H), 4.13-4.05 (m, 4H), 3.95-3.90 (m, 2H), 3.76-3.72 (m, 2H), 3.64-3.58 (m, 2H), 2.11-2.04 (m, 2H), 1.75-1.65 (m, 1H), 1.52 (s, 9H), 1.18 (t, J=7.2 Hz, 1H).

### Tert-butyl (4-(6-bromo-8-fluoro-2-(methylthio)-4-oxo-3,4-dihydroquinazolin-7-yl)-3-cyano-7-fluorobenzo[b]thiophen-2-yl)carbamate (Int-ie)

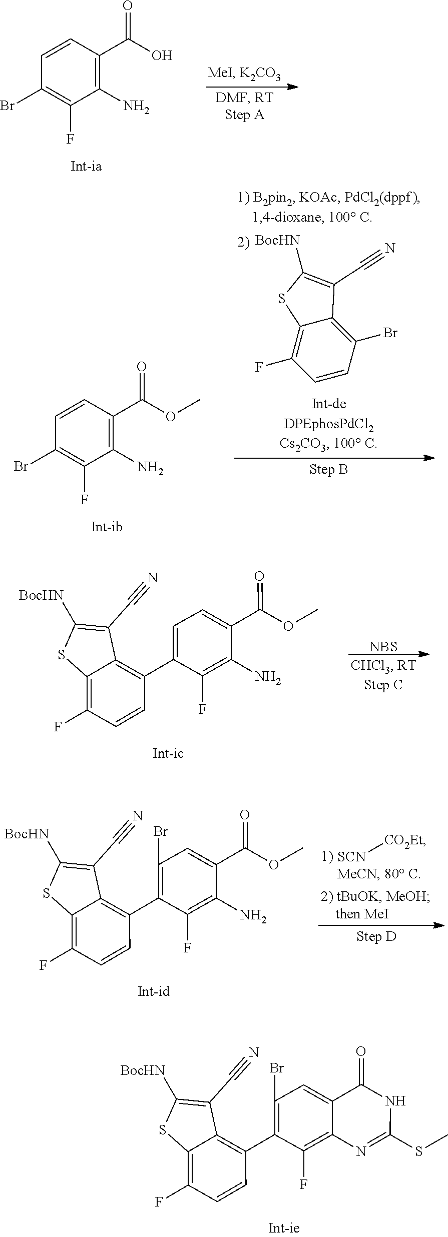

### Step A: Methyl 2-amino-4-bromo-3-fluorobenzoate (Int-ib)

To a mixture of 2-amino-4-bromo-3-fluorobenzoic acid (Int-ia) (3.00 g, 12.8 mmol) and potassium carbonate (3.54 g, 25.6 mmol) in N,N-dimethylformamide (15 mL) was added iodomethane (0.958 mL, 15.4 mmol), the mixture was stirred at room temperature for 2 h. To the reaction mixture was added water (100 mL) dropwise, and the precipitate was filtered, rinsed with water (20 mL) and dried under reduced pressure to give methyl 2-amino-4-bromo-3-fluorobenzoate (Int-ib). ESI-MS m/z [M+H]⁺ 248, 250. ¹H NMR (400 MHz, Chloroform-d) δ 7.53 (dd, J=8.8, 1.6 Hz, 1H), 6.79 (dd, J=8.8, 6.4 Hz, 1H), 5.93 (br s, 2H), 3.90 (s, 3H).

### Step B: Methyl 2-amino-4-(2-((tert-butoxycarbonyl)amino)-3-cyano-7-fluorobenzo[b]thiophen-4-yl)-3-fluorobenzoate (Int-ic)

To a mixture of methyl 2-amino-4-bromo-3-fluorobenzoate (Int-ib) (1.50 g, 6.05 mmol), bis(pinacolato)diboron (3.07 g, 12.1 mmol), and potassium acetate (2.37 g, 24.2 mmol) in 1,4-dioxane (20 mL) was added [1,1′-bis(diphenylphosphino)ferrocene]palladium(II) dichloride (885 mg, 1.21 mmol), the mixture was stirred at 100° C. for 3 h. The mixture was diluted with ethyl acetate (30 mL) and filtered and concentrated under reduced pressure. The residue was purified by column chromatography on silica gel (ethyl acetate in hexane, 0-40% gradient) to give a pinacol boronate.

To a mixture of the pinacol boronate (1.19 g, 4.04 mmol) and tert-butyl N-(4-bromo-3-cyano-7-fluoro-benzothiophen-2-yl)carbamate (Int-de) (1.00 g, 2.69 mmol) in 1,4-dioxane (10 mL) and water (1 mL) was added dichloro[bis(2-(diphenylphosphino)phenyl)ether]palladium(II) (96.4 mg, 0.135 mmol) and cesium carbonate (2.19 g, 6.73 mmol), and the mixture was stirred at 100° C. for 10 min. The reaction mixture was diluted with ethyl acetate (100 mL) and water (30 mL), and the organic layer was separated and washed with water (20 mL) and concentrated under reduced pressure. The residue was purified by column chromatography on silica gel (ethyl acetate in hexane, 0-40% gradient) to give methyl 2-amino-4-(2-((tert-butoxycarbonyl)amino)-3-cyano-7-fluorobenzo[b]thiophen-4-yl)-3-fluorobenzoate (Int-ic). ESI-MS m/z [M+H]⁺ 460. ¹H NMR (400 MHz, Chloroform-d) δ 7.85 (s, 1H), 7.77 (dd, J=8.4, 1.6 Hz, 1H), 7.33-7.29 (m, 1H), 7.11 (dd, J=9.2, 8.4 Hz, 1H), 6.61 (dd, J=8.4, 6.8, 1H), 5.95 (br s, 2H), 3.94 (s, 3H), 1.60 (s, 9H).

### Step C: Methyl 2-amino-5-bromo-4-(2-((tert-butoxycarbonyl)amino)-3-cyano-7-fluorobenzo[b]thiophen-4-yl)-3-fluorobenzoate (Int-id)

To a mixture of methyl 2-amino-4-(2-((tert-butoxycarbonyl)amino)-3-cyano-7-fluorobenzo[b]thiophen-4-yl)-3-fluorobenzoate (Int-ic) (410 mg, 0.892 mmol) in chloroform (30 mL) was added N-bromosuccinimide (159 mg, 0.892 mmol), the mixture was stirred at room temperature for 3 h. The reaction was quenched by addition of methanol (10 mL) and aqueous solution of sodium sulfite, and extracted with chloroform (30 mL). The extract was washed with water (20 mL) and concentrated under reduced pressure. The residue was purified by column chromatography on silica gel (ethyl acetate in hexane, 0-40% gradient) to give methyl 2-amino-5-bromo-4-(2-((tert-butoxycarbonyl)amino)-3-cyano-7-fluorobenzo[b]thiophen-4-yl)-3-fluorobenzoate (Int-id). ESI-MS m/z [M+H]⁺ 538, 540. ¹H NMR (400 MHz, Chloroform-d) δ 8.04 (d, J=2.0 Hz, 1H), 7.83 (br s, 1H), 7.26 (dd, J=8.8, 5.2 Hz, 1H), 7.14 (dd, J=8.8, 8.4 Hz, 1H), 5.93 (br s, 2H), 3.95 (s, 3H), 1.59 (s, 9H).

### Step D: Tert-butyl (4-(6-bromo-8-fluoro-2-(methylthio)-4-oxo-3,4-dihydroquinazolin-7-yl)-3-cyano-7-fluorobenzo[b]thiophen-2-yl)carbamate (Int-ie)

To a mixture of methyl 2-amino-5-bromo-4-(2-((tert-butoxycarbonyl)amino)-3-cyano-7-fluorobenzo[b]thiophen-4-yl)-3-fluorobenzoate (Int-id) (520 mg, 0.966 mmol) in acetonitrile (15 mL) was added ethyl N-(thioxomethylene)carbamate (0.328 mL, 2.90 mmol), the mixture was stirred at 80° C. for 2 h. After the mixture was cooled to room temperature, methanol (10 mL) was added to the mixture. A solvent was evaporated under reduced pressure. To the residue was added methanol (20 mL) and potassium tert-butoxide (2.90 mL, 2.90 mmol, 1M in tetrahydrofuran), the mixture was stirred at 70° C. for 1 h. The mixture was cooled to room temperature, and iodomethane (0.180 mL, 2.90 mmol) was added to the mixture. The reaction mixture was stirred at room temperature for 2 h and acidified by addition of aqueous hydrochloric acid (2.9 mL, 1M). Methanol was removed under reduced pressure, and to the mixture was added acetonitrile (2.5 mL) and water (2.5 mL), and the mixture was stirred at room temperature for 1 h. Precipitate was collected by filtration and dried to give tert-butyl (4-(6-bromo-8-fluoro-2-(methylthio)-4-oxo-3,4-dihydroquinazolin-7-yl)-3-cyano-7-fluorobenzo[b]thiophen-2-yl)carbamate (Int-ie). ESI-MS m/z [M+H]⁺ 579, 581. ¹H NMR (400 MHz, DMSO-d6) δ 13.08 (s, 1H), 11.82 (s, 1H), 8.11 (d, J=1.2 Hz, 1H), 7.43 (s, 1H), 7.42 (s, 1H), 2.59 (s, 3H), 1.52 (s, 9H). 1,4-oxazocane (Int-Id)

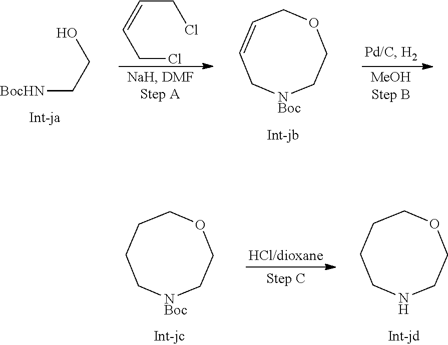

### Step A: Tert-butyl (Z)-2,3,5,8-tetrahydro-4H-1,4-oxazocine-4-carboxylate (Int-jb)

To a solution of (Z)-1,4-dichlorobut-2-ene (2.326 g, 18.61 mmol) in DMF (30 mL) was added tert-butyl (2-hydroxyethyl)carbamate (3 g, 18.61 mmol), sodium hydride (1.861 g, 46.5 mmol, 60%) at 0° C. The mixture was stirred at 25° C. for 12 h. LCMS showed the reaction was completed. The mixture was cooled, diluted with water (30 mL), extracted with EtOAc (3×30 mL), dried over Na₂SO₄, filtered and the solvent was evaporated under reduced pressure to give the crude product. The crude product was purified by flash silica gel chromatography (ISCO®; 80 g SepaFlash® Silica Flash Column, Eluent of 0-30% EtOAc/Pet. ether gradient at 40 mL/min) to give tert-butyl (Z)-2,3,5,8-tetrahydro-4H-1,4-oxazocine-4-carboxylate (Int-jb). MS (ESI) [M+H-tBu]⁺: m/z 158.2

### Step B: Tert-butyl 1,4-oxazocane-4-carboxylate (Int-ic)

To a solution of tert-butyl (Z)-2,3,5,8-tetrahydro-4H-1,4-oxazocine-4-carboxylate (60 mg, 0.281 mmol) in MeOH (1 mL) was added wet Pd/C (2.99 mg, 0.028 mmol, 10%) at 25° C. under H₂ atmosphere (15 psi). The mixture was stirred at 25° C. for 1 h. TLC showed the reaction was completed. The mixture was filtered and the solvent was evaporated under reduced pressure to give the crude product tert-butyl 1,4-oxazocane-4-carboxylate (Int-jc). ¹H NMR (400 MHz, CDCl₃) δ 3.78-3.59 (m, 4H), 3.52-3.32 (m, 4H), 1.85-1.73 (m, 2H), 1.64 (tt, J=5.6, 10.9 Hz, 2H), 1.47 (s, 9H).

### Step C: 1,4-oxazocane (Int-id)

A mixture of tert-butyl 1,4-oxazocane-4-carboxylate (40 mg, 0.186 mmol) and HCl/dioxane (1 mL, 4N) was stirred at 25° C. for 1 h. TLC showed the reaction was completed. The mixture was evaporated under reduced pressure to give the crude product 1,4-oxazocane (Int-jd). ¹H NMR (400 MHz, MeOD) δ 3.80-3.72 (m, 2H), 3.66 (t, J=5.7 Hz, 2H), 3.23-3.20 (m, 2H), 3.15 (t, J=5.5 Hz, 2H), 1.93-1.71 (m, 4H).

### 4-(tert-butoxy)-6-chloro-8-fluoro-2-(methylthio)quinazoline (Int-kg)

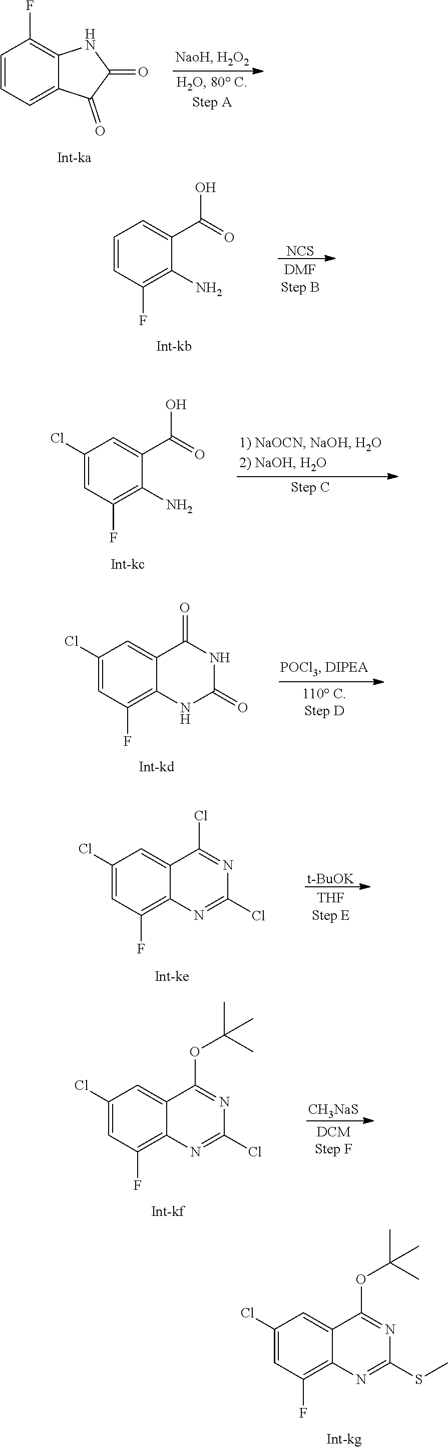

### Step A: 2-amino-3-fluorobenzoic acid (Int-kb)

Water (32L) was added to a reactor at 25° C. 7-fluoroindoline-2,3-dione (3.2 kg, 19 mol) and NaOH (4.6 kg, 116 mol) were added to the reactor and the mixture was stirred at 75-80° C. for 1 hr. H₂O₂(1.5 kg, 13 mol, 30% purity) was added to the reaction slowly at 75-80° C. over a period of 2 h. The mixture was stirred at 75-80° C. for 1 h. When the reaction was complete by TLC, the reaction mixture was added slowly over the course of 2 h at 0-10° C. to a separate vessel containing Na₂SO₃ (10 kg) and H₂O (80 L). 1 N HCl (120 L) was added to the reaction mixture to form a precipitate. The solids were filtered, and the filter cake was washed with water (10 L). The filter cake was dried to give 2-amino-3-fluorobenzoic acid (Int-kb) which was used in the next step without further purification.

### Step B: 2-amino-5-chloro-3-fluorobenzoic acid (Int-kc)

DMF (14 L) was added to 2-amino-3-fluorobenzoic acid (Int-kb) (2.0 kg, 12 mol). NCS (1.8 kg, 14 mol) was added, and the reaction mixture was stirred at RT for 12 h. The reaction was monitored using HPLC. Slowly the reaction mixture was transferred to another flask with H₂O (28 L) 0-10° C. over 2 h. The reaction mixture was filtered, and the filter cake was washed with H₂O (10 L) and then dried to give 2-amino-5-chloro-3-fluorobenzoic acid (Int-kc). ¹H NMR: (400 MHz, DMSO-d6) δ: 7.41-7.47 (m, 1H), 7.50-7.53 (m, 1H).

### Step C: 6-chloro-8-fluoroquinazoline-2,4(1H,3H)-dione (Int-kd)

H₂O (24 L) was added to 2-amino-5-chloro-3-fluorobenzoic acid (Int-kc) (2.4 kg, 12 mol). NaOH (0.7 kg, 17 mol) and NaOCN (1.8 kg, 27 mol) were added to the reaction and the mixture was stirred between 15-35° C. for 1 h. 1 N HCl was added to adjust the pH between 5.8-6.3 and the reaction mixture was stirred for 4 h between 15-35° C. NaOH (1.6 kg, 41 mol) was added, and the reaction mixture was stirred for 2 h between 15-35° C. The reaction mixture was filtered, and the filter cake was washed with H₂O (10 L). The filter cake was re-suspended in H₂O (24 L) at 15-35° C. and 1 M HCl was added to adjust the pH between 1-2. The mixture was stirred for 1 h and then filtered. The filter cake was washed with H₂O (10 L) and resuspended in acetone (12 L) at 15-35° C. and then filtered. The filter cake was again washed with MeOH (5.0 L), dried under vacuum between 40-45° C. for 12 h to give 6-chloro-8-fluoroquinazoline-2,4(1H,3H)-dione (Int-kd). ¹H NMR (400 MHz, DMSO-d6) δ 7.63-7.69 (m, 1H), 7.77-7.85 (m, 1H).

### Step D: 2,4,6-trichloro-8-fluoroquinazoline (Int-ke)

POCl₃ (11 kg, 77 mol) was added to 6-chloro-8-fluoroquinazoline-2,4(1H,3H)-dione (Int-kd) (1.8 kg, 8.6 mol) at 25° C. DIPEA (0.5 kg, 4.1 mmol) was added, and the reaction mixture was stirred for 30 mins. The reaction mixture was warmed to 100-110° C. and stirred for 30 h. The reaction was monitored via HPLC. After completion, the reaction was concentrated under reduced pressure to about 2.0 L. H₂O (50 L) was added and slowly the reaction mixture was transferred to another reactor at 10-30° C. over 2 h and then stirred for 30 mins. The aqueous solution was extracted with the MTBE (3×10 L). The organic layers were combined and washed with brine (2×10 L), dried over Na₂SO₄, filtered and concentrated under reduced pressure to give a residue. This residue was vacuum dried to give 2,4,6-trichloro-8-fluoroquinazoline (Int-ke). ¹H NMR (400 MHz, CDCl₃) δ 7.57-7.65 (m, 1H), 7.98 (s, 1H).

### Step E: 4-(tert-butoxy)-2,6-dichloro-8-fluoroquinazoline (Int-kf)

THE (15 L) was added to 2,4,6-trichloro-8-fluoroquinazoline (Int-ke) (1.5 kg, 5.9 mol) at 20° C. t-BuOK (0.7 kg, 6.5 mol, 1 M in THF) was added in batches to the reaction between −10-5° C. over 30 mins and the reaction was stirred for another 30 mins. The reaction was warmed to RT and stirred for 3 h. 0.5 M HCl (2.0 L) was added to the reaction and then was transferred to another reactor at 0-10° C. over 10 mins. H₂O (15 L) was added, and the aqueous solution was extracted solution with MTBE (3×5.0 L). The organic layers were combined and washed with brine (5.0 L), dried over Na₂SO₄, filtered and concentrated under reduced pressure to give a residue. This was re-crystallized from MeOH (5.0 L) at 25° C. to give 4-(tert-butoxy)-2,6-dichloro-8-fluoroquinazoline (Int-kf). ¹H NMR (400 MHz, CDCl₃) δ 1.68 (s, 9H), 7.39-7.47 (m, 1H), 7.72-7.79 (m, 1H).

### Step F: 4-(tert-butoxy)-6-chloro-8-fluoro-2-(methylthio)quinazoline (Int-kg)

DCM (3.5 L) was added to 4-(tert-butoxy)-2,6-dichloro-8-fluoroquinazoline (Int-kf) (500 g, 1.7 mol) at RT. CH₃NaS (179 g, 2.6 mol) was added slowly to the reaction mixture at 10-20° C. in portions over 30 mins and stirred for 2 h. H₂O (7.0 L) was added and slowly the reaction was transferred to another reactor at 0-10° C. over 10 mins. The reaction was then filtered, and the filter bed was washed with MeOH (5.0 L). The crude residue obtained was purified by reverse phase HPLC to give 4-(tert-butoxy)-6-chloro-8-fluoro-2-(methylthio)quinazoline (Int-kg). ¹H NMR (400 MHz, CDCl3) δ 1.65 (s, 9H), 2.54-2.59 (m, 3H), 7.29-7.37 (m, 1H), 7.62-7.71 (m, 1H). 4-bromo-6-methyl-1-(tetrahydro-2H-pyran-2-yl)-5-(trifluoromethyl)-1H-indazole (Int-lf)

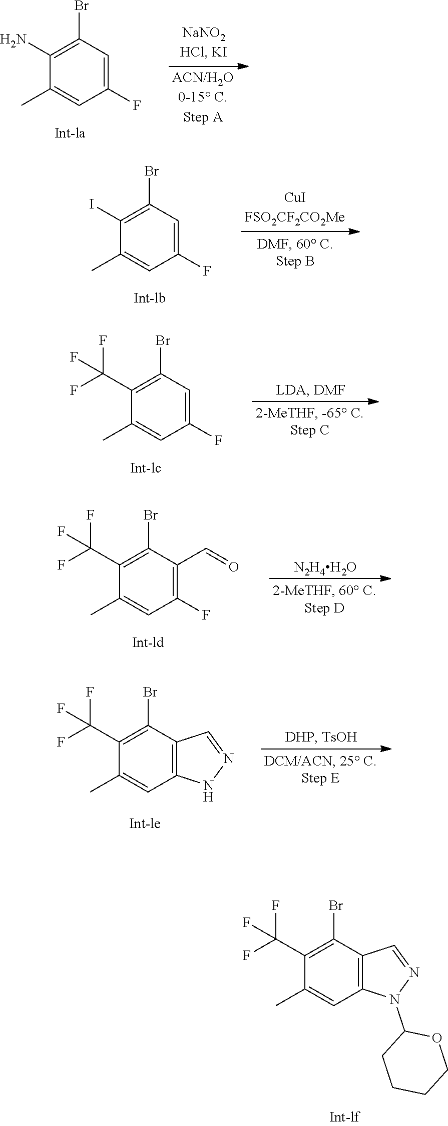

### Step A: 1-bromo-5-fluoro-2-iodo-3-methylbenzene (Int-lb)

2-Bromo-4-fluoro-6-methylaniline (200 g, 0.983 mol) was dissolved in aq. MeCN (800 mL). The resulting mixture was cooled to 0° C. Concentrated HCl (12 M, 245 mL) was added into the reaction mixture while maintaining the reaction temperature at 0° C. A solution of NaNO₂ (81.1 g, 1.18 mol) in water (400 mL) was added dropwise into the reaction mixture maintaining the reaction temperature at 0° C. The resulting mixture was stirred for 0.5 h at 0° C. Then a solution of KI (195 g, 1.18 mol) in water (400 mL) was added dropwise into the reaction mixture at 0° C. The resulting mixture was warmed up to room temperature and stirred for 12 h at 20° C. This reaction was repeated in one additional batch using the above conditions. The two batches of reactions were combined. The product mixture was adjusted to pH 8-9 with aq. NaOH and the aqueous phase was extracted with EtOAc (2 L×2). The organic phase was dried over Na₂SO₄, filtered, and concentrated. The residue obtained was purified by column chromatography (SiO₂, Petroleum ether:Ethyl acetate=1:0 to 0: 1) to afford 1-bromo-5-fluoro-2-iodo-3-methylbenzene (Int-lb). ¹H NMR (400 MHz, CDCl₃) δ 7.27-7.22 (m, 1H), 6.95 (dd, J=8.8, 2.4 Hz, 1H), 2.56 (s, 3H).

### Step B: 1-bromo-5-fluoro-3-methyl-2-(trifluoromethyl)benzene (Int-lc)

1-Bromo-5-fluoro-2-iodo-3-methylbenzene (Int-lb) (100 g, 0.317 mol) was dissolved in DMF (1.50 L). To this mixture were added CuI (514 g, 2.70 mol) and methyl 2,2-difluoro-2-(fluorosulfonyl)acetate (518 g, 2.70 mol) at 25° C. The reaction mixture was heated and stirred for 12 h at 60° C. This reaction was repeated in 3 additional batches using the above conditions. The four batches of reactions were combined and quenched with water (24 L). The mixture was extracted with petroleum ether (8.00 L×2). The combined organic layers were washed with brine (4 L×2) and dried over Na₂SO₄. The dried solution was filtered and the filtrate was concentrated in vacuo to afford the crude material containing 1-bromo-5-fluoro-3-methyl-2-(trifluoromethyl)benzene (Int-ic), which was used directly in the next step without purification.

### Step C: 2-bromo-6-fluoro-4-methyl-3-(trifluoromethyl)benzaldehyde (Int-ld)

1-Bromo-5-fluoro-3-methyl-2-(trifluoromethyl)benzene (Int-ic) (100 g, 0.382 mol) was dissolved in 2-MeTHF (500 mL). The reaction mixture was cooled down to −65° C. A 2 M solution of LDA (213 mL, 426 mmol) was added into the mixture at −65° C. The reaction mixture was stirred for 0.5 h at −65° C. To this mixture was added dropwise DMF (31.2 g, 0.420 mol) at −65° C. The reaction mixture was stirred for 2 h at −65° C. This reaction was repeated in 2 additional batches using the above conditions. The three batches of reactions were combined. The reaction mixture pH was adjusted to 3-4 by using 1 M HCl and the aqueous phase was extracted with 2-MeTHF (500 mL×2). The organic phase was dried over Na₂SO₄, filtered, and concentrated to obtain 2-bromo-6-fluoro-4-methyl-3-(trifluoromethyl)benzaldehyde (Int-ld), which was used in the next step without further purification.

### Step D: 4-bromo-6-methyl-5-(trifluoromethyl)-1H-indazole (Int-le)

2-Bromo-6-fluoro-4-methyl-3-(trifluoromethyl)benzaldehyde (Int-ld) (100 g, 0.351 mol) was dissolved in 2-MeTHF (800 mL). To this mixture was added N₂H₄H₂O (53.7 g, 1.05 mol) at 25° C. The mixture was heated and stirred for 2 h at 60° C. The product mixture was quenched with water (400 mL) and extracted with EtOAc (200 mL×2). The combined organic layers were washed with brine (200 mL) and dried over Na₂SO₄. The dried solution was filtered and the filtrate was concentrated in vacuo to give the residue. This reaction was repeated in 2 additional batches using the above conditions. The three batches of reactions were combined. The residue obtained was triturated with DCM (100 mL) at 15° C. for 2 h. The solid was collected by filtration to afford 4-bromo-6-methyl-5-(trifluoromethyl)-1H-indazole (Int-le). ¹H NMR (400 MHz, CDCl₃) δ 10.61-10.20 (m, 1H), 8.20 (d, J=0.8 Hz, 1H), 7.34 (d, J=0.6 Hz, 1H), 2.67-2.63 (m, 3H).

### Step E: 4-bromo-6-methyl-1-(tetrahydro-2H-pyran-2-yl)-5-(trifluoromethyl)-1H-indazole (Int-lf)

4-Bromo-6-methyl-5-(trifluoromethyl)-1H-indazole (Int-le) (60.0 g, 0.215 mol) was dissolved in DCM (240 mL) and MeCN (240 mL). DHP (21.7 g, 0.258 mol) and TsOH·H₂O (8.18 g, 0.043 mol) were added to the mixture at 20° C. The reaction mixture was stirred for 12 h at 20° C. Water (200 mL) was added to the product mixture. The resulting mixture was extracted with DCM (200 mL×2). The combined organic layers were washed with brine (200 mL) and dried over Na₂SO₄. The dried solution was filtered and the filtrate was concentrated under reduced pressure. The residue obtained was purified by column chromatography (SiO₂, Petroleum ether:Ethyl acetate=1:0 to 0:1) to afford 4-bromo-6-methyl-1-(tetrahydro-2H-pyran-2-yl)-5-(trifluoromethyl)-1H-indazole (Int-if). ¹H NMR (400 MHz, CDCl₃-d) δ 8.12 (s, 1H), 7.44 (s, 1H), 5.69 (dd, J=3, 9 Hz, 1H), 4.09-3.94 (m, 1H), 3.81-3.69 (m, 1H), 2.69-2.63 (m, 3H), 2.56-2.43 (m, 1H), 2.19-2.14 (m, 1H), 2.12-2.04 (m, 1H), 1.87-1.73 (m, 2H), 1.71-1.63 (m, 1H).

### 1.5-oxazocane (Int-mg)

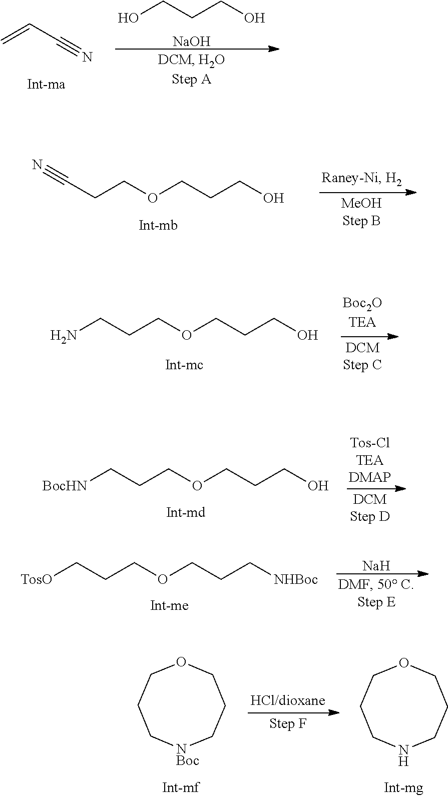

### Step A: 3-(3-hydroxypropoxy)propanenitrile (Int-mb)

To a solution of acrylonitrile (Int-ma) (3.03 g, 57.1 mmol) in DCM (30 mL) and H₂O (30 mL) was added propane-1,3-diol (13.04 g, 171 mmol) and NaOH (8.68 g, 217 mmol) at 25° C. The mixture was stirred at 25° C. for 12 h. The mixture was diluted with water (30 mL), extracted with DCM (3×60 mL), dried over Na₂SO₄, filtered and the solvent was evaporated under reduced pressure to give the crude product. The crude product was purified by flash silica gel chromatography (ISCO®; 40 g SepaFlash® Silica Flash Column, Eluent of 0-30% EtOAc/Pet. ether gradient @40 mL/min) to give 3-(3-hydroxypropoxy)propanenitrile (Int-mb). ¹H NMR (400 MHz, CDCl₃) δ 7.28 (s, 1H), 4.08-3.93 (m, 1H), 3.82-3.75 (m, 1H), 3.84-3.75 (m, 1H), 3.73-3.65 (m, 1H), 3.73-3.64 (m, 1H), 2.68-2.59 (m, 2H), 1.96 (br s, 1H), 1.92-1.80 (m, 2H).

### Step B: 3-(3-aminopropoxy)propan-1-ol (Int-mc)

To a solution of 3-(3-hydroxypropoxy)propanenitrile (Int-mb) (2.7 g, 20.9 mmol) in MeOH (30 mL) was added Raney-Ni (0.123 g, 2.09 mmol) and aqueous ammonia (9.47 mL, 41.8 mmol, 28%) at 25° C. under H₂ atmosphere (50 psi). The mixture was stirred at 25° C. for 12 h. The mixture was filtered and the solvent was evaporated under reduced pressure to give the crude product 3-(3-aminopropoxy)propan-1-ol (Int-mc), which was used in the next step without further purification.

### Step C: tert-butyl (3-(3-hydroxypropoxy)propyl)carbamate (Int-md)

To a solution of 3-(3-aminopropoxy)propan-1-ol (Int-mc) (2.8 g, 21.02 mmol) in DCM (30 mL) was added TEA (3.52 mL, 25.2 mmol), di-tert-butyl dicarbonate (5.05 g, 23.12 mmol) at 25° C. The mixture was stirred at 25° C. for 1 h. The mixture was diluted with water (30 mL), extracted with DCM (3×30 mL), dried over Na₂SO₄, filtered and the solvent was evaporated under reduced pressure to give the crude product. The crude product was purified by flash silica gel chromatography (ISCO®; 80 g SepaFlash® Silica Flash Column, Eluent of 0˜30% EtOAc/Pet. ether gradient at 40 mL/min) to give tert-butyl (3-(3-hydroxypropoxy)propyl)carbamate (Int-md). ¹H NMR (400 MHz, CDCl₃) δ 3.83-3.73 (m, 2H), 3.64-3.55 (m, 2H), 3.53-3.44 (m, 2H), 3.22 (br s, 2H), 1.87-1.68 (m, 4H), 1.47-1.41 (m, 9H).

### Step D: 3-(3-((tert-butoxycarbonyl)amino)propoxy)propyl 4-methylbenzenesulfonate (Int-me)

To a solution of tert-butyl (3-(3-hydroxypropoxy)propyl)carbamate (Int-md) (4.4 g, 18.86 mmol) in DCM (40 mL) was added Tos-Cl (8.09 g, 42.4 mmol), TEA (7.89 mL, 56.6 mmol) and DMAP (0.691 g, 5.66 mmol) at 25° C. The mixture was stirred at 25° C. for 4 h. The mixture was diluted with water (40 mL), extracted with DCM (3×40 mL), dried over Na₂SO₄, filtered and the solvent was evaporated under reduced pressure to give the crude product. The crude product was purified by flash silica gel chromatography (ISCO®; 120 g SepaFlash® Silica Flash Column, Eluent of 0˜30% EtOAc/Pet. ether gradient at 40 mL/min) to give 3-(3-((tert-butoxycarbonyl)amino)propoxy)propyl 4-methylbenzenesulfonate (Int-me). ¹H NMR (400 MHz, CDCl₃) δ 7.79 (d, J=7.6 Hz, 2H), 7.35 (d, J=7.9 Hz, 2H), 4.14-4.09 (m, 3H), 3.45-3.34 (m, 4H), 3.14 (br d, J=5.8 Hz, 2H), 2.45 (s, 3H), 1.89 (q, J=5.8 Hz, 2H), 1.67 (q, J=6.1 Hz, 2H), 1.43 (s, 9H).

### Step E: Tert-butyl 1,5-oxazocane-5-carboxylate (Int-mf)

To a solution of 3-(3-((tert-butoxycarbonyl)amino)propoxy)propyl 4-methylbenzenesulfonate (5.2 g, 13.42 mmol) in DMF (50 mL) was added NaH (1.073 g, 26.8 mmol, 60%) at 0° C. The mixture was stirred at 50° C. for 2 h. The mixture was cooled, diluted with water (50 mL), extracted with EtOAc (3×50 mL), dried over Na₂SO₄, filtered and the solvent was evaporated under reduced pressure to give the crude product. The crude product was purified by flash silica gel chromatography (ISCO®; 80 g SepaFlash® Silica Flash Column, Eluent of 0˜30% EtOAc/Pet. ether gradient at 40 mL/min) to give tert-butyl 1,5-oxazocane-5-carboxylate (Int-mf). MS (ESI) [M+H-tBu]⁺: m/z 160.2

### Step F: 1,5-oxazocane (Int-mg)

A solution of tert-butyl 1,5-oxazocane-5-carboxylate (Int-mf) (50 mg, 0.232 mmol) and HCl gas in dioxane (0.5 mL, 2.000 mmol, 4 N) was stirred at 25° C. for 1 h. The mixture was evaporated under reduced pressure to give the crude product 1,5-oxazocane (Int-mg) as an HCl salt, which was used in the next step without further purification. MS (ESI) [M+H]⁺: m/z 116.2 4-(tert-butoxy)-6,8-difluoro-2-(((2R,7aS)-2-fluorotetrahydro-1H-pyrrolizin-7a(5H)-yl)methoxy)guinazoline (Int-nc)

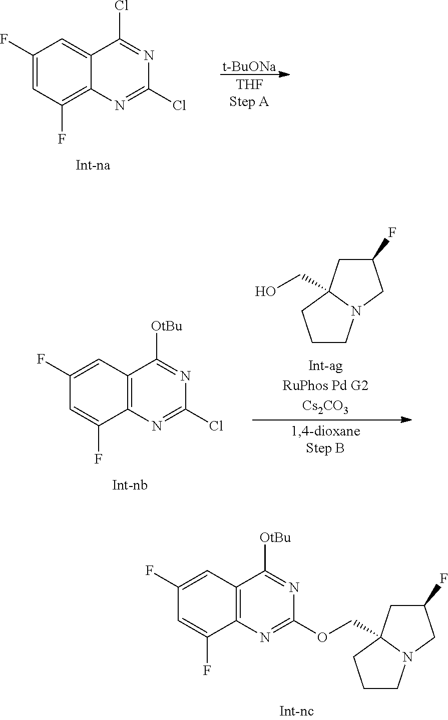 [SMILES: "t-BuONa" ; Fc1cc(F)c2nc(Cl)nc(Cl)c2c1 ; CC(C)(C)Oc1nc(Cl)nc2c(F)cc(F)cc12 ; OC[C@@]12CCCN1C[C@H](F)C2 ; CC(C)(C)Oc1nc(OC[C@@]23CCCN2C[C@H](F)C3)nc2c(F)cc(F)cc12]

### Step A: 4-(tert-butoxy)-2-chloro-6,8-difluoroquinazoline (Int-nb)

A solution of sodium tert-butoxide (33.9 g, 352 mmol) in tetrahydrofuran (400 mL) was added to a solution of 2,4-dichloro-6,8-difluoroquinazoline (Int-na) (79.0 g, 336 mmol) in tetrahydrofuran (800 mL) at 0° C. dropwise. The mixture was warmed to 20° C. and was stirred at that temperature for 4 hours. The mixture was then diluted with ethyl acetate (1.00 L), and the diluted solution was washed sequentially with water (500 mL) and saturated aqueous sodium chloride solution (500 mL). The organic layer was then concentrated under reduced pressure, and the crude residue was purified by flash-column chromatography (eluting with 100:1 v/v petroleum ether-ethyl acetate initially, grading to 10:1 v/v petroleum ether-ethyl acetate) to provide 4-(tert-butoxy)-2-chloro-6,8-difluoroquinazoline (Int-nb). ESI-MS m/z [M+H-tBu]⁺217.

### Step B: 4-(tert-butoxy)-6,8-difluoro-2-(((2R,7aS)-2-fluorotetrahydro-1H-pyrrolizin-7a(5H)-yl)methoxy)quinazoline (Int-nc)

((2R,7aS)-2-fluorotetrahydro-1H-pyrrolizin-7a(5H)-yl)methanol (Int-ag) (3.79 g, 23.8 mmol), cesium carbonate (11.9 g, 36.6 mmol), and RuPhos Pd G2 (1.42 g, 1.83 mmol) were added to a solution of 4-(tert-butoxy)-2-chloro-6,8-difluoroquinazoline (Int-nb) (5.00 g, 18.3 mmol) in 1,4-dioxane (50.0 mL) under an inert atmosphere at 20° C. The mixture was then heated to 80° C. with stirring for 15 h. The mixture was cooled to 20° C. before it was filtered, and the filter cake was rinsed with ethyl acetate (3×15.0 mL). The combined filtrates were concentrated under reduced pressure. The residue was purified by flash-column chromatography (eluting with 100:1 v/v petroleum ether-ethyl acetate initially, grading to 5:1 v/v petroleum ether-ethyl acetate) to provide 4-(tert-butoxy)-6,8-difluoro-2-(((2R,7aS)-2-fluorotetrahydro-1H-pyrrolizin-7a(5H)-yl)methoxy)quinazoline (Int-nc). ESI-MS m/z [M+H]⁺ 396.

### 6-chloro-N,N-bis(4-methoxybenzyl)-4-methyl-5-(trifluoromethyl)pyridin-2-amine (Int-oc)

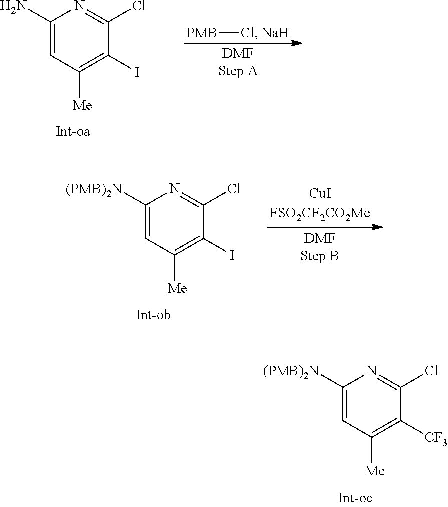

### Step A: 6-chloro-5-iodo-N,N-bis(4-methoxybenzyl)-4-methylpyridin-2-amine (Int-ob)

6-Chloro-5-iodo-4-methylpyridin-2-amine (Int-oa) (133 g, 1.24 mol) was added to a stirred mixture containing sodium hydride (60% w/w mineral oil dispersion, 49.5 g, 1.24 mol) in N,N-dimethylformamide (1.33 L) at 5-10° C. The mixture was then warmed to 25° C. and stirred at that temperature for 1 hour. Next, the mixture was cooled to 0-5° C. and 4-methoxybenzyl chloride (194 g, 1.24 mol) was added. The mixture was again warmed to 25° C. and stirred at that temperature for 5 hours. The reaction mixture was then carefully treated with water (7.00 L) and ethyl acetate (5.00 L). The layers were agitated, then separated. The aqueous phase was extracted with fresh ethyl acetate (3×5.00 L). The combined organic extracts were washed with saturated aqueous sodium chloride solution (5.00 L), dried over sodium sulfate, filtered, and concentrated under reduced pressure. The residue was triturated with methyl tert-butyl ether (1.00 L) for 1 hour at 25° C. to provide 6-chloro-5-iodo-N,N-bis(4-methoxybenzyl)-4-methylpyridin-2-amine (Int-ob). ¹H NMR (400 MHz, CDCl₃) δ (d, J=8.55 Hz, 4H), 6.86 (d, J=8.55 Hz, 4H), 6.26 (s, 1H), 4.64 (s, 4H), 3.81 (s, 6H), 2.31 (s, 3H). ESI-MS m/z [M+H]⁺ 509.

### Step B: 6-chloro-N,N-bis(4-methoxybenzyl)-4-methyl-5-(trifluoromethyl)pyridin-2-amine (Int-oc)

A solution containing 6-chloro-5-iodo-N,N-bis(4-methoxybenzyl)-4-methylpyridin-2-amine (Int-ob) (350 g, 688 mmol) and N,N-dimethylformamide (2.45 L) was treated with copper(I) iodide (262 g, 1.38 mol) and methyl fluorosulfonyldifluoroacetate (264 g, 1.38 mol) at 25° C. The mixture was heated with stirring to 90° C. for 16 hours. The mixture was then cooled to 25° C. before it was diluted with water (6.00 L). The mixture was then extracted with ethyl acetate (2×3.00 L). The combined extracts were filtered, and the filter cake was rinsed with fresh ethyl acetate (1.00 L). The combined filtrates were then washed with saturated aqueous sodium chloride solution (2×5.00 L), dried over sodium sulfate, filtered, and concentrated under reduced pressure. The residue was purified by flash-column chromatography (eluting with petroleum ether initially, then grading to ethyl acetate, then eluting with dichloromethane, then grading to 5:1 v/v dichloromethane-methanol). The semi-purified product thus obtained was then subjected to prep-HPLC (column: Phenomenex Luna C18 250×150 mm, 15 m particle size; eluting with 90% v/v acetonitrile-10 mM aqueous ammonium bicarbonate) to provide 6-chloro-N,N-bis(4-methoxybenzyl)-4-methyl-5-(trifluoromethyl)pyridin-2-amine (Int-oc). ¹H NMR (400 MHz, CDCl₃) δ 7.15 (br d, J=8.60 Hz, 4H), 6.87 (d, J=8.82 Hz, 4H), 6.17 (s, 1H), 4.67 (s, 4H), 3.81 (s, 6H), 2.33 (q, J=2.57 Hz, 3H). ESI-MS m/z [M+H]⁺ 451.

### 7-(6-amino-4-methyl-3-(trifluoromethyl)pyridin-2-yl)-6-chloro-8-fluoro-2-(((2R,7aS)-2-fluorotetrahydro-1H-pyrrolizin-7a(5H)-yl)methoxy)quinazolin-4-ol (Int-pe)

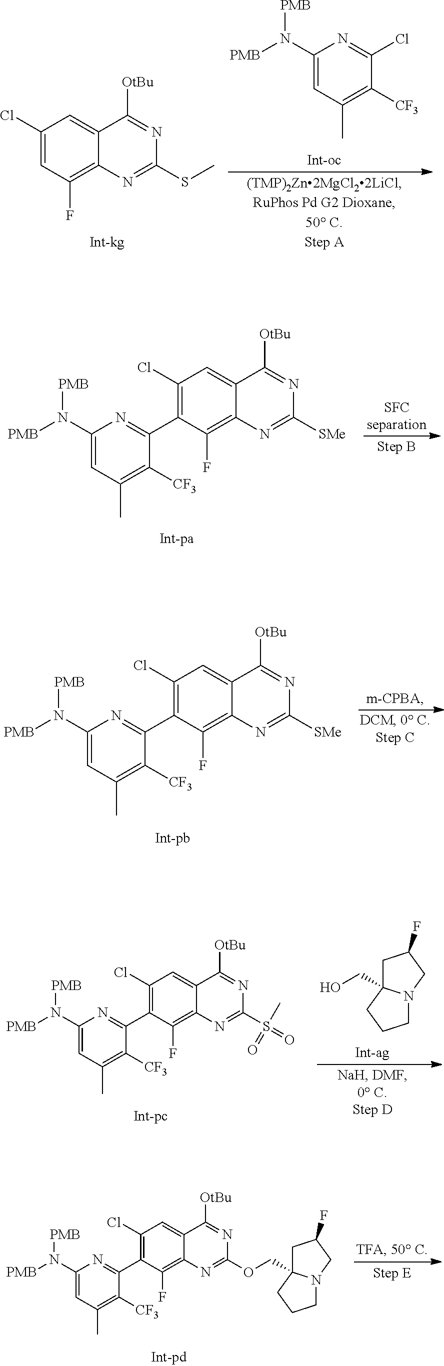

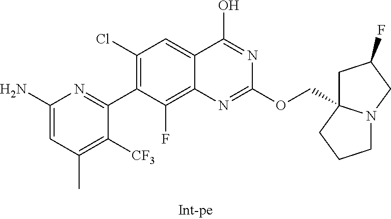 [SMILES: Cc1cc(N)nc(-c2c(Cl)cc3c(O)nc(OC[C@@]45CCCN4C[C@H](F)C5)nc3c2F)c1C(F)(F)F]

### Step A: 6-(4-(tert-butoxy)-6-chloro-8-fluoro-2-(methylthio)quinazolin-7-yl)-N,N-bis(4-methoxybenzyl)-4-methyl-5-(trifluoromethyl)pyridin-2-amine (Int-pa)

A solution of 4-(tert-butoxy)-6-chloro-8-fluoro-2-(methylthio)quinazoline (Int-kg) (32 g, 106 mmol) in (TMP)₂Zn 2MgCl₂ 2LiC1 (532 mL, 213 mmol) (0.4 M in THF) was stirred at 50° C. for 3 h. After cooled to room temperature, chloro(2-dicyclohexylphosphino-2′,6′-diisopropoxy-1,1′-biphenyl)[2-(2′-amino-1,1′-biphenyl)]palladium(II) (12.40 g, 15.96 mmol) in dioxane and 6-chloro-N,N-bis(4-methoxybenzyl)-4-methyl-5-(trifluoromethyl)pyridin-2-amine (Int-oc) (48.0 g, 106 mmol) in THE (200 mL) was added to the reaction solution in a glove box, and the mixture was stirred at 60° C. for 12 h. The reaction mixture was quenched with sat. aqueous NaHCO₃ (300 mL), extracted with EtOAc (3×500 mL), washed with brine, and dried over Na₂SO₄. The organic layer was filtered and the filtrate was concentrated under reduced pressure, and the residue was purified by flash silica gel chromatography (Eluent of 0-18% EtOAc/Pet. ether gradient at 80 mL/min) to provide 6-(4-(tert-butoxy)-6-chloro-8-fluoro-2-(methylthio)quinazolin-7-yl)-N,N-bis(4-methoxybenzyl)-4-methyl-5-(trifluoromethyl)pyridin-2-amine (Int-pa). MS (ESI) [M+H]⁺: m/z 715.6

### Step B: 6-(4-(tert-butoxy)-6-chloro-8-fluoro-2-(methylthio)quinazolin-7-yl)-N,N-bis(4-methoxybenzyl)-4-methyl-5-(trifluoromethyl)pyridin-2-amine (Int-pb)

The racemic 6-(4-(tert-butoxy)-6-chloro-8-fluoro-2-(methylthio)quinazolin-7-yl)-N,N-bis(4-methoxybenzyl)-4-methyl-5-(trifluoromethyl)pyridin-2-amine (Int-pa) (42 g, 58.7 mmol) was separated by preparative SFC (Column: DAICEL CHIRALPAK AS (250 mm×50 mm, 10 um), Condition 0.1% NH₃H₂O IPA, Begin B 30 End B 30 Gradient Time (min) 100 100% B Hold Time 100, Flow Rate (mL/min) 200, Injections 900) to give 6-(4-(tert-butoxy)-6-chloro-8-fluoro-2-(methylthio)quinazolin-7-yl)-N,N-bis(4-methoxybenzyl)-4-methyl-5-(trifluoromethyl)pyridin-2-amine (Int-pb, the second eluting isomer from SFC). ¹H NMR (400 MHz, CDCl₃) δ 7.80 (d, J=1.2 Hz, 1H), 7.07 (d, J=8.6 Hz, 4H), 6.77 (d, J=8.6 Hz, 4H), 6.32 (s, 1H), 4.69 (br d, J=15.7 Hz, 2H), 4.44 (d, J=16.0 Hz, 2H), 3.72 (s, 6H), 2.57 (s, 3H), 2.34 (s, 3H), 1.66 (s, 9H). MS (ESI) [M+H]⁺: m/z 715.6.

### Step C: 6-(4-(tert-butoxy)-6-chloro-8-fluoro-2-(methylsulfonyl)quinazolin-7-yl)-N,N-bis(4-methoxybenzyl)-4-methyl-5-(trifluoromethyl)pyridin-2-amine (Int-pc)

To a solution of 6-(4-(tert-butoxy)-6-chloro-8-fluoro-2-(methylthio)quinazolin-7-yl)-N,N-bis(4-methoxybenzyl)-4-methyl-5-(trifluoromethyl)pyridin-2-amine (Int-pb) (860 mg, 1.20 mmol) in DCM (10 mL) was added m-CPBA (519 mg, 2.405 mmol) (85% w/w) at 0° C. The reaction was stirred at 0° C. for 1 h. The reaction solution was quenched with sat. NaHCO₃ (3 mL), and the resulting mixture was extracted with DCM (30 mL×3). The combined organic phase was dried over anhydrous Na₂SO₄, filtered and concentrated in vacuo. The residue was purified by flash silica gel chromatography (ISCO®, 12 g Agela Flash Column, Pet. ether/EtOAc=3/1, 30 mins, 40 mL/min) to give 6-(4-(tert-butoxy)-6-chloro-8-fluoro-2-(methylsulfonyl)quinazolin-7-yl)-N,N-bis(4-methoxybenzyl)-4-methyl-5-(trifluoromethyl)pyridin-2-amine (Int-pc). MS (ESI) [M+H]⁺: m/z 747.2.

### Step D: 6-(4-(tert-butoxy)-6-chloro-8-fluoro-2-(((2R,7aS)-2-fluorotetrahydro-1H-pyrrolizin-7a(5H)-yl)methoxy)quinazolin-7-yl)-N,N-bis(4-methoxybenzyl)-4-methyl-5-(trifluoromethyl)pyridin-2-amine (Int-pd)

To a solution of ((2R,7aS)-2-fluorotetrahydro-1H-pyrrolizin-7a(5H)-yl)methanol (Int-ag) (298 mg, 1.874 mmol) in THE (10 mL) was added NaH (74.9 mg, 1.87 mmol) (60% in mineral oil) at 0° C. under N₂ atmosphere. The mixture was stirred at 0° C. for 10 min, then 6-(4-(tert-butoxy)-6-chloro-8-fluoro-2-(methylsulfonyl)quinazolin-7-yl)-N,N-bis(4-methoxybenzyl)-4-methyl-5-(trifluoromethyl)pyridin-2-amine (Int-pc) (700 mg, 0.937 mmol) was added to the above mixture, and the mixture was stirred at 0° C. for 2 h. The mixture was quenched with water (2 mL) and extracted with EtOAc (30 mL×3), dried over Na₂SO₄, filtered and the solvent was evaporated under reduced pressure to give the crude product 6-(4-(tert-butoxy)-6-chloro-8-fluoro-2-(((2R,7aS)-2-fluorotetrahydro-1H-pyrrolizin-7a(5H)-yl)methoxy)quinazolin-7-yl)-N,N-bis(4-methoxybenzyl)-4-methyl-5-(trifluoromethyl)pyridin-2-amine (Int-pd). MS (ESI) [M+H]⁺: m/z 826.4

### Step E: 7-(6-amino-4-methyl-3-(trifluoromethyl)pyridin-2-yl)-6-chloro-8-fluoro-2-(((2R,7aS)-2-fluorotetrahydro-1H-pyrrolizin-7a(5H)-yl)methoxy)quinazolin-4-ol (Int-Pe)

A solution of 6-(4-(tert-butoxy)-6-chloro-8-fluoro-2-(((2R,7aS)-2-fluorotetrahydro-1H-pyrrolizin-7a(5H)-yl)methoxy)quinazolin-7-yl)-N,N-bis(4-methoxybenzyl)-4-methyl-5-(trifluoromethyl)pyridin-2-amine (Int-pd) (1.89 g, 2.29 mmol) in TFA (15 mL, 195 mmol) was stirred at 50° C. for 15 h. The solvent was evaporated under reduced pressure to give the residue, which was diluted with DCM (200 mL), basified with saturated sodium bicarbonate aqueous solution until pH 7. The organic layer was separated and dried over Na₂SO₄, filtered and the solvent was evaporated under reduced pressure. The crude product was purified by reverse preparative HPLC (Column: Boston Green ODS 150×30 mm×5 um; Condition: water (0.2% FA)-ACN; Begin B—End B: 18—38; Gradient Time (min): 10; 100% B Hold Time (min): 2; FlowRate (mL/min): 25) to give 7-(6-amino-4-methyl-3-(trifluoromethyl)pyridin-2-yl)-6-chloro-8-fluoro-2-(((2R,7aS)-2-fluorotetrahydro-1H-pyrrolizin-7a(5H)-yl)methoxy)quinazolin-4-ol (Int-pe). MS (ESI) [M+H]⁺: m/z 530.2.

### EXAMPLES

### Example 1: 2-amino-4-(4-(azepan-1-yl)-6-chloro-8-fluoro-2-(((2R,7aS)-2-fluorotetrahydro-1H-pyrrolizin-7a(5H)-yl)methoxy)quinazolin-7-yl)-7-fluorobenzo[b]thiophene-3-carbonitrile (Ex. 1)

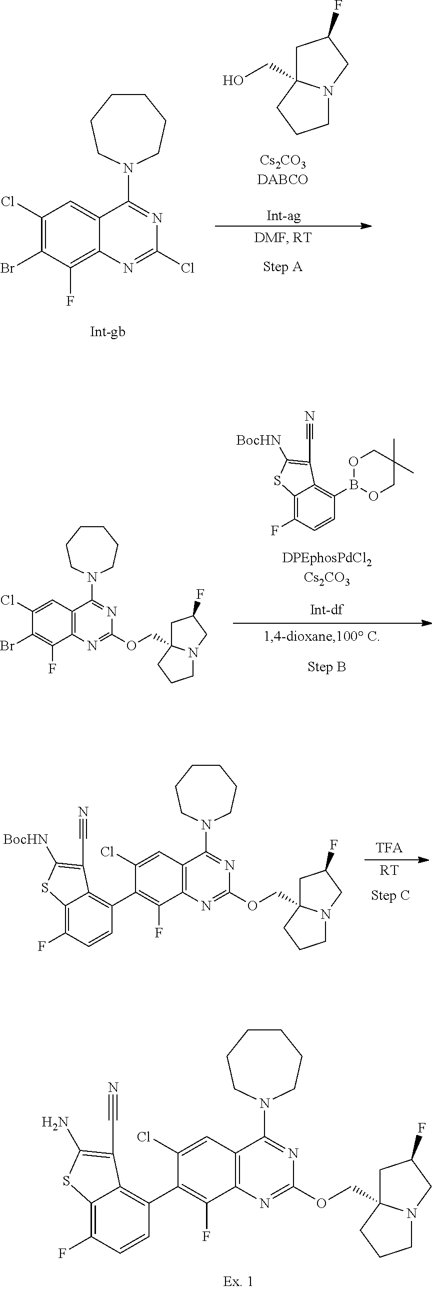

### Step A: 4-(azepan-1-yl)-7-bromo-6-chloro-8-fluoro-2-(((2R,7aS)-2-fluorotetrahydro-1H-pyrrolizin-7a(5H)-yl)methoxy)guinazoline

To a solution of 4-(azepan-1-yl)-7-bromo-2,6-dichloro-8-fluoroquinazoline (Int-gb) (233 mg, 0.59 mmol), ((2R,7aS)-2-fluorotetrahydro-1H-pyrrolizin-7a(5H)-yl)methanol (Int-ag) (188 mg, 1.18 mmol), cesium carbonate (578 mg, 1.77 mmol) and 1,4-diazabicyclo[2.2.2]octane (13.3 mg, 0.12 mmol) in N,N-dimethylformamide (2.3 ml) was stirred at room temperature for 14 h under nitrogen. The reaction mixture was added water and the suspension was filtered and the solid was washed with water. The filter cake was dried under reduced pressure to obtain 4-(azepan-1-yl)-7-bromo-6-chloro-8-fluoro-2-(((2R,7aS)-2-fluorotetrahydro-1H-pyrrolizin-7a(5H)-yl)methoxy)quinazoline. ESI-MS m/z [M+H]⁺ 515, 517, 519. ¹H NMR (400 MHz, DMSO-d6) δ 8.01 (d, J=1.9 Hz, 1H), 5.34-5.19 (m, 1H), 4.09-3.97 (m, 2H), 3.89-3.86 (m, 4H), 3.08-3.00 (m, 3H), 2.84-2.81 (m, 1H), 2.11-2.10 (m, 1H), 2.03-1.96 (m, 2H), 1.88-1.74 (m, 7H), 1.55 (br s, 4H).

### Step B: Tert-butyl (4-(4-(azepan-1-yl)-6-chloro-8-fluoro-2-(((2R,7aS)-2-fluorotetrahydro-1H-pyrrolizin-7a(5H)-yl)methoxy)quinazolin-7-yl)-3-cyano-7-fluorobenzo[b]thiophen-2-yl)carbamate

To a solution of 4-(azepan-1-yl)-7-bromo-6-chloro-8-fluoro-2-(((2R,7aS)-2-fluorotetrahydro-1H-pyrrolizin-7a(5H)-yl)methoxy)quinazoline (100 mg, 0.19 mmol), tert-butyl (3-cyano-4-(5,5-dimethyl-1,3,2-dioxaborinan-2-yl)-7-fluorobenzo[b]thiophen-2-yl)carbamate (Int-df) (157 mg, 0.39 mmol) and cesium carbonate (158 mg, 0.49 mmol) in 1,4-dioxane (5.0 ml) was added dichloro[bis(2-(diphenylphosphino)phenyl)ether]palladium(II) (27.8 mg, 0.039 mmol) at room temperature. The mixture was degassed under reduced pressure and purged with nitrogen several times. The mixture was stirred at 100° C. for 1 h under nitrogen atmosphere. The reaction mixture was cooled to room temperature and diluted with water and ethyl acetate. The organic layer was dried over MgSO₄, filtered and concentrated in vacuo. The residue was purified by reverse phase HPLC (MeCN/water with 0.1% formic acid) to obtain tert-butyl (4-(4-(azepan-1-yl)-6-chloro-8-fluoro-2-(((2R,7aS)-2-fluorotetrahydro-1H-pyrrolizin-7a(5H)-yl)methoxy)quinazolin-7-yl)-3-cyano-7-fluorobenzo[b]thiophen-2-yl)carbamate. ESI-MS m/z [M+H]⁺ 727, 729, 731. ¹H NMR (400 MHz, CDCl₃) δ 7.89 (d, J=1.6 Hz, 1H), 7.31-7.28 (m, 1H), 7.17-7.12 (m, 1H), 5.34-5.20 (m, 1H), 4.27-4.22 (m, 1H), 4.16-4.09 (m, 1H), 4.00-3.89 (m, 4H), 3.27-3.11 (m, 3H), 2.99-2.94 (m, 1H), 2.29 (br s, 1H), 2.26-2.14 (m, 2H), 2.05-1.79 (m, 7H), 1.70-1.69 (m, 4H), 1.57 (s, 9H).

### Step C: 2-amino-4-(4-(azepan-1-yl)-6-chloro-8-fluoro-2-(((2R,7aS)-2-fluorotetrahydro-1H-pyrrolizin-7a(5H)-yl)methoxy)quinazolin-7-yl)-7-fluorobenzo[b]thiophene-3-carbonitrile (Ex. 1)

To a solution of tert-butyl (4-(4-(azepan-1-yl)-6-chloro-8-fluoro-2-(((2R,7aS)-2-fluorotetrahydro-1H-pyrrolizin-7a(5H)-yl)methoxy)quinazolin-7-yl)-3-cyano-7-fluorobenzo[b]thiophen-2-yl)carbamate (61 mg, 0.084 mmol) in dichloromethane (1.2 ml) was added trifluoroacetic acid (0.62 ml, 8.39 mmol) at room temperature under an atmosphere of nitrogen. The mixture was stirred at room temperature for 1 h under nitrogen atmosphere. The reaction mixture was basified with saturated sodium bicarbonate aqueous solution. The organic layer was dried over MgSO₄, filtered and concentrated in vacuo. The residue was purified by reverse phase HPLC (MeCN/water with 0.1% formic acid) to obtain 2-amino-4-(4-(azepan-1-yl)-6-chloro-8-fluoro-2-(((2R,7aS)-2-fluorotetrahydro-1H-pyrrolizin-7a(5H)-yl)methoxy)quinazolin-7-yl)-7-fluorobenzo[b]thiophene-3-carbonitrile (Ex. 1). ESI-MS m/z [M+H]⁺ 627, 629. ¹H NMR (400 MHz, CDCl₃) δ 7.90-7.86 (m, 1H), 7.20-7.16 (m, 1H), 7.01-6.96 (m, 1H), 6.11-6.01 (m, 2H), 5.33-5.19 (m, 1H), 4.26-4.21 (m, 1H), 4.16-4.10 (m, 1H), 3.98-3.86 (m, 4H), 3.27-3.16 (m, 3H), 3.01-2.95 (m, 1H), 2.28-2.27 (m, 1H), 2.24-2.11 (m, 2H), 2.01-1.83 (m, 7H), 1.68 (br s, 4H).

Compounds in the table below were synthesized via a similar route as Ex. 1 using 7-bromo-2,4,6-trichloro-8-fluoroquinazoline (Int-ga) and azepanes or the corresponding amines.

| | | | | ESI-MS |
|---|---|---|---|---|
| Ex. | Structure | Name | NMR | [M + H]⁺ |
| 2 | 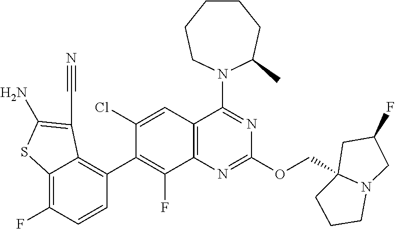 [SMILES: C[C@@H]1CCCCCN1c1nc(OC[C@@]23CCCN2C[C@H](F)C3)nc2c(F)c(-c3ccc(F)c4sc(N)c(C#N)c34)c(Cl)cc12] | 2-amino-4-(6- chloro-8-fluoro-2- (((2R,7aS)-2- fluorotetrahydro- 1H-pyrrolizin- 7a(5H)- yl)methoxy)-4- ((R)-2- methylazepan-1- yl)quinazolin-7- yl)-7- fluorobenzo[b]thio- phene-3- carbonitrile | ¹H NMR (500 MHz, CDCl₃) δ 7.95-7.90 (m, 1H), 7.20-7.14 (m, 1 H), 7.00-6.94 (m, 1H), 6.37-6.21 (m, 1H), 6.00-5.82 (m, 1H), 5.31-5.20 (m, 1H), 4.85-4.76 (m, 1H), 4.23-4.18 (m, 2H), 4.12-4.10 (m, 1H), 3.37-3.17 (m, 4H), 2.99-2.96 (m, 1H), 2.31-2.12 (m, 4H), 2.06-1.82 | 641, 643 |
| | | | (m, 7H), 1.50-1.60 | |
| | | | (m, 1H), 1.46-1.37 | |
| | | | (m, 1H), 1.33-1.23 | |
| | | | (m, 4H). | |
| 3 | 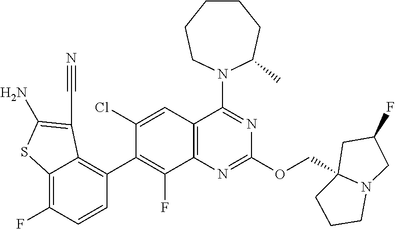 [SMILES: C[C@H]1CCCCCN1c1nc(OC[C@@]23CCCN2C[C@H](F)C3)nc2c(F)c(-c3ccc(F)c4sc(N)c(C#N)c34)c(Cl)cc12] | 2-amino-4-(6- chloro-8-fluoro-2- (((2R,7aS)-2- fluorotetrahydro- 1H-pyrrolizin- 7a(5H)- yl)methoxy)-4- ((S)-2- methylazepan-1- yl)quinazolin-7- yl)-7- fluorobenzo[b]thio- phene-3- carbonitrile | ¹H NMR (400 MHz, CDCl₃) δ 7.93 (s, 1H), 7.21-7.15 (m, 1 H), 7.01-6.96 (m, 1H), 6.17 (br s, 1H), 5.89 (br s, 1H), 5.32- 5.21 (m, 1H), 4.86- 4.77 (m, 1H), 4.25- 4.21 (m, 2H), 4.11- 4.07 (m, 1H), 3.38- 3.14 (m, 4H), 3.01- 2.95 (m, 1H), 2.28- 2.16 (m, 4H), 2.08- 1.83 (m, 7H), 1.59- | 641, 643 |
| | | | 1.51 (m, 1H), 1.47- | |
| | | | 1.24 (m, 5H). | |
| 4 | 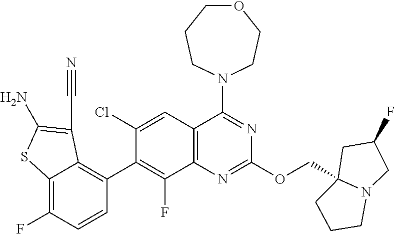 [SMILES: N#Cc1c(N)sc2c(F)ccc(-c3c(Cl)cc4c(N5CCCOCC5)nc(OC[C@@]56CCCN5C[C@H](F)C6)nc4c3F)c12] | 2-amino-4-(6- chloro-8-fluoro-2- (((2R,7aS)-2- fluorotetrahydro- 1H-pyrrolizin- 7a(5H)- yl)methoxy)-4- (1,4-oxazepan-4- yl)quinazolin-7- yl)-7- fluorobenzo[b]thio- phene-3- carbonitrile | ¹H NMR (400 MHz, CDCl₃) δ 7.82 (d, J = 1.5 Hz, 1H), 7.22- 7.18 (m, 1H), 7.03- 6.99 (m, 1H), 5.79- 5.76 (m, 2H), 5.34- 5.20 (m, 1H), 4.27- 4.22 (m, 1H), 4.16- 3.97 (m, 7H), 3.87- 3.85 (m, 2H), 3.29- 3.17 (m, 3H), 3.01 2.95 (m, 1H), 2.29- 2.12 (m, 5H), 1.97- 1.81 (m, 3H). | 629, 631 |
| 5 | 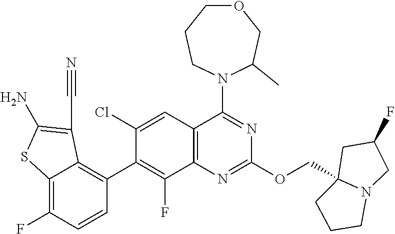 [SMILES: CC1COCCCN1c1nc(OC[C@@]23CCCN2C[C@H](F)C3)nc2c(F)c(-c3ccc(F)c4sc(N)c(C#N)c34)c(Cl)cc12] | 2-amino-4-(6- chloro-8-fluoro-2- (((2R,7aS)-2- fluorotetrahydro- 1H-pyrrolizin- 7a(5H)- yl)methoxy)-4-(3- methyl-1,4- oxazepan-4- yl)quinazolin-7- yl)-7- fluorobenzo[b]thio- phene-3- carbonitrile | ¹H NMR (400 MHz, CDCl₃) δ 7.83-7.79 (m, 1H), 7.21-7.15 (m, 1 H), 7.01-6.96 (m, 1H), 6.14-6.13 (m, 1H), 6.01-5.96 (m, 1H), 5.33-5.20 (m, 1H), 4.86-4.74 (m, 1H), 4.44-3.98 (m, 5H), 3.75-3.52 (m, 3H), 3.28-3.12 (m, 3H), 3.01-2.95 (m, 1H), 2.50-2.35 (m, 1H), 2.28-2.11 (m, 3H), 2.03-1.86 | 643, 645 |
| | | | (m, 4H), 1.41-1.38 | |
| | | | (m, 3H). | |
| 6 | 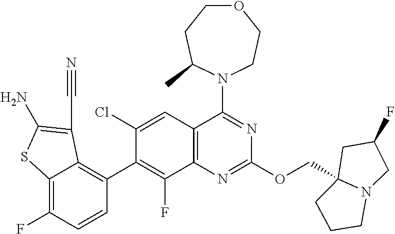 [SMILES: C[C@H]1CCOCCN1c1nc(OC[C@@]23CCCN2C[C@H](F)C3)nc2c(F)c(-c3ccc(F)c4sc(N)c(C#N)c34)c(Cl)cc12] | 2-amino-4-(6- chloro-8-fluoro-2- (((2R,7aS)-2- fluorotetrahydro- 1H-pyrrolizin- 7a(5H)- yl)methoxy)-4- ((S)-5-methyl-1,4- oxazepan-4- yl)quinazolin-7- yl)-7- fluorobenzo[b]thio- phene-3- carbonitrile | ¹H NMR (400 MHz, CDCl₃) δ 7.77-7.75 (m, 1H), 7.22-7.16 (m, 1 H), 7.04-6.98 (m, 1H), 5.89 (br s, 1H), 5.68 (br s, 1H), 5.33-5.20 (m, 1H), 4.82-4.70 (m, 1H), 4.41-4.20 (m, 1H), 4.13-4.06 (m, 2H), 4.02-3.95 (m, 2H), 3.60-3.39 (m, 2H), 3.27-3.11 (m, 3H), 3.06-2.96 (m, 1H), 2.37-2.13 (m, 4H), | 643, 645 |
| | | | 2.05-1.80 (m, 4H), | |
| | | | 1.50-1.47 (m, 3H). | |
| 7 | 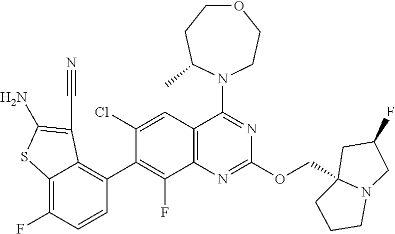 [SMILES: C[C@@H]1CCOCCN1c1nc(OC[C@@]23CCCN2C[C@H](F)C3)nc2c(F)c(-c3ccc(F)c4sc(N)c(C#N)c34)c(Cl)cc12] | 2-amino-4-(6- chloro-8-fluoro-2- (((2R,7aS)-2- fluorotetrahydro- 1H-pyrrolizin- 7a(5H)- yl)methoxy)-4- ((R)-5-methyl-1,4- oxazepan-4- yl)quinazolin-7- yl)-7- fluorobenzo[b]thio- phene-3- carbonitrile | ¹H NMR (400 MHz, CDCl₃) δ 7.77-7.75 (m, 1H), 7.22-7.15 (m, 1 H), 7.04-6.97 (m, 1H), 5.99 (br s, 1H), 5.70 (br s, 1H), 5.34-5.20 (m, 1H), 4.82-4.70 (m, 1H), 4.41-4.37 (m, 1H), 4.25-4.18 (m, 1H), 4.11-4.06 (m, 2H), 4.01-3.94 (m, 2H), 3.60-3.39 (m, 2H), 3.29-3.12 (m, 3H), 3.01-2.95 (m, 1H), | 643, 645 |
| | | | 2.37-2.11 (m, 4H), | |
| | | | 2.05-1.79 (m, 4H), | |
| | | | 1.50-1.47 (m, 3H). | |
| 8 | 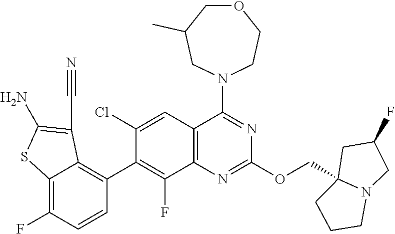 [SMILES: CC1COCCN(c2nc(OC[C@@]34CCCN3C[C@H](F)C4)nc3c(F)c(-c4ccc(F)c5sc(N)c(C#N)c45)c(Cl)cc23)C1] | 2-amino-4-(6- chloro-8-fluoro-2- (((2R,7aS)-2- fluorotetrahydro- 1H-pyrrolizin- 7a(5H)- yl)methoxy)-4-(6- methyl-1,4- oxazepan-4- yl)quinazolin-7- yl)-7- fluorobenzo[b]thio- phene-3- carbonitrile | ¹H NMR (400 MHz, CDCl₃) δ 7.82-7.78 (m, 1H), 7.23-7.18 (m, 1 H), 7.03-6.99 (m, 1H), 5.79-5.75 (m, 2H), 5.34-5.20 (m, 1H), 4.32-4.04 (m, 5H), 3.98-3.89 (m, 2H), 3.85-3.78 (m, 1H), 3.50-3.38 (m, 2H), 3.28-3.17 (m, 3H), 3.01-2.95 (m, 1H), 2.54-2.40 (m, 1H), 2.32-2.10 (m, 3H), 1.96-1.84 | 643, 645 |
| | | | (m, 3H), 1.02-1.00 | |
| | | | (m, 3H). | |
| 9 | 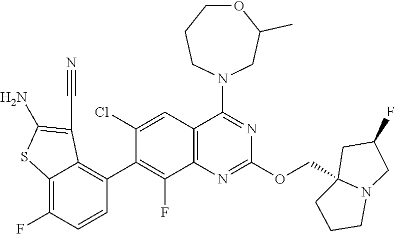 [SMILES: CC1CN(c2nc(OC[C@@]34CCCN3C[C@H](F)C4)nc3c(F)c(-c4ccc(F)c5sc(N)c(C#N)c45)c(Cl)cc23)CCCO1] | 2-amino-4-(6- chloro-8-fluoro-2- (((2R,7aS)-2- fluorotetrahydro- 1H-pyrrolizin- 7a(5H)- yl)methoxy)-4-(2- methyl-1,4- oxazepan-4- yl)quinazolin-7- yl)-7- fluorobenzo[b]thio- phene-3- carbonitrile | ¹H NMR (400 MHz, CDCl₃) δ 7.81-7.80 (m, 1H), 7.22-7.18 (m, 1 H), 7.03-6.99 (m, 1H), 5.84-5.76 (m, 2H), 5.34-5.21 (m, 1H), 4.46-4.36 (m, 1H), 4.27-4.10 (m, 4H), 4.00-3.90 (m, 2H), 3.65-3.56 (m, 1H), 3.44-3.17 (m, 4H), 3.01-2.95 (m, 1H), 2.33-2.12 (m, 5H), 1.97-1.81 (m, 3H), 1.32-1.26 | 643, 645 |
| | | | (m, 3H). | |
| 10 | 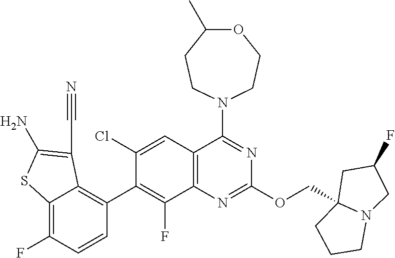 [SMILES: CC1CCN(c2nc(OC[C@@]34CCCN3C[C@H](F)C4)nc3c(F)c(-c4ccc(F)c5sc(N)c(C#N)c45)c(Cl)cc23)CCO1] | 2-amino-4-(6- chloro-8-fluoro-2- (((2R,7aS)-2- fluorotetrahydro- 1H-pyrrolizin- 7a(5H)- yl)methoxy)-4-(7- methyl-1,4- oxazepan-4- yl)quinazolin-7- yl)-7- fluorobenzo[b]thio- phene-3- carbonitrile | ¹H NMR (400 MHz, CDCl₃) δ 7.82-7.79 (m, 1H), 7.20-7.17 (m, 1 H), 7.02-6.97 (m, 1H), 5.89 (br s, 1H), 5.82 (br s, 1H), 5.33-5.19 (m, 1H), 4.26-4.10 (m, 5H), 3.97-3.70 (m, 4H), 3.27-3.11 (m, 3H), 3.00-2.94 (m, 1H), 2.31-1.83 (m, 8H), 1.30-1.26 (m, 3H). | 643, 645 |
| 11 | 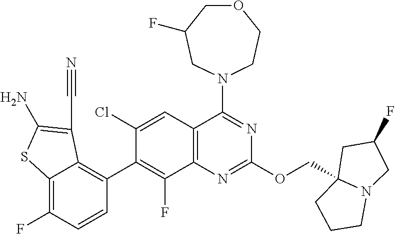 [SMILES: N#Cc1c(N)sc2c(F)ccc(-c3c(Cl)cc4c(N5CCOCC(F)C5)nc(OC[C@@]56CCCN5C[C@H](F)C6)nc4c3F)c12] | 2-amino-4-(6- chloro-8-fluoro-4- (6-fluoro-1,4- oxazepan-4-yl)-2- (((2R,7aS)-2- fluorotetrahydro- 1H-pyrrolizin- 7a(5H)- yl)methoxy)quinazo- lin-7-yl)-7- fluorobenzo[b]thio- phene-3- carbonitrile | ¹H NMR (400 MHz, CDCl₃) δ 7.90-7.85 (m, 1H), 7.23-7.19 (m, 1 H), 7.05-7.00 (m, 1H), 5.45 (br s, 2H), 5.34-5.08 (m, 2H), 4.44-4.33 (m, 1H), 4.29-3.77 (m, 9H), 3.26-3.11 (m, 3H), 3.00-2.94 (m, 1H), 2.26-1.80 (m, 6H). | 647, 649 |
| 12 | 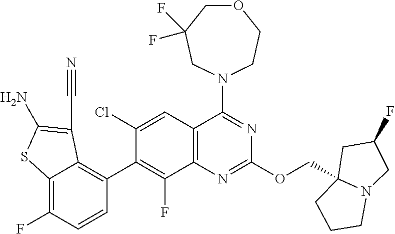 [SMILES: N#Cc1c(N)sc2c(F)ccc(-c3c(Cl)cc4c(N5CCOCC(F)(F)C5)nc(OC[C@@]56CCCN5C[C@H](F)C6)nc4c3F)c12] | 2-amino-4-(6- chloro-4-(6,6- difluoro-1,4- oxazepan-4-yl)-8- fluoro-2- (((2R,7aS)-2- fluorotetrahydro- 1H-pyrrolizin- 7a(5H)- yl)methoxy)quinazo- lin-7-yl)-7- fluorobenzo[b]thio- phene-3- carbonitrile | ¹H NMR (400 MHz, CDCl₃) δ 7.85 (s, 1H), 7.23-7.19 (m, 1 H), 7.07-7.01 (m, 1H), 5.48 (br s, 2H), 5.35-5.22 (m, 2H), 4.58-4.47 (m, 1H), 4.34-3.95 (m, 9H), 3.28-3.13 (m, 3H), 3.01-2.96 (m, 1H), 2.27-2.12 (m, 3H), 1.97-1.85 (m, 3H). | 665, 667 |
| 13 | 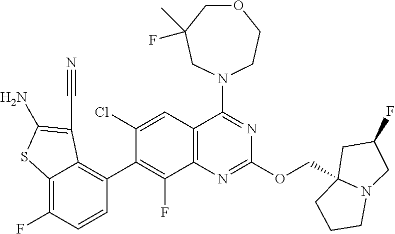 [SMILES: CC1(F)COCCN(c2nc(OC[C@@]34CCCN3C[C@H](F)C4)nc3c(F)c(-c4ccc(F)c5sc(N)c(C#N)c45)c(Cl)cc23)C1] | 2-amino-4-(6- chloro-8-fluoro-4- (6-fluoro-6- methyl-1,4- oxazepan-4-yl)-2- (((2R,7aS)-2- fluorotetrahydro- 1H-pyrrolizin- 7a(5H)- yl)methoxy)quinazo- lin-7-yl)-7- fluorobenzo[b]thio- phene-3- carbonitrile | ¹H NMR (400 MHz, CDCl₃) δ 8.00-7.95 (m, 1H), 7.24-7.19 (m, 1H), 7.06-7.01 (m, 1H), 5.56-5.54 (m, 2H), 5.34-5.21 (m, 2H), 4.54-3.67 (m, 10H), 3.27- 3.13 (m, 3H), 3.01- 2.95 (m, 1H), 2.27- 2.10 (m, 3H), 1.97- 1.86 (m, 3H), 1.50- 1.41 (m, 3H). | 661, 663 |
| 14 | 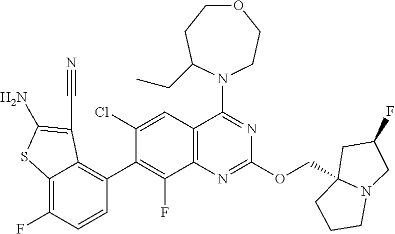 [SMILES: CCC1CCOCCN1c1nc(OC[C@@]23CCCN2C[C@H](F)C3)nc2c(F)c(-c3ccc(F)c4sc(N)c(C#N)c34)c(Cl)cc12] | 2-amino-4-(6- chloro-4-(5-ethyl- 1,4-oxazepan-4- yl)-8-fluoro-2- (((2R,7aS)-2- fluorotetrahydro- 1H-pyrrolizin- 7a(5H)- yl)methoxy)quinazo- lin-7-yl)-7- fluorobenzo[b]thio- phene-3- carbonitrile | ¹H NMR (400 MHz, CDCl₃) δ 7.71 (s, 1H), 7.24-7.09 (m, 1H), 7.04-6.99 (m, 1H), 5.68-5.22 (m, 2H), 5.33-5.20 (m, 1H), 4.54-4.41 (m, 2H), 4.24-3.81 (m, 5H), 3.58-3.43 (m, 2H), 3.26-3.15 (m, 3H), 3.00-2.94 (m, 1H), 2.47-2.40 (m, 1H), 2.12-2.27 (m, 3H), 2.06-1.74 (m, 6H), 1.06-10.98 (m, | 657, 659 |
| | | | 3H). | |
| 15 | 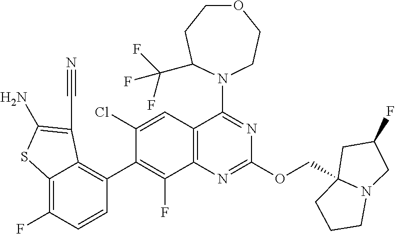 [SMILES: N#Cc1c(N)sc2c(F)ccc(-c3c(Cl)cc4c(N5CCOCCC5C(F)(F)F)nc(OC[C@@]56CCCN5C[C@H](F)C6)nc4c3F)c12] | 2-amino-4-(6- chloro-8-fluoro-2- (((2R,7aS)-2- fluorotetrahydro- 1H-pyrrolizin- 7a(5H)- yl)methoxy)-4-(5- (trifluoromethyl)- 1,4-oxazepan-4- yl)quinazolin-7- yl)-7- fluorobenzo[b]thio- phene-3- carbonitrile | ¹H NMR (400 MHz, CDCl₃) δ 7.81-7.78 (m, 1H), 7.27-7.01 (m, 2H), 5.88-5.68 (m, 1H), 5.52-5.23 (m, 2H), 4.43-4.12 (m, 4H), 4.00-3.89 (m, 1H), 3.83-3.65 (m, 2H), 3.38-3.13 (m, 4H), 3.01-2.98 (m, 1H), 2.60-2.50 (m, 1H), 2.41-2.14 (m, 5H), 1.97-1.86 (m, 3H). | 697, 699 |
| 16 | 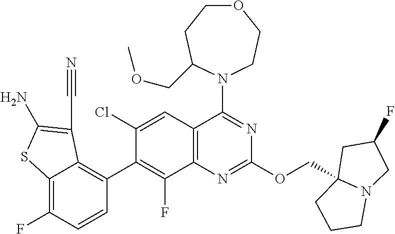 [SMILES: COCC1CCOCCN1c1nc(OC[C@@]23CCCN2C[C@H](F)C3)nc2c(F)c(-c3ccc(F)c4sc(N)c(C#N)c34)c(Cl)cc12] | 2-amino-4-(6- chloro-8-fluoro-2- (((2R,7aS)-2- fluorotetrahydro- 1H-pyrrolizin- 7a(5H)- yl)methoxy)-4-(5- (methoxymethyl)- 1,4-oxazepan-4- yl)quinazolin-7- yl)-7- fluorobenzo[b]thio- phene-3- carbonitrile | ¹H NMR (400 MHz, CDCl₃) δ 7.96-7.90 (m, 1H), 7.22-7.16 (m, 1H), 7.03-6.98 (m, 1H), 5.80-5.67 (m, 2H), 5.33-5.19 (m, 1H), 4.94-4.85 (m, 1H), 4.48-4.44 (m, 1H), 4.25-4.00 (m, 4H), 3.92-3.85 (m, 1H), 3.77-3.67 (m, 3H), 3.58-3.37 (m, 4H), 3.26-3.10 (m, 3H), 3.00-2.94 (m, 1H), 2.36-2.09 | 673, 675 |
| | | | (m, 5H), 1.96-1.84 | |
| | | | (m, 3H). | |
| 17 | 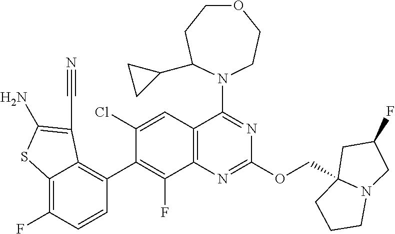 [SMILES: N#Cc1c(N)sc2c(F)ccc(-c3c(Cl)cc4c(N5CCOCCC5C5CC5)nc(OC[C@@]56CCCN5C[C@H](F)C6)nc4c3F)c12] | 2-amino-4-(6- chloro-4-(5- cyclopropyl-1,4- oxazepan-4-yl)-8- fluoro-2- (((2R,7aS)-2- fluorotetrahydro- 1H-pyrrolizin- 7a(5H)- yl)methoxy)quinazo- lin-7-yl)-7- fluorobenzo[b]thio- phene-3- carbonitrile | ¹H NMR (400 MHz, CDCl₃) δ 7.90-7.82 (m, 1H), 7.23-7.20 (m, 1H), 7.04-7.00 (m, 1H), 5.65-5.60 (m, 2H), 5.33-5.20 (m, 1H), 4.56-4.44 (m, 1H), 4.31-3.94 (m, 5H), 3.84-3.62 (m, 3H), 3.28-3.13 (m, 3H), 3.01-2.95 (m, 1H), 2.48-2.36 (m, 1H), 2.29-2.10 (m, 4H), 1.96-1.84 (m, 3H), 1.44-1.33 | 669, 671 |
| | | | (m, 1H), 0.72-0.15 | |
| | | | (m, 4H). | |

### Example 18: 2-amino-4-(6-chloro-8-fluoro-2-(((2R,7aS)-2-fluorotetrahydro-111-pyrrolizin-7a(5H)-yl)methoxy)-4-(1,4-oxazepan-4-yl)quinazolin-7-yl)-7-fluorobenzo[b]thiophene-3-carbonitrile (Ex. 18)

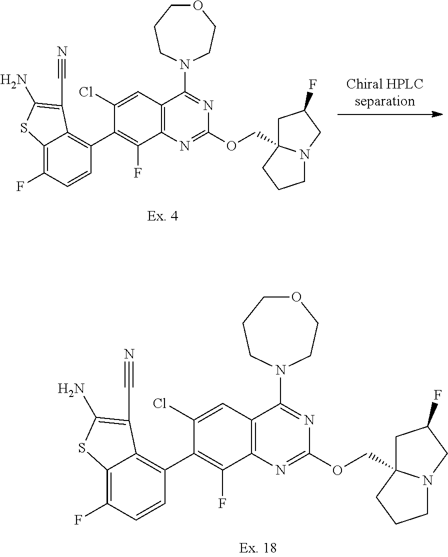 [SMILES: N#Cc1c(N)sc2c(F)ccc(-c3c(Cl)cc4c(N5CCCOCC5)nc(OC[C@@]56CCCN5C[C@H](F)C6)nc4c3F)c12 ; N#Cc1c(N)sc2c(F)ccc(-c3c(Cl)cc4c(N5CCCOCC5)nc(OC[C@@]56CCCN5C[C@H](F)C6)nc4c3F)c12]

The racemic 2-amino-4-(6-chloro-8-fluoro-2-(((2R,7aS)-2-fluorotetrahydro-1H-pyrrolizin-7a(SN)-yl)methoxy)-4-(1,4-oxazepan-4-yl)quinazolin-7-yl)-7-fluorobenzo[b]thiophene-3-carbonitrile (Ex. 4) (78 mg, 0.124 mmol) was separated by preparative chiral HPLC (Column CHIRALPAK® IC; 0.1% triethylamine, 40% 2-propanol in hexane) to give 2-amino-4-(6-chloro-8-fluoro-2-(((2R,7aS)-2-fluorotetrahydro-1H-pyrrolizin-7a(5H)-yl)methoxy)-4-(1,4-oxazepan-4-yl)quinazolin-7-yl)-7-fluorobenzo[b]thiophene-3-carbonitrile (Ex. 18, the second eluting isomer from chiral HPLC). ESI-MS m/z [M+H]⁺ 629, 631. ¹H NMR (400 MHz, CDCl₃) δ 7.80 (s, 1H), 7.18-7.14 (m, 1H), 7.01-6.94 (m, 1H), 6.41 (br s, 2H), 5.42-5.21 (m, 1H), 4.31-4.28 (m, 1H), 4.20-4.18 (m, 1H), 4.08-3.83 (m, 8H), 3.63-3.02 (m, 4H), 2.36-1.95 (m, 3H).

### Example 19: 2-amino-4-(6-chloro-8-fluoro-2-(((2R,7aS)-2-fluorotetrahydro-1H-pyrrolizin-7a(5H)-yl)methoxy)-4-((S)-5-methyl-1,4-oxazepan-4-yl)quinazolin-7-yl)-7-fluorobenzo[b]thiophene-3-carbonitrile (Ex. 19)

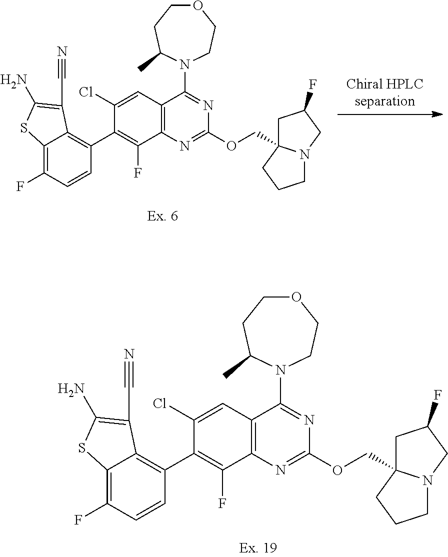 [SMILES: C[C@H]1CCOCCN1c1nc(OC[C@@]23CCCN2C[C@H](F)C3)nc2c(F)c(-c3ccc(F)c4sc(N)c(C#N)c34)c(Cl)cc12 ; C[C@H]1CCOCCN1c1nc(OC[C@@]23CCCN2C[C@H](F)C3)nc2c(F)c(-c3ccc(F)c4sc(N)c(C#N)c34)c(Cl)cc12]

The racemic 2-amino-4-(6-chloro-8-fluoro-2-(((2R,7aS)-2-fluorotetrahydro-1H-pyrrolizin-7a(5H)-yl)methoxy)-4-((S)-5-methyl-1,4-oxazepan-4-yl)quinazolin-7-yl)-7-fluorobenzo[b]thiophene-3-carbonitrile (Ex. 6) (36 mg, 0.056 mmol) was separated by preparative chiral HPLC (Column CHIRALPAK® ID; 0.1% triethylamine, 30% 2-propanol in hexane) to give 2-amino-4-(6-chloro-8-fluoro-2-(((2R,7aS)-2-fluorotetrahydro-1H-pyrrolizin-7a(5H)-yl)methoxy)-4-((S)-5-methyl-1,4-oxazepan-4-yl)quinazolin-7-yl)-7-fluorobenzo[b]thiophene-3-carbonitrile (Ex. 19, the second eluting isomer from chiral HPLC). ESI-MS m/z [M+H]⁺ 643, 645. ¹H ¹H NMR (400 MHz, CDCl₃) δ 7.76 (d, J=1.5 Hz, 1H), 7.23-7.20 (m, 1H), 7.05-7.00 (m, 1H), 5.61 (br s, 2H), 5.33-5.20 (m, 1H), 4.82-4.73 (m, 1H), 4.42-4.38 (m, 1H), 4.22-4.20 (m, 1H), 4.12-3.95 (m, 4H), 3.60-3.45 (m, 2H), 3.32-3.11 (m, 3H), 3.00-2.95 (m, 1H), 2.38-2.14 (m, 4H), 2.03-1.74 (m, 4H), 1.48 (d, J=6.4 Hz, 3H).

### Example 20: 2-amino-7-fluoro-4-(8-fluoro-2-(((2R,7aS)-2-fluorotetrahydro-1H-pyrrolizin-7a(5H)-yl)methoxy)-4-(1,4-oxazepan-4-yl)pyrido[4,3-d]pyrimidin-7-yl)benzo[b]thiophene-3-carbonitrile (Ex. 20)

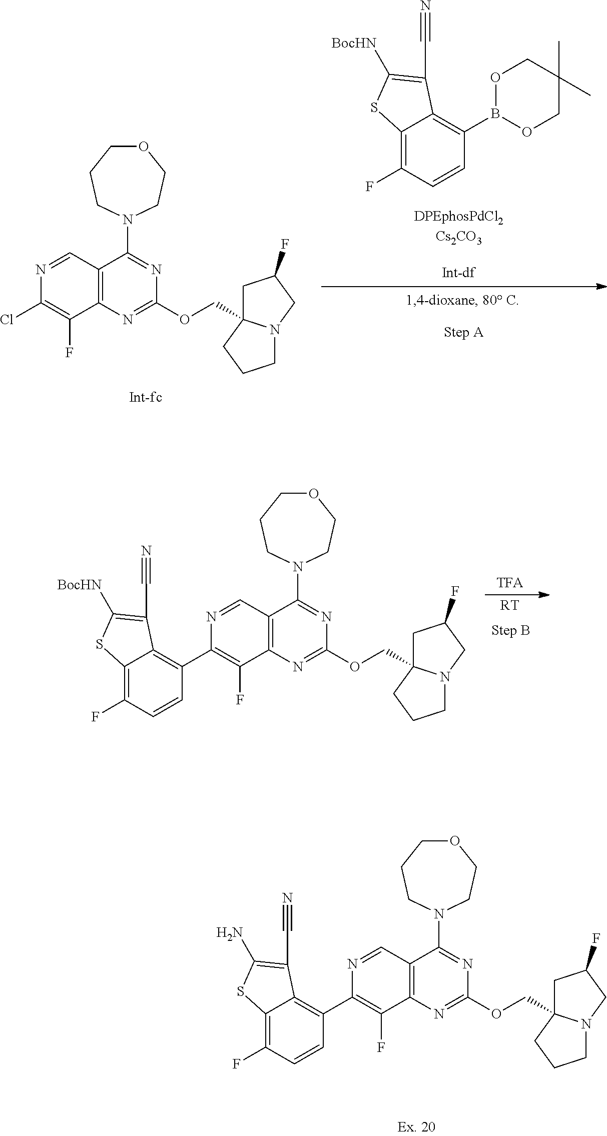

### Step A: Tert-butyl (3-cyano-7-fluoro-4-(8-fluoro-2-(((2R,7aS)-2-fluorotetrahydro-1H-pyrrolizin-7a(5H)-yl)methoxy)-4-(1,4-oxazepan-4-yl)pyrido[4,3-d]pyrimidin-7-yl)benzo[b]thiophen-2-yl)carbamate

To a solution of 4-(7-chloro-8-fluoro-2-(((2R,7aS)-2-fluorotetrahydro-1H-pyrrolizin-7a(5H)-yl)methoxy)pyrido[4,3-d]pyrimidin-4-yl)-1,4-oxazepane (Int-fc) (150 mg, 0.34 mmol), tert-butyl (3-cyano-4-(5,5-dimethyl-1,3,2-dioxaborinan-2-yl)-7-fluorobenzo[b]thiophen-2-yl)carbamate (Int-df) (276 mg, 0.68 mmol) and cesium carbonate (333 mg, 1.02 mmol) in 1,4-dioxane (7.5 ml) was added dichloro[bis(2-(diphenylphosphino)phenyl)ether]palladium(II) (48.8 mg, 0.068 mmol) at room temperature. The mixture was degassed under reduced pressure and purged with nitrogen several times. The mixture was stirred at 80° C. for 2 h under nitrogen atmosphere. The reaction mixture was cooled to room temperature and diluted with water and ethyl acetate. The organic layer was dried over MgSO₄, filtered and concentrated in vacuo. The residue was purified by flash NH-silica chromatography (eluent, 0-50% MeOH in chloroform) to obtain tert-butyl (3-cyano-7-fluoro-4-(8-fluoro-2-(((2R,7aS)-2-fluorotetrahydro-1H-pyrrolizin-7a(5H)-yl)methoxy)-4-(1,4-oxazepan-4-yl)pyrido[4,3-d]pyrimidin-7-yl)benzo[b]thiophen-2-yl)carbamate. ESI-MS m z [M+H]⁺ 696. ¹H NMR (400 MHz, CDCl₃) δ 9.13 (s, 1H), 7.01-7.59 (m, 2H), 5.33-5.20 (m, 1H), 4.28-4.26 (m, 1H), 4.16-4.10 (m, 5H), 3.98 (br s, 2H), 3.85 (br s, 2H), 3.22-3.11 (m, 4H), 2.98-2.96 (m, 1H), 2.32-2.18 (m, 5H), 1.95-1.84 (m, 3H), 1.59-1.54 (m, 9H).

### Step B: 2-amino-7-fluoro-4-(8-fluoro-2-(((2R,7aS)-2-fluorotetrahydro-1H-pyrrolizin-7a(5H)-yl)methoxy)-4-(1,4-oxazepan-4-yl)pyrido[4,3-d]pyrimidin-7-yl)benzo[b]thiophene-3-carbonitrile (Ex. 20)

To a solution of tert-butyl (3-cyano-7-fluoro-4-(8-fluoro-2-(((2R,7aS)-2-fluorotetrahydro-1H-pyrrolizin-7a(5H)-yl)methoxy)-4-(1,4-oxazepan-4-yl)pyrido[4,3-d]pyrimidin-7-yl)benzo[b]thiophen-2-yl)carbamate (85 mg, 0.123 mmol) in dichloromethane (4.3 ml) was added trifluoroacetic acid (0.46 ml, 6.14 mmol) at room temperature under nitrogen atmosphere. The mixture was stirred at room temperature for 1 h under nitrogen atmosphere. The reaction mixture was basified with saturated sodium bicarbonate aqueous solution. The organic layer was dried over MgSO₄, filtered and concentrated in vacuo. The residue was purified by reverse phase HPLC (MeCN/water with 0.1% formic acid) to obtain 2-amino-7-fluoro-4-(8-fluoro-2-(((2R,7aS)-2-fluorotetrahydro-1H-pyrrolizin-7a(5H)-yl)methoxy)-4-(1,4-oxazepan-4-yl)pyrido[4,3-d]pyrimidin-7-yl)benzo[b]thiophene-3-carbonitrile (Ex. 20). ESI-MS m/z [M+H]⁺ 596. ¹H NMR (400 MHz, CDCl₃) δ 9.11 (s, 1H), 7.50-7.46 (m, 1H), 7.05-7.00 (m, 1H), 5.68 (br s, 2H), 5.35-5.21 (m, 1H), 4.28-4.26 (m, 1H), 4.17-4.13 (m, 5H), 4.00-3.98 (m, 2H), 3.87-3.84 (m, 2H), 3.29-3.13 (m, 3H), 3.02-2.96 (m, 1H), 2.33-2.15 (m, 5H), 1.99-1.80 (m, 3H).

Compounds in the table below were synthesized via a similar route as Ex. 20 using 2,4,7-trichloro-8-fluoropyrido[4,3-d]pyrimidine (Int-fa), 1,4-oxazepane hydrochloride salt or the corresponding amine hydrochloride salts, and ((2R,7aS)-2-fluorotetrahydro-1H-pyrrolizin-7a(5H)-yl)methanol (Int-ag) or the corresponding amine hydrochloride salt.

| | | | | ESI-MS |
|---|---|---|---|---|
| Ex. | Structure | Name | NMR | [M + H]⁺ |
| 21 | 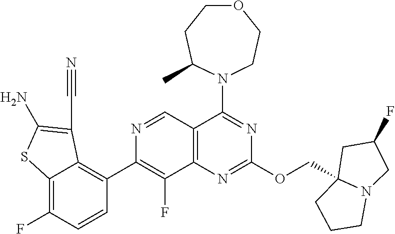 [SMILES: C[C@H]1CCOCCN1c1nc(OC[C@@]23CCCN2C[C@H](F)C3)nc2c(F)c(-c3ccc(F)c4sc(N)c(C#N)c34)ncc12] | 2-amino-7-fluoro-4- (8-fluoro-2- (((2R,7aS)-2- fluorotetrahydro-1H- pyrrolizin-7a(5H)- yl)methoxy)-4-((S)-5- methyl-1,4-oxazepan- 4-yl)pyrido[4,3- d]pyrimidin-7- yl)benzo[b]thiophene- 3-carbonitrile | ¹H NMR (400 MHz, CDCl₃) δ 9.03 (s, 1H), 7.53- 7.49 (m, 1H), 7.07-7.01 (m, 1H), 5.35-5.21 (m, 2H), 4.85- 3.92 (m, 8H), 3.69-2.94 (m, 6H), 2.37-1.81 (m, 8H), 1.55- 1.50 (m, 3H). | 610 |
| 22 | 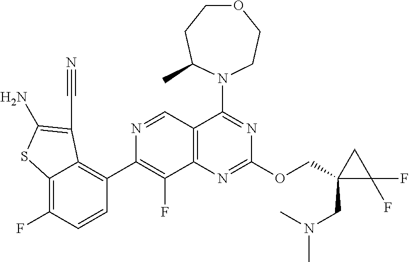 [SMILES: C[C@H]1CCOCCN1c1nc(OC[C@]2(CN(C)C)CC2(F)F)nc2c(F)c(-c3ccc(F)c4sc(N)c(C#N)c34)ncc12] | 2-amino-4-(2-(((R)-1- ((dimethylamino)meth- yl)-2,2- difluorocyclopropyl) methoxy)-8-fluoro-4- ((S)-5-methyl-1,4- oxazepan-4- yl)pyrido[4,3- d]pyrimidin-7-yl)-7- fluorobenzo[b]thiophene- 3-carbonitrile | ¹H NMR (400 MHz, CDCl₃) δ 9.05 (s, 1H), 7.53- 7.48 (m, 1H), 7.07-7.01 (m, 1H), 5.45 (br s, 2H), 4.81-4.76 (m, 2H), 4.48- 4.43 (m, 2H), 4.16-4.13 (m, 1H), 4.02-3.94 (m, 2H), 3.63- 3.57 (m, 1H), 3.44-3.39 (m, 1H), 2.98-2.94 (m, 1H), 2.42- | 616 |
| | | | 2.22 (m, 7H), | |
| | | | 2.08-1.98 (m, | |
| | | | 1H), 1.64-1.51 | |
| | | | (m, 5H), 1.27- | |
| | | | 1.21 (m, 1H). | |

### Example 23: 2-amino-5,7-difluoro-4-(8-fluoro-2-(((2R,7aS)-2-fluorotetrahydro-1H-pyrrolizin-7a(5H)-yl)methoxy)-4-(1,4-oxazepan-4-yl)pyrido[4,3-d]pyrimidin-7-yl)benzo[b]thiophene-3-carbonitrile (Ex. 23)

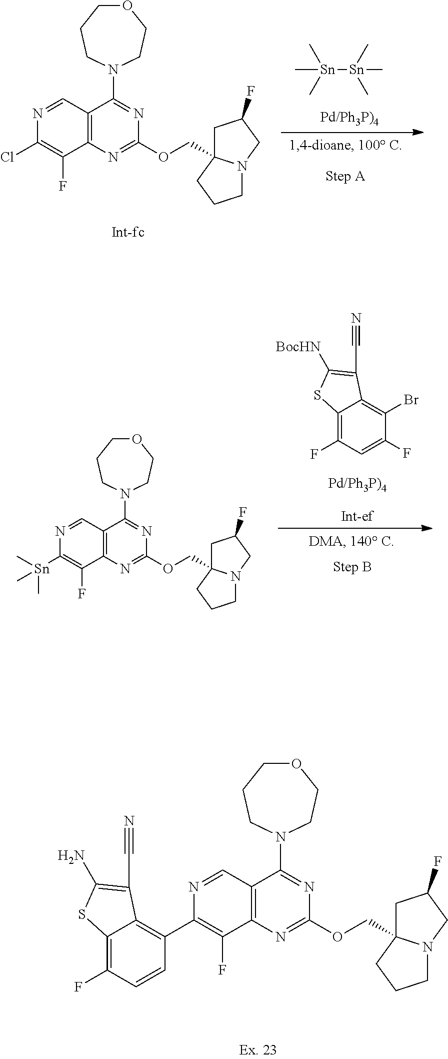

### Step A: 4-(8-fluoro-2-(((2R,7aS)-2-fluorotetrahydro-1H-pyrrolizin-7a(5H)-yl)methoxy)-7-(trimethylstannyl)pyrido[4,3-d]pyrimidin-4-yl)-1,4-oxazepane

To a solution of 4-(7-chloro-8-fluoro-2-(((2R,7aS)-2-fluorotetrahydro-1H-pyrrolizin-7a(5H)-yl)methoxy)pyrido[4,3-d]pyrimidin-4-yl)-1,4-oxazepane (Int-fc) (10 mg, 0.023 mmol), hexamethyldistannane (8.2 mg, 0.025 mmol) in 1,4-dioxane (0.2 ml) was added tetrakis(triphenylphosphine)palladium(0) (2.6 mg, 0.0023 mmol) at room temperature. The mixture was degassed under reduced pressure and purged with nitrogen several times. The mixture was stirred at 100° C. for 3 h under nitrogen atmosphere. The reaction mixture was cooled to room temperature and concentrated in vacuo to obtain 4-(8-fluoro-2-(((2R,7aS)-2-fluorotetrahydro-1H-pyrrolizin-7a(5H)-yl)methoxy)-7-(trimethylstannyl)pyrido[4,3-d]pyrimidin-4-yl)-1,4-oxazepane, which was used in the next step without further purification.

### Step B: 2-amino-5,7-difluoro-4-(8-fluoro-2-(((2R,7aS)-2-fluorotetrahydro-1H-pyrrolizin-7a(5H)-yl)methoxy)-4-(1,4-oxazepan-4-yl)pyrido[4,3-d]pyrimidin-7-yl)benzo[b]thiophene-3-carbonitrile (Ex. 23)

To a solution of 4-(8-fluoro-2-(((2R,7aS)-2-fluorotetrahydro-1H-pyrrolizin-7a(5H)-yl)methoxy)-7-(trimethylstannyl)pyrido[4,3-d]pyrimidin-4-yl)-1,4-oxazepane (12 mg, 0.023 mmol), tert-butyl (4-bromo-3-cyano-5,7-difluorobenzo[b]thiophen-2-yl)carbamate (Int-ef) (9.7 mg, 0.025 mmol) in N,N-dimethylacetamide (0.26 ml) was added tetrakis(triphenylphosphine)palladium(0) (2.6 mg, 0.0023 mmol) at room temperature. The mixture was degassed under reduced pressure and purged with nitrogen several times. The mixture was stirred at 140° C. for 1 h under microwave irradiation. The reaction mixture was quenched with saturated potassium fluoride aqueous solution and extracted with ethyl acetate. The combined organic layer was washed with water and brine. The organic layer was dried over MgSO₄, filtered and concentrated in vacuo. The residue was purified by reverse phase HPLC (MeCN/water with 0.1% formic acid) to obtain 2-amino-5,7-difluoro-4-(8-fluoro-2-(((2R,7aS)-2-fluorotetrahydro-1H-pyrrolizin-7a(5H)-yl)methoxy)-4-(1,4-oxazepan-4-yl)pyrido[4,3-d]pyrimidin-7-yl)benzo[b]thiophene-3-carbonitrile (Ex. 23). ESI-MS m/z [M+H]⁺ 614. ¹H NMR (400 MHz, CDCl₃) δ 9.12 (s, 1H), 7.73-6.85 (m, 1H), 5.36-5.20 (m, 1H), 4.31-3.63 (m, 10H), 3.28-2.92 (m, 4H), 2.37-1.80 (m, 8H).

### Example 24: 2-amino-4-(6-chloro-2-(((R)-1-((dimethylamino)methyl)-2,2-difluorocyclopropyl)methoxy)-8-fluoro-4-(1,4-oxazepan-4-yl)quinazolin-7-yl)-7-fluorobenzo[b]thiophene-3-carbonitrile (Ex. 24)

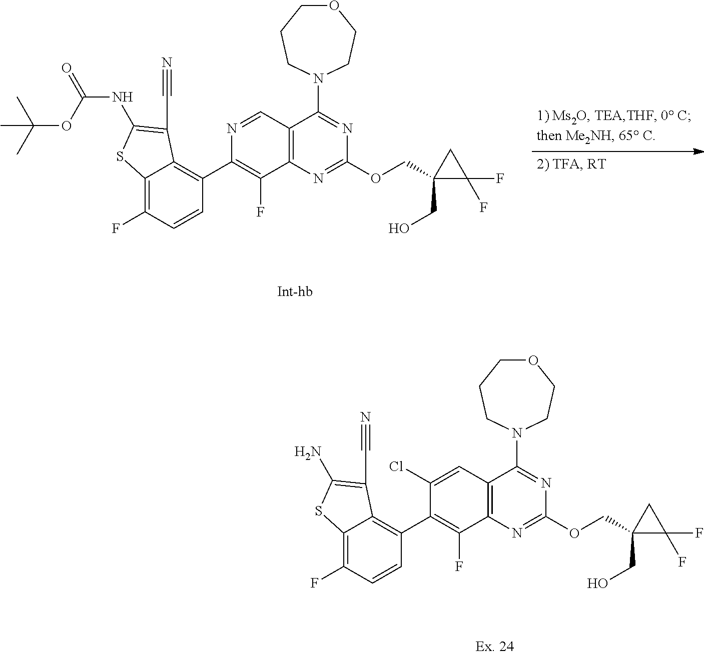 [SMILES: CC(C)(C)OC(=O)Nc1sc2c(F)ccc(-c3ncc4c(N5CCCOCC5)nc(OC[C@]5(CO)CC5(F)F)nc4c3F)c2c1C#N ; N#Cc1c(N)sc2c(F)ccc(-c3c(Cl)cc4c(N5CCCOCC5)nc(OC[C@]5(CO)CC5(F)F)nc4c3F)c12]

To a solution of tert-butyl (4-(6-chloro-2-(((R)-2,2-difluoro-1-(hydroxymethyl)cyclopropyl)methoxy)-8-fluoro-4-(1,4-oxazepan-4-yl)quinazolin-7-yl)-3-cyano-7-fluorobenzo[b]thiophen-2-yl)carbamate (Int-hb) (20.0 mg, 0.0282 mmol) in THF (0.3 mL) were added methanesulfonic anhydride (9.84 mg, 0.0565 mmol) and triethylamine (0.0119 mL, 0.0847 mmol) at 0° C. After stirring the mixture at 0° C. for 1 h, to the reaction mixture was added dimethylamine (0.282 mL, 0.565 mmol, 2M in THF). After stirring the mixture at 65° C. for 22 h, the reaction mixture was cooled to room temperature, quenched with H₂O, extracted with CHCl₃, washed with brine, dried over Na₂SO₄, filtered and concentrated in vacuo to afford an amine compound.

To a solution of the amine compound in dichloromethane (0.5 mL) was added TFA (0.1 mL). After stirring the mixture at room temperature for 2 h, the mixture was diluted with CHCl₃, washed with saturated aqueous NaHCO₃ and concentrated under reduced pressure. The residue was purified by reverse phase HPLC (MeCN/water with 0.1% formic acid) to afford 2-amino-4-(6-chloro-2-(((R)-1-((dimethylamino)methyl)-2,2-difluorocyclopropyl)methoxy)-8-fluoro-4-(1,4-oxazepan-4-yl)quinazolin-7-yl)-7-fluorobenzo[b]thiophene-3-carbonitrile (Ex. 24). ESI-MS m/z [M+H]⁺ 635, 637. ¹H NMR (400 MHz, DMSO-d₆) δ 8.10 (s, 2H), 7.98 (s, 1H), 7.27-7.22 (m, 1H), 7.17-7.11 (m, 1H), 4.49-4.37 (m, 2H), 4.10-4.01 (m, 4H), 3.92-3.87 (m, 2H), 3.72 (dd, J=5.2, 4.8 Hz, 2H), 2.65-2.58 (m, 1H), 2.35-2.29 (m, 1H), 2.15 (s, 6H), 2.09-2.01 (m, 2H), 1.82-1.73 (m, 1H), 1.51-1.42 (m, 1H).

Compounds in the table below were synthesized via a similar route as Ex. 24 using tert-butyl (4-(6-chloro-2-(((R)-2,2-difluoro-1-(hydroxymethyl)cyclopropyl)methoxy)-8-fluoro-4-(1,4-oxazepan-4-yl)quinazolin-7-yl)-3-cyano-7-fluorobenzo[b]thiophen-2-yl)carbamate (Int-hb) and corresponding amines.

| | | | | ESI-MS |
|---|---|---|---|---|
| Ex. | Structure | Name | NMR | [M + H]⁺ |
| 25 | 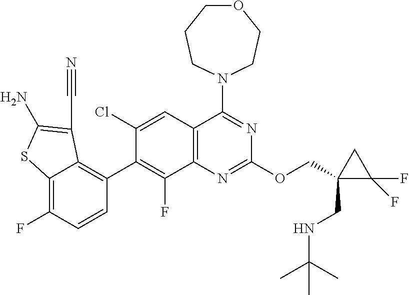 [SMILES: CC(C)(C)NC[C@@]1(COc2nc(N3CCCOCC3)c3cc(Cl)c(-c4ccc(F)c5sc(N)c(C#N)c45)c(F)c3n2)CC1(F)F] | 2-amino-4-(2-(((R)-1- ((tert- butylamino)methyl)- 2,2- difluorocyclopropyl) methoxy)-6-chloro-8- fluoro-4-(1,4- oxazepan-4- yl)quinazolin-7-yl)-7- fluorobenzo[b]thiophene- 3-carbonitrile | ¹H NMR (400 MHz, DMSO-d₆) δ 8.10 (s, 2H), 7.98 (s, 1H), 7.26-7.21 (m, 1H), 7.17-7.11 (m, 1H), 4.65-4.59 (m, 1H), 4.35-4.27 (m, 1H), 4.10-4.00 (m, 4H), 3.92-3.86 (m, 2H), 3.73-3.68 (m, 2H), 2.75-2.59 (m, 2H), 2.09-2.01 (m, 2H), 1.65-1.43 (m, 3H), 0.95 (s, 9H). | 663, 665 |
| 26 | 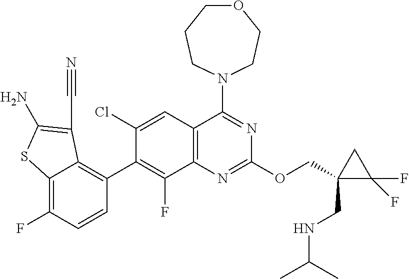 [SMILES: CC(C)NC[C@@]1(COc2nc(N3CCCOCC3)c3cc(Cl)c(-c4ccc(F)c5sc(N)c(C#N)c45)c(F)c3n2)CC1(F)F] | 2-amino-4-(6-chloro- 2-(((R)-2,2-difluoro- 1- ((isopropylamino)meth- yl)cyclopropyl)meth- oxy)-8-fluoro-4-(1,4- oxazepan-4- yl)quinazolin-7-yl)-7- fluorobenzo[b]thiophene- 3-carbonitrile | ¹H NMR (400 MHz, DMSO-d₆) δ 8.10 (s, 2H), 7.98 (s, 1H), 7.26-7.21 (m, 1H), 7.17-7.11 (m, 1H), 4.60 (d, J = 11.6 Hz, 1H), 4.34 (d, J = 11.6 Hz, 1H), 4.10- 4.02 (m, 4H), 3.92- 3.87 (m, 2H), 3.72 (dd, J = 5.2, 5.2 Hz, 2H), 2.77-2.66 (m, 3H), 2.09-2.01 (m, 2H), 1.77-1.42 (m, 3H), 0.96-0.89 (m, 6H). | 649, 651 |
| 27 | 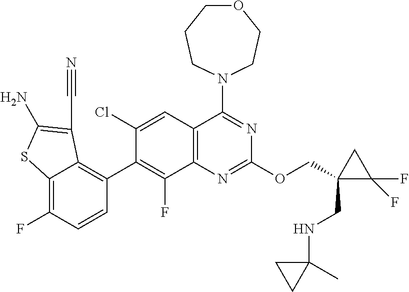 [SMILES: CC1(NC[C@@]2(COc3nc(N4CCCOCC4)c4cc(Cl)c(-c5ccc(F)c6sc(N)c(C#N)c56)c(F)c4n3)CC2(F)F)CC1] | 2-amino-4-(6-chloro- 2-(((R)-2,2-difluoro- 1-(((1- methylcyclopropyl)a- mino)methyl)cyclopro- pyl)methoxy)-8- fluoro-4-(1,4- oxazepan-4- yl)quinazolin-7-yl)-7- fluorobenzo[b]thiophene- 3-carbonitrile | ¹H NMR (400 MHz, DMSO-d₆) δ 8.10 (s, 2H), 7.98 (s, 1H), 7.26-7.21 (m, 1H), 7.17-7.11 (m, 1H), 4.59-4.50 (m, 1H), 4.32-4.26 (m, 1H), 4.10-4.02 (m, 4H), 3.92-3.87 (m, 2H), 3.71 (dd, J = 5.2, 5.2 Hz, 2H), 2.86-2.70 (m, 2H), 2.27-2.14 (m, 1H), 2.09-2.01 (m, 2H), 1.66-1.55 (m, 1H), 1.51-1.42 (m, 1H), 1.14-1.09 (m, 3H), 0.44-0.33 (m, 2H), 0.27-0.19 | 661, 663 |
| | | | (m, 2H). | |
| 28 | 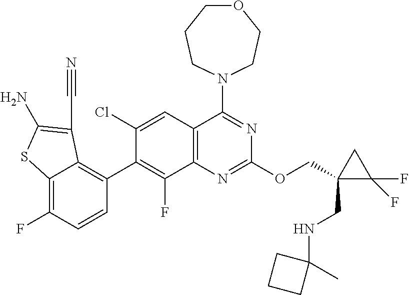 [SMILES: CC1(NC[C@@]2(COc3nc(N4CCCOCC4)c4cc(Cl)c(-c5ccc(F)c6sc(N)c(C#N)c56)c(F)c4n3)CC2(F)F)CCC1] | 2-amino-4-(6-chloro- 2-(((R)-2,2-difluoro- 1-(((1- methylcyclobutyl)ami- no)methyl)cyclopropyl) methoxy)-8- fluoro-4-(1,4- oxazepan-4- yl)quinazolin-7-yl)-7- fluorobenzo[b]thiophene- 3-carbonitrile | ¹H NMR (400 MHz, DMSO-d₆) δ 8.10 (s, 2H), 7.98 (s, 1H), 7.26-7.21 (m, 1H), 7.17-7.11 (m, 1H), 4.63 (d, J = 11.2 Hz, 1H), 4.40-4.29 (m, 3H), 4.16-4.12 (m, 2H), 4.10-4.02 (m, 4H), 3.92-3.86 (m, 2H), 3.71 (dd, J = 5.2, 5.2 Hz, 2H), 2.84-2.36 (m, 5H), 2.10-2.01 (m, 2H), 1.70-1.50 (m, 2H), 1.29 (s, 3H) | 675, 677 |
| 29 | 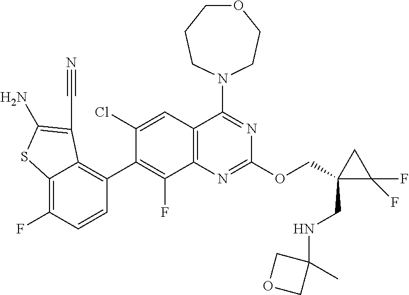 [SMILES: CC1(NC[C@@]2(COc3nc(N4CCCOCC4)c4cc(Cl)c(-c5ccc(F)c6sc(N)c(C#N)c56)c(F)c4n3)CC2(F)F)COC1] | 2-amino-4-(6-chloro- 2-(((R)-2,2-difluoro- 1-(((3-methyloxetan- 3- yl)amino)methyl)cyclo- propyl)methoxy)-8- fluoro-4-(1,4- oxazepan-4- yl)quinazolin-7-yl)-7- fluorobenzo[b]thiophene- 3-carbonitrile | ¹H NMR (400 MHz, DMSO-d₆) δ 8.10 (s, 2H), 7.98 (s, 1H), 7.26-7.20 (m, 1H), 7.17-7.11 (m, 1H), 4.66-4.60 (m, 1H), 4.35-4.31 (m, 1H), 4.10-4.01 (m, 4H), 3.92-3.86 (m, 2H), 3.71 (dd, J = 5.2, 5.2 Hz, 2H), 2.71-2.54 (m, 2H), 2.09-2.01 (m, 2H), 1.88-1.75 (m, 2H), 1.67-1.425 (m, 5H), 1.10 (s, 3H). | 677, 679 |
| 30 | 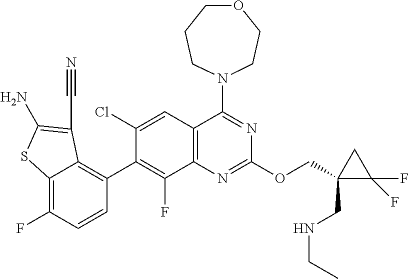 [SMILES: CCNC[C@@]1(COc2nc(N3CCCOCC3)c3cc(Cl)c(-c4ccc(F)c5sc(N)c(C#N)c45)c(F)c3n2)CC1(F)F] | 2-amino-4-(6-chloro- 2-(((R)-1- ((ethylamino)methyl)- 2,2- difluorocyclopropyl) methoxy)-8-fluoro-4- (1,4-oxazepan-4- yl)quinazolin-7-yl)-7- fluorobenzo[b]thiophene- 3-carbonitrile | ¹H NMR (400 MHz, DMSO-d₆) δ 8.10 (s, 2H), 7.98 (s, 1H), 7.27-7.11 (m, 2H), 4.70-4.52 (m, 1H), 4.47-4.30 (m, 1H), 4.10-4.00 (m, 4H), 3.93-3.86 (m, 2H), 3.75-3.69 (m 2H), 3.68-3.51 (m, 2H), 2.85-2.60 (m, 2H), 2.09-2.01 (m, 2H), 1.80-1.42 (m, 3H), 1.27 (t, J = 7.2 Hz, 3H). | 635, 637 |
| 31 | 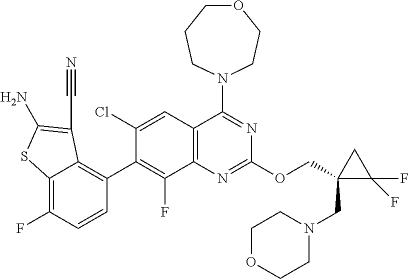 [SMILES: N#Cc1c(N)sc2c(F)ccc(-c3c(Cl)cc4c(N5CCCOCC5)nc(OC[C@]5(CN6CCOCC6)CC5(F)F)nc4c3F)c12] | 2-amino-4-(6-chloro- 2-(((R)-2,2-difluoro- 1- (morpholinomethyl)cy- clopropyl)methoxy)- 8-fluoro-4-(1,4- oxazepan-4- yl)quinazolin-7-yl)-7- fluorobenzo[b]thiophene- 3-carbonitrile | ¹H NMR (400 MHz, CDCl₃) δ 7.85 (d, J = 1.8 Hz, 1H), 7.24- 7.21 (m, 1H), 7.07- 7.02 (m, 1H), 5.39 (s, 2H), 4.84-4.75 (m, 1H), 4.49-4.41 (m, 1H), 4.16-4.05 (m, 4H), 4.01-3.99 (m, 2H), 3.92-3.83 (m, 2H), 3.72-3.63 (m, 4H), 3.07-0.69 (m, 10H) | 677, 679 |

### Example 32: 2-amino-4-(6-chloro-2-(((R)-2,2-difluoro-1-(hydroxymethyl)cyclopropyl)methoxy)-8-fluoro-4-(1,4-oxazepan-4-yl)quinazolin-7-yl)-7-fluorobenzo[b]thiophene-3-carbonitrile (Ex. 32)

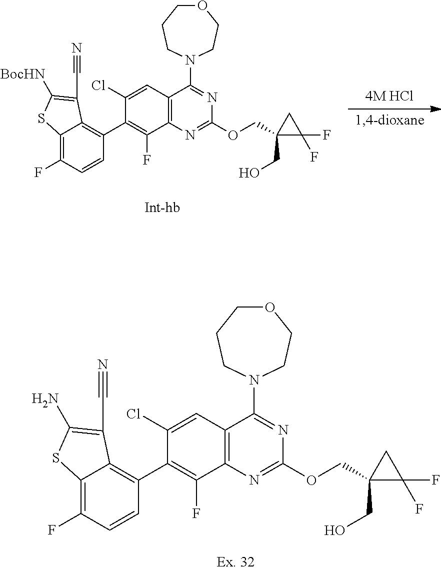 [SMILES: CC(C)(C)OC(=O)Nc1sc2c(F)ccc(-c3c(Cl)cc4c(N5CCCOCC5)nc(OC[C@]5(CO)CC5(F)F)nc4c3F)c2c1C#N ; N#Cc1c(N)sc2c(F)ccc(-c3c(Cl)cc4c(N5CCCOCC5)nc(OC[C@]5(CO)CC5(F)F)nc4c3F)c12]

To a solution of tert-butyl (4-(6-chloro-2-(((R)-2,2-difluoro-1-(hydroxymethyl)cyclopropyl)methoxy)-8-fluoro-4-(1,4-oxazepan-4-yl)quinazolin-7-yl)-3-cyano-7-fluorobenzo[b]thiophen-2-yl)carbamate (Int-hb) (95 mg, 0.13 mmol) in acetonitrile (0.95 ml) was added 4M-HCl in 1,4-dioxane (1.0 ml, 4.02 mmol) at room temperature under nitrogen atmosphere. The mixture was stirred at room temperature for 6 h under nitrogen atmosphere. The reaction mixture was basified with saturated sodium bicarbonate aqueous solution. The organic layer was dried over MgSO₄, filtered and concentrated in vacuo. The residue was purified by flash silica chromatography (eluent, 0-20% MeOH in EtOAc) to obtain 2-amino-4-(6-chloro-2-(((R)-2,2-difluoro-1-(hydroxymethyl)cyclopropyl)methoxy)-8-fluoro-4-(1,4-oxazepan-4-yl)quinazolin-7-yl)-7-fluorobenzo[b]thiophene-3-carbonitrile (Ex. 32). ESI-MS m/z [M+H]⁺ 608, 610. 1H NMR (400 MHz, CDCl3) δ 7.88 (d, J=1.5 Hz, 1H), 7.25-7.22 (m, 1H), 7.07-7.02 (m, 1H), 5.39-5.37 (m, 2H), 4.80-4.75 (m, 1H), 4.66-4.62 (m, 1H), 4.19-4.06 (m, 4H), 4.02-3.99 (m, 2H), 3.90-3.85 (m, 2H), 3.71-3.63 (m, 2H), 2.25-2.19 (m, 2H), 1.53-1.26 (m, 2H).

### Example 33: 2-amino-4-(6-chloro-2-(((R)-2,2-difluoro-1-(hydroxymethyl)cyclopropyl)methoxy)-8-fluoro-4-(1,4-oxazepan-4-yl)quinazolin-7-yl)-7-fluorobenzo[b]thiophene-3-carbonitrile (Ex. 33)

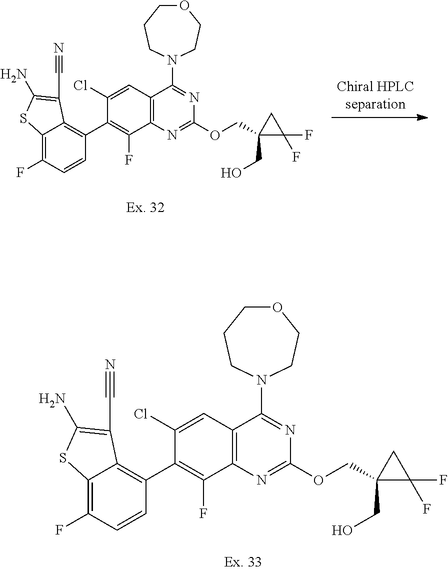 [SMILES: N#Cc1c(N)sc2c(F)ccc(-c3c(Cl)cc4c(N5CCCOCC5)nc(OC[C@]5(CO)CC5(F)F)nc4c3F)c12 ; N#Cc1c(N)sc2c(F)ccc(-c3c(Cl)cc4c(N5CCCOCC5)nc(OC[C@]5(CO)CC5(F)F)nc4c3F)c12]

The racemic 2-amino-4-(6-chloro-2-(((R)-2,2-difluoro-1-(hydroxymethyl)cyclopropyl)methoxy)-8-fluoro-4-(1,4-oxazepan-4-yl)quinazolin-7-yl)-7-fluorobenzo[b]thiophene-3-carbonitrile (Ex. 32) (18 mg, 0.03 mmol) was separated by preparative chiral HPLC (Column CHIRALPAK(R IG; 0.1% triethylamine, 40% EtOH in hexane) to give 2-amino-4-(6-chloro-2-(((R)-2,2-difluoro-1-(hydroxymethyl)cyclopropyl)methoxy)-8-fluoro-4-(1,4-oxazepan-4-yl)quinazolin-7-yl)-7-fluorobenzo[b]thiophene-3-carbonitrile (Ex. 33, the second eluting isomer from chiral HPLC). ESI-MS m/z [M+H]⁺ 608, 610. 1H NMR (400 MHz, CDCl3) δ 7.87 (d, J=1.6 Hz, 1H), 7.24-7.21 (m, 1H), 7.05-7.00 (m, 1H), 5.51-5.09 (m, 3H), 4.81-4.59 (m, 2H), 4.20-4.07 (m, 4H), 4.01-3.98 (m, 2H), 3.89-3.87 (m, 2H), 3.68-3.65 (m, 2H), 2.24-2.16 (m, 2H), 1.52-1.31 (m, 2H).

### Example 34: 2-amino-7-fluoro-4-(8-fluoro-2-(((2R,7aS)-2-fluorotetrahydro-1H-pyrrolizin-7a(5H)-yl)methoxy)-4-(1,4-oxazepan-4-yl)-6-(trifluoromethyl)quinazolin-7-yl)benzo[b]thiophene-3-carbonitrile (Ex. 34)

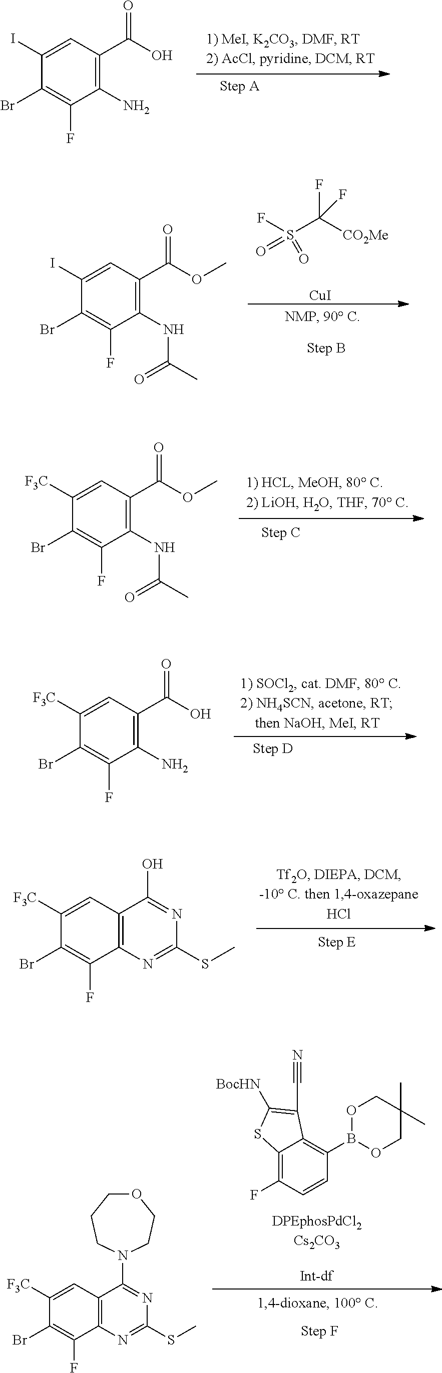

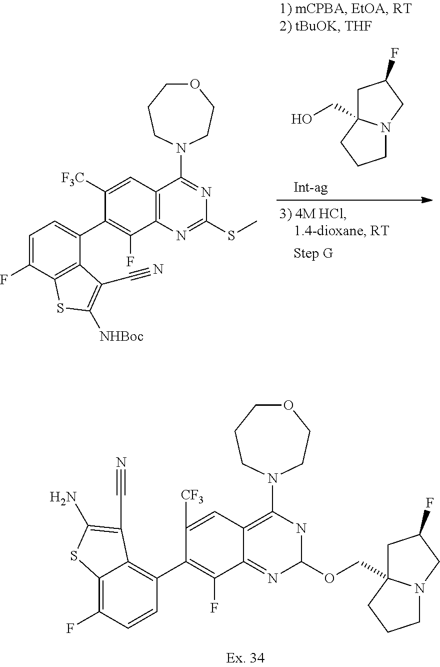

### Step A: Methyl 2-acetamido-4-bromo-3-fluoro-5-iodobenzoate

To a mixture of 2-amino-4-bromo-3-fluoro-5-iodobenzoic acid (3.00 g, 8.34 mmol) and potassium carbonate (3.46 g, 25.0 mmol) in N,N-dimethylformamide (30 mL) was added iodomethane (0.57 mL, 9.17 mmol), and the mixture was stirred at room temperature for 2 h. Additional iodomethane (0.10 mL, 1.61 mmol) was added to the reaction mixture, and the mixture was stirred at room temperature for additional 1 h. The reaction mixture was diluted with ethyl acetate (50 mL), and water (30 mL) was added to the mixture. The organic layer was separated and washed with water (10 mL). The solvent was evaporated under reduced pressure to give a crude ester, which was used in the next step without purification.

To a mixture of the crude ester and pyridine (2.02 mL, 25.0 mmol) in dichloromethane (30 mL) was added acetyl chloride (0.773 mL, 10.8 mmol) dropwise, and the reaction mixture was stirred at room temperature for 2 days. After the reaction was completed, the solvent was evaporated under reduced pressure and the mixture was diluted with ethyl acetate (100 mL). The mixture was washed with water (30 mL) and the solvent was concentrated under reduced pressure to give a precipitate. A precipitate was suspended in acetonitrile (30 mL) at 80° C. for 30 min. After the mixture was cooled to room temperature, a precipitate was filtered and dried under reduced pressure to give methyl 2-acetamido-4-bromo-3-fluoro-5-iodobenzoate. ESI-MS m/z [M+H]⁺ 416, 418. ¹H NMR (400 MHz, DMSO-d6) δ 10.02 (s, 1H), 8.04 (d, J=1.6 Hz, 1H), 3.77 (s, 3H), 2.05 (s, 3H).

### Step B: Methyl 2-acetamido-4-bromo-3-fluoro-5-(trifluoromethyl)benzoate

To a mixture of methyl 2-acetamido-4-bromo-3-fluoro-5-iodobenzoate (1.95 g, 4.69 mmol) and copper(I) iodide (536 mg, 2.81 mmol) in N-methyl-2-pyrrolidone (20 mL) was added methyl 2,2-difluoro-2-fluorosulfonyl acetate (1.78 mL, 14.1 mmol), and the mixture was stirred at 90° C. for 2.5 h. The reaction mixture was cooled to room temperature and diluted with ethyl acetate (60 mL). The mixture was washed with water (20 mL) and brine (20 mL), and the solvent was evaporated under reduced pressure. The residue was purified by column chromatography on silica gel (ethyl acetate in hexane, 10-65% gradient) to give methyl 2-acetamido-4-bromo-3-fluoro-5-(trifluoromethyl)benzoate. ESI-MS m/z [M+H]⁺ 358, 360. ¹H NMR (400 MHz, DMSO-d6) δ 10.39 (s, 1H), 7.89 (d, J=1.2 Hz, 1H), 3.80 (s, 3H), 2.11 (s, 3H).

### Step C: 2-amino-4-bromo-3-fluoro-5-(trifluoromethyl)benzoic acid

A solution of methyl 2-acetamido-4-bromo-3-fluoro-5-(trifluoromethyl)benzoate (1.36 g, 3.08 mmol) in 10% hydrochloride in MeOH (20 mL) was stirred at 80° C. for 4 h. After the reaction was completed, the solvent was evaporated under reduced pressure and a crude ester was dried in vacuo.

To a mixture of the crude ester in tetrahydrofuran (20 mL) and water (5 mL) was added lithium hydroxide monohydrate (797 mg, 19.0 mmol), the mixture was stirred at 70° C. for 3 h. The reaction mixture was neutralized with a solution of hydrochloric acid (2M) and extracted with ethyl acetate (50 mL). The extracts were washed with water (10 mL) and concentrated to give 2-amino-4-bromo-3-fluoro-5-(trifluoromethyl)benzoic acid. ESI-MS m/z [M+H]⁺ 302, 304. ¹H NMR (400 MHz, DMSO-d6) δ 13.62 (br s, 1H), 7.90 (s, 1H), 7.46 (br s, 2H).

### Step D: 7-bromo-8-fluoro-2-(methylthio)-6-(trifluoromethyl)quinazolin-4-ol

To a mixture of 2-amino-4-bromo-3-fluoro-5-(trifluoromethyl)benzoic acid (250 mg, 0.828 mmol) in thionyl chloride (5.0 mL, 68.9 mmol) was added N,N-dimethylformamide (0.05 mL), and the mixture was stirred at 80° C. for 4 h. The mixture was evaporated and dried under reduced pressure to give a crude acid chloride.

To a mixture of ammonium thiocyanate (88.2 mg, 1.16 mmol) in acetone (3.0 mL) was added a solution of the crude acid chloride in acetone (3.0 mL) dropwise, and the mixture was stirred at room temperature for 30 min. The solvent was evaporated under reduced pressure. To a residue was added tetrahydrofuran (3.0 mL), aqueous solution of sodium hydroxide (0.90 mL, 2 M) and iodomethane (0.05 mL, 0.08 mmol) at room temperature. To the mixture was added additional aqueous solution of sodium hydroxide (0.90 mL, 2 M), and the mixture was stirred at room temperature for 15 min. To the reaction mixture was added ethyl acetate (20 mL), water (10 mL) and hydrochloric acid (1.8 mL, 2 M), and extracted with ethyl acetate. The extract was washed with water and concentrated under reduce pressure to give 7-bromo-8-fluoro-2-(methylthio)-6-(trifluoromethyl)quinazolin-4-ol. ESI-MS m/z [M+H]⁺ 357, 359. ¹H NMR (400 MHz, DMSO-d6) δ 13.24 (br s, 1H), 8.12 (d, J=1.2 Hz, 1H), 2.63 (s, 3H).

### Step E: 4-(7-bromo-8-fluoro-2-(methylthio)-6-(trifluoromethyl)quinazolin-4-yl)-1,4-oxazepane

To a mixture of 7-bromo-8-fluoro-2-(methylthio)-6-(trifluoromethyl)quinazolin-4-ol (270 mg, 0.756 mmol) in dichloromethane (5 mL) was added N,N-diisopropylethylamine (0.386 mL, 2.27 mmol) and trifluoromethanesulfonic anhydride (0.198 mL, 1.21 mmol) at −10° C., the mixture was stirred at −10° C. for 30 min. To the mixture was added 1,4-oxazepane hydrochloride (260 mg, 1.89 mmol) and N,N-diisopropylethylamine (0.386 mL, 2.27 mmol), the mixture was stirred at room temperature for 1 h. The mixture was diluted with ethyl acetate, and washed with water and concentrated under reduced pressure. The residue was purified by column chromatography on silica gel (ethyl acetate in hexane, 10-40% gradient) to give 4-(7-bromo-8-fluoro-2-(methylthio)-6-(trifluoromethyl)quinazolin-4-yl)-1,4-oxazepane. ESI-MS m/z [M+H]⁺ 440, 442. ¹H NMR (400 MHz, Chloroform-d) δ 8.04 (d, J=0.8 Hz, 1H), 4.11-4.06 (m, 4H), 4.01-3.98 (m, 2H), 3.87-3.84 (m, 2H), 2.63 (s, 3H), 2.25-2.17 (m, 2H).

### Step F: Tert-butyl (3-cyano-7-fluoro-4-(8-fluoro-2-(methylthio)-4-(1,4-oxazepan-4-yl)-6-(trifluoromethyl)quinazolin-7-yl)benzo[b]thiophen-2-yl)carbamate

To a mixture of 4-(7-bromo-8-fluoro-2-(methylthio)-6-(trifluoromethyl)quinazolin-4-yl)-1,4-oxazepane (170 mg, 0.386 mmol) in 1,4-dioxane (2.00 mL) was added tert-butyl (3-cyano-4-(5,5-dimethyl-1,3,2-dioxaborinan-2-yl)-7-fluorobenzo[b]thiophen-2-yl)carbamate (Int-df) (156 mg, 0.386 mmol), dichloro[bis(2-(diphenylphosphino)phenyl)ether]palladium(II) (55.3 mg, 0.0772 mmol) and cesium carbonate (315 mg, 0.965 mmol), and the mixture was stirred at 100° C. for 1.5 h. The mixture was diluted with ethyl acetate, and washed with water and concentrated under reduced pressure. The residue was purified by column chromatography on silica gel (ethyl acetate in hexane, 10-40% gradient) to give tert-butyl (3-cyano-7-fluoro-4-(8-fluoro-2-(methylthio)-4-(1,4-oxazepan-4-yl)-6-(trifluoromethyl)quinazolin-7-yl)benzo[b]thiophen-2-yl)carbamate. ESI-MS m/z [M+H]⁺ 652. ¹H NMR (400 MHz, Chloroform-d) δ 8.14 (s, 1H), 7.68 (br s, 1H), 7.33-7.30 (m, 1H), 7.17-7.13 (m, 1H), 4.17-4.12 (m, 4H), 4.04-4.01 (m, 2H), 3.90-3.88 (m, 2H), 2.63 (s, 3H), 2.27-2.22 (m, 2H), 1.58 (s, 9H).

### Step G: 2-amino-7-fluoro-4-(8-fluoro-2-(((2R,7aS)-2-fluorotetrahydro-1H-pyrrolizin-7a(5H)-yl)methoxy)-4-(1,4-oxazepan-4-yl)-6-(trifluoromethyl)quinazolin-7-yl)benzo[b]thiophene-3-carbonitrile (Ex. 34)

To a mixture of tert-butyl (3-cyano-7-fluoro-4-(8-fluoro-2-(methylthio)-4-(1,4-oxazepan-4-yl)-6-(trifluoromethyl)quinazolin-7-yl)benzo[b]thiophen-2-yl)carbamate (40.0 mg, 0.0614 mmol) in ethyl acetate (3 mL) was added m-chloroperoxybenzoic acid (30.3 mg, 0.123 mmol, contains 30% water) at 0° C., and the mixture was stirred at room temperature for 2 h. The mixture was diluted with ethyl acetate, and washed with water and concentrated under reduced pressure. A crude sulfone was obtained and used in the next step without purification.

To a mixture of ((2R,7aS)-2-fluorotetrahydro-1H-pyrrolizin-7a(5H)-yl)methanol (Int-ag) (19.5 mg, 0.123 mmol) in tetrahydrofuran (1 mL) was added potassium tert-butoxide (0.294 mL, 0.294 mmol, 1M in tetrahydrofuran) and a solution of the crude sulfone in tetrahydrofuran (1 mL) at 0° C., the mixture was stirred at room temperature for 1 h. The mixture was diluted with ethyl acetate, and washed with water and concentrated under reduced pressure. A crude amine was obtained and used in the next step without purification.

To a mixture of the crude amine in dichloromethane (2 mL) and methanol (0.1 mL) was added hydrochloride (2.0 mL, 4 M in 1,4-dioxane), the mixture was stirred at room temperature for 10 h. The mixture was diluted with ethyl acetate, and aqueous sodium bicarbonate was added to the mixture. The organic layer was separated, washed with water and concentrated under reduce pressure. The residue was purified by column chromatography on NH-silica gel (methanol in ethyl acetate, 0-20% gradient) to give 2-amino-7-fluoro-4-(8-fluoro-2-(((2R,7aS)-2-fluorotetrahydro-1H-pyrrolizin-7a(5H)-yl)methoxy)-4-(1,4-oxazepan-4-yl)-6-(trifluoromethyl)quinazolin-7-yl)benzo[b]thiophene-3-carbonitrile (Ex. 34). ESI-MS m/z [M+H]⁺ 663. ¹H NMR (400 MHz, C DMSO-d6) δ 8.20 (s, 1H), 8.08 (br s, 2H), 7.26-7.22 (m, 1H), 7.16-7.12 (m, 1H), 5.28 (d, J=54 Hz, 1H), 4.14-4.03 (m, 6H), 3.95-3.92 (m, 2H), 3.77-3.73 (m, 2H), 3.16-2.99 (m, 3H), 2.86-2.79 (m, 1H), 2.20-1.72 (m, 8H).

### Example 35: 2-amino-7-fluoro-4-(8-fluoro-2-(((2R,7aS)-2-fluorotetrahydro-1H-pyrrolizin-7a(5H)-yl)methoxy)-4-(1,4-oxazepan-4-yl)-6-(trifluoromethyl)quinazolin-7-yl)benzo[b]thiophene-3-carbonitrile (Ex. 35)

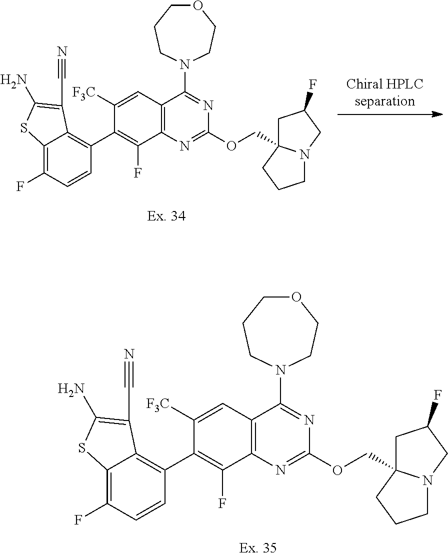 [SMILES: N#Cc1c(N)sc2c(F)ccc(-c3c(C(F)(F)F)cc4c(N5CCCOCC5)nc(OC[C@@]56CCCN5C[C@H](F)C6)nc4c3F)c12 ; N#Cc1c(N)sc2c(F)ccc(-c3c(C(F)(F)F)cc4c(N5CCCOCC5)nc(OC[C@@]56CCCN5C[C@H](F)C6)nc4c3F)c12]

The racemic 2-amino-7-fluoro-4-(8-fluoro-2-(((2R,7aS)-2-fluorotetrahydro-1H-pyrrolizin-7a(5H)-yl)methoxy)-4-(1,4-oxazepan-4-yl)-6-(trifluoromethyl)quinazolin-7-yl)benzo[b]thiophene-3-carbonitrile (Ex. 34) (171 mg, 0.26 mmol) was separated by preparative chiral HPLC (Column CHIRALPAK® IC; 0.1% triethylamine, 50% 2-propanol in hexane) to give 2-amino-7-fluoro-4-(8-fluoro-2-(((2R,7aS)-2-fluorotetrahydro-1H-pyrrolizin-7a(5H)-yl)methoxy)-4-(1,4-oxazepan-4-yl)-6-(trifluoromethyl)quinazolin-7-yl)benzo[b]thiophene-3-carbonitrile (Ex. 35, the first eluting isomer from chiral HPLC). ESI-MS m/z [M+H]⁺ 663. ¹H NMR (400 MHz, CDCl3) δ 8.11 (s, 1H), 7.19 (dd, J=8.3 Hz, 1H), 6.99 (dd, J=9.1, 8.5 Hz, 1H), 5.68 (br s, 2H), 5.33-5.19 (m, 1H), 4.26 (d, J=10.3 Hz, 1H), 4.17-3.98 (m, 7H), 3.89-3.86 (m, 2H), 3.28-3.11 (m, 3H), 3.00-2.94 (m, 1H), 2.27-2.13 (m, 5H), 1.98-1.80 (m, 3H).

### Example 36: 4-(4-(3-oxa-6-azabicyclo[3.1.1]heptan-6-yl)-6-bromo-8-fluoro-2-(((2R,7aS)-2-fluorotetrahydro-1H-pyrrolizin-7a(5H)-yl)methoxy)quinazolin-7-yl)-2-amino-7-fluorobenzo[b]thiophene-3-carbonitrile (Ex. 36)

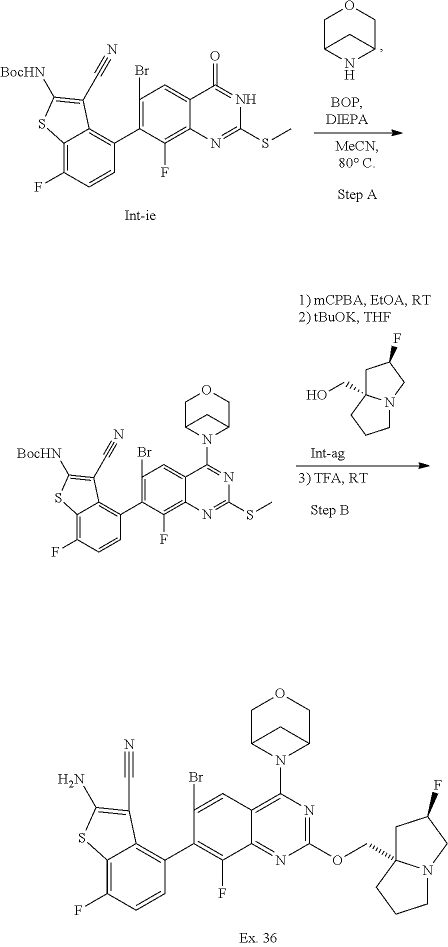

### Step A: Tert-butyl (4-(4-(3-oxa-6-azabicyclo[3.1.1]heptan-6-yl)-6-bromo-8-fluoro-2-(methylthio)quinazolin-7-yl)-3-cyano-7-fluorobenzo[b]thiophen-2-yl)carbamate

To a mixture of tert-butyl (4-(6-bromo-8-fluoro-2-(methylthio)-4-oxo-3,4-dihydroquinazolin-7-yl)-3-cyano-7-fluorobenzo[b]thiophen-2-yl)carbamate (Int-ie) (78.0 mg, 0.135 mmol), benzotriazol-1-yloxytris(dimethylamino)phosphonium hexafluorophosphate (119 mg, 0.269 mmol) and 3-oxa-6-azabicyclo[3.1.1]heptane (20.0 mg, 0.202 mmol) in acetonitrile (2 mL) was added N,N-diisopropylethylamine (0.137 mL, 0.808 mmol), the mixture was stirred at 80° C. for 1 h. The mixture was concentrated under reduced pressure and the residue was purified by column chromatography on silica gel (ethyl acetate in hexane, 30-70% gradient) to give tert-butyl (4-(4-(3-oxa-6-azabicyclo[3.1.1]heptan-6-yl)-6-bromo-8-fluoro-2-(methylthio)quinazolin-7-yl)-3-cyano-7-fluorobenzo[b]thiophen-2-yl)carbamate. ESI-MS m/z [M+H]⁺ 660, 662. ¹H NMR (400 MHz, Chloroform-d) δ 7.80 (d, J=1.6 Hz, 1H), 7.79 (br s, 1H), 7.31-7.26 (m, 1H), 7.19-7.15 (m, 1H), 4.93 (d, J=6 Hz, 2H), 4.50-4.35 (m, 2H), 4.03-3.95 (m, 2H), 3.00 (dd, J=14.0, 6.4 Hz, 1H), 2.61 (s, 3H), 2.13 (d, J=8.4 Hz, 1H), 1.59 (s, 9H).

### Step B: 4-(4-(3-oxa-6-azabicyclo[3.1.1]heptan-6-yl)-6-bromo-8-fluoro-2-(((2R,7aS)-2-fluorotetrahydro-1H-pyrrolizin-7a(5H)-yl)methoxy)quinazolin-7-yl)-2-amino-7-fluorobenzo[b]thiophene-3-carbonitrile (Ex. 36)

To a mixture of tert-butyl (4-(4-(3-oxa-6-azabicyclo[3.1.1]heptan-6-yl)-6-bromo-8-fluoro-2-(methylthio)quinazolin-7-yl)-3-cyano-7-fluorobenzo[b]thiophen-2-yl)carbamate (90.0 mg, 0.136 mmol) in ethyl acetate (3 mL) was added m-chloroperoxybenzoic acid (40.3 mg, 0.163 mmol, contains 30% water) at 0° C., and the mixture was stirred at room temperature for 30 min. The mixture was diluted with ethyl acetate, and washed with water and concentrated under reduced pressure. A crude sulfone was obtained and used in the next step without purification.

To a mixture of ((2R,7aS)-2-fluorotetrahydro-1H-pyrrolizin-7a(5H)-yl)methanol (Int-ag) (32.5 mg, 0.204 mmol) in tetrahydrofuran (1 mL) was added potassium tert-butoxide (0.409 mL, 0.409 mmol, 1 M in tetrahydrofuran) and a solution of the crude sulfone in tetrahydrofuran (1 mL) at 0° C., the mixture was stirred at room temperature for 1 h. The mixture was diluted with ethyl acetate, and washed with water and concentrated under reduced pressure. A crude amine was obtained and used in the next step without purification.

The crude amine was treated with trifluoroacetic acid (2 mL), and the mixture was stirred at room temperature for 0.5 h. Trifluoroacetic acid was evaporated under reduced pressure and the residue was purified by column chromatography on NH-silica gel (Methanol in ethyl acetate, 0-20% gradient) to 4-(4-(3-oxa-6-azabicyclo[3.1.1]heptan-6-yl)-6-bromo-8-fluoro-2-(((2R,7aS)-2-fluorotetrahydro-1H-pyrrolizin-7a(5H)-yl)methoxy)quinazolin-7-yl)-2-amino-7-fluorobenzo[b]thiophene-3-carbonitrile (Ex. 36). ESI-MS m/z [M+H]⁺ 671, 673. ¹H NMR (400 MHz, DMSO-d6) δ 8.11 (br s, 2H), 7.96 (d, J=0.8 Hz, 1H), 7.27-7.22 (m, 1H), 7.18-7.12 (m, 1H), 5.27 (d, J=54.0 Hz, 1H), 5.20-4.80 (m, 2H), 4.31-3.91 (m, 2H), 4.09-3.99 (m, 2H), 3.95-3.87 (m, 2H), 3.13-2.75 (m, 5H), 2.19-2.12 (m, 1H), 2.07-1.98 (m, 2H), 1.94-1.91 (m, 1H), 1.87-1.73 (m, 3H).

Compounds in the table below were synthesized via a similar route as Ex. 36 using tert-butyl (4-(6-bromo-8-fluoro-2-(methylthio)-4-oxo-3,4-dihydroquinazolin-7-yl)-3-cyano-7-fluorobenzo[b]thiophen-2-yl)carbamate (Int-ie), corresponding amines and amino alcohols.

| | | | | ESI-MS |
|---|---|---|---|---|
| Ex. | Structure | Name | NMR | [M + H]⁺ |
| 37 | 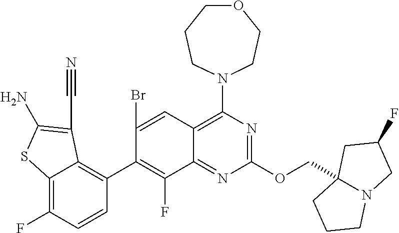 [SMILES: N#Cc1c(N)sc2c(F)ccc(-c3c(Br)cc4c(N5CCCOCC5)nc(OC[C@@]56CCCN5C[C@H](F)C6)nc4c3F)c12] | 2-amino-4-(6- bromo-8-fluoro-2- (((2R,7aS)-2- fluorotetrahydro- 1H-pyrrolizin- 7a(5H)- yl)methoxy)-4-(1,4- oxazepan-4- yl)quinazolin-7-yl)- 7- fluorobenzo[b]thiophene- 3-carbonitrile | ¹H NMR (400 MHz, CDCl₃) δ 8.01-8.00 (m, 1H), 7.20-7.16 (m, 1H), 7.03-6.89 (m, 1H), 5.60-5.57 (m, 2H), 5.33-5.20 (m, 1H), 4.26-4.20 (m, 1H), 4.14-4.07 (m, 5H), 3.98-3.96 (m, 2H), 3.87-3.84 (m, 2H), 3.26-3.32 (m, 2H), 3.15-3.14 (m, 1H), 2.96-2.94 (m, 1H), 2.20-2.14 | 673, 675 |
| | | | (m, 4H), 1.94-1.87 | |
| | | | (m, 4H) | |
| 38 | 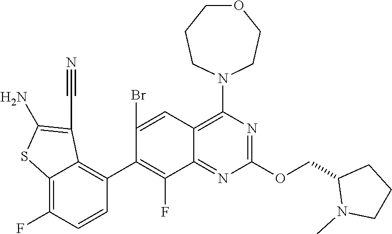 [SMILES: CN1CCC[C@H]1COc1nc(N2CCCOCC2)c2cc(Br)c(-c3ccc(F)c4sc(N)c(C#N)c34)c(F)c2n1] | 2-amino-4-(6- bromo-8-fluoro-2- (((S)-1- methylpyrrolidin-2- yl)methoxy)-4-(1,4- oxazepan-4- yl)quinazolin-7-yl)- 7- fluorobenzo[b]thiophene- 3-carbonitrile | ¹H NMR (400 MHz, CDCl₃) δ 8.016- 8.013 (m, 1H), 7.21-7.17 (m, 1H), 7.05-7.00 (m, 1H), 5.43 (br, 2H), 4.55- 4.50 (m, 1H), 4.30- 4.24 (m, 1H), 4.10- 4.06 (m, 4H), 3.99- 3.97 (m, 2H), 3.87- 3.85 (m, 2H), 3.11- 3.07 (m, 1H), 2.73- 2.70 (m, 1H), 2.49 (s, 3H), 2.29-2.26 (m, 1H), 2.20-2.18 | 629, 631 |
| | | | (m, 2H), 2.04-2.02 | |
| | | | (m, 1H), 1.80-1.76 | |
| | | | (m, 3H) | |

### 6-(6-chloro-8-fluoro-2-(((2R,7aS)-2-fluorotetrahydro-1H-pyrrolizin-7a(5H)-yl)methoxy)-4-(1,4-oxazocan-4-yl)quinazolin-7-yl)-4-methyl-5-(trifluoromethyl)pyridin-2-amine (Ex. 39)

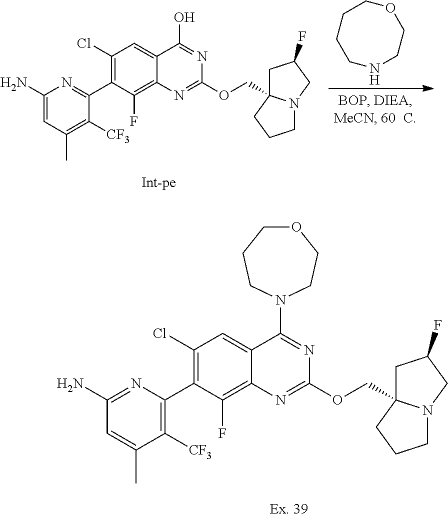

To a solution of 7-(6-amino-4-methyl-3-(trifluoromethyl)pyridin-2-yl)-6-chloro-8-fluoro-2-(((2R,7aS)-2-fluorotetrahydro-1H-pyrrolizin-7a(5H)-yl)methoxy)quinazolin-4-ol (Int-pe) (20 mg, 0.038 mmol) in MeCN (0.5 mL) was added benzotriazol-1-yloxytris(dimethylamino)phosphonium hexafluorophosphate (33.4 mg, 0.075 mmol), the mixture was stirred at 25° C. for 30 min, then 1,4-oxazocane (13.04 mg, 0.113 mmol) and N,N-diisopropylethylamine (0.033 mL, 0.189 mmol) was added at 25° C. The mixture was stirred at 60° C. for 1 h. LCMS showed the reaction was completed. The mixture was cooled, filtered and the solvent was evaporated under reduced pressure. The residue was purified by reverse preparative HPLC (Column: Boston Prime C18 150×30 mm×5 um; Condition: Water (0.05% NH₃H₂O+10 mM NH₄HCO₃)-ACN; Begin B—End B: 60—90; Gradient Time (min): 10; 100% B Hold Time (min): 2; FlowRate (mL/min): 25) to give 6-(6-chloro-8-fluoro-2-(((2R,7aS)-2-fluorotetrahydro-1H-pyrrolizin-7a(5H)-yl)methoxy)-4-(1,4-oxazocan-4-yl)quinazolin-7-yl)-4-methyl-5-(trifluoromethyl)pyridin-2-amine (Ex. 39). ¹H NMR (400 MHz, MeOD) δ 7.97 (d, J=1.4 Hz, 1H), 6.62 (s, 1H), 5.45-5.18 (m, 1H), 4.33-4.23 (m, 3H), 4.21-4.16 (m, 1H), 4.13-3.99 (m, 4H), 3.78 (t, J=5.5 Hz, 2H), 3.30-3.15 (m, 3H), 2.85 (s, 1H), 2.47 (d, J=1.4 Hz, 3H), 2.39-2.09 (m, 3H), 2.07-1.94 (m, 4H), 1.92-1.84 (m, 1H), 1.67 (br d, J=4.7 Hz, 2H). MS (ESI) [M+H]⁺: m/z 627.3.

Compounds in the table below were synthesized via a similar route as Ex. 39 using 7-(6-amino-4-methyl-3-(trifluoromethyl)pyridin-2-yl)-6-chloro-8-fluoro-2-(((2R,7a,S)-2-fluorotetrahydro-1H-pyrrolizin-7a(5N)-yl)methoxy)quinazolin-4-ol (Int-pe) and corresponding amines.

| | | | | ESI-MS |
|---|---|---|---|---|
| Ex. | Structure | Name | NMR | [M + H]⁺ |
| 40 | 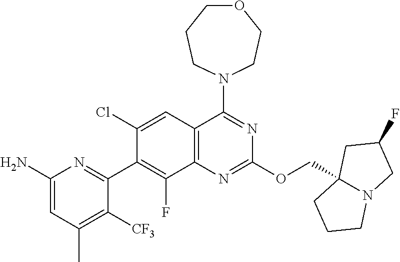 [SMILES: Cc1cc(N)nc(-c2c(Cl)cc3c(N4CCCOCC4)nc(OC[C@@]45CCCN4C[C@H](F)C5)nc3c2F)c1C(F)(F)F] | 6-(6-chloro-8-fluoro- 2-(((2R,7aS)-2- fluorotetrahydro-1H- pyrrolizin-7a(5H)- yl)methoxy)-4-(1,4- oxazepan-4- yl)quinazolin-7-yl)-4- methyl-5- (trifluoromethyl)pyridin- 2-amine | ¹H NMR (400 MHz, MeOD) δ 7.98 (d, J = 1.5 Hz, 1H), 6.60 (s, 1H), 5.44-5.17 (m, 1H), 4.30-4.11 (m, 6H), 4.08-3.96 (m, 2H), 3.88-3.76 (m, 2H), 3.29-3.12 (m, 3H), 3.01 (dd, J = 9.4, 5.6 Hz, 1H), 2.45 (d, J = 1.3 Hz, 3H), 2.39- 2.09 (m, 5H), 2.05- 1.82 (m, 3H) | 613.3 |
| 41 | 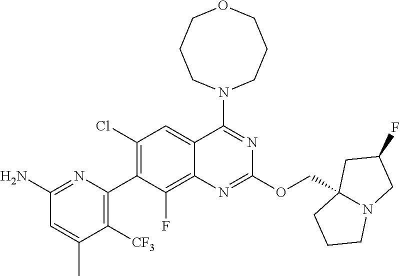 [SMILES: Cc1cc(N)nc(-c2c(Cl)cc3c(N4CCCOCCC4)nc(OC[C@@]45CCCN4C[C@H](F)C5)nc3c2F)c1C(F)(F)F] | 6-(6-chloro-8-fluoro- 2-(((2R,7aS)-2- fluorotetrahydro-1H- pyrrolizin-7a(5H)- yl)methoxy)-4-(1,5- oxazocan-5- yl)quinazolin-7-yl)-4- methyl-5- (trifluoromethyl)pyridin- 2-amine | ¹H NMR (400 MHz, MeOD) δ 8.07 (d, J = 1.2 Hz, 1H), 6.62 (s, 1H), 5.41-5.23 (m, 1H), 4.29-4.26 (m, 1H), 4.25-4.14 (m, 5H), 3.86 (t, J = 5.5 Hz, 4H), 3.30-3.15 (m, 3H), 3.01 (dt, J = 5.7, 9.4 Hz, 1H), 2.46 (br d, J = 1.4 Hz, 3H), 2.27- 2.21 (m, 1H), 2.18- 2.13 (m, 4H), 2.02- 1.95 (m, 2H), 1.92- 1.85 (m, 1H), 1.16 (br d, J = 6.4 Hz, 2H). | 627.2 |
| 42 | 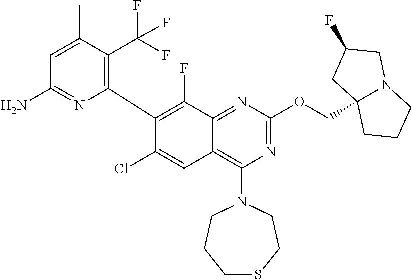 [SMILES: Cc1cc(N)nc(-c2c(Cl)cc3c(N4CCCSCC4)nc(OC[C@@]45CCCN4C[C@H](F)C5)nc3c2F)c1C(F)(F)F] | 6-(6-chloro-8-fluoro- 2-(((2R,7aS)-2- fluorotetrahydro-1H- pyrrolizin-7a(5H)- yl)methoxy)-4-(1,4- thiazepan-4- yl)quinazolin-7-yl)-4- methyl-5- (trifluoromethyl)pyridin- 2-amine | | 629.2 |
| 43 | 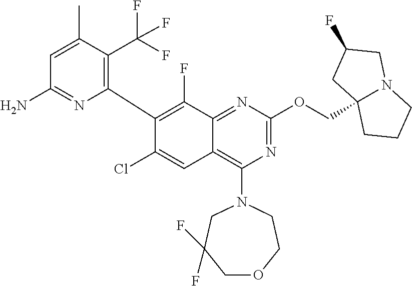 [SMILES: Cc1cc(N)nc(-c2c(Cl)cc3c(N4CCOCC(F)(F)C4)nc(OC[C@@]45CCCN4C[C@H](F)C5)nc3c2F)c1C(F)(F)F] | 6-(6-chloro-4-(6,6- difluoro-1,4- oxazepan-4-yl)-8- fluoro-2-(((2R,7aS)-2- fluorotetrahydro-1H- pyrrolizin-7a(5H)- yl)methoxy)quinazolin- 7-yl)-4-methyl-5- (trifluoromethyl)pyridin- 2-amine | | 649.2 |

### 4-(6,8-difluoro-2-(((2R,7aS)-2-fluorotetrahydro-1H-pyrrolizin-7a(5H)-yl)methoxy)-7-(6-methyl-5-(trifluoromethyl)-1H-indazol-4-yl)quinazolin-4-yl)-1,4-oxazepane (Ex. 44)

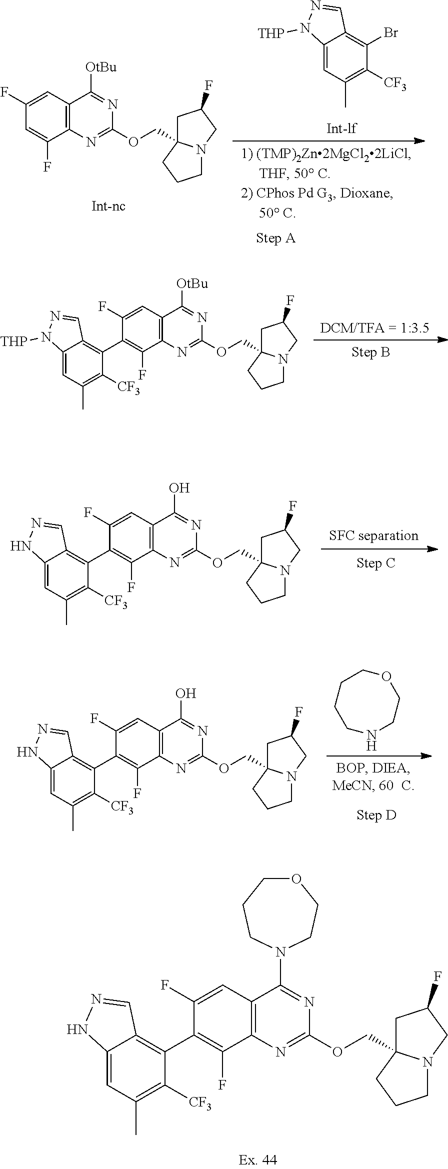

Step A: 4-(tert-butoxy)-6,8-difluoro-2-(((2R,7aS)-2-fluorotetrahydro-1H-pyrrolizin-7a(5H)-yl)methoxy)-7-(6-methyl-1-(tetrahydro-2H-pyran-2-yl)-5-(trifluoromethyl)-1H-indazol-4-yl)quinazoline

To a mixture of 4-(tert-butoxy)-6,8-difluoro-2-(((2R,7aS)-2-fluorotetrahydro-1H-pyrrolizin-7a(5H)-yl)methoxy)quinazoline (Int-nc) (1.8 g, 4.55 mmol) was added (TMP)₂Zn 2MgCl₂·2LiC1 (41.4 mL, 13.66 mmol) (0.33 M in THF) at 25° C. under N₂, and the mixture was stirred at 50° C. for 2 h. Then to the reaction mixture was added a solution of 4-bromo-6-methyl-1-(tetrahydro-2H-pyran-2-yl)-5-(trifluoromethyl)-1H-indazole (Int-if) (2.480 g, 6.83 mmol) and CPhos Pd G3 (0.367 g, 0.455 mmol) in 1,4-dioxane (30 mL) at 25° C. The reaction was stirred at 50° C. for 40 h. The reaction mixture was diluted with EtOAc (60 mL), and saturated NaHCO₃ solution (50 mL) was added to the mixture. The mixture was filtered and the filtrate was dried over Na₂SO₄, then the solvent was evaporated under reduced pressure to give the crude product. The crude product was purified by flash silica gel chromatography (ISCO®; 40 g SepaFlash® Silica Flash Column, eluent of 33% ethyl acetate in petroleum ether gradient at 30 mL/min) to give 4-(tert-butoxy)-6,8-difluoro-2-(((2R,7aS)-2-fluorotetrahydro-1H-pyrrolizin-7a(5H)-yl)methoxy)-7-(6-methyl-1-(tetrahydro-2H-pyran-2-yl)-5-(trifluoromethyl)-1H-indazol-4-yl)quinazoline. MS (ESI) [M+H]⁺: m/z 678.3

### Step B: 6,8-difluoro-2-(((2R,7aS)-2-fluorotetrahydro-1H-pyrrolizin-7a(5H)-yl)methoxy)-7-(6-methyl-5-(trifluoromethyl)-1H-indazol-4-yl)quinazolin-4-ol

To a solution of TFA (3 mL, 38.9 mmol) in DCM (10 mL) was added 4-(tert-butoxy)-6,8-difluoro-2-(((2R,7aS)-2-fluorotetrahydro-1H-pyrrolizin-7a(5H)-yl)methoxy)-7-(6-methyl-1-(tetrahydro-2H-pyran-2-yl)-5-(trifluoromethyl)-1H-indazol-4-yl)quinazoline (900 mg, 1.33 mmol) at 25° C. The reaction was stirred at 25° C. for 2 h and monitored by LCMS that showed the starting material was consumed and desired MS was formed. The solvent was evaporated under reduced pressure to give the crude which was dissolved in EtOAc (30 mL), and washed by saturated aqueous NaHCO₃ (3×10 mL). The organic layer was dried over sodium sulfate, filtered and concentrated in vacuum to give 6,8-difluoro-2-(((2R,7aS)-2-fluorotetrahydro-1H-pyrrolizin-7a(5H)-yl)methoxy)-7-(6-methyl-5-(trifluoromethyl)-1H-indazol-4-yl)quinazolin-4-ol. MS (ESI) [M+H]⁺: m/z 538.2

### Step C: 6,8-difluoro-2-(((2R,7aS)-2-fluorotetrahydro-1H-pyrrolizin-7a(5H)-yl)methoxy)-7-(6-methyl-5-(trifluoromethyl)-1H-indazol-4-yl)quinazolin-4-ol

The racemic 6,8-difluoro-2-(((2R,7aS)-2-fluorotetrahydro-1H-pyrrolizin-7a(5H)-yl)methoxy)-7-(6-methyl-5-(trifluoromethyl)-1H-indazol-4-yl)quinazolin-4-ol (3.10 g, 5.77 mmol) was separated by preparative SFC (Column: DAICEL CHIRALPAK AD (250 mm×50 mm, 10 um) Condition 0.1% NH₃H₂O EtOH Begin B 60 End B 60 Gradient Time (min) 100% B Hold Time (min) FlowRate (mL/min) 200) to give 6,8-difluoro-2-(((2R,7aS)-2-fluorotetrahydro-1H-pyrrolizin-7a(5H)-yl)methoxy)-7-(6-methyl-5-(trifluoromethyl)-1H-indazol-4-yl)quinazolin-4-ol (the second eluting isomer from SFC). ¹H NMR (400 MHz, MeOD) δ 7.78-7.70 (m, 1H), 7.68 (s, 1H), 7.59 (s, 1H), 5.52-5.25 (m, 1H), 4.50-4.36 (m, 2H), 3.63-3.40 (m, 3H), 3.16 (dt, J=5.9, 9.8 Hz, 1H), 2.70 (br d, J=1.5 Hz, 3H), 2.47-2.28 (m, 2H), 2.25-2.16 (m, 1H), 2.15-2.04 (m, 2H), 2.02-1.90 (m, 1H). MS (ESI) [M+H]⁺: m/z 538.21

### Step D: 4-(6,8-difluoro-2-(((2R,7aS)-2-fluorotetrahydro-1H-pyrrolizin-7a(5H)-yl)methoxy)-7-(6-methyl-5-(trifluoromethyl)-1H-indazol-4-yl)quinazolin-4-yl)-1,4-oxazepane (Ex. 44)

To a solution of 6,8-difluoro-2-(((2R,7aS)-2-fluorotetrahydro-1H-pyrrolizin-7a(5H)-yl)methoxy)-7-(6-methyl-5-(trifluoromethyl)-1H-indazol-4-yl)quinazolin-4-ol (50 mg, 0.093 mmol) (used active peak 2) in MeCN (0.5 mL) was added BOP (82 mg, 0.186 mmol) at 25° C. The mixture was stirred at 25° C. for 30 min, and then 1,4-oxazepane hydrochloride (25.6 mg, 0.186 mmol) and DIEA (0.097 mL, 0.558 mmol) was added at 25° C. Then the reaction was stirred at 60° C. for 4 h and monitored by LCMS that showed the starting material was disappeared and desired MS was found. The mixture was filtered and the filtrate was purified by preparative HPLC (Column Welch Xtimate C18 150×25 mm×5 um Condition water (10 mM HCOONH4)-ACN Begin B 40 End B 70 Gradient Time (min) 11 100% B Hold Time 2 Flow Rate (mL/min) 25 Injections 2) to give 4-(6,8-difluoro-2-(((2R,7aS)-2-fluorotetrahydro-1H-pyrrolizin-7a(5H)-yl)methoxy)-7-(6-methyl-5-(trifluoromethyl)-1H-indazol-4-yl)quinazolin-4-yl)-1,4-oxazepane (Ex. 44). ¹H NMR (400 MHz, MeOD) δ 7.79 (dd, J=1.7, 10.6 Hz, 1H), 7.68 (s, 1H), 7.61 (s, 1H), 5.41-5.19 (m, 1H), 4.31-4.23 (m, 1H), 4.22-4.14 (m, 5H), 4.05-3.99 (m, 2H), 3.87-3.81 (m, 2H), 3.29-3.15 (m, 3H), 3.00 (dd, J=5.7, 9.4 Hz, 1H), 2.71 (d, J=1.7 Hz, 3H), 2.39-2.09 (m, 5H), 2.04-1.83 (m, 3H). MS (ESI) [M+H]⁺ m/z: 621.2.

### (1R,7S,8S)-4-(7-(6-amino-4-methyl-3-(trifluoromethyl)pyridin-2-yl)-6,8-difluoro-2-(((2R,7aS)-2-fluorotetrahydro-1H-pyrrolizin-7a(5H)-yl)methoxy)quinazolin-4-yl)-4-azabicyclo[5.1.0]octane-8-carbonitrile (Ex. 45)

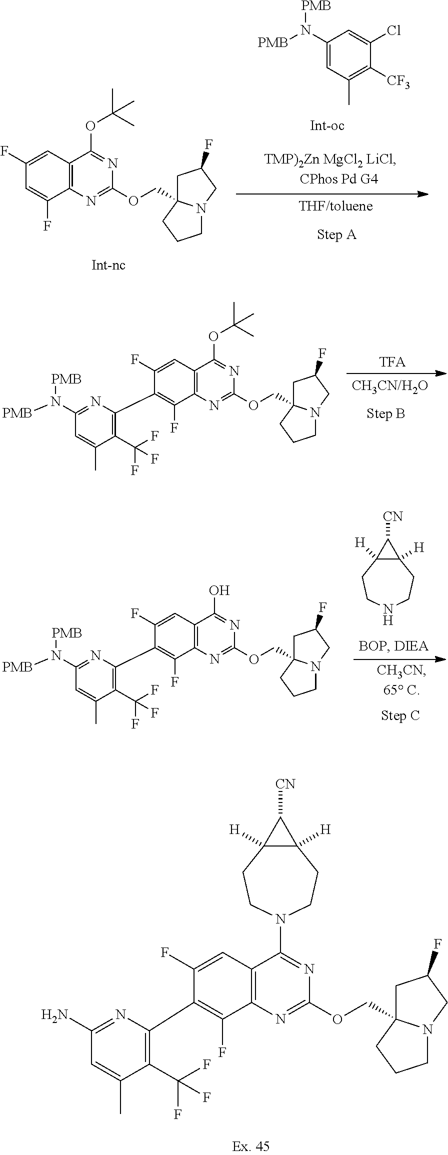

### Step A: 6-(4-(tert-butoxy)-6,8-difluoro-2-(((2R,7aS)-2-fluorotetrahydro-1H-pyrrolizin-7a(5H)-yl)methoxy)quinazolin-7-yl)-N,N-bis(4-methoxybenzyl)-4-methyl-5-(trifluoromethyl)pyridin-2-amine

A solution of 4-(tert-butoxy)-6,8-difluoro-2-(((2R,7aS)-2-fluorotetrahydro-1H-pyrrolizin-7a(5H)-yl)methoxy)quinazoline (Int-nc) (500 mg, 1.3 mmol) in tetrahydrofuran (6322 μl) was added to a solution of bis(2,2,6,6-tetramethylpiperidinyl)zinc-lithium chloride-magnesium chloride 1:1:1 complex solution in tetrahydrofuran-toluene (0.2 M, 12 mL, 2.5 mmol). The resulting mixture was stirred under inert atmosphere at 25° C. for 1 hour. In a separate flask, 6-chloro-N,N-bis(4-methoxybenzyl)-4-methyl-5-(trifluoromethyl)pyridin-2-amine (Int-oc) (1.1 g, 2.5 mmol), CPhos Pd G4 (160 mg, 0.19 mmol), and 1,4-dioxane (6.3 mL) were combined. This mixture was then added dropwise to the aryl-zine solution prepared above and the resulting reaction mixture was stirred at 25° C. for 2 hours. The mixture was diluted with ethyl acetate, and the diluted organic mixture was washed with saturated aqueous sodium chloride solution. The washed organic solution was dried over sodium sulfate, filtered, and concentrated. The crude residue was purified by flash-column chromatography (eluting with hexanes initially, grading to 100% ethyl acetate) to afford 6-(4-(tert-butoxy)-6,8-difluoro-2-(((2R,7aS)-2-fluorotetrahydro-1H-pyrrolizin-7a(5H)-yl)methoxy)quinazolin-7-yl)-N,N-bis(4-methoxybenzyl)-4-methyl-5-(trifluoromethyl)pyridin-2-amine. ESI-MS m/z [M+H]⁺ 810.

### Step B: 7-(6-(bis(4-methoxybenzyl)amino)-4-methyl-3-(trifluoromethyl)pyridin-2-yl)-6,8-difluoro-2-(((2R,7aS)-2-fluorotetrahydro-1H-pyrrolizin-7a(5H)-yl)methoxy)quinazolin-4-ol

6-(4-(tert-Butoxy)-6,8-difluoro-2-(((2R,7aS)-2-fluorotetrahydro-1H-pyrrolizin-7a(5H)-yl)methoxy)quinazolin-7-yl)-N,N-bis(4-methoxybenzyl)-4-methyl-5-(trifluoromethyl)pyridin-2-amine (840 mg, 1.0 mmol) was dissolved in acetonitrile (6.5 mL), water (1.6 mL), and trifluoroacetic acid (82 μL). The resulting solution was stirred at 25° C. for 2 hours. The mixture was then basified with the addition of saturated aqueous sodium bicarbonate solution, and the basified mixture was extracted with ethyl acetate. The combined organic extracts were washed with saturated aqueous sodium chloride solution, dried over sodium sulfate, filtered, and concentrated under reduced pressure to provide 7-(6-(bis(4-methoxybenzyl)amino)-4-methyl-3-(trifluoromethyl)pyridin-2-yl)-6,8-difluoro-2-(((2R,7aS)-2-fluorotetrahydro-1H-pyrrolizin-7a(5H)-yl)methoxy)quinazolin-4-ol. ESI-MS m/z [M+H]⁺ 754.

### Step C: (1R,7S,8S)-4-(7-(6-amino-4-methyl-3-(trifluoromethyl)pyridin-2-yl)-6,8-difluoro-2-(((2R,7aS)-2-fluorotetrahydro-1H-pyrrolizin-7a(5H)-yl)methoxy)quinazolin-4-yl)-4-azabicyclo[5.1.0]octane-8-carbonitrile (Ex. 45)

Benzotriazol-1-yloxytris(dimethylamino)phosphonium hexafluorophosphate (18 mg, 0.040 mmol) was added to a stirred solution of 7-(6-(bis(4-methoxybenzyl)amino)-4-methyl-3-(trifluoromethyl)pyridin-2-yl)-6,8-difluoro-2-(((2R,7aS)-2-fluorotetrahydro-1H-pyrrolizin-7a(5H)-yl)methoxy)quinazolin-4-ol (20 mg, 0.027 mmol) and N,N-diisopropylethylamine (14 L, 0.080 mmol) in acetonitrile (260 L) at 23° C. The mixture was stirred for 15 min before (1R,7S,8S)-4-azabicyclo[5.1.0]octane-8-carbonitrile hydrochloride (9.2 mg, 0.053 mmol) was added. The mixture was then heated with stirring to 65° C. After 2 h, the mixture was concentrated under reduced pressure. The residue was redissolved in trifluoroacetic acid (1.5 mL) and the solution was warmed to 50° C. for 2 h. The mixture was then concentrated under reduced pressure, and the residue was purified by reverse-phase HPLC (acetonitrile-water containing 0.1% v/v trifluoroacetic acid) to provide (1R,7S,8S)-4-(7-(6-amino-4-methyl-3-(trifluoromethyl)pyridin-2-yl)-6,8-difluoro-2-(((2R,7aS)-2-fluorotetrahydro-1H-pyrrolizin-7a(5H)-yl)methoxy)quinazolin-4-yl)-4-azabicyclo[5.1.0]octane-8-carbonitrile trifluoroacetic acid salt (1:2) (Ex. 45). ¹H NMR (500 MHz, CD₃OD) δ 7.66 (d, J=10.1 Hz, 1H), 6.67 (s, 1H) 5.58 (d, J=51.9 Hz, 1H), 4.72-4.60 (m, 2H), 4.16-4.06 (m, 2H), 4.04-3.85 (m, 5H), 3.50-3.47 (m, 1H), 2.75-2.59 (m, 4H), 2.48 (s, 3H), 2.45-2.42 (m, 1H), 2.39-2.33 (m, 2H), 2.18 (br s, 1H), 1.94-1.83 (m, 4H), 1.67 (t, J=4.8 Hz, 1H). ¹⁹F NMR (470 MHz, CD₃OD) δ −56.4 (s, 3F), −77.5 (s, 6F). ESI-MS m/z [M+H]⁺ 632.

### 2-amino-7-fluoro-4-(2-(((2R,7aS)-2-fluorotetrahydro-1H-pyrrolizin-7a(5H)-yl)methoxy)-4-(1,4-oxazepan-4-yl)-6-(trifluoromethyl)pyrido[3,2-d]pyrimidin-7-yl)benzo[b]thiophene-3-carbonitrile (Ex. 46)

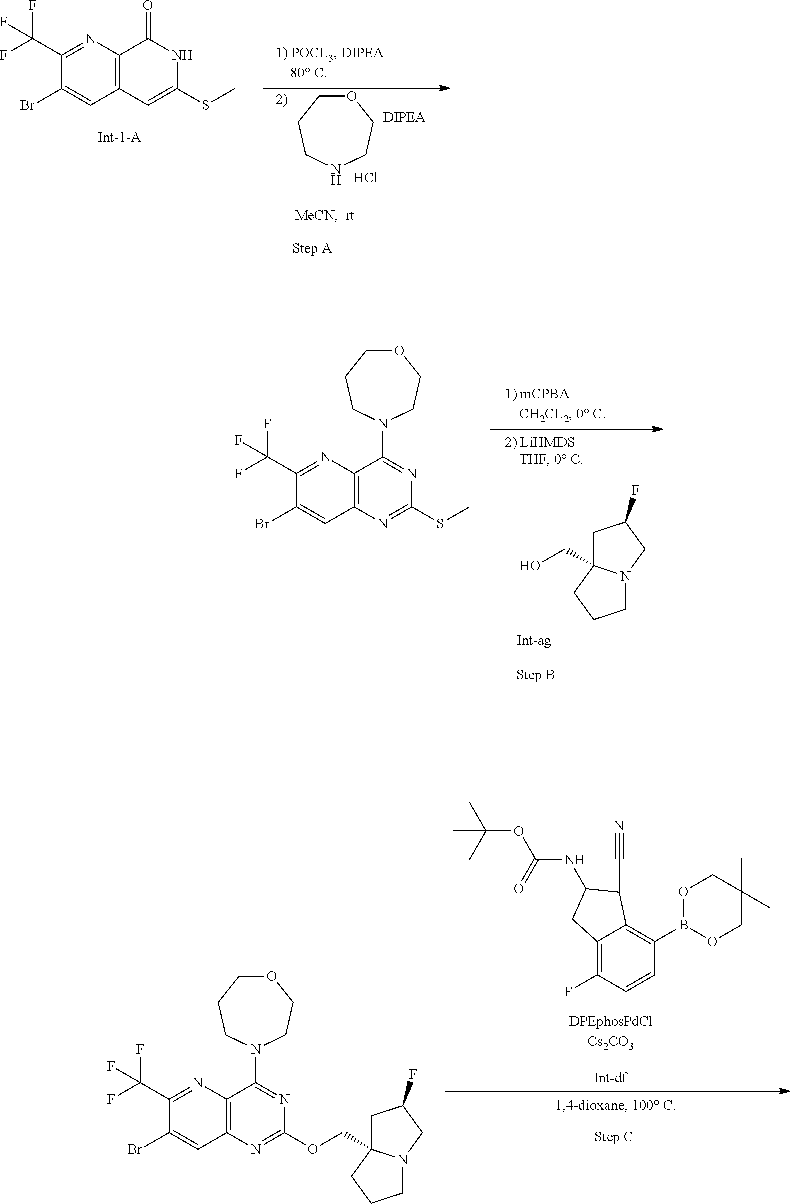

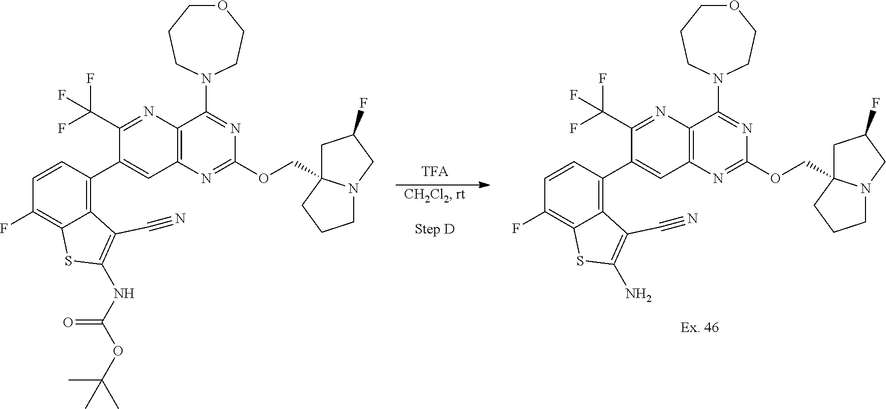 [SMILES: CC(C)(C)OC(=O)Nc1sc2c(F)ccc(-c3cc4nc(OC[C@@]56CCCN5C[C@H](F)C6)nc(N5CCCOCC5)c4nc3C(F)(F)F)c2c1C#N ; N#Cc1c(N)sc2c(F)ccc(-c3cc4nc(OC[C@@]56CCCN5C[C@H](F)C6)nc(N5CCCOCC5)c4nc3C(F)(F)F)c12]

### Step A: 4-(7-bromo-2-(methylthio)-6-(trifluoromethyl)pyrido[3,2-d]pyrimidin-4-yl)-1,4-oxazepane

To a solution of 7-bromo-2-(methylthio)-6-(trifluoromethyl)pyrido[3,2-d]pyrimidin-4(3H)-one (400 mg, 1.18 mmol) in POCl₃ (0.160 ml, 1.76 mmol) was added N,N-diisopropylethylamine (0.600 ml, 3.53 mmol) at room temperature. The mixture was stirred at 80° C. for 1 h under nitrogen atmosphere. The reaction mixture was cooled to room temperature and concentrated in vacuo. The residue was azeotroped with toluene and dried under reduced pressure for 4 h to afford 7-bromo-4-chloro-2-(methylthio)-6-(trifluoromethyl)pyrido[3,2-d]pyrimidine. The crude material was used directly in the next step without further purification. MS (ESI): m/z (M+H)+358, 360.

To a stirred mixture of crude 7-bromo-4-chloro-2-(methylthio)-6-(trifluoromethyl)pyrido[3,2-d]pyrimidine and N,N-diisopropylethylamine (0.600 ml, 3.53 mmol) in MeCN (20.0 mL) was added 1,4-oxazepane hydrochloride (194 mg, 1.41 mmol) at 0° C. After being stirred for 20 minutes, to the mixture was added water, and diluted with ethyl acetate. The organic layer was dried over Na₂SO₄, filtered and concentrated in vacuo. The residue was purified by flash silica chromatography (5-20%, EtOAc gradient in hexane) to obtain 4-(7-bromo-2-(methylthio)-6-(trifluoromethyl)pyrido[3,2-d]pyrimidin-4-yl)-1,4-oxazepane (167 mg). ESI-MS m/z [M+H]⁺ 423, 425. ¹H NMR (400 MHz, CDCl₃) δ 8.25 (d, J=0.5 Hz, 1H), 4.65-4.62 (m, 2H), 4.20-4.11 (m, 2H), 3.96-3.92 (m, 2H), 3.79-3.74 (m, 2H), 2.57-2.52 (m, 3H), 2.13-2.04 (m, 2H).

### Step B: 4-(7-bromo-2-(((2R,7aS)-2-fluorotetrahydro-1H-pyrrolizin-7a(5H)-yl)methoxy)-6-(trifluoromethyl)pyrido[3,2-d]pyrimidin-4-yl)-1,4-oxazepane

To a stirred solution of 4-(7-bromo-2-(methylthio)-6-(trifluoromethyl)pyrido[3,2-d]pyrimidin-4-yl)-1,4-oxazepane (167 mg, 0.350 mmol) in DCM (5.00 mL) was added m-chloroperoxybenzoic acid (105 mg, 0.192 mmol, 65 wt %) at 0° C. After 40 minutes, to the mixture was added saturated aqueous sodium bicarbonate and the mixture was extracted with chloroform. The organic extract was washed with brine, dried over sodium sulfate, filtered, and concentrated to give 4-(7-bromo-2-(methylsulfinyl)-6-(trifluoromethyl)pyrido[3,2-d]pyrimidin-4-yl)-1,4-oxazepane. The crude material was used directly in the next step without further purification. MS (ESI): m/z (M+H)+439, 441.

To a stirred solution of ((2R,7aS)-2-fluorotetrahydro-1H-pyrrolizin-7a(5H)-yl)methanol (Int-ag) (126 mg, 0.791 mmol) in THE (3.30 mL) was added lithium bis(trimethylsilyl)amide (0.730 mL, 0.952 mmol, 1.0 M in THF) at 0° C. and the resulting mixture was stirred at 0° C. for 15 minutes. Then to the mixture was added crude 4-(7-bromo-2-(methylsulfinyl)-6-(trifluoromethyl)pyrido[3,2-d]pyrimidin-4-yl)-1,4-oxazepane in THE (3.30 mL) dropwise. After 30 minutes, to the mixture was added water and the mixture was extracted with EtOAc. The combined organic phase was dried over sodium sulfate, filtered, and concentrated. The residue was purified by flash NH-silica gel chromatography (20-60%, EtOAc gradient in hexane) to give 4-(7-bromo-2-(((2R,7aS)-2-fluorotetrahydro-1H-pyrrolizin-7a(5H)-yl)methoxy)-6-(trifluoromethyl)pyrido[3,2-d]pyrimidin-4-yl)-1,4-oxazepane. MS (ESI): m/z (M+H)+534, 536. ¹H NMR (400 MHz, CDCl₃) δ 8.21 (s, 1H), 5.34-5.20 (m, 1H), 4.67-4.63 (m, 2H), 4.18-4.04 (m, 4H), 3.97-3.91 (m, 2H), 3.77 (t, J=5.6 Hz, 2H), 3.27-3.10 (m, 3H), 3.00-2.94 (m, 1H), 2.31-2.20 (m, 1H), 2.14-2.04 (m, 4H), 1.98-1.81 (m, 3H).

### Step C: tert-butyl (3-cyano-7-fluoro-4-(2-(((2R,7aS)-2-fluorotetrahydro-1H-pyrrolizin-7a(5H)-yl)methoxy)-4-(1,4-oxazepan-4-yl)-6-(trifluoromethyl)pyrido[3,2-d]pyrimidin-7-yl)benzo[b]thiophen-2-yl)carbamate

To a solution of 4-(7-bromo-2-(((2R,7aS)-2-fluorotetrahydro-1H-pyrrolizin-7a(5H)-yl)methoxy)-6-(trifluoromethyl)pyrido[3,2-d]pyrimidin-4-yl)-1,4-oxazepane (89.5 mg, 0.17 mmol), tert-butyl (3-cyano-4-(5,5-dimethyl-1,3,2-dioxaborinan-2-yl)-7-fluorobenzo[b]thiophen-2-yl)carbamate (Int-df) (128 mg, 0.251 mmol) and cesium carbonate (246 mg, 0.753 mmol) in 1,4-dioxane (3.40 ml) was added dichloro[bis(2-(diphenylphosphino)phenyl)ether]palladium(II) (60.0 mg, 0.084 mmol) at room temperature. The mixture was degassed under reduced pressure and purged with nitrogen several times. The mixture was stirred at 100° C. for 40 minutes under nitrogen atmosphere. The reaction mixture was cooled to room temperature and diluted with water and ethyl acetate. The organic layer was dried over Na₂SO₄, filtered and concentrated in vacuo. The residue was purified by flash NH-silica gel chromatography (0-20%, MeOH gradient in EtOAc) to obtain tert-butyl (3-cyano-7-fluoro-4-(2-(((2R,7aS)-2-fluorotetrahydro-1H-pyrrolizin-7a(5H)-yl)methoxy)-4-(1,4-oxazepan-4-yl)-6-(trifluoromethyl)pyrido[3,2-d]pyrimidin-7-yl)benzo[b]thiophen-2-yl)carbamate (95.1 mg). ESI-MS m/z [M+H]⁺ 746.

### Step D: 2-amino-7-fluoro-4-(2-(((2R,7aS)-2-fluorotetrahydro-1H-pyrrolizin-7a(5H)-yl)methoxy)-4-(1,4-oxazepan-4-yl)-6-(trifluoromethyl)pyrido[3,2-d]pyrimidin-7-vl)benzo[b]thiophene-3-carbonitrile (Ex. 46)

To a solution of tert-butyl (3-cyano-7-fluoro-4-(2-(((2R,7aS)-2-fluorotetrahydro-1H-pyrrolizin-7a(5H)-yl)methoxy)-4-(1,4-oxazepan-4-yl)-6-(trifluoromethyl)pyrido[3,2-d]pyrimidin-7-yl)benzo[b]thiophen-2-yl)carbamate (95.1 mg, 0.132 mmol) in dichloromethane (1.50 ml) was added trifluoroacetic acid (0.50 ml, 7.00 mmol) at room temperature under nitrogen atmosphere. The mixture was stirred at room temperature for 1 h under nitrogen atmosphere. The reaction mixture was basified with saturated sodium bicarbonate aqueous solution. The organic layer was dried over Na₂SO₄, filtered and concentrated in vacuo. The residue was purified by reverse phase HPLC (MeCN/water with 0.1% formic acid) to obtain 2-amino-7-fluoro-4-(2-(((2R,7aS)-2-fluorotetrahydro-1H-pyrrolizin-7a(5H)-yl)methoxy)-4-(1,4-oxazepan-4-yl)-6-(trifluoromethyl)pyrido[3,2-d]pyrimidin-7-yl)benzo[b]thiophene-3-carbonitrile (Ex. 46) (49.4 mg). ESI-MS m/z [M+H]⁺ 646. ¹H NMR (400 MHz, CDCl₃) δ 7.83 (s, 1H), 7.18 (dd, J=8.3, 5.0 Hz, 1H), 6.98 (dd, J=9.1, 8.4 Hz, 1H), 5.42-5.21 (m, 3H), 4.85-4.65 (m, 2H), 4.27-3.76 (m, 7H), 3.64 (td, J=12.2, 6.2 Hz, 1H), 3.27-3.12 (m, 3H), 3.01-2.95 (m, 1H), 2.28-2.13 (m, 5H), 1.95-1.84 (m, 3H).

Compounds in the table below were synthesized via similar routes as described in the Examples above.

| | | | | ESI-MS |
|---|---|---|---|---|
| Ex. | Structure | Name | NMR | [M + H]+ |
| 47 | 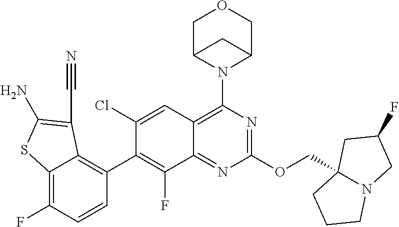 [SMILES: N#Cc1c(N)sc2c(F)ccc(-c3c(Cl)cc4c(N5C6COCC5C6)nc(OC[C@@]56CCCN5C[C@H](F)C6)nc4c3F)c12] | 4-(4-(3-oxa-6- azabicyclo[3.1.1]heptan- 6-yl)-6-chloro-8-fluoro- 2-(((2R,7aS)-2- fluorotetrahydro-1H- pyrrolizin-7a(5H)- yl)methoxy)quinazolin- 7-yl)-2-amino-7- fluorobenzo[b]thiophene- 3-carbonitrile | ¹H NMR (400 MHz, CHLOROFORM- D) d 7.58 (d, J = 1.5 Hz, 1H), 7.19 (dd, J = 8.3, 5.0 Hz, 1H), 7.01 (t, J = 8.8 Hz, 1H), 5.69 (s, 2H), 5.26 (dd, J = 53.8, 1.5 Hz, 1H), 4.91 (d, J = 5.8 Hz, 2H), 4.41 (s, 2H), 4.18 (dd, J = 49.4, 10.4 Hz, 2H), 3.96 (t, J = | 627, 629 |
| | | | 9.9 Hz, 2H), 3.32- | |
| | | | 3.11 (m, 3H), 2.99- | |
| | | | 2.89 (m, 2H), 2.30- | |
| | | | 2.03 (m, 4H), 1.97- | |
| | | | 1.82 (m, 3H) | |
| 48 | 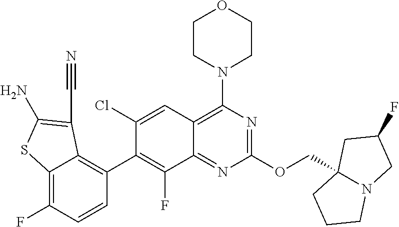 [SMILES: N#Cc1c(N)sc2c(F)ccc(-c3c(Cl)cc4c(N5CCOCC5)nc(OC[C@@]56CCCN5C[C@H](F)C6)nc4c3F)c12] | 2-amino-4-(6-chloro-8- fluoro-2-(((2R,7aS)-2- fluorotetrahydro-1H- pyrrolizin-7a(5H)- yl)methoxy)-4- morpholinoquinazolin- 7-yl)-7- fluorobenzo[b]thiophene- 3-carbonitrile | ¹H NMR (400 MHz, CHLOROFORM- D) d 7.72 (d, J = 1.5 Hz, 1H), 7.21 (dd, J = 8.3, 5.0 Hz, 1H), 7.03 (t, J = 8.8 Hz, 1H), 5.47 (s, 2H), 5.35-5.21 (m, 1H), 4.29-4.13 (m, 2H), 3.90-3.79 (m, 8H), 3.27-3.17 (m, 3H), 3.00-2.94 (m, 1H), 2.28-2.11 (m, 3H), | 615, 616 |
| | | | 2.05-1.84 (m, 3H) | |
| 49 | 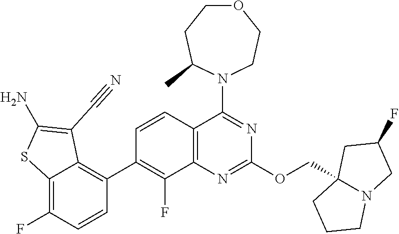 [SMILES: C[C@H]1CCOCCN1c1nc(OC[C@@]23CCCN2C[C@H](F)C3)nc2c(F)c(-c3ccc(F)c4sc(N)c(C#N)c34)ccc12] | 2-amino-7-fluoro-4-(8- fluoro-2-(((2R,7aS)-2- fluorotetrahydro-1H- pyrrolizin-7a(5H)- yl)methoxy)-4-((S)-5- methyl-1,4-oxazepan-4- yl)quinazolin-7- yl)benzo[b]thiophene-3- carbonitrile | 1H-NMR (400 MHz, CDCl3) δ 7.67 (dd, J = 8.8, 3.5 Hz, 1H), 7.23- 7.12 (m, 2H), 7.02- 6.93 (m, 1H), 6.56 (s, 1H), 6.33 (s, 1H), 5.36-5.22 (m, 1H), 4.89-4.79 (m, 1H), 4.45 (d, J = 15.0 Hz, 1H), 4.25 (dd, J = 10.0, 7.8 Hz, 1H), 4.17-4.09 (m, 2H), 4.05-3.96 | 609 |
| | | | (m, 2H), 3.61-3.40 | |
| | | | (m, 2H), 3.32-3.22 | |
| | | | (m, 2H), 3.19 (d, J = | |
| | | | 5.8 Hz, 1H), 3.03 | |
| | | | 2.97 (m, 1H), 2.37- | |
| | | | 2.31 (m, 2H), 2.26- | |
| | | | 2.13 (m, 2H), 2.07- | |
| | | | 1.92 (m, 4H), 1.54- | |
| | | | 1.48 (m, 3H) | |
| 50 | 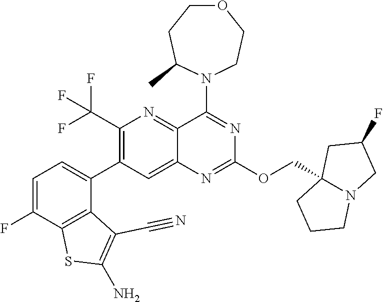 [SMILES: C[C@H]1CCOCCN1c1nc(OC[C@@]23CCCN2C[C@H](F)C3)nc2cc(-c3ccc(F)c4sc(N)c(C#N)c34)c(C(F)(F)F)nc12] | 2-amino-7-fluoro-4-(2- (((2R,7aS)-2- fluorotetrahydro-1H- pyrrolizin-7a(5H)- yl)methoxy)-4-((S)-5- methyl-1,4-oxazepan-4- yl)-6- (trifluoromethyl)pyrido[3,2- d]pyrimidin-7- yl)benzo[b]thiophene-3- carbonitrile | ¹H NMR (400 MHz, CDCl₃) δ 7.83 (d, J = 2.3 Hz, 1H), 7.18-7.12 (m, 1H), 7.01-6.95 (m, 1H), 5.38-5.21 (m, 3H), 4.25-4.18 (m, 1H), 4.10-3.94 (m, 2H), 3.65 (td, J = 12.2, 6.1 Hz, 1H), 3.58-3.42 (m, 2H), 3.30-3.12 (m, 3H), 3.01-2.94 (m, 1H), 2.29-2.13 (m, 4H), 2.04-1.90 (m, 4H), 1.34-1.25 (m, 3H) | 660 |
| 51 | 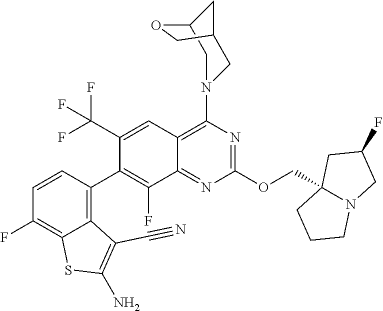 [SMILES: N#Cc1c(N)sc2c(F)ccc(-c3c(C(F)(F)F)cc4c(N5CC6COC(C6)C5)nc(OC[C@@]56CCCN5C[C@H](F)C6)nc4c3F)c12] | 4-(4-((1R,5S)-6-oxa-3- azabicyclo[3.2.1]octan- 3-yl)-8-fluoro-2- (((2R,7aS)-2- fluorotetrahydro-1H- pyrrolizin-7a(5H)- yl)methoxy)-6- (trifluoromethyl)quinazo- lin-7-yl)-2-amino-7- fluorobenzo[b]thiophene- 3-carbonitrile | | 675 |
| 52 | 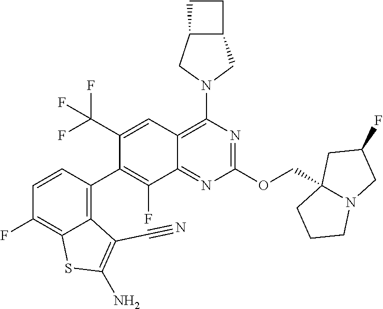 [SMILES: N#Cc1c(N)sc2c(F)ccc(-c3c(C(F)(F)F)cc4c(N5C[C@H]6CC[C@H]6C5)nc(OC[C@@]56CCCN5C[C@H](F)C6)nc4c3F)c12] | 4-(4-((1R,5S)-3- azabicyclo[3.2.0]heptan- 3-yl)-8-fluoro-2- (((2R,7aS)-2- fluorotetrahydro-1H- pyrrolizin-7a(5H)- yl)methoxy)-6- (trifluoromethyl)quinazo- lin-7-yl)-2-amino-7- fluorobenzo[b]thiophene- 3-carbonitrile | | 659 |
| 53 | 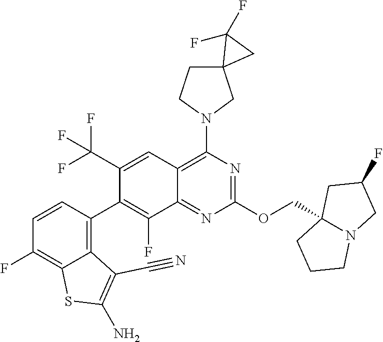 [SMILES: N#Cc1c(N)sc2c(F)ccc(-c3c(C(F)(F)F)cc4c(N5CCC6(C5)CC6(F)F)nc(OC[C@@]56CCCN5C[C@H](F)C6)nc4c3F)c12] | 2-amino-4-(4-(1,1- difluoro-5- azaspiro[2.4]heptan-5- yl)-8-fluoro-2- (((2R,7aS)-2- fluorotetrahydro-1H- pyrrolizin-7a(5H)- yl)methoxy)-6- (trifluoromethyl)quinazo- lin-7-yl)-7- fluorobenzo[b]thiophene- 3-carbonitrile | | 695 |

### Assays

### Procedure for SOS-Catalyzed Nucleotide Exchange Assay for KRAS-G12C/D/V, G13D, HRAS, and NRAS (Procedure A)

Recombinant KRAS-G12C protein used in this assay has an additional triple mutation (C51S/C80L/C118S), whereas KRAS-G12D/V, G13D, HRAS and NRAS are in context of their WT protein sequence background. Specifically, the SOS-catalyzed nucleotide exchange assay utilizes a preformed TR-FRET complex containing a specific biotinylated RAS protein (KRAS-G12C/V/D, G13D, HRAS, NRAS; described above) with Bodipy-GDP, and Terbium-streptavidin. Compounds are preincubated with this complex for 60 minutes. Subsequently, recombinant human SOS protein and unlabeled GTP are added to initiate the exchange reaction. Small molecule inhibitors stabilize the Bodipy-GDP complex whereas the untreated protein rapidly exchanges Bodipy-GDP for unlabeled GTP resulting in reduced TR-FRET signal.

To assemble the preformed TR-FRET complexes, each biotinylated RAS protein is diluted to 2 μM in an EDTA Buffer (20 mM HEPES pH 7.5, 50 mM sodium chloride, 10 mM EDTA, and 0.01% Tween) and incubated at room temperature for one hour. This mixture is then further diluted to 90 nM in an Assay Buffer (20 mM HEPES pH 7.5, 150 mM sodium chloride, 10 mM magnesium chloride, and 0.005% Tween) containing 15 nM of Terbium-Streptavidin (Invitrogen, catalog #PV3577) and 900 nM of Bodipy-GDP (Invitrogen, catalog #G22360) and incubated at room temperature for six hours. It should be noted that this preformed TR-FRET complex for each of the RAS protein were made ahead of time, aliquoted and stored at −80° C. until the day of the experiment.

Each test compound (10 mM stock in DMSO) is diluted in DMSO to make a final-10-point, 3-fold dilution and is acoustically dispensed into a 384-well assay plate (Corning, catalog #3820) using an Echo 550 (Labcyte). Each well of the assay plate receives 3 μL of a specific 3× RAS preformed TR-FRET complex and 3 μL of Assay Buffer and is incubated at room temperature for 60 minutes (preincubation time). Each well then receives 3 μL of 3× recombinant human SOS protein and GTP (Sigma, G8877) in Assay Buffer and is incubated at room temperature for 30 minutes (G13D), 60 minutes (G12C/D, H/NRAS) or 90 minutes for G12V. The final reaction in each well of 9 L consists of 3 mM GTP, specific Ras and SOS proteins in the following concentrations: KRAS-G12C/SOS=3 nM/40 nM, KRAS-G12D/SOS=1 nM/40 nM, KRAS-G12V/SOS=1.25 nM/160 nM, KRAS-G13D/SOS=1.25 nM/0 nM, HRAS & NRAS=1.25 nM/40 nM.

The time-resolved fluorescence resonance energy transfer (TR-FRET) signal is measured on an Envision (PerkinElmer) plate reader: Excitation filter=340 nm; emission1=495 nm; emission2=520 nm; dichroic mirror=D400/D505; delay time=100 ms. The signal of each well is determined as the ratio of the emission at 520 nm to that at 495 nm. Percent effect of each well is determined after normalization to control wells containing DMSO (no effect) or a saturating concentration of inhibitor (max effect). The apparent effect as a function of compound concentration is fit to a four-parameter logistic equation.

### Procedure for SOS-Catalyzed Nucleotide Exchange Assay for KRAS-G12C/D/V (Procedure B)

Recombinant KRAS G12C (amino acids 1-169, SEQ ID NO:8), KRAS G12D (amino acids 1-169, SEQ ID NO:9), KRAS G12V (amino acids 1-169, SEQ ID NO:10) and SOS1 (amino acids 564-1049, SEQ ID NO:11) proteins were expressed in E. coli and purified by affinity chromatography. To prepare each BODIPY™ FL GDP-bound KRAS mutant protein, 50 μM KRAS mutant proteins were incubated with 0.5 mM BODIPY™ FL GDP (Invitrogen, G22360) in a loading buffer (20 mM Tris-HCl pH 7.5, 50 mM NaCl, 1 mM DTT and 2.5 mM EDTA) for 1 hour on ice. After the incubation, MgCl₂ was added to a final concentration of 10 mM, followed by an incubation at room temperature for 30 minutes. The mixtures were allowed to pass through a NAP-5 column to remove free nucleotides and purified BODIPY™ FL GDP-bound KRAS G12C, G12D and G12V proteins were used for compound evaluation.

The inhibitory activity of compounds on recombinant KRAS mutants is measured by the displacement of the bound BODIPY™ GDP. Specifically, 2.5 nM of each BODIPY™ FL GDP-bound KRAS mutant complex was incubated with various concentrations of compound in a reaction buffer (20 mM Tris-HCl pH 7.5, 100 mM NaCl, 1 mM MgCl₂, 2 mM DTT, 0.1% Tween 20) at 25° C. for 1 hour. After the incubation, recombinant SOS1 and GMPPNP (Jena Bioscience GmbH, NU-401) were added and incubated at room temperature for 30 minutes to proceed SOS1-dependent GDP-GTP exchange reaction on KRAS mutants. Displacement of BODIPY™ FL GDP by Guanosine-5′-[(β,γ)-imido]triphosphate, Tetralithium salt (GMPPNP) was measured by calculating the ratio of fluorescence intensities of BODIPY™ FL before and after the exchange reaction. Percent Inhibition was calculated by setting the fluorescence ratio from the reaction without test compound (DMSO control) and the fluorescence ratio from the reaction without SOS1 and GMPPNP as 0% and 100% inhibition, respectively. Dose response curves were analyzed using a 4-parameter logistic model to calculate IC₅₀ values.

### Procedure for Cellular Phospho-ERK Assay in G12D and G12V Cell Lines

PANC08.13 cells (ATCC® CRL-2551™), containing homozygous KRAS-G12D activating mutation, were cultured in growth medium made of RPMI1640-GlutaMAX™—I (ThermoFisher Scientific 61870) containing 15% heat inactivated fetal bovine serum (ThermoFisher Scientific 10091148)). SW620 cells (ATCC® CRL-227™), containing homozygous KRAS-G12V activating mutation, were cultured in growth medium that contains RPMI 1640-GlutaMAX™—I (ThermoFisher Scientific 61870) containing 10% heat inactivated fetal bovine serum (ThermoFisher Scientific 10091148).

Cells for the assay were harvested in growth medium after TrypLE (ThermoFisher scientific 12604021) digestion and were seeded in a 384-well collagen coated cell culture plate (Corning 356702) at a density of 10,000-15,000 cells/20 uL/well, and incubated at 37° C., 5% CO₂ overnight. The compound (with 10 mM stock concentration) dose-response titrations were prepared [30 μM final ERK detection assay concentration and 1:3 dilutions, 10-point dose response] and appropriate amounts (270 nL) of test compounds were dispensed in a 384-well intermediate plate using an Echo 550 liquid handler. 30 uL/well of RPMI medium 1640-GlutaMAX™—I was added to the intermediate plate and the contents of the intermediate plate (10 uL/well) were then transferred to the 384-well collagen coated cell culture plate, which was incubated at 37° C., 5% CO₂ for 2 hours. After removal of medium from the collagen coated cell culture plate, cells were lysed in lysis buffer from Alpha SureFire® Ultra™ Multiplex p-ERK and total ERK assay kit (PerkinElmer MPSU-PTERK) containing Halt™ Protease and Phosphatase inhibitor cocktail (ThermoFisher Scientific 78446) at room temperature with constant shaking at 300 rpm for 30 minutes. The cell lysates were then transferred to an OptiPlate-384 plate (PerkinElmer 6005620), and the phosphorylation of ERK (p-ERK) and total ERK levels were detected by Alpha SureFire® Ultra™ Multiplex p-EEK kit and total ERK assay kit (PerkinElmer MPSU-PTERK) following the manufacturer's protocol. Assay plates were read on a EnVision Multimode Plate Reader (PerkinElmer), and the ratio of p-ERK vs. total ERK in each well was used as the final readout. Dose response curves were analyzed using a 4-parameter logistic model to calculate IC₅₀ values using Spotfire software. The results of this assay are presented in the table below.

| | KRAS | KRAS | KRAS | KRAS | | | PANC | |
|---|---|---|---|---|---|---|---|---|
| | G12D | G12C | G12V | G13D | HRAS | NRAS | 0813 | SW620 |
| | GNE | GNE | GNE | GNE | GNE | GNE | pERK | pERK |
| | IC50 | IC50 | IC50 | IC50 | IC50 | IC50 | IC50 | IC50 |
| Ex. | (nM) | (nM) | (nM) | (nM) | (nM) | (nM) | (nM) | (nM) |
| 1 | 4.382 | 12.97 | 4.5 | 12.27 | >11060 | >11060 | 68.8 | 74.55 |
| 2 | 8.61 | | 13.25 | | | | 371.8 | 407.6 |
| 3 | 113.4 | 30.91 | 40.35 | 54.58 | >11060 | >11060 | 776.1 | 806.4 |
| 4 | 7.625 | 15.49 | 3.33 | 7.623 | >11060 | >11060 | 56.21 | 48.65 |
| 5 | 15.18 | 18.01 | 6.149 | 13.35 | >11060 | >11060 | 122.6 | 119.8 |
| 6 | 3.781 | 9.162 | 3.147 | 2.403 | 2193 | 9495 | 33.93 | 18.8 |
| 7 | 9.182 | 13.05 | 5.999 | 8.551 | >11060 | >11060 | 191.6 | 67.41 |
| 8 | 9.917 | 20.8 | 7.593 | 12.84 | >11060 | >11060 | 137.6 | 103.6 |
| 9 | 24.4 | 23.12 | 6.174 | 8.741 | >11060 | >11060 | 155.4 | 101.1 |
| 10 | 700.1 | 47.31 | 41.72 | 110.1 | >11060 | >11060 | 2601 | 537.9 |
| 11 | 8.273 | 18.24 | 6.4 | 6.852 | >11060 | >11060 | 39.9 | 21.9 |
| 12 | 34.2 | 19.52 | 11.58 | 12.91 | >11060 | >11060 | 75.4 | 32.9 |
| 13 | 46.74 | 32.22 | 10.37 | 21.94 | >11060 | >11060 | 207.6 | 152.3 |
| 14 | 6.318 | | 6.185 | | | | 153.6 | |
| 15 | 93.97 | | 20.09 | | | | 463.7 | |
| 16 | 25.24 | 14.74 | 9.584 | 10.11 | >11060 | >11060 | 273.9 | |
| 17 | 188.2 | 51.08 | 44.11 | 63.66 | >11060 | >11060 | 2261 | |
| 18 | 5.338 | 2.812 | 2.912 | | | | | |
| 19 | 2.396 | 6.065 | 2.321 | 2.365 | 2071 | 4310 | 34.19 | 15.04 |
| 20 | 294.1 | 75.47 | 60.01 | 93.1 | >11060 | >11060 | 1422 | 510.5 |
| 21 | 135.6 | 20.79 | 63.6 | | | | | |
| 22 | 252.8 | 22.6 | 49.45 | 44.77 | >11060 | >11060 | 413 | |
| 23 | 139.7 | 33.36 | 76.89 | | | | | |
| 24 | 51.8 | 30.41 | 9.516 | 28.83 | >11060 | >11060 | 232.7 | 93.09 |
| 25 | 247 | 28.64 | 17.72 | 52.89 | >11060 | >11060 | 1307 | 406.6 |
| 26 | 177.1 | 54.9 | 22.56 | 59.42 | >11060 | >11060 | 991.6 | 356.9 |
| 27 | 4980 | 242.6 | 148.5 | 846.2 | >11060 | >11060 | | |
| 28 | 1854 | 78.17 | 99.54 | 255.4 | >11060 | >11060 | 2418 | 866.8 |
| 29 | 2359 | 389.9 | 273.4 | 936.5 | >11060 | >11060 | | |
| 30 | 205.7 | | 33 | | | | 848.7 | 305.4 |
| 31 | 2538 | 1182 | 152.2 | 748 | >11060 | >11060 | | |
| 32 | 11110 | 884.9 | 3441 | 798 | >11060 | >11060 | | |
| 33 | 4877 | 1025 | 2234 | | | | | |
| 34 | 3.433 | 1.797 | 1.669 | | | | | |
| 35 | 2.329 | 5.509 | 1.24 | 1.127 | 1173 | >11060 | 12.25 | |
| 36 | 9.339 | 37.09 | 12.35 | 20.17 | >11060 | >11060 | 102.9 | |
| 37 | 8.273 | | 3.545 | | | | 48.89 | |
| 38 | 47.1 | 30.49 | 8.971 | 17.23 | >11060 | >11060 | 300.3 | |
| 39 | 514.7 | 63.14 | 32.18 | 145 | >11060 | >11060 | 1318 | 421.6 |
| 40 | 350.9 | 83.27 | 78.21 | 204.3 | >11060 | >11060 | 806.6 | 627.9 |
| 41 | 124.6 | 56.49 | 46.42 | 75.45 | >11060 | >11060 | 348.4 | 511.5 |
| 42 | 272.5 | | 59.06 | | | | | |
| 43 | 927.2 | | 91.84 | | | | | |
| 44 | 504.7 | 96.35 | 235.4 | 365 | >11060 | >11060 | 1305 | 901.4 |
| 45 | 4466 | 1751 | 1490 | 11060 | >11060 | >11060 | | |
| 46 | 40.9 | | 8.90 | | | | | |
| 47 | 23.42 | | 12.19 | | | | | |
| 48 | 70.88 | | 8.89 | | | | | |
| 49 | 476.9 | | 160.1 | | | | | |
| 50 | 30.53 | | 7.6 | | | | | |
| 51 | 23.66 | | 4.518 | | | | | |
| 52 | 210.4 | | 43.13 | | | | | |
| 53 | 972.1 | | 84.67 | | | | | |

For Example Nos. 1-17, 19-20, 22, 24-32, and 35-53 nucleotide exchange assays for KRAS G12D, G12C, G12V, and G13D were performed according to Procedure A. For Example Nos. 18, 21, 23, and 33-34, nucleotide exchange assays for KRAS G12D, G12C, and G12V were performed according to Procedure B.

SEQUENCES (Procedure A)
SEQ ID NO: 1-Recombinant KRAS G12C
GLNDIFEAQKIEWHETEYKLVVVGACGVGKSALTIQLIQNHFVDEYDPTIEDSYRKQ
VVIDGETSLLDILDTAGQEEYSAMRDQYMRTGEGFLLVFAINNTKSFEDIHHYREQIK
RVKDSEDVPMVLVGNKSDLPSRTVDTKQAQDLARSYGIPFIETSAKTRQGVDDAFYT
LVREIRKHKEK
SEQ ID NO: 2-Recombinant KRAS G12D
GLNDIFEAQKIEWHETEYKLVVVGADGVGKSALTIQLIQNHFVDEYDPTIEDSYRKQ
VVIDGETCLLDILDTAGQEEYSAMRDQYMRTGEGFLCVFAINNTKSFEDIHHYREQI
KRVKDSEDVPMVLVGNKCDLPSRTVDTKQAQDLARSYGIPFIETSAKTRQGVDDAF
YTLVREIRKHKEK
SEQ ID NO: 3-Recombinant KRAS G12V
GLNDIFEAQKIEWHETEYKLVVVGAVGVGKSALTIQLIQNHFVDEYDPTIEDSYRKQ
VVIDGETCLLDILDTAGQEEYSAMRDQYMRTGEGFLCVFAINNTKSFEDIHHYREQI
KRVKDSEDVPMVLVGNKCDLPSRTVDTKQAQDLARSYGIPFIETSAKTRQGVDDAF
YTLVREIRKHKEK
SEQ ID NO: 4-Recombinant KRAS G13D
GLNDIFEAQKIEWHETEYKLVVVGAGDVGKSALTIQLIQNHFVDEYDPTIEDSYRKQ
VVIDGETCLLDILDTAGQEEYSAMRDQYMRTGEGFLCVFAINNTKSFEDIHHYREQI
KRVKDSEDVPMVLVGNKCDLPSRTVDTKQAQDLARSYGIPFIETSAKTRQGVDDAF
YTLVREIRKHKEK
SEQ ID NO: 5-HRAS
GGGGSHMTEYKLVVVGAGGVGKSALTIQLIQNHFVDEYDPTIEDSYRKQVVIDGET
CLLDILDTAGQEEYSAMRDQYMRTGEGFLCVFAINNTKSFEDIHQYREQIKRVKDSD
DVPMVLVGNKCDLAARTVESRQAQDLARSYGIPYIETSAKTRQGVEDAFYTLVREIR
QH
SEQ ID NO: 6-NRAS
GGGGMTEYKLVVVGAGGVGKSALTIQLIQNHFVDEYDPTIEDSYRKQVVIDGETCLL
DILDTAGQEEYSAMRDQYMRTGEGFLCVFAINNSKSFADINLYREQIKRVKDSDDVP
MVLVGNKCDLPTRTVDTKQAHELAKSYGIPFIETSAKTRQGVEDAFYTLVREIRQYR
MKKLN
SEQ ID NO: 7-Recombinant Human SOS Protein
MGSSHHHHHHSGENLYFQGSSGLNDIFEAQKIEWHESSEEQMRLPSADVYRFAEPDS
EENIIFEENMQPKAGIPIIKAGTVIKLIERLTYHMYADPNFVRTFLTTYRSFCKPQELLS
LIIERFEIPEPEPTEADRIAIENGDQPLSAELKRFRKEYIQPVQLRVLNVCRHWVEHHF
YDFERDAYLLQRMEEFIGTVRGKAMKKWVESITKIIQRKKIARDNGPGHNITFQSSPP
TVEWHISRPGHIETFDLLTLHPIEIARQLTLLESDLYRAVQPSELVGSVWTKEDKEINS
PNLLKMIRHTTNLTLWFEKCIVETENLEERVAVVSRIIEILQVFQELNNFNGVLEVVSA
MNSSPVYRLDHTFEQIPSRQKKILEEAHELSEDHYKKYLAKLRSINPPCVPFFGIYLTN
ILKTEEGNPEVLKRHGKELINFSKRRKVAEITGEIQQYQNQPYCLRVESDIKRFFENLN
PMGNSMEKEFTDYLFNKSLEIEPRNPKPLPRFPKKYSYPLKSPGVRPSNPRPGT
SEQUENCES (Procedure B)
SEQ ID NO: 8-KRAS G12C (amino acids 1-169, N-terminal His-tag)
MASSHHHHHHSSENLYFQGMTEYKLVVVGACGVGKSALTIQLIQNHFVDEYDPTIE
DSYRKQVVIDGETCLLDILDTAGQEEYSAMRDQYMRTGEGFLCVFAINNTKSFEDI
HHYREQIKRVKDSEDVPMVLVGNKCDLPSRTVDTKQAQDLARSYGIPFIETSAKTR
QGVDDAFYTLVREIRKHKEK
SEQ ID NO: 9-KRAS G12D (amino acids 1-169, N-terminal His-tag)
MASSHHHHHHSSENLYFQGMTEYKLVVVGADGVGKSALTIQLIQNHFVDEYDPTI
EDSYRKQVVIDGETCLLDILDTAGQEEYSAMRDQYMRTGEGFLCVFAINNTKSFED
IHHYREQIKRVKDSEDVPMVLVGNKCDLPSRTVDTKQAQDLARSYGIPFIETSAKTR
QGVDDAFYTLVREIRKHKEK
SEQ ID NO: 10-KRAS G12V (amino acids 1-169, N-terminal His-tag)
MGSSHHHHHHSSGLVPRGSHMASMTGGQQMGRGSENLYFQGMTEYKLVVVGAV
GVGKSALTIQLIQNHFVDEYDPTIEDSYRKQVVIDGETCLLDILDTAGQEEYSAMRD
QYMRTGEGFLCVFAINNTKSFEDIHHYREQIKRVKDSEDVPMVLVGNKCDLPSRTV
DTKQAQDLARSYGIPFIETSAKTRQGVDDAFYTLVREIRKHKEK
SEQ ID NO: 11-SOS1 (amino acids 564-1049, No tag)
GEEQMRLPSADVYRFAEPDSEENIIFEENMQPKAGIPIIKAGTVIKLIERLTYHMYADP
NFVRTFLTTYRSFCKPQELLSLIIERFEIPEPEPTEADRIAIENGDQPLSAELKRFRKEYI
QPVQLRVLNVCRHWVEHHFYDFERDAYLLQRMEEFIGTVRGKAMKKWVESITKIIQ
RKKIARDNGPGHNITFQSSPPTVEWHISRPGHIETFDLLTLHPIEIARQLTLLESDLYRA
VQPSELVGSVWTKEDKEINSPNLLKMIRHTTNLTLWFEKCIVETENLEERVAVVSRII
EILQVFQELNNFNGVLEVVSAMNSSPVYRLDHTFEQIPSRQKKILEEAHELSEDHYKK
YLAKLRSINPPCVPFFGIYLTNILKTEEGNPEVLKRHGKELINFSKRRKVAEITGEIQQY
QNQPYCLRVESDIKRFFENLNPMGNSMEKEFTDYLFNKSLEIEPRNPKPLPRFPKKYS
YPLKSPGVRPSNPRPGT

1. A compound selected from the group consisting of: 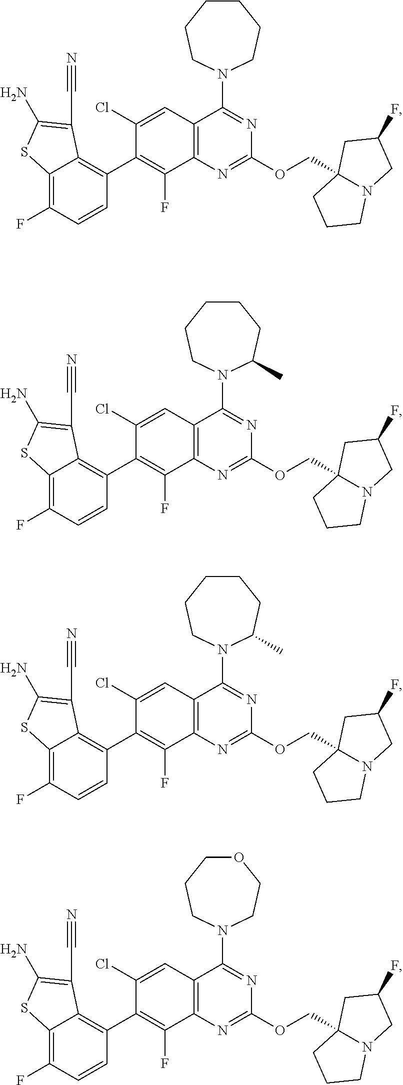 [SMILES: N#Cc1c(N)sc2c(F)ccc(-c3c(Cl)cc4c(N5CCCCCC5)nc(OC[C@@]56CCCN5C[C@H](F)C6)nc4c3F)c12 ; C[C@@H]1CCCCCN1c1nc(OC[C@@]23CCCN2C[C@H](F)C3)nc2c(F)c(-c3ccc(F)c4sc(N)c(C#N)c34)c(Cl)cc12 ; C[C@H]1CCCCCN1c1nc(OC[C@@]23CCCN2C[C@H](F)C3)nc2c(F)c(-c3ccc(F)c4sc(N)c(C#N)c34)c(Cl)cc12 ; N#Cc1c(N)sc2c(F)ccc(-c3c(Cl)cc4c(N5CCCOCC5)nc(OC[C@@]56CCCN5C[C@H](F)C6)nc4c3F)c12] 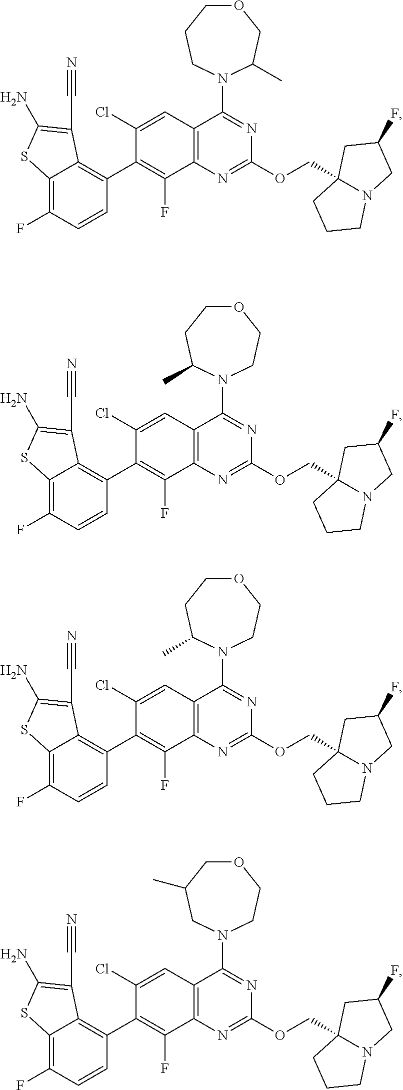 [SMILES: CC1COCCCN1c1nc(OC[C@@]23CCCN2C[C@H](F)C3)nc2c(F)c(-c3ccc(F)c4sc(N)c(C#N)c34)c(Cl)cc12 ; C[C@H]1CCOCCN1c1nc(OC[C@@]23CCCN2C[C@H](F)C3)nc2c(F)c(-c3ccc(F)c4sc(N)c(C#N)c34)c(Cl)cc12 ; C[C@@H]1CCOCCN1c1nc(OC[C@@]23CCCN2C[C@H](F)C3)nc2c(F)c(-c3ccc(F)c4sc(N)c(C#N)c34)c(Cl)cc12 ; CC1COCCN(c2nc(OC[C@@]34CCCN3C[C@H](F)C4)nc3c(F)c(-c4ccc(F)c5sc(N)c(C#N)c45)c(Cl)cc23)C1] 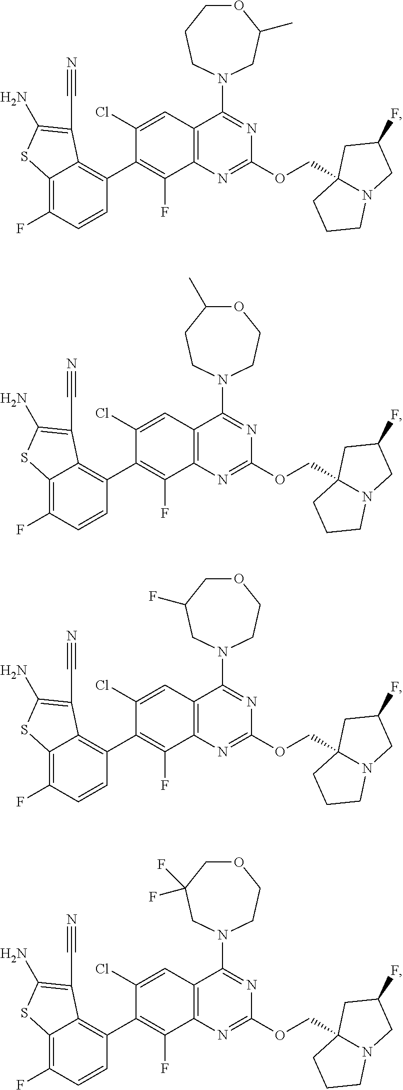 [SMILES: CC1CN(c2nc(OC[C@@]34CCCN3C[C@H](F)C4)nc3c(F)c(-c4ccc(F)c5sc(N)c(C#N)c45)c(Cl)cc23)CCCO1 ; CC1CCN(c2nc(OC[C@@]34CCCN3C[C@H](F)C4)nc3c(F)c(-c4ccc(F)c5sc(N)c(C#N)c45)c(Cl)cc23)CCO1 ; N#Cc1c(N)sc2c(F)ccc(-c3c(Cl)cc4c(N5CCOCC(F)C5)nc(OC[C@@]56CCCN5C[C@H](F)C6)nc4c3F)c12 ; N#Cc1c(N)sc2c(F)ccc(-c3c(Cl)cc4c(N5CCOCC(F)(F)C5)nc(OC[C@@]56CCCN5C[C@H](F)C6)nc4c3F)c12] 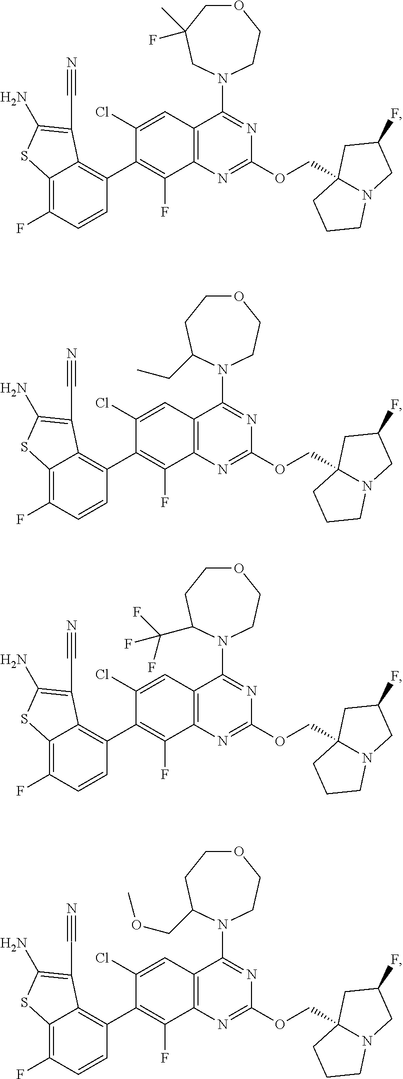 [SMILES: CC1(F)COCCN(c2nc(OC[C@@]34CCCN3C[C@H](F)C4)nc3c(F)c(-c4ccc(F)c5sc(N)c(C#N)c45)c(Cl)cc23)C1 ; CCC1CCOCCN1c1nc(OC[C@@]23CCCN2C[C@H](F)C3)nc2c(F)c(-c3ccc(F)c4sc(N)c(C#N)c34)c(Cl)cc12 ; N#Cc1c(N)sc2c(F)ccc(-c3c(Cl)cc4c(N5CCOCCC5C(F)(F)F)nc(OC[C@@]56CCCN5C[C@H](F)C6)nc4c3F)c12 ; COCC1CCOCCN1c1nc(OC[C@@]23CCCN2C[C@H](F)C3)nc2c(F)c(-c3ccc(F)c4sc(N)c(C#N)c34)c(Cl)cc12] 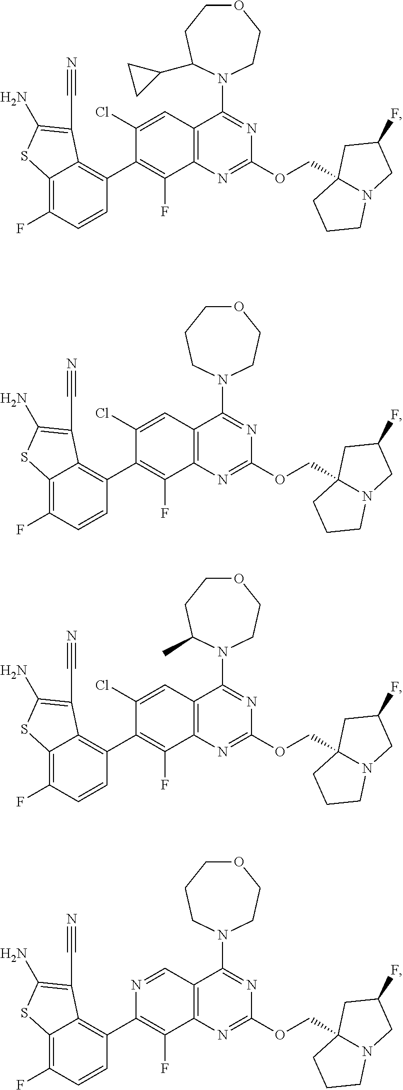 [SMILES: N#Cc1c(N)sc2c(F)ccc(-c3c(Cl)cc4c(N5CCOCCC5C5CC5)nc(OC[C@@]56CCCN5C[C@H](F)C6)nc4c3F)c12 ; N#Cc1c(N)sc2c(F)ccc(-c3c(Cl)cc4c(N5CCCOCC5)nc(OC[C@@]56CCCN5C[C@H](F)C6)nc4c3F)c12 ; C[C@H]1CCOCCN1c1nc(OC[C@@]23CCCN2C[C@H](F)C3)nc2c(F)c(-c3ccc(F)c4sc(N)c(C#N)c34)c(Cl)cc12 ; N#Cc1c(N)sc2c(F)ccc(-c3ncc4c(N5CCCOCC5)nc(OC[C@@]56CCCN5C[C@H](F)C6)nc4c3F)c12] 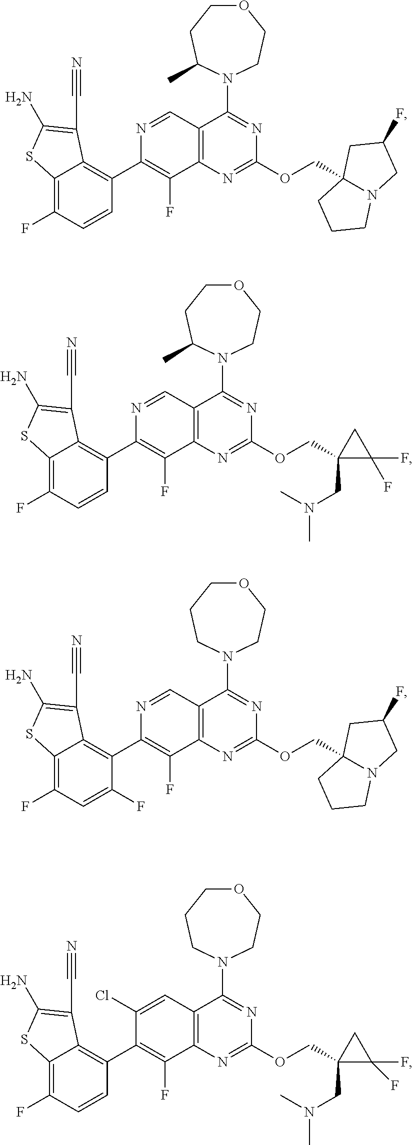 [SMILES: C[C@H]1CCOCCN1c1nc(OC[C@@]23CCCN2C[C@H](F)C3)nc2c(F)c(-c3ccc(F)c4sc(N)c(C#N)c34)ncc12 ; C[C@H]1CCOCCN1c1nc(OC[C@]2(CN(C)C)CC2(F)F)nc2c(F)c(-c3ccc(F)c4sc(N)c(C#N)c34)ncc12 ; N#Cc1c(N)sc2c(F)cc(F)c(-c3ncc4c(N5CCCOCC5)nc(OC[C@@]56CCCN5C[C@H](F)C6)nc4c3F)c12 ; CN(C)C[C@@]1(COc2nc(N3CCCOCC3)c3cc(Cl)c(-c4ccc(F)c5sc(N)c(C#N)c45)c(F)c3n2)CC1(F)F] 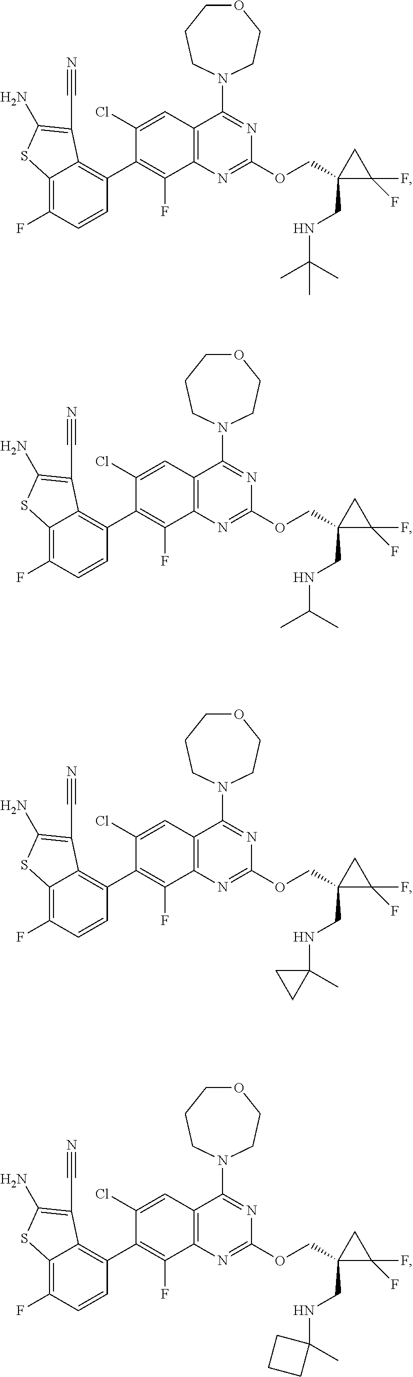 [SMILES: CC(C)(C)NC[C@@]1(COc2nc(N3CCCOCC3)c3cc(Cl)c(-c4ccc(F)c5sc(N)c(C#N)c45)c(F)c3n2)CC1(F)F ; CC(C)NC[C@@]1(COc2nc(N3CCCOCC3)c3cc(Cl)c(-c4ccc(F)c5sc(N)c(C#N)c45)c(F)c3n2)CC1(F)F ; CC1(NC[C@@]2(COc3nc(N4CCCOCC4)c4cc(Cl)c(-c5ccc(F)c6sc(N)c(C#N)c56)c(F)c4n3)CC2(F)F)CC1 ; CC1(NC[C@@]2(COc3nc(N4CCCOCC4)c4cc(Cl)c(-c5ccc(F)c6sc(N)c(C#N)c56)c(F)c4n3)CC2(F)F)CCC1] 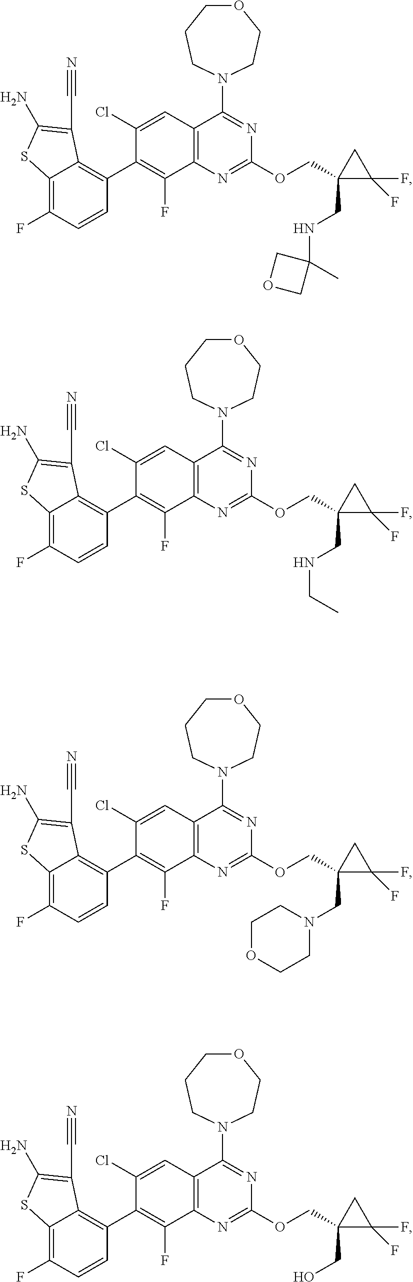 [SMILES: CC1(NC[C@@]2(COc3nc(N4CCCOCC4)c4cc(Cl)c(-c5ccc(F)c6sc(N)c(C#N)c56)c(F)c4n3)CC2(F)F)COC1 ; CCNC[C@@]1(COc2nc(N3CCCOCC3)c3cc(Cl)c(-c4ccc(F)c5sc(N)c(C#N)c45)c(F)c3n2)CC1(F)F ; N#Cc1c(N)sc2c(F)ccc(-c3c(Cl)cc4c(N5CCCOCC5)nc(OC[C@]5(CN6CCOCC6)CC5(F)F)nc4c3F)c12 ; N#Cc1c(N)sc2c(F)ccc(-c3c(Cl)cc4c(N5CCCOCC5)nc(OC[C@]5(CO)CC5(F)F)nc4c3F)c12] 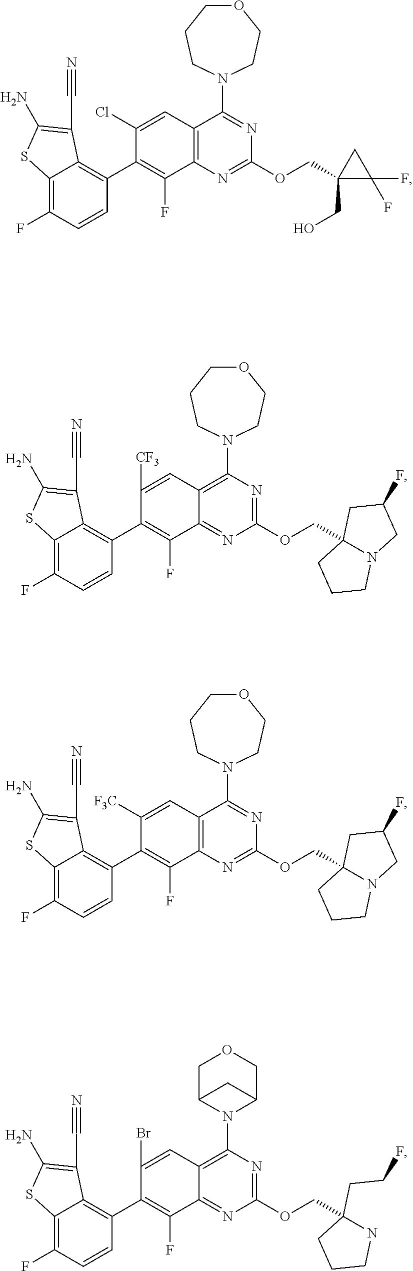 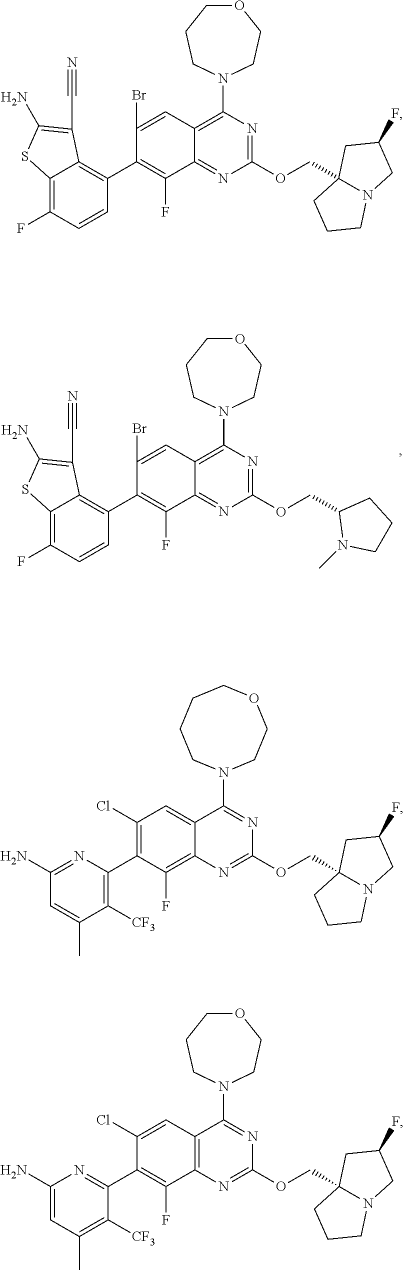 [SMILES: N#Cc1c(N)sc2c(F)ccc(-c3c(Br)cc4c(N5CCCOCC5)nc(OC[C@@]56CCCN5C[C@H](F)C6)nc4c3F)c12 ; CN1CCC[C@H]1COc1nc(N2CCCOCC2)c2cc(Br)c(-c3ccc(F)c4sc(N)c(C#N)c34)c(F)c2n1 ; Cc1cc(N)nc(-c2c(Cl)cc3c(N4CCCCOCC4)nc(OC[C@@]45CCCN4C[C@H](F)C5)nc3c2F)c1C(F)(F)F ; Cc1cc(N)nc(-c2c(Cl)cc3c(N4CCCOCC4)nc(OC[C@@]45CCCN4C[C@H](F)C5)nc3c2F)c1C(F)(F)F] 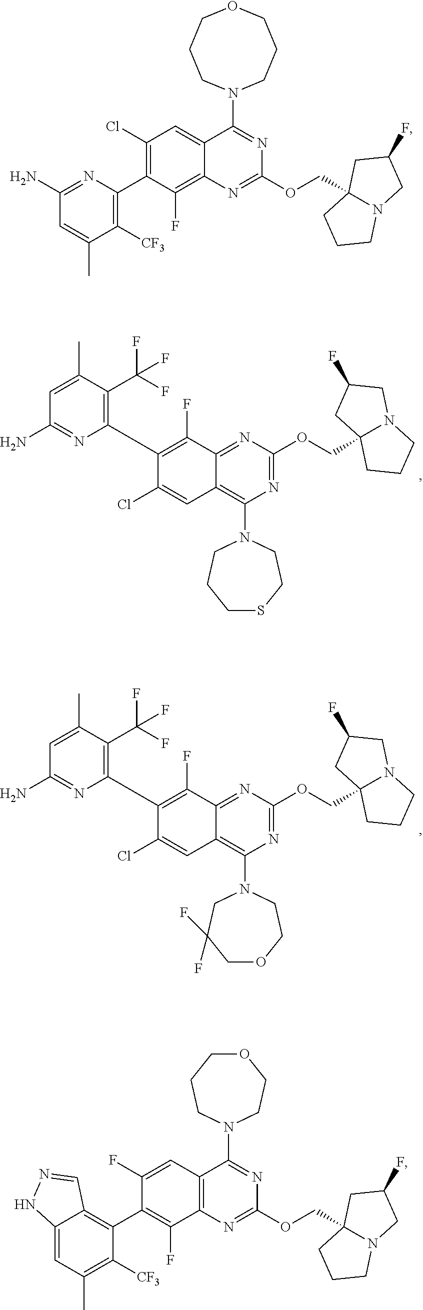 [SMILES: Cc1cc(N)nc(-c2c(Cl)cc3c(N4CCCOCCC4)nc(OC[C@@]45CCCN4C[C@H](F)C5)nc3c2F)c1C(F)(F)F ; Cc1cc(N)nc(-c2c(Cl)cc3c(N4CCCSCC4)nc(OC[C@@]45CCCN4C[C@H](F)C5)nc3c2F)c1C(F)(F)F ; Cc1cc(N)nc(-c2c(Cl)cc3c(N4CCOCC(F)(F)C4)nc(OC[C@@]45CCCN4C[C@H](F)C5)nc3c2F)c1C(F)(F)F ; Cc1cc2[nH]ncc2c(-c2c(F)cc3c(N4CCCOCC4)nc(OC[C@@]45CCCN4C[C@H](F)C5)nc3c2F)c1C(F)(F)F] 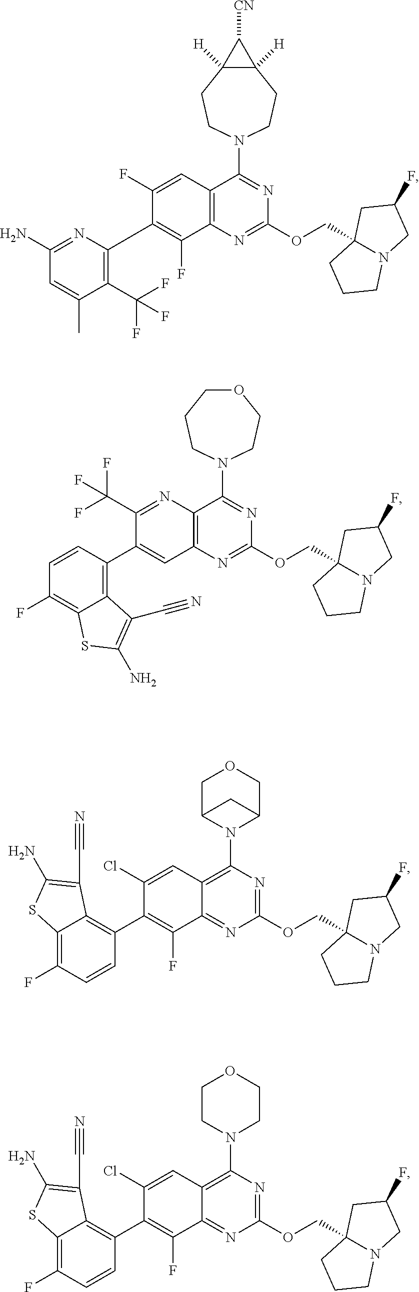 [SMILES: Cc1cc(N)nc(-c2c(F)cc3c(N4CC[C@@H]5[C@@H](C#N)[C@@H]5CC4)nc(OC[C@@]45CCCN4C[C@H](F)C5)nc3c2F)c1C(F)(F)F ; N#Cc1c(N)sc2c(F)ccc(-c3cc4nc(OC[C@@]56CCCN5C[C@H](F)C6)nc(N5CCCOCC5)c4nc3C(F)(F)F)c12 ; N#Cc1c(N)sc2c(F)ccc(-c3c(Cl)cc4c(N5C6COCC5C6)nc(OC[C@@]56CCCN5C[C@H](F)C6)nc4c3F)c12 ; N#Cc1c(N)sc2c(F)ccc(-c3c(Cl)cc4c(N5CCOCC5)nc(OC[C@@]56CCCN5C[C@H](F)C6)nc4c3F)c12] 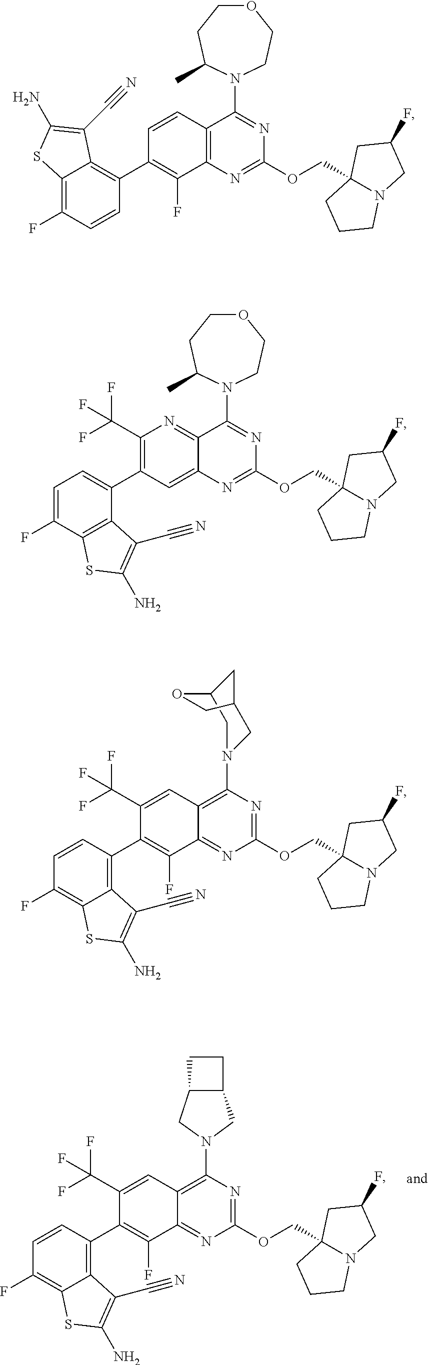 [SMILES: C[C@H]1CCOCCN1c1nc(OC[C@@]23CCCN2C[C@H](F)C3)nc2c(F)c(-c3ccc(F)c4sc(N)c(C#N)c34)ccc12 ; C[C@H]1CCOCCN1c1nc(OC[C@@]23CCCN2C[C@H](F)C3)nc2cc(-c3ccc(F)c4sc(N)c(C#N)c34)c(C(F)(F)F)nc12 ; N#Cc1c(N)sc2c(F)ccc(-c3c(C(F)(F)F)cc4c(N5CC6COC(C6)C5)nc(OC[C@@]56CCCN5C[C@H](F)C6)nc4c3F)c12 ; N#Cc1c(N)sc2c(F)ccc(-c3c(C(F)(F)F)cc4c(N5C[C@H]6CC[C@H]6C5)nc(OC[C@@]56CCCN5C[C@H](F)C6)nc4c3F)c12] 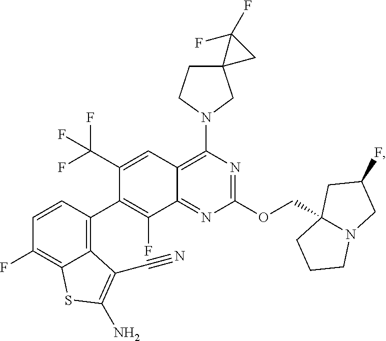 [SMILES: N#Cc1c(N)sc2c(F)ccc(-c3c(C(F)(F)F)cc4c(N5CCC6(C5)CC6(F)F)nc(OC[C@@]56CCCN5C[C@H](F)C6)nc4c3F)c12]
or a pharmaceutically acceptable salt thereof.

2. A pharmaceutical composition comprising the compound of claim 1 or the pharmaceutically acceptable salt thereof, and a pharmaceutically acceptable carrier.

3. A pharmaceutical composition comprising the compound of claim 1 or the pharmaceutically acceptable salt thereof, an additional anti-cancer agent, and a pharmaceutically acceptable carrier.

4. A method of inhibiting KRAS-G12D protein comprising contacting KRAS-G12D protein with the compound of claim 1, or the pharmaceutically acceptable salt thereof, to inhibit the activity of the KRAS-G12D protein.

5. A method of inhibiting KRAS-G12C protein comprising contacting KRAS-G12C protein with the compound of claim 1, or the pharmaceutically acceptable salt thereof, to inhibit the activity of the KRAS-G12C protein.

6. A method of inhibiting KRAS-G12V protein comprising contacting KRAS-G12V protein with the compound of claim 1, or the pharmaceutically acceptable salt thereof, to inhibit the activity of the KRAS-G12V protein.

7. A method of inhibiting KRAS-G13D protein comprising contacting KRAS-G13D protein with the compound of claim 1, or the pharmaceutically acceptable salt thereof, to inhibit the activity of the KRAS-G13D protein.

8. A method of treating cancer comprising administering a therapeutically effective amount of the compound of claim 1, or the pharmaceutically acceptable salt thereof, to a subject in need of such treatment.

9. The method of claim 8, further comprising administering an additional active agent to the subject.

10.-16. (canceled)
